SEQUENCE LISTING (16 sequences)

SEQ ID NO: 1 - protein, 440 aa, Homo sapiens [sequence omitted]

SEQ ID NO: 2 - protein, 829 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTDEPRLSASAPLVIHFLKAPPAPALTLAPVPSQYLCTPRQDHGLIVSGPSKDPDLASGHGPYSFTLGPNPTVQRDWRLQTLNGSHAYLTLALHWVEPREHIIPVVVSHNAQMWQLLVRVIVCRCNVEGQCMRKVGRMKGMPTKLSAVGILVGTLVAIGIFLILIFTHWTMSRKKDPDQPADSVPLKATV

SEQ ID NO: 3 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 4 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 5 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 6 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 7 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 8 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 9 - protein, 784 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 10 - protein, 154 aa, Homo sapiens
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 11 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 12 - protein, 22152 aa, Homo sapiens
  MOD_RES (13877)..(13878): Any amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 13 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 14 - protein, 393 aa, Homo sapiens [sequence omitted]

SEQ ID NO: 15 - protein, 10 aa, Homo sapiens
IRGRERFEMF

SEQ ID NO: 16 - protein, 10 aa, Artificial Sequence
  Description of Artificial Sequence Synthetic peptide
DGTSFQKENC

(19) United States
(12) Patent Application Publication
(10) Pub. No.: US 2015/0204892 A1
(43) Pub. Date: Jul. 23, 2015

(54) METHODS AND COMPOSITIONS FOR DIAGNOSIS AND PROGNOSIS OF RENAL INJURY AND RENAL FAILURE

(71) Applicant: Astute Medical, Inc., San Diego, CA (US)

(72) Inventors: Joseph Anderberg, Encinitas, CA (US); Jeff Gray, Solana Beach, CA (US); Paul McPherson, Encinitas, CA (US); Kevin Nakamura, Cardiff by the Sea, CA (US); James Patrick Kampf, San Diego, CA (US)

(21) Appl. No.: 14/675,344

(22) Filed: Mar. 31, 2015

Related U.S. Application Data
(62) Division of application No. 13/393,075, filed on Apr. 9, 2012, now abandoned.
(60) Provisional application No. 61/238,134, filed on Aug. 28, 2009.
(60) Provisional application No. 61/238,120, filed on Aug. 28, 2009.
(60) Provisional application No. 61/238,129, filed on Aug. 28, 2009.
(60) Provisional application No. 61/238,118, filed on Aug. 28, 2009.
(60) Provisional application No. 61/238,123, filed on Aug. 28, 2009.
(60) Provisional application No. 61/238,127, filed on Aug. 28, 2009.
(60) Provisional application No. 61/238,121, filed on Aug. 28, 2009.
(60) Provisional application No. 61/238,128, filed on Aug. 28, 2009.
(60) Provisional application No. 61/238,125, filed on Aug. 28, 2009.
(60) Provisional application No. 61/238,115, filed on Aug. 28, 2009.
(60) Provisional application No. 61/244,002, filed on Sep. 18, 2009.
(60) Provisional application No. 61/243,993, filed on Sep. 18, 2009.
(60) Provisional application No. 61/243,991, filed on Sep. 18, 2009.
(60) Provisional application No. 61/243,997, filed on Sep. 18, 2009.

(51) Int. Cl. G01N 33/92

(52) U.S. Cl. CPC G01N 33/92; G01N 2333/775; G01N 2800/50

(57) ABSTRACT

The present invention relates to methods and compositions for monitoring, diagnosis, prognosis, and determination of treatment regimens in subjects suffering from or suspected of having a renal injury. In particular, the invention relates to using assays that detect one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, Carcinoembryonic antigen-related cell adhesion molecule 5, and Cellular tumor antigen p53 as diagnostic and prognostic biomarker assays in renal injuries.

### CROSS-REFERENCE TO RELATED APPLICATIONS

This application is a divisional of application Ser. No. 13/393,075, filed Apr. 9, 2012, which is a U.S. national phase application of International Application No. PCT/US2010/046910, filed Aug. 27, 2010, which designated the U.S. and claims the benefit of priority to U.S. Provisional Patent Application Nos. 61/238,115 filed Aug. 28, 2009, 61/238,118 filed Aug. 28, 2009, 61/238,120 filed Aug. 28, 2009, 61/238,121 filed Aug. 28, 2009, 61/238,123 filed Aug. 28, 2009, 61/238,125 filed Aug. 28, 2009, 61/238,127 filed Aug. 28, 2009, 61/238,129 filed Aug. 28, 2009, 61/238,134 filed Aug. 28, 2009, 61/243,991 filed Sep. 18, 2009, 61/243,997 filed Sep. 18, 2009, 61/244,002 filed Sep. 18, 2009, 61/243,993 filed Sep. 18, 2009, and 61/238,128 filed Aug. 28, 2009, each of which is hereby incorporated in its entirety including all tables, figures and claims.

### SEQUENCE LISTING

The instant application contains a Sequence Listing which has been submitted in ASCII format via EFS-Web and is hereby incorporated by reference in its entirety. Said ASCII copy, created on Mar. 31, 2015, is named AST1140DV_SeqListing.txt and is 282 kilobytes in size.

### BACKGROUND OF THE INVENTION

The following discussion of the background of the invention is merely provided to aid the reader in understanding the invention and is not admitted to describe or constitute prior art to the present invention.

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., McGraw Hill, New York, pages 1741-1830, which are hereby incorporated by reference in their entirety. Renal disease and/or injury may be acute or chronic. Acute and chronic kidney disease are described as follows (from Current Medical Diagnosis & Treatment 2008, 47^{th} Ed, McGraw Hill, New York, pages 785-815, which are hereby incorporated by reference in their entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates about 5% of hospital admissions, 4-15% of cardiopulmonary bypass surgeries, and up to 30% of intensive care admissions. ARF may be categorized as prerenal, intrinsic renal, or postrenal in causation. Intrinsic renal disease can be further divided into glomerular, tubular, interstitial, and vascular abnormalities. Major causes of ARF are described in the following table, which is adapted from the Merck Manual, 17^{th} ed., Chapter 222, and which is hereby incorporated by reference in their entirety:

| Type | Risk Factors |
|---|---|
| Prerenal | |
| ECF volume depletion | Excessive diuresis, hemorrhage, GI losses, loss of |
| | intravascular fluid into the extravascular space (due to |
| | ascites, peritonitis, pancreatitis, or burns), loss of skin |
| | and mucus membranes, renal salt- and water-wasting |
| | states |
| Low cardiac output | Cardiomyopathy, MI, cardiac tamponade, pulmonary |
| | embolism, pulmonary hypertension, positive-pressure |
| | mechanical ventilation |
| Low systemic vascular | Septic shock, liver failure, antihypertensive drugs |
| resistance | |
| Increased renal vascular | NSAIDs, cyclosporines, tacrolimus, hypercalcemia, |
| resistance | anaphylaxis, anesthetics, renal artery obstruction, renal |
| | vein thrombosis, sepsis, hepatorenal syndrome |
| Decreased efferent | ACE inhibitors or angiotensin II receptor blockers |
| arteriolar tone (leading to | |
| decreased GFR from | |
| reduced glomerular | |
| transcapillary pressure, | |
| especially in patients with | |
| bilateral renal artery | |
| stenosis) | |
| Intrinsic Renal | |
| Acute tubular injury | Ischemia (prolonged or severe prerenal state): surgery, |
| | hemorrhage, arterial or venous obstruction; Toxins: |
| | NSAIDs, cyclosporines, tacrolimus, aminoglycosides, |
| | foscarnet, ethylene glycol, hemoglobin, myoglobin, |
| | ifosfamide, heavy metals, methotrexate, radiopaque |
| | contrast agents, streptozotocin |
| Acute glomerulonephritis | ANCA-associated: Crescentic glomerulonephritis, |
| | polyarteritis nodosa, Wegener's granulomatosis; Anti- |
| | GBM glomerulonephritis: Goodpasture's syndrome; |
| | Immune-complex: Lupus glomerulonephritis, |
| | postinfectious glomerulonephritis, cryoglobulinemic |
| | glomerulonephritis |
| Acute tubulointerstitial | Drug reaction (eg, β-lactams, NSAIDs, sulfonamides, |
| nephritis | ciprofloxacin, thiazide diuretics, furosemide, phenytoin, |
| | allopurinol, pyelonephritis, papillary necrosis |
| Acute vascular | Vasculitis, malignant hypertension, thrombotic |
| nephropathy | microangiopathies, scleroderma, atheroembolism |
| Infiltrative diseases | Lymphoma, sarcoidosis, leukemia |
| Postrenal | |
| Tubular precipitation | Uric acid (tumor lysis), sulfonamides, triamterene, |
| | acyclovir, indinavir, methotrexate, ethylene glycol |
| | ingestion, myeloma protein, myoglobin |
| Ureteral obstruction | Intrinsic: Calculi, clots, sloughed renal tissue, fungus |
| | ball, edema, malignancy, congenital defects; Extrinsic: |
| | Malignancy, retroperitoneal fibrosis, ureteral trauma |
| | during surgery or high impact injury |
| Bladder obstruction | Mechanical: Benign prostatic hyperplasia, prostate |
| | cancer, bladder cancer, urethral strictures, phimosis, |
| | paraphimosis, urethral valves, obstructed indwelling |
| | urinary catheter; Neurogenic: Anticholinergic drugs, |
| | upper or lower motor neuron lesion |

In the case of ischemic ARF, the course of the disease may be divided into four phases. During an initiation phase, which lasts hours to days, reduced perfusion of the kidney is evolving into injury. Glomerular ultrafiltration reduces, the flow of filtrate is reduced due to debris within the tubules, and back leakage of filtrate through injured epithelium occurs. Renal injury can be mediated during this phase by reperfusion of the kidney. Initiation is followed by an extension phase which is characterized by continued ischemic injury and inflammation and may involve endothelial damage and vascular congestion. During the maintenance phase, lasting from 1 to 2 weeks, renal cell injury occurs, and glomerular filtration and urine output reaches a minimum. A recovery phase can follow in which the renal epithelium is repaired and GFR gradually recovers. Despite this, the survival rate of subjects with ARF may be as low as about 60%.

Acute kidney injury caused by radiocontrast agents (also called contrast media) and other nephrotoxins such as cyclosporine, antibiotics including aminoglycosides and anticancer drugs such as cisplatin manifests over a period of days to about a week. Contrast induced nephropathy (CIN, which is AKI caused by radiocontrast agents) is thought to be caused by intrarenal vasoconstriction (leading to ischemic injury) and from the generation of reactive oxygen species that are directly toxic to renal tubular epithelial cells. CIN classically presents as an acute (onset within 24-48 h) but reversible (peak 3-5 days, resolution within 1 week) rise in blood urea nitrogen and serum creatinine.

A commonly reported criteria for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16: 3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum creatinine rise.

One study (Lassnigg et all, J Am Soc Nephrol 15:1597-1605, 2004, hereby incorporated by reference in its entirety) investigated both increases and decreases in serum creatinine. Patients with a mild fall in serum creatinine of −0.1 to −0.3 mg/dL following heart surgery had the lowest mortality rate. Patients with a larger fall in serum creatinine (more than or equal to −0.4 mg/dL) or any increase in serum creatinine had a larger mortality rate. These findings caused the authors to conclude that even very subtle changes in renal function (as detected by small creatinine changes within 48 hours of surgery) seriously effect patient's outcomes. In an effort to reach consensus on a unified classification system for using serum creatinine to define AKI in clinical trials and in clinical practice, Bellomo et al., Crit Care. 8(4):R204-12, 2004, which is hereby incorporated by reference in its entirety, proposes the following classifications for stratifying AKI patients:

“Risk”: serum creatinine increased 1.5 fold from baseline OR urine production of <0.5 ml/kg body weight/hr for 6 hours;
“Injury”: serum creatinine increased 2.0 fold from baseline OR urine production <0.5 ml/kg/hr for 12 h;
“Failure”: serum creatinine increased 3.0 fold from baseline OR creatinine >355 μmol (with a rise of >44) or urine output below 0.3 ml/kg/hr for 24 h or anuria for at least 12 hours;
And included two clinical outcomes:
“Loss”: persistent need for renal replacement therapy for more than four weeks.
“ESRD”: end stage renal disease—the need for dialysis for more than 3 months.

These criteria are called the RIFLE criteria, which provide a useful clinical tool to classify renal status. As discussed in Kellum, Crit. Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies.

More recently, Mehta et al., Crit. Care 11:R31 (doi:10.1186.cc5713), 2007, hereby incorporated by reference in its entirety, proposes the following similar classifications for stratifying AKI patients, which have been modified from RIFLE:

“Stage I”: increase in serum creatinine of more than or equal to 0.3 mg/dL (≧26.4 μmol/L) or increase to more than or equal to 150% (1.5-fold) from baseline OR urine output less than 0.5 mL/kg per hour for more than 6 hours;
“Stage II”: increase in serum creatinine to more than 200% (>2-fold) from baseline OR urine output less than 0.5 mL/kg per hour for more than 12 hours;
“Stage III”: increase in serum creatinine to more than 300% (>3-fold) from baseline OR serum creatinine ≧354 μmol accompanied by an acute increase of at least 44 μmol OR urine output less than 0.3 mL/kg per hour for 24 hours or anuria for 12 hours.

The CIN Consensus Working Panel (McCollough et al, Rev Cardiovasc Med. 2006; 7(4):177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

Although serial measurement of serum creatinine over a period of days is an accepted method of detecting and diagnosing AKI and is considered one of the most important tools to evaluate AKI patients, serum creatinine is generally regarded to have several limitations in the diagnosis, assessment and monitoring of AKI patients. The time period for serum creatinine to rise to values (e.g., a 0.3 mg/dL or 25% rise) considered diagnostic for AKI can be 48 hours or longer depending on the definition used. Since cellular injury in AKI can occur over a period of hours, serum creatinine elevations detected at 48 hours or longer can be a late indicator of injury, and relying on serum creatinine can thus delay diagnosis of AKI. Furthermore, serum creatinine is not a good indicator of the exact kidney status and treatment needs during the most acute phases of AKI when kidney function is changing rapidly. Some patients with AKI will recover fully, some will need dialysis (either short term or long term) and some will have other detrimental outcomes including death, major adverse cardiac events and chronic kidney disease. Because serum creatinine is a marker of filtration rate, it does not differentiate between the causes of AKI (pre-renal, intrinsic renal, post-renal obstruction, atheroembolic, etc) or the category or location of injury in intrinsic renal disease (for example, tubular, glomerular or interstitial in origin). Urine output is similarly limited, Knowing these things can be of vital importance in managing and treating patients with AKI.

These limitations underscore the need for better methods to detect and assess AKI, particularly in the early and subclinical stages, but also in later stages when recovery and repair of the kidney can occur. Furthermore, there is a need to better identify patients who are at risk of having an AKI.

### BRIEF SUMMARY OF THE INVENTION

It is an object of the invention to provide methods and compositions for evaluating renal function in a subject. As described herein, measurement of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, Carcinoembryonic antigen-related cell adhesion molecule 5, and Cellular tumor antigen p53 (referred to herein as a “kidney injury marker”) can be used for diagnosis, prognosis, risk stratification, staging, monitoring, categorizing and determination of further diagnosis and treatment regimens in subjects suffering or at risk of suffering from an injury to renal function, reduced renal function, and/or acute renal failure (also called acute kidney injury).

The kidney injury markers of the present invention may be used, individually or in panels comprising a plurality of kidney injury markers, for risk stratification (that is, to identify subjects at risk for a future injury to renal function, for future progression to reduced renal function, for future progression to ARF, for future improvement in renal function, etc.); for diagnosis of existing disease (that is, to identify subjects who have suffered an injury to renal function, who have progressed to reduced renal function, who have progressed to ARF, etc.); for monitoring for deterioration or improvement of renal function; and for predicting a future medical outcome, such as improved or worsening renal function, a decreased or increased mortality risk, a decreased or increased risk that a subject will require renal replacement therapy (i.e., hemodialysis, peritoneal dialysis, hemofiltration, and/or renal transplantation, a decreased or increased risk that a subject will recover from an injury to renal function, a decreased or increased risk that a subject will recover from ARF, a decreased or increased risk that a subject will progress to end stage renal disease, a decreased or increased risk that a subject will progress to chronic renal failure, a decreased or increased risk that a subject will suffer rejection of a transplanted kidney, etc.

In a first aspect, the present invention relates to methods for evaluating renal status in a subject. These methods comprise performing an assay method that is configured to detect one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, Carcinoembryonic antigen-related cell adhesion molecule 5, and Cellular tumor antigen p53 in a body fluid sample obtained from the subject. The assay result(s), for example measured concentration(s) of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, is/are then correlated to the renal status of the subject. This correlation to renal status may include correlating the assay result(s) to one or more of risk stratification, diagnosis, prognosis, staging, classifying and monitoring of the subject as described herein. Thus, the present invention utilizes one or more kidney injury markers of the present invention for the evaluation of renal injury.

In certain embodiments, the methods for evaluating renal status described herein are methods for risk stratification of the subject; that is, assigning a likelihood of one or more future changes in renal status to the subject. In these embodiments, the assay result(s) is/are correlated to one or more such future changes. The following are preferred risk stratification embodiments.

In preferred risk stratification embodiments, these methods comprise determining a subject's risk for a future injury to renal function, and the assay result(s) is/are correlated to a likelihood of such a future injury to renal function. For example, the measured concentration(s) may each be compared to a threshold value. For a “positive going” kidney injury marker, an increased likelihood of suffering a future injury to renal function is assigned to the subject when the measured concentration is above the threshold, relative to a likelihood assigned when the measured concentration is below the threshold. For a “negative going” kidney injury marker, an increased likelihood of suffering a future injury to renal function is assigned to the subject when the measured concentration is below the threshold, relative to a likelihood assigned when the measured concentration is above the threshold.

In other preferred risk stratification embodiments, these methods comprise determining a subject's risk for future reduced renal function, and the assay result(s) is/are correlated to a likelihood of such reduced renal function. For example, the measured concentrations may each be compared to a threshold value. For a “positive going” kidney injury marker, an increased likelihood of suffering a future reduced renal function is assigned to the subject when the measured concentration is above the threshold, relative to a likelihood assigned when the measured concentration is below the threshold. For a “negative going” kidney injury marker, an increased likelihood of future reduced renal function is assigned to the subject when the measured concentration is below the threshold, relative to a likelihood assigned when the measured concentration is above the threshold.

In still other preferred risk stratification embodiments, these methods comprise determining a subject's likelihood for a future improvement in renal function, and the assay result(s) is/are correlated to a likelihood of such a future improvement in renal function. For example, the measured concentration(s) may each be compared to a threshold value. For a “positive going” kidney injury marker, an increased likelihood of a future improvement in renal function is assigned to the subject when the measured concentration is below the threshold, relative to a likelihood assigned when the measured concentration is above the threshold. For a “negative going” kidney injury marker, an increased likelihood of a future improvement in renal function is assigned to the subject when the measured concentration is above the threshold, relative to a likelihood assigned when the measured concentration is below the threshold.

In yet other preferred risk stratification embodiments, these methods comprise determining a subject's risk for progression to ARF, and the result(s) is/are correlated to a likelihood of such progression to ARF. For example, the measured concentration(s) may each be compared to a threshold value. For a “positive going” kidney injury marker, an increased likelihood of progression to ARF is assigned to the subject when the measured concentration is above the threshold, relative to a likelihood assigned when the measured concentration is below the threshold. For a “negative going” kidney injury marker, an increased likelihood of progression to ARF is assigned to the subject when the measured concentration is below the threshold, relative to a likelihood assigned when the measured concentration is above the threshold.

And in other preferred risk stratification embodiments, these methods comprise determining a subject's outcome risk, and the assay result(s) is/are correlated to a likelihood of the occurrence of a clinical outcome related to a renal injury suffered by the subject. For example, the measured concentration(s) may each be compared to a threshold value. For a “positive going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is above the threshold, relative to a likelihood assigned when the measured concentration is below the threshold. For a “negative going” kidney injury marker, an increased likelihood of one or more of: acute kidney injury, progression to a worsening stage of AKI, mortality, a requirement for renal replacement therapy, a requirement for withdrawal of renal toxins, end stage renal disease, heart failure, stroke, myocardial infarction, progression to chronic kidney disease, etc., is assigned to the subject when the measured concentration is below the threshold, relative to a likelihood assigned when the measured concentration is above the threshold.

In such risk stratification embodiments, preferably the likelihood or risk assigned is that an event of interest is more or less likely to occur within 180 days of the time at which the body fluid sample is obtained from the subject. In particularly preferred embodiments, the likelihood or risk assigned relates to an event of interest occurring within a shorter time period such as 18 months, 120 days, 90 days, 60 days, 45 days, 30 days, 21 days, 14 days, 7 days, 5 days, 96 hours, 72 hours, 48 hours, 36 hours, 24 hours, 12 hours, or less. A risk at 0 hours of the time at which the body fluid sample is obtained from the subject is equivalent to diagnosis of a current condition.

In preferred risk stratification embodiments, the subject is selected for risk stratification based on the pre-existence in the subject of one or more known risk factors for prerenal, intrinsic renal, or postrenal ARF. For example, a subject undergoing or having undergone major vascular surgery, coronary artery bypass, or other cardiac surgery; a subject having pre-existing congestive heart failure, preeclampsia, eclampsia, diabetes mellitus, hypertension, coronary artery disease, proteinuria, renal insufficiency, glomerular filtration below the normal range, cirrhosis, serum creatinine above the normal range, or sepsis; or a subject exposed to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin are all preferred subjects for monitoring risks according to the methods described herein. This list is not meant to be limiting. By “pre-existence” in this context is meant that the risk factor exists at the time the body fluid sample is obtained from the subject. In particularly preferred embodiments, a subject is chosen for risk stratification based on an existing diagnosis of injury to renal function, reduced renal function, or ARF.

In other embodiments, the methods for evaluating renal status described herein are methods for diagnosing a renal injury in the subject; that is, assessing whether or not a subject has suffered from an injury to renal function, reduced renal function, or ARF. In these embodiments, the assay result(s), for example measured concentration(s) of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, is/are correlated to the occurrence or nonoccurrence of a change in renal status. The following are preferred diagnostic embodiments.

In preferred diagnostic embodiments, these methods comprise diagnosing the occurrence or nonoccurrence of an injury to renal function, and the assay result(s) is/are correlated to the occurrence or nonoccurrence of such an injury. For example, each of the measured concentration(s) may be compared to a threshold value. For a positive going marker, an increased likelihood of the occurrence of an injury to renal function is assigned to the subject when the measured concentration is above the threshold (relative to the likelihood assigned when the measured concentration is below the threshold); alternatively, when the measured concentration is below the threshold, an increased likelihood of the nonoccurrence of an injury to renal function may be assigned to the subject (relative to the likelihood assigned when the measured concentration is above the threshold). For a negative going marker, an increased likelihood of the occurrence of an injury to renal function is assigned to the subject when the measured concentration is below the threshold (relative to the likelihood assigned when the measured concentration is above the threshold); alternatively, when the measured concentration is above the threshold, an increased likelihood of the nonoccurrence of an injury to renal function may be assigned to the subject (relative to the likelihood assigned when the measured concentration is below the threshold).

In other preferred diagnostic embodiments, these methods comprise diagnosing the occurrence or nonoccurrence of reduced renal function, and the assay result(s) is/are correlated to the occurrence or nonoccurrence of an injury causing reduced renal function. For example, each of the measured concentration(s) may be compared to a threshold value. For a positive going marker, an increased likelihood of the occurrence of an injury causing reduced renal function is assigned to the subject when the measured concentration is above the threshold (relative to the likelihood assigned when the measured concentration is below the threshold); alternatively, when the measured concentration is below the threshold, an increased likelihood of the nonoccurrence of an injury causing reduced renal function may be assigned to the subject (relative to the likelihood assigned when the measured concentration is above the threshold). For a negative going marker, an increased likelihood of the occurrence of an injury causing reduced renal function is assigned to the subject when the measured concentration is below the threshold (relative to the likelihood assigned when the measured concentration is above the threshold); alternatively, when the measured concentration is above the threshold, an increased likelihood of the nonoccurrence of an injury causing reduced renal function may be assigned to the subject (relative to the likelihood assigned when the measured concentration is below the threshold).

In yet other preferred diagnostic embodiments, these methods comprise diagnosing the occurrence or nonoccurrence of ARF, and the assay result(s) is/are correlated to the occurrence or nonoccurrence of an injury causing ARF. For example, each of the measured concentration(s) may be compared to a threshold value. For a positive going marker, an increased likelihood of the occurrence of ARF is assigned to the subject when the measured concentration is above the threshold (relative to the likelihood assigned when the measured concentration is below the threshold); alternatively, when the measured concentration is below the threshold, an increased likelihood of the nonoccurrence of ARF may be assigned to the subject (relative to the likelihood assigned when the measured concentration is above the threshold). For a negative going marker, an increased likelihood of the occurrence of ARF is assigned to the subject when the measured concentration is below the threshold (relative to the likelihood assigned when the measured concentration is above the threshold); alternatively, when the measured concentration is above the threshold, an increased likelihood of the nonoccurrence of ARF may be assigned to the subject (relative to the likelihood assigned when the measured concentration is below the threshold).

In still other preferred diagnostic embodiments, these methods comprise diagnosing a subject as being in need of renal replacement therapy, and the assay result(s) is/are correlated to a need for renal replacement therapy. For example, each of the measured concentration(s) may be compared to a threshold value. For a positive going marker, an increased likelihood of the occurrence of an injury creating a need for renal replacement therapy is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal replacement therapy may be assigned to the subject (relative to the likelihood assigned when the measured concentration is above the threshold). For a negative going marker, an increased likelihood of the occurrence of an injury creating a need for renal replacement therapy is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal replacement therapy may be assigned to the subject (relative to the likelihood assigned when the measured concentration is below the threshold).

In still other preferred diagnostic embodiments, these methods comprise diagnosing a subject as being in need of renal transplantation, and the assay result(s) is/are correlated to a need for renal transplantation. For example, each of the measured concentration(s) may be compared to a threshold value. For a positive going marker, an increased likelihood of the occurrence of an injury creating a need for renal transplantation is assigned to the subject when the measured concentration is above the threshold (relative to the likelihood assigned when the measured concentration is below the threshold); alternatively, when the measured concentration is below the threshold, an increased likelihood of the nonoccurrence of an injury creating a need for renal transplantation may be assigned to the subject (relative to the likelihood assigned when the measured concentration is above the threshold). For a negative going marker, an increased likelihood of the occurrence of an injury creating a need for renal transplantation is assigned to the subject when the measured concentration is below the threshold (relative to the likelihood assigned when the measured concentration is above the threshold); alternatively, when the measured concentration is above the threshold, an increased likelihood of the nonoccurrence of an injury creating a need for renal transplantation may be assigned to the subject (relative to the likelihood assigned when the measured concentration is below the threshold).

In still other embodiments, the methods for evaluating renal status described herein are methods for monitoring a renal injury in the subject; that is, assessing whether or not renal function is improving or worsening in a subject who has suffered from an injury to renal function, reduced renal function, or ARF. In these embodiments, the assay result(s), for example measured concentration(s) of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, is/are correlated to the occurrence or nonoccurrence of a change in renal status. The following are preferred monitoring embodiments.

In preferred monitoring embodiments, these methods comprise monitoring renal status in a subject suffering from an injury to renal function, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other preferred monitoring embodiments, these methods comprise monitoring renal status in a subject suffering from reduced renal function, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In yet other preferred monitoring embodiments, these methods comprise monitoring renal status in a subject suffering from acute renal failure, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In other additional preferred monitoring embodiments, these methods comprise monitoring renal status in a subject at risk of an injury to renal function due to the pre-existence of one or more known risk factors for prerenal, intrinsic renal, or postrenal ARF, and the assay result(s) is/are correlated to the occurrence or nonoccurrence of a change in renal status in the subject. For example, the measured concentration(s) may be compared to a threshold value. For a positive going marker, when the measured concentration is above the threshold, a worsening of renal function may be assigned to the subject; alternatively, when the measured concentration is below the threshold, an improvement of renal function may be assigned to the subject. For a negative going marker, when the measured concentration is below the threshold, a worsening of renal function may be assigned to the subject; alternatively, when the measured concentration is above the threshold, an improvement of renal function may be assigned to the subject.

In still other embodiments, the methods for evaluating renal status described herein are methods for classifying a renal injury in the subject; that is, determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE stage. In these embodiments, the assay result(s), for example measured concentration(s) of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, is/are correlated to a particular class and/or subclass. The following are preferred classification embodiments.

In preferred classification embodiments, these methods comprise determining whether a renal injury in a subject is prerenal, intrinsic renal, or postrenal; and/or further subdividing these classes into subclasses such as acute tubular injury, acute glomerulonephritis acute tubulointerstitial nephritis, acute vascular nephropathy, or infiltrative disease; and/or assigning a likelihood that a subject will progress to a particular RIFLE stage, and the assay result(s) is/are correlated to the injury classification for the subject. For example, the measured concentration may be compared to a threshold value, and when the measured concentration is above the threshold, a particular classification is assigned; alternatively, when the measured concentration is below the threshold, a different classification may be assigned to the subject.

A variety of methods may be used by the skilled artisan to arrive at a desired threshold value for use in these methods. For example, the threshold value may be determined from a population of normal subjects by selecting a concentration representing the 75^{th}, 85^{th}, 90^{th}, 95^{th}, or 99^{th} percentile of a kidney injury marker measured in such normal subjects. Alternatively, the threshold value may be determined from a “diseased” population of subjects, e.g., those suffering from an injury or having a predisposition for an injury (e.g., progression to ARF or some other clinical outcome such as death, dialysis, renal transplantation, etc.), by selecting a concentration representing the 75^{th}, 85^{th}, 90^{th}, 95^{th}, or 99^{th} percentile of a kidney injury marker measured in such subjects. In another alternative, the threshold value may be determined from a prior measurement of a kidney injury marker in the same subject; that is, a temporal change in the level of a kidney injury marker in the subject may be used to assign risk to the subject.

The foregoing discussion is not meant to imply, however, that the kidney injury markers of the present invention must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result which is determined by combining individual markers may be treated as if it is itself a marker; that is, a threshold may be determined for the composite result as described herein for individual markers, and the composite result for an individual patient compared to this threshold.

The ability of a particular test to distinguish two populations can be established using ROC analysis. For example, ROC curves established from a “first” subpopulation which is predisposed to one or more future changes in renal status, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more kidney injury markers, or a composite of such markers, may be treated as continuous variables. For example, any particular concentration can be converted into a corresponding probability of a future reduction in renal function for the subject, the occurrence of an injury, a classification, etc. In yet another alternative, a threshold that can provide an acceptable level of specificity and sensitivity in separating a population of subjects into “bins” such as a “first” subpopulation (e.g., which is predisposed to one or more future changes in renal status, the occurrence of an injury, a classification, etc.) and a “second” subpopulation which is not so predisposed. A threshold value is selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;
a specificity of greater than 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;
a sensitivity of greater than 0.5, preferably at least about 0.6, more preferably at least about 0.7, still more preferably at least about 0.8, even more preferably at least about 0.9 and most preferably at least about 0.95, with a corresponding specificity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;
at least about 75% sensitivity, combined with at least about 75% specificity;
a positive likelihood ratio (calculated as sensitivity/(1-specificity)) of greater than 1, at least about 2, more preferably at least about 3, still more preferably at least about 5, and most preferably at least about 10; or
a negative likelihood ratio (calculated as (1-sensitivity)/specificity) of less than 1, less than or equal to about 0.5, more preferably less than or equal to about 0.3, and most preferably less than or equal to about 0.1.

The term “about” in the context of any of the above measurements refers to +/−5% of a given measurement.

Multiple thresholds may also be used to assess renal status in a subject. For example, a “first” subpopulation which is predisposed to one or more future changes in renal status, the occurrence of an injury, a classification, etc., and a “second” subpopulation which is not so predisposed can be combined into a single group. This group is then subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer one or more future changes in renal status in comparison to someone in the first tertile. The third tertile is also assigned an odds ratio that is relative to that first tertile.

In certain embodiments, the assay method is an immunoassay. Antibodies for use in such assays will specifically bind a full length kidney injury marker of interest, and may also bind one or more polypeptides that are “related” thereto, as that term is defined hereinafter. Numerous immunoassay formats are known to those of skill in the art. Preferred body fluid samples are selected from the group consisting of urine, blood, serum, saliva, tears, and plasma.

The foregoing method steps should not be interpreted to mean that the kidney injury marker assay result(s) is/are used in isolation in the methods described herein. Rather, additional variables or other clinical indicia may be included in the methods described herein. For example, a risk stratification, diagnostic, classification, monitoring, etc. method may combine the assay result(s) with one or more variables measured for the subject selected from the group consisting of demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a glomerular filtration rate, an estimated glomerular filtration rate, a urine production rate, a serum or plasma creatinine concentration, a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, a renal failure index calculated as urine sodium/(urine creatinine/plasma creatinine), a serum or plasma neutrophil gelatinase (NGAL) concentration, a urine NGAL concentration, a serum or plasma cystatin C concentration, a serum or plasma cardiac troponin concentration, a serum or plasma BNP concentration, a serum or plasma NTproBNP concentration, and a serum or plasma proBNP concentration. Other measures of renal function which may be combined with one or more kidney injury marker assay result(s) are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

When more than one marker is measured, the individual markers may be measured in samples obtained at the same time, or may be determined from samples obtained at different (e.g., an earlier or later) times. The individual markers may also be measured on the same or different body fluid samples. For example, one kidney injury marker may be measured in a serum or plasma sample and another kidney injury marker may be measured in a urine sample. In addition, assignment of a likelihood may combine an individual kidney injury marker assay result with temporal changes in one or more additional variables.

In various related aspects, the present invention also relates to devices and kits for performing the methods described herein. Suitable kits comprise reagents sufficient for performing an assay for at least one of the described kidney injury markers, together with instructions for performing the described threshold comparisons.

In certain embodiments, reagents for performing such assays are provided in an assay device, and such assay devices may be included in such a kit. Preferred reagents can comprise one or more solid phase antibodies, the solid phase antibody comprising antibody that detects the intended biomarker target(s) bound to a solid support. In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter.

Detectable labels may include molecules that are themselves detectable (e.g., fluorescent moieties, electrochemical labels, ecl (electrochemical luminescence) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.).

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

### DETAILED DESCRIPTION OF THE INVENTION

The present invention relates to methods and compositions for diagnosis, differential diagnosis, risk stratification, monitoring, classifying and determination of treatment regimens in subjects suffering or at risk of suffering from injury to renal function, reduced renal function and/or acute renal failure through measurement of one or more kidney injury markers. In various embodiments, a measured concentration of one or more biomarkers selected from the group consisting of Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, or one or more markers related thereto, are correlated to the renal status of the subject.

For purposes of this document, the following definitions apply:

As used herein, an “injury to renal function” is an abrupt (within 14 days, preferably within 7 days, more preferably within 72 hours, and still more preferably within 48 hours) measurable reduction in a measure of renal function. Such an injury may be identified, for example, by a decrease in glomerular filtration rate or estimated GFR, a reduction in urine output, an increase in serum creatinine, an increase in serum cystatin C, a requirement for renal replacement therapy, etc. “Improvement in Renal Function” is an abrupt (within 14 days, preferably within 7 days, more preferably within 72 hours, and still more preferably within 48 hours) measurable increase in a measure of renal function. Preferred methods for measuring and/or estimating GFR are described hereinafter.

As used herein, “reduced renal function” is an abrupt (within 14 days, preferably within 7 days, more preferably within 72 hours, and still more preferably within 48 hours) reduction in kidney function identified by an absolute increase in serum creatinine of greater than or equal to 0.1 mg/dL (>8.8 μmol), a percentage increase in serum creatinine of greater than or equal to 20% (1.2-fold from baseline), or a reduction in urine output (documented oliguria of less than 0.5 ml/kg per hour).

As used herein, “acute renal failure” or “ARF” is an abrupt (within 14 days, preferably within 7 days, more preferably within 72 hours, and still more preferably within 48 hours) reduction in kidney function identified by an absolute increase in serum creatinine of greater than or equal to 0.3 mg/dl (>26.4 μ=1/1), a percentage increase in serum creatinine of greater than or equal to 50% (1.5-fold from baseline), or a reduction in urine output (documented oliguria of less than 0.5 ml/kg per hour for at least 6 hours). This term is synonymous with “acute kidney injury” or “AKI.”

As used herein, the term “tumor necrosis factor receptor superfamily member 10B” refers to one or more polypeptides present in a biological sample that are derived from the tumor necrosis factor receptor superfamily member 10B precursor (Swiss-Prot O14763 (SEQ ID NO: 1)).

```
                10 20 30 40
MEQRGQNAPA ASGARKRHGP GPREARGARP GPRVPKTLVL
                50 60 70 80
VVAAVLLLVS AESALITQQD LAPQQRAAPQ QKRSSPSEGL
                90 100 110 120
CPPGHHISED GRDCISCKYG QDYSTHWNDL LFCLRCTRCD
              130 140 150 160
SGEVELSPCT TTRNTVCQCE EGTFREEDSP EMCRKCRTGC
              170 180 190 200
PRGMVKVGDC TPWSDIECVH KESGTKHSGE APAVEETVTS
              210 220 230 240
SPGTPASPCS LSGIIIGVTV AAVVLIVAVF VCKSLLWKKV
              250 260 270 280
LPYLKGICSG GGGDPERVDR SSQRPGAEDN VLNEIVSILQ
              290 300 310 320
PTQVPEQEME VQEPAEPTGV NMLSPGESEH LLEPAEAERS
              330 340 350 360
QRRRLLVPAN EGDPTETLRQ CFDDFADLVP FDSWEPLMRK
              370 380 390 400
LGLMDNEIKV AKAEAAGHRD TLYTMLIKWV NKTGRDASVH
              410 420 430 440
TLLDALETLG ERLAKQKIED HLLSSGKFMY LEGNADSAMS
```

Most preferably, the tumor necrosis factor receptor superfamily member 10B assay detects one or more soluble forms of tumor necrosis factor receptor superfamily member 10B. Tumor necrosis factor receptor superfamily member 10B is a single-pass type I membrane protein having a large extracellular domain, most or all of which is present in soluble forms of tumor necrosis factor receptor superfamily member 10B generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in tumor necrosis factor receptor superfamily member 10B:

| Residues | Length | Domain ID |
|---|---|---|
| 1-55 | 55 | signal sequence |
| 56-440 | 385 | tumor necrosis factor receptor |
| | | superfamily member 10B |
| 56-210 | 155 | extracellular |
| 211-231 | 21 | transmembrane |
| 232-440 | 209 | cytoplasmic |

As used herein, the term “cadherin-16” refers to one or more polypeptides present in a biological sample that are derived from the cadherin-16 precursor (Swiss-Prot O75309 (SEQ ID NO: 2)).

```
                10 20 30 40
MVPAWLWLLC VSVPQALPKA QPAELSVEVP ENYGGNFPLY
                50 60 70 80
LTKLPLPREG AEGQIVLSGD SGKATEGPFA MDPDSGFLLV
                90 100 110 120
TRALDREEQA EYQLQVTLEM QDGHVLWGPQ PVLVHVKDEN
              130 140 150 160
DQVPHFSQAI YRARLSRGTR PGIPFLFLEA SDRDEPGTAN
              170 180 190 200
SDLRFHILSQ APAQPSPDMF QLEPRLGALA LSPKGSTSLD
              210 220 230 240
HALERTYQLL VQVKDMGDQA SGHQATATVE VSIIESTWVS
              250 260 270 280
LEPIHLAENL KVLYPHHMAQ VHWSGGDVHY HLESHPPGPF
              290 300 310 320
EVNAEGNLYV TRELDREAQA EYLLQVRAQN SHGEDYAAPL
              330 340 350 360
ELHVLVMDEN DNVPICPPRD PTVSIPELSP PGTEVTRLSA
              370 380 390 400
EDADAPGSPN SHVVYQLLSP EPEDGVEGRA FQVDPTSGSV
              410 420 430 440
TLGVLPLRAG QNILLLVLAM DLAGAEGGFS STCEVEVAVT
              450 460 470 480
DINDHAPEFI TSQIGPISLP EDVEPGTLVA MLTAIDADLE
              490 500 510 520
PAFRLMDFAI ERGDTEGTFG LDWEPDSGHV RLRLCKNLSY
              530 540 550 560
EAAPSHEVVV VVQSVAKLVG PGPGPGATAT VTVLVERVMP
              570 580 590 600
PPKLDQESYE ASVPISAPAG SFLLTIQPSD PISRTLRFSL
              610 620 630 640
VNDSEGWLCI EKFSGEVHTA QSLQGAQPGD TYTVLVEAQD
              650 660 670 680
TDEPRLSASA PLVIHFLKAP PAPALTLAPV PSQYLCTPRQ
              690 700 710 720
DHGLIVSGPS KDPDLASGHG PYSFTLGPNP TVQRDWRLQT
              730 740 750 760
LNGSHAYLTL ALHWVEPREH IIPVVVSHNA QMWQLLVRVI
              770 780 790 800
VCRCNVEGQC MRKVGRMKGM PTKLSAVGIL VGTLVAIGIF
              810 820
LILIFTHWTM SRKKDPDQPA DSVPLKATV
```

Most preferably, the cadherin-16 assay detects one or more soluble forms of cadherin-16. Cadherin-16 is a single-pass type I membrane protein having a large extracellular domain, most or all of which is present in soluble forms of cadherin-16 generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in cadherin-16:

| Residues | Length | Domain ID |
|---|---|---|
| 1-18 | 18 | signal sequence |
| 19 | 829 | cadherin-16 |
| 19-786 | 768 | extracellular |
| 787-807 | 22 | transmembrane |
| 808-829 | 22 | cytoplasmic |

As used herein, the term “caspase-9” refers to one or more polypeptides present in a biological sample that are derived from the caspase-9 precursor (Swiss-Prot P55211 (SEQ ID NO: 3)).

```
                10 20 30 40
MDEADRRLLR RCRLRLVEEL QVDQLWDALL SRELFRPHMI
                50 60 70 80
EDIQRAGSGS RRDQARQLII DLETRGSQAL PLFISCLEDT
                90 100 110 120
GQDMLASFLR TNRQAAKLSK PTLENLTPVV LRPEIRKPEV
              130 140 150 160
LRPETPRPVD IGSGGFGDVG ALESLRGNAD LAYILSMEPC
              170 180 190 200
GHCLIINNVN FCRESGLRTR TGSNIDCEKL RRRFSSLHFM
              210 220 230 240
VEVKGDLTAK KMVLALLELA QQDHGALDCC VVVILSHGCQ
              250 260 270 280
ASHLQFPGAV YGTDGCPVSV EKIVNIFNGT SCPSLGGKPK
              290 300 310 320
LFFIQACGGE QKDHGFEVAS TSPEDESPGS NPEPDATPFQ
              330 340 350 360
EGLRTFDQLD AISSLPTPSD IFVSYSTFPG FVSWRDPKSG
              370 380 390 400
SWYVETLDDI FEQWAHSEDL QSLLLRVANA VSVKGIYKQM
              410
PGCFNFLRKK LFFKTS
```

The following domains have been identified in caspase-9:

| Residues | Length | Domain ID |
|---|---|---|
| 1-315 | 315 | caspase-9 p35 subunit |
| 316-330 | 15 | pro-peptide |
| 331-416 | 86 | caspase-9 subunit p10 |

As used herein, the term “Bcl2 antagonist of cell death” refers to one or more polypeptides present in a biological sample that are derived from the Bcl2 antagonist of cell death precursor (Swiss-Prot Q92934 (SEQ ID NO: 4)).

```
                10 20 30 40
MFQIPEFEPS EQEDSSSAER GLGPSPAGDG PSGSGKHHRQ
                50 60 70 80
APGLLWDASH QQEQPTSSSH HGGAGAVEIR SRHSSYPAGT
                90 100 110 120
EDDEGMGEEP SPFRGRSRSA PPNLWAAQRY GRELRRMSDE
              130 140 150 160
FVDSFKKGLP RPKSAGTATQ MRQSSSWTRV FQSWWDRNLG
RGSSAPSQ
```

As used herein, the term “caspase-1” refers to one or more polypeptides present in a biological sample that are derived from the caspase-1 precursor (Swiss-Prot P29466 (SEQ ID NO: 5)).

```
                10 20 30 40
MADKVLKEKR KLFIRSMGEG TINGLLDELL QTRVLNKEEM
                50 60 70 80
EKVKRENATV MDKTRALIDS VIPKGAQACQ ICITYICEED
                90 100 110 120
SYLAGTLGLS ADQTSGNYLN MQDSQGVLSS FPAPQAVQDN
              130 140 150 160
PAMPTSSGSE GNVKLCSLEE AQRIWKQKSA EIYPIMDKSS
              170 180 190 200
RTRLALIICN EEFDSIPRRT GAEVDITGMT MLLQNLGYSV
              210 220 230 240
DVKKNLTASD MTTELEAFAH RPEHKTSDST FLVFMSHGIR
              250 260 270 280
EGICGKKHSE QVPDILQLNA IFNMLNTKNC PSLKDKPKVI
              290 300 310 320
IIQACRGDSP GVVWFKDSVG VSGNLSLPTT EEFEDDAIKK
              330 340 350 360
AHIEKDFIAF CSSTPDNVSW RHPTMGSVFI GRLIEHMQEY
              370 380 390 400
ACSCDVEEIF RKVRFSFEQP DGRAQMPTTE RVTLTRCFYL
FPGH
```

The following domains have been identified in caspase-1:

| Residues | Length | Domain ID |
|---|---|---|
| 1-119 | 119 | pro-peptide |
| 120-297 | 178 | caspase-1 p20 subunit |
| 298-316 | 19 | pro-peptide |
| 317-404 | 88 | caspase-1 p10 subunit |

As used herein, the terms “epithelial cadherin” or “Cadherin-1” refers to one or more polypeptides present in a biological sample that are derived from the epithelial cadherin precursor (Swiss-Prot P12830 (SEQ ID NO: 6)).

```
                10 20 30 40
MGPWSRSLSA LLLLLQVSSW LCQEPEPCHP GFDAESYTFT
                50 60 70 80
VPRRHLERGR VLGRVNFEDC TGRQRTAYFS LDTRFKVGTD
                90 100 110 120
GVITVKRPLR FHNPQIHFLV YAWDSTYRKF STKVTLNTVG
              130 140 150 160
HHHRPPPHQA SVSGIQAELL TFPNSSPGLR RQKRDWVIPP
              170 180 190 200
ISCPENEKGP FPKNLVQIKS NKDKEGKVFY SITGQGADTP
              210 220 230 240
PVGVFIIERE TGWLKVTEPL DRERIATYTL FSHAVSSNGN
              250 260 270 280
AVEDPMEILI TVTDQNDNKP EFTQEVFKGS VMEGALPGTS
              290 300 310 320
VMEVTATDAD DDVNTYNAAI AYTILSQDPE LPDKNMFTIN
              330 340 350 360
RNTGVISVVT TGLDRESFPT YTLVVQAADL QGEGLSTTAT
              370 380 390 400
AVITVTDTND NPPIFNPTTY KGQVPENEAN VVITTLKVTD
              410 420 430 440
ADAPNTPAWE AVYTILNDDG GQFVVTTNPV NNDGILKTAK
              450 460 470 480
GLDFEAKQQY ILHVAVTNVV PFEVSLTTST ATVTVDVLDV
              490 500 510 520
NEAPIFVPPE KRVEVSEDFG VGQEITSYTA QEPDTFMEQK
              530 540 550 560
ITYRIWRDTA NWLEINPDTG AISTRAELDR EDFEHVKNST
              570 580 590 600
YTALIIATDN GSPVATGTGT LLLILSDVND NAPIPEPRTI
              610 620 630 640
FFCERNPKPQ VINIIDADLP PNTSPFTAEL THGASANWTI
              650 660 670 680
QYNDPTQESI ILKPKMALEV GDYKINLKLM DNQNKDQVTT
              690 700 710 720
LEVSVCDCEG AAGVCRKAQP VEAGLQIPAI LGILGGILAL
              730 740 750 760
LILILLLLLF LRRRAVVKEP LLPPEDDTRD NVYYYDEEGG
              770 780 790 800
GEEDQDFDLS QLHRGLDARP EVTRNDVAPT LMSVPRYLPR
              810 820 830 840
PANPDEIGNF IDENLKAADT DPTAPPYDSL LVFDYEGSGS
              850 860 870 880
EAASLSSLNS SESDKDQDYD YLNEWGNRFK KLADMYGGGE
DD
```

Most preferably, the epithelial cadherin assay detects one or more soluble forms of epithelial cadherin. Epithelial cadherin is a single-pass type I membrane protein having a large extracellular domain, most or all of which is present in soluble forms of epithelial cadherin generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in epithelial cadherin:

| Residues | Length | Domain ID |
|---|---|---|
| 1-22 | 22 | signal sequence |
| 23-154 | 132 | pro-peptide |
| 155-882 | 728 | epithelial cadherin |
| 155-709 | 555 | extracellular |
| 710-730 | 21 | transmembrane |
| 731-882 | 152 | cytoplasmic |

As used herein, the term “poly [ADP-ribose] polymerase 1” refers to one or more polypeptides present in a biological sample that are derived from the poly [ADP-ribose] polymerase 1 precursor (Swiss-Prot 09874 (SEQ ID NO: 7)).

```
                10 20 30 40
MAESSDKLYR VEYAKSGRAS CKKCSESIPK DSLRMAIMVQ
                50 60 70 80
SPMFDGKVPH WYHFSCFWKV GHSIRHPDVE VDGFSELRWD
                90 100 110 120
DQQKVKKTAE AGGVTGKGQD GIGSKAEKTL GDFAAEYAKS
              130 140 150 160
NRSTCKGCME KIEKGQVRLS KKMVDPEKPQ LGMIDRWYHP
              170 180 190 200
GCFVKNREEL GFRPEYSASQ LKGFSLLATE DKEALKKQLP
              210 220 230 240
GVKSEGKRKG DEVDGVDEVA KKKSKKEKDK DSKLEKALKA
              250 260 270 280
QNDLIWNIKD ELKKVCSTND LKELLIFNKQ QVPSGESAIL
              290 300 310 320
DRVADGMVFG ALLPCEECSG QLVFKSDAYY CTGDVTAWTK
              330 340 350 360
CMVKTQTPNR KEWVTPKEFR EISYLKKLKV KKQDRIFPPE
              370 380 390 400
TSASVAATPP PSTASAPAAV NSSASADKPL SNMKILTLGK
              410 420 430 440
LSRNKDEVKA MIEKLGGKLT GTANKASLCI STKKEVEKMN
              450 460 470 480
KKMEEVKEAN IRVVSEDFLQ DVSASTKSLQ ELFLAHILSP
              490 500 510 520
WGAEVKAEPV EVVAPRGKSG AALSKKSKGQ VKEEGINKSE
              530 540 550 560
KRMKLTLKGG AAVDPDSGLE HSAHVLEKGG KVFSATLGLV
              570 580 590 600
DIVKGTNSYY KLQLLEDDKE NRYWIFRSWG RVGTVIGSNK
              610 620 630 640
LEQMPSKEDA IEHFMKLYEE KTGNAWHSKN FTKYPKKFYP
              650 660 670 680
LEIDYGQDEE AVKKLTVNPG TKSKLPKPVQ DLIKMIFDVE
              690 700 710 720
SMKKAMVEYE IDLQKMPLGK LSKRQIQAAY SILSEVQQAV
              730 740 750 760
SQGSSDSQIL DLSNRFYTLI PHDFGMKKPP LLNNADSVQA
              770 780 790 800
KVEMLDNLLD IEVAYSLLRG GSDDSSKDPI DVNYEKLKTD
              810 820 830 840
IKVVDRDSEE AEIIRKYVKN THATTHNAYD LEVIDIFKIE
              850 860 870 880
REGECQRYKP FKQLHNRRLL WHGSRTTNFA GILSQGLRIA
              890 900 910 920
PPEAPVTGYM FGKGIYFADM VSKSANYCHT SQGDPIGLIL
              930 940 950 960
LGEVALGNMY ELKHASHISK LPKGKHSVKG LGKTTPDPSA
              970 980 990 1000
NISLDGVDVP LGTGISSGVN DTSLLYNEYI VYDIAQVNLK
            1010
YLLKLKFNFK TSLW
```

The following domains have been identified in poly [ADP-ribose] polymerase 1:

| Residues | Length | Domain ID |
|---|---|---|
| 1 | 1 | initiator methionine |
| 2-1014 | 1013 | poly [ADP-ribose] polymerase 1 |

Poly [ADP-ribose] polymerase 1 can be cleaved by many caspases in vitro and is one of the main cleavage targets of caspase-3 in vivo. The cleavage occurs between Asp(214) and Gly(215), which separates PARP's N-terminal DNA binding domain (24 kDa) from its C-terminal catalytic domain (89 kDa). Suitable assays may recognize only the large fragment of poly [ADP-ribose] polymerase 1 (89 kDa) but not the full length poly [ADP-ribose] polymerase 1, may recognize only the small fragment of poly [ADP-ribose] polymerase 1 (24 kDa) but not the full length poly [ADP-ribose] polymerase 1, may recognize only full length poly [ADP-ribose] polymerase 1, or may recognize one fragment and the full length full length poly [ADP-ribose] polymerase 1.

As used herein, the term “cyclin-dependent kinase inhibitor 1” refers to one or more polypeptides present in a biological sample that are derived from the cyclin-dependent kinase inhibitor 1 precursor (Swiss-Prot P38936 (SEQ ID NO: 8)).

```
                10 20 30 40
MSEPAGDVRQ NPCGSKACRR LFGPVDSEQL SRDCDALMAG
                50 60 70 80
CIQEARERWN FDFVTETPLE GDFAWERVRG LGLPKLYLPT
                90 100 110 120
GPRRGRDELG GGRRPGTSPA LLQGTAEEDH VDLSLSCTLV
              130 140 150 160
PRSGEQAEGS PGGPGDSQGR KRRQTSMTDF YHSKRRLIFS
KRKP
```

The following domains have been identified in cyclin-dependent kinase inhibitor 1:

| Residues | Length | Domain |
|---|---|---|
| 1 | 1 | initiator methionine |
| 2-164 | 163 | cyclin-dependent kinase inhibitor 1 |

As used herein, the term “cadherin-5” refers to one or more polypeptides present in a biological sample that are derived from the cadherin-5 precursor (Swiss-Prot P33151 (SEQ ID NO: 9)).

```
                10 20 30 40
MQRLMMLLAT SGACLGLLAV AAVAAAGANP AQRDTHSLLP
                50 60 70 80
THRRQKRDWI WNQMHIDEEK NTSLPHHVGK IKSSVSRKNA
                90 100 110 120
KYLLKGEYVG KVFRVDAETG DVFAIERLDR ENISEYHLTA
              130 140 150 160
VIVDKDTGEN LETPSSFTIK VHDVNDNWPV FTHRLFNASV
              170 180 190 200
PESSAVGTSV ISVTAVDADD PTVGDHASVM YQILKGKEYF
              210 220 230 240
AIDNSGRIIT ITKSLDREKQ ARYEIVVEAR DAQGLRGDSG
              250 260 270 280
TATVLVTLQD INDNFPFFTQ TKYTFVVPED TRVGTSVGSL
              290 300 310 320
FVEDPDEPQN RMTKYSILRG DYQDAFTIET NPAHNEGIIK
              330 340 350 360
PMKPLDYEYI QQYSFIVEAT DPTIDLRYMS PPAGNRAQVI
              370 380 390 400
INITDVDEPP IFQQPFYHFQ LKENQKKPLI GTVLAMDPDA
              410 420 430 440
ARHSIGYSIR RTSDKGQFFR VTKKGDIYNE KELDREVYPW
              450 460 470 480
YNLTVEAKEL DSTGTPTGKE SIVQVHIEVL DENDNAPEFA
              490 500 510 520
KPYQPKVCEN AVHGQLVLQI SAIDKDITPR NVKFKFTLNT
              530 540 550 560
ENNFTLTDNH DNTANITVKY GQFDREHTKV HFLPVVISDN
              570 580 590 600
GMPSRTGTST LTVAVCKCNE QGEFTFCEDM AAQVGVSIQA
              610 620 630 640
VVAILLCILT ITVITLLIFL RRRLRKQARA HGKSVPEIHE
              650 660 670 680
QLVTYDEEGG GEMDTTSYDV SVLNSVRRGG AKPPRPALDA
              690 700 710 720
RPSLYAQVQK PPRHAPGAHG GPGEMAAMIE VKKDEADHDG
              730 740 750 760
DGPPYDTLHI YGYEGSESIA ESLSSLGTDS SDSDVDYDFL
              770 780
NDWGPRFKML AELYGSDPRE ELLY
```

Most preferably, the cadherin-5 assay detects one or more soluble forms of cadherin-5. Cadherin-5 is a single-pass type I membrane protein having a large extracellular domain, most or all of which is present in soluble forms of cadherin-5 generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in cadherin-5:

| Residues | Length | Domain ID |
|---|---|---|
| 1-25 | 251 | signal sequence |
| 26-47 | 22 | pro-peptide |
| 48-784 | 737 | cadherin-5 |
| 48-599 | 522 | extracellular |
| 600-620 | 21 | transmembrane |
| 621-784 | 164 | cytoplasmic |

As used herein, the term “Myoglobin” refers to one or polypeptides present in a biological sample that are derived from the Myoglobin precursor (Swiss-Prot P02144 (SEQ ID NO: 10)).

```
                10 20 30 40
MGLSDGEWQL VLNVWGKVEA DIPGHGQEVL IRLFKGHPET
                50 60 70 80
LEKFDKFKHL KSEDEMKASE DLKKHGATVL TALGGILKKK
                90 100 110 120
GHHEAEIKPL AQSHATKHKI PVKYLEFISE CIIQVLQSKH
              130 140 150
PGDFGADAQG AMNKALELFR KDMASNYKEL GFQG
```

The following domains have been identified in Myoglobin:

| Residues | Length | Domain ID |
|---|---|---|
| 1 | 1 | Initiator Methionine |
| 2-154 | 153 | Myoglobin |

As used herein, the term “Apolipoprotein A-II” refers to one or polypeptides present in a biological sample that are derived from the Apolipoprotein A-II precursor (Swiss-Prot P02652 (SEQ ID NO: 11)).

```
                10 20 30 40
MKLLAATVLL LTICSLEGAL VRRQAKEPCV ESLVSQYFQT
                50 60 70 80
VTDYGKDLME KVKSPELQAE AKSYFEKSKE QLTPLIKKAG
                90 100
TELVNFLSYF VELGTQPATQ
```

The following domains have been identified in Apolipoprotein A-II:

| Residues | Length | Domain ID |
|---|---|---|
| 1-18 | 18 | Signal sequence |
| 19-23 | 5 | Propeptide |
| 24-100 | 77 | Apolipoprotein A-II |
| 24-99 | 76 | Apolipoprotein A-II(1-76) |

As used herein, the term “Mucin-16” refers to one or polypeptides present in a biological sample that are derived from the Mucin-16 precursor (Swiss-Prot Q8WXI7 (SEQ ID NO: 12)).

```
              10 20 30 40
MLKPSGLPGS SSPTRSLMTG SRSTKATPEM DSGLTGATLS
                50 60 70 80
PKTSTGAIVV TEHTLPFTSP DKTLASPTSS VVGRTTQSLG
                90 100 110 120
VMSSALPEST SRGMTHSEQR TSPSLSPQVN GTPSRNYPAT
              130 140 150 160
SMVSGLSSPR TRTSSTEGNF TKEASTYTLT VETTSGPVTE
              170 180 190 200
KYTVPTETST TEGDSTETPW DTRYIPVKIT SPMKTFADST
              210 220 230 240
ASKENAPVSM TPAETTVTDS HTPGRTNPSF GTLYSSFLDL
              250 260 270 280
SPKGTPNSRG ETSLELILST TGYPFSSPEP GSAGHSRIST
              290 300 310 320
SAPLSSSASV LDNKISETSI FSGQSLTSPL SPGVPEARAS
              330 340 350 360
TMPNSAIPFS MTLSNAETSA ERVRSTISSL GTPSISTKQT
              370 380 390 400
AETILTFHAF AETMDIPSTH IAKTLASEWL GSPGTLGGTS
              410 420 430 440
TSALTTTSPS TTLVSEETNT HHSTSGKETE GTLNTSMTPL
              450 460 470 480
ETSAPGEESE MTATLVPTLG FTTLDSKIRS PSQVSSSHPT
              490 500 510 520
RELRTTGSTS GRQSSSTAAH GSSDILRATT SSTSKASSWT
              530 540 550 560
SESTAQQFSE PQHTQWVETS PSMKTERPPA STSVAAPITT
              570 580 590 600
SVPSVVSGFT TLKTSSTKGI WLEETSADTL IGESTAGPTT
              610 620 630 640
HQFAVPTGIS MTGGSSTRGS QGTTHLLTRA TASSETSADL
              650 660 670 680
TLATNGVPVS VSPAVSKTAA GSSPPGGTKP SYTMVSSVIP
              690 700 710 720
ETSSLQSSAF REGTSLGLTP LNTRHPFSSP EPDSAGHTKI
              730 740 750 760
STSIPLLSSA SVLEDKVSAT STFSHHKATS SITTGTPEIS
              770 780 790 800
TKTKPSSAVL SSMTLSNAAT SPERVRNATS PLTHPSPSGE
              810 820 830 840
ETAGSVLTLS TSAETTDSPN IHPTGTLTSE SSESPSTLSL
              850 860 870 880
PSVSGVKTTF SSSTPSTHLF TSGEETEETS NPSVSQPETS
              890 900 910 920
VSRVRTTLAS TSVPTPVFPT MDTWPTRSAQ FSSSHLVSEL
              930 940 950 960
RATSSTSVTN STGSALPKIS HLTGTATMSQ TNRDTFNDSA
              970 980 990 1000
APQSTTWPET SPRFKTGLPS ATTTVSTSAT SLSATVMVSK
            1010 1020 1030 1040
FTSPATSSME ATSIREPSTT ILTTETTNGP GSMAVASTNI
            1050 1060 1070 1080
PIGKGYITEG RLDTSHLPIG TTASSETSMD FTMAKESVSM
            1090 1100 1110 1120
SVSPSQSMDA AGSSTPGRTS QFVDTFSDDV YHLTSREITI
            1130 1140 1150 1160
PRDGTSSALT PQMTATHPPS PDPGSARSTW LGILSSSPSS
            1170 1180 1190 1200
PTPKVTMSST FSTQRVTTSM IMDTVETSRW NMPNLPSTTS
            1210 1220 1230 1240
LTPSNIPTSG AIGKSTLVPL DTPSPATSLE ASEGGLPTLS
            1250 1260 1270 1280
TYPESTNTPS IHLGAHASSE SPSTIKLTMA SVVKPGSYTP
            1290 1300 1310 1320
LTFPSIETHI HVSTARMAYS SGSSPEMTAP GETNTGSTWD
            1330 1340 1350 1360
PTTYITTTDP KDTSSAQVST PHSVRTLRTT ENHPKTESAT
            1370 1380 1390 1400
PAAYSGSPKI SSSPNLTSPA TKAWTITDTT EHSTQLHYTK
            1410 1420 1430 1440
LAEKSSGFET QSAPGPVSVV IPTSPTIGSS TLELTSDVPG
            1450 1460 1470 1480
EPLVLAPSEQ TTITLPMATW LSTSLTEEMA STDLDISSPS
            1490 1500 1510 1520
SPMSTFAIFP PMSTPSHELS KSEADTSAIR NTDSTTLDQH
            1530 1540 1550 1560
LGIRSLGRTG DLTTVPITPL TTTWTSVIEH STQAQDTLSA
            1570 1580 1590 1600
TMSPTHVTQS LKDQTSIPAS ASPSHLTEVY PELGTQGRSS
            1610 1620 1630 1640
SEATTFWKPS TDTLSREIET GPTNIQSTPP MDNTTTGSSS
            1650 1660 1670 1680
SGVTLGIAHL PIGTSSPAET STNMALERRS STATVSMAGT
            1690 1700 1710 1720
MGLLVTSAPG RSISQSLGRV SSVLSESTTE GVTDSSKGSS
            1730 1740 1750 1760
PRLNTQGNTA LSSSLEPSYA EGSQMSTSIP LTSSPTTPDV
            1770 1780 1790 1800
EFIGGSTFWT KEVTTVMTSD ISKSSARTES SSATLMSTAL
            1810 1820 1830 1840
GSTENTGKEK LRTASMDLPS PTPSMEVTPW ISLTLSNAPN
            1850 1860 1870 1880
TTDSLDLSHG VHTSSAGTLA TDRSLNTGVT RASRLENGSD
            1890 1900 1910 1920
TSSKSLSMGN STHTSMTDTE KSEVSSSIHP RPETSAPGAE
            1930 1940 1950 1960
TTLTSTPGNR AISLTLPFSS IPVEEVISTG ITSGPDINSA
            1970 1980 1990 2000
PMTHSPITPP TIVWTSTGTI EQSTQPLHAV SSEKVSVQTQ
            2010 2020 2030 2040
STPYVNSVAV SASPTHENSV SSGSSTSSPY SSASLESLDS
            2050 2060 2070 2080
TISRRNAITS WLWDLTTSLP TTTWPSTSLS EALSSGHSGV
            2090 2100 2110 2120
SNPSSTTTEF PLFSAASTSA AKQRNPETET HGPQNTAAST
            2130 2140 2150 2160
LNTDASSVTG LSETPVGASI SSEVPLPMAI TSRSDVSGLT
            2170 2180 2190 2200
SESTANPSLG TASSAGTKLT RTISLPTSES LVSFRMNKDP
            2210 2220 2230 2240
WTVSIPLGSH PTTNTETSIP VNSAGPPGLS TVASDVIDTP
            2250 2260 2270 2280
SDGAESIPTV SFSPSPDTEV TTISHFPEKT THSFRTISSL
            2290 2300 2310 2320
THELTSRVTP IPGDWMSSAM STKPTGASPS ITLGERRTIT
            2330 2340 2350 2360
SAAPTTSPIV LTASFTETST VSLDNETTVK TSDILDARKT
            2370 2380 2390 2400
NELPSDSSSS SDLINTSIAS STMDVTKTAS ISPTSISGMT
            2410 2420 2430 2440
ASSSPSLFSS DRPQVPTSTT ETNTATSPSV SSNTYSLDGG
            2450 2460 2470 2480
SNVGGTPSTL PPFTITHPVE TSSALLAWSR PVRTFSTMVS
            2490 2500 2510 2520
TDTASGENPT SSNSVVTSVP APGTWASVGS TTDLPAMGFL
            2530 2540 2550 2560
KTSPAGEAHS LLASTIEPAT AFTPHLSAAV VTGSSATSEA
            2570 2580 2590 2600
SLLTTSESKA IHSSPQTPTT PTSGANWETS ATPESLLVVT
            2610 2620 2630 2640
ETSDTTLTSK ILVTDTILFS TVSTPPSKFP STGTLSGASF
            2650 2660 2670 2680
PTLLPDTPAI PLTATEPTSS LATSFDSTPL VTIASDSLGT
            2690 2700 2710 2720
VPETTLTMSE TSNGDALVLK TVSNPDRSIP GITIQGVTES
            2730 2740 2750 2760
PLHPSSTSPS KIVAPRNTTY EGSITVALST LPAGTTGSLV
            2770 2780 2790 2800
FSQSSENSET TALVDSSAGL ERASVMPLTT GSQGMASSGG
            2810 2820 2830 2840
IRSGSTHSTG TKTFSSLPLT MNPGEVTAMS EITTNRLTAT
            2850 2860 2870 2880
QSTAPKGIPV KPTSAESGLL TPVSASSSPS KAFASLTTAP
            2890 2900 2910 2920
PSTWGIPQST LTFEFSEVPS LDTKSASLPT PGQSLNTIPD
            2930 2940 2950 2960
SDASTASSSL SKSPEKNPRA RMMTSTKAIS ASSFQSTGFT
            2970 2980 2990 3000
ETPEGSASPS MAGHEPRVPT SGTGDPRYAS ESMSYPDPSK
            3010 3020 3030 3040
ASSAMTSTSL ASKLTTLFST GQAARSGSSS SPISLSTEKE
            3050 3060 3070 3080
TSFLSPTAST SRKTSLFLGP SMARQPNILV HLQTSALTLS
            3090 3100 3110 3120
PTSTLNMSQE EPPELTSSQT IAEEEGTTAE TQTLTFTPSE
            3130 3140 3150 3160
TPTSLLPVSS PTEPTARRKS SPETWASSIS VPAKTSLVET
            3170 3180 3190 3200
TDGTLVTTIK MSSQAAQGNS TWPAPAEETG TSPAGTSPGS
            3210 3220 3230 3240
PEVSTTLKIM SSKEPSISPE IRSTVRNSPW KTPETTVPME
            3250 3260 3270 3280
TTVEPVTLQS TALGSGSTSI SHLPTGTTSP TKSPTENMLA
            3290 3300 3310 3320
TERVSLSPSP PEAWTNLYSG TPGGTRQSLA TMSSVSLESP
            3330 3340 3350 3360
TARSITGTGQ QSSPELVSKT TGMEFSMWHG STGGTTGDTH
            3370 3380 3390 3400
VSLSTSSNIL EDPVTSPNSV SSLTDKSKHK TETWVSTTAI
            3410 3420 3430 3440
PSTVLNNKIM AAEQQTSRSV DEAYSSTSSW SDQTSGSDIT
            3450 3460 3470 3480
LGASPDVTNT LYITSTAQTT SLVSLPSGDQ GITSLTNPSG
            3490 3500 3510 3520
GKTSSASSVT SPSIGLETLR ANVSAVKSDI APTAGHLSQT
            3530 3540 3550 3560
SSPAEVSILD VTTAPTPGIS TTITTMGTNS ISTTTPNPEV
            3570 3580 3590 3600
GMSTMDSTPA TERRTTSTEH PSTWSSTAAS DSWTVTDMTS
            3610 3620 3630 3640
NLKVARSPGT ISTMHTTSFL ASSTELDSMS TPHGRITVIG
            3650 3660 3670 3680
TSLVTPSSDA SAVKTETSTS ERTLSPSDTT ASTPISTFSR
            3690 3700 3710 3720
VQRMSISVPD ILSTSWTPSS TEAEDVPVSM VSTDHASTKT
            3730 3740 3750 3760
DPNTPLSTFL FDSLSTLDWD TGRSLSSATA TTSAPQGATT
            3770 3780 3790 3800
PQELTLETMI SPATSQLPFS IGHITSAVTP AAMARSSGVT
            3810 3820 3830 3840
FSRPDPTSKK AEQTSTQLPT TTSAHPGQVP RSAATTLDVI
            3850 3860 3870 3880
PHTAKTPDAT FQRQGQTALT TEARATSDSW NEKEKSTPSA
            3890 3900 3910 3920
PWITEMMNSV SEDTIKEVTS SSSVLKDPEY AGHKLGIWDD
            3930 3940 3950 3960
FIPKFGKAAH MRELPLLSPP QDKEAIHPST NTVETTGWVT
            3970 3980 3990 4000
SSEHASHSTI PAHSASSKLT SPVVTTSTRE QAIVSMSTTT
            4010 4020 4030 4040
WPESTRARTE PNSFLTIELR DVSPYMDTSS TTQTSIISSP
            4050 4060 4070 4080
GSTAITKGPR TEITSSKRIS SSFLAQSMRS SDSPSEAITR
            4090 4100 4110 4120
LSNFPAMTES GGMILAMQTS PPGATSLSAP TLDTSATASW
            4130 4140 4150 4160
TGTPLATTQR FTYSEKTTLF SKGPEDTSQP SPPSVEETSS
            4170 4180 4190 4200
SSSLVPIHAT TSPSNILLTS QGHSPSSTPP VTSVFLSETS
            4210 4220 4230 4240
GLGKTTDMSR ISLEPGTSLP PNLSSTAGEA LSTYEASRDT
            4250 4260 4270 4280
KAIHHSADTA VTNMEATSSE YSPIPGHTKP SKATSPLVTS
            4290 4300 4310 4320
HIMGDITSST SVFGSSETTE IETVSSVNQG LQERSTSQVA
            4330 4340 4350 4360
SSATETSTVI THVSSGDATT HVTKTQATFS SGTSISSPHQ
            4370 4380 4390 4400
FITSTNTFTD VSTNPSTSLI MTESSGVTIT TQTGPTGAAT
            4410 4420 4430 4440
QGPYLLDTST MPYLTETPLA VTPDFMQSEK TTLISKGPKD
            4450 4460 4470 4480
VTWTSPPSVA ETSYPSSLTP FLVTTIPPAT STLQGQHTSS
            4490 4500 4510 4520
PVSATSVLTS GLVKTTDMLN TSMEPVTNSP QNLNNPSNEI
            4530 4540 4550 4560
LATLAATTDI ETIHPSINKA VTNMGTASSA HVLHSTLPVS
            4570 4580 4590 4600
SEPSTATSPM VPASSMGDAL ASISIPGSET TDIEGEPTSS
            4610 4620 4630 4640
LTAGRKENST LQEMNSTTES NIILSNVSVG AITEATKMEV
            4650 4660 4670 4680
PSFDATFIPT PAQSTKFPDI FSVASSRLSN SPPMTISTHM
            4690 4700 4710 4720
TTTQTGSSGA TSKIPLALDT STLETSAGTP SVVTEGFAHS
            4730 4740 4750 4760
KITTAMNNDV KDVSQTNPPF QDEASSPSSQ APVLVTTLPS
            4770 4780 4790 4800
SVAFTPQWHS TSSPVSMSSV LTSSLVKTAG KVDTSLETVT
            4810 4820 4830 4840
SSPQSMSNTL DDISVTSAAT TDIETTHPSI NTVVTNVGTT
            4850 4860 4870 4880
GSAFESHSTV SAYPEPSKVT SPNVTTSTME DTTISRSIPK
            4890 4900 4910 4920
SSKTTRTETE TTSSLTPKLR ETSISQEITS STETSTVPYK
            4930 4940 4950 4960
ELTGATTEVS RTDVTSSSST SFPGPDQSTV SLDISTETNT
            4970 4980 4990 5000
RLSTSPIMTE SAEITITTQT GPHGATSQDT FTMDPSNTTP
            5010 5020 5030 5040
QAGIHSAMTH GFSQLDVTTL MSRIPQDVSW TSPPSVDKTS
            5050 5060 5070 5080
SPSSFLSSPA MTTPSLISST LPEDKLSSPM TSLLTSGLVK
            5090 5100 5110 5120
ITDILRTRLE PVTSSLPNFS STSDKILATS KDSKDTKEIF
            5130 5140 5150 5160
PSINTEETNV KANNSGHESH SPALADSETP KATTQMVITT
            5170 5180 5190 5200
TVGDPAPSTS MPVHGSSETT NIKREPTYFL TPRLRETSTS
            5210 5220 5230 5240
QESSFPTDTS FLLSKVPTGT ITEVSSTGVN SSSKISTPDH
            5250 5260 5270 5280
DKSTVPPDTF TGEIPRVFTS SIKTKSAEMT ITTQASPPES
            5290 5300 5310 5320
ASHSTLPLDT STTLSQGGTH STVTQGFPYS EVTTLMGMGP
            5330 5340 5350 5360
GNVSWMTTPP VEETSSVSSL MSSPAMTSPS PVSSTSPQSI
            5370 5380 5390 5400
PSSPLPVTAL PTSVLVTTTD VLGTTSPESV TSSPPNLSSI
            5410 5420 5430 5440
THERPATYKD TAHTEAAMHH STNTAVTNVG TSGSGHKSQS
            5450 5460 5470 5480
SVLADSETSK ATPLMSTTST LGDTSVSTST PNISQTNQIQ
            5490 5500 5510 5520
TEPTASLSPR LRESSTSEKT SSTTETNTAF SYVPTGAITQ
            5530 5540 5550 5560
ASRTEISSSR TSISDLDRPT IAPDISTGMI TRLFTSPIMT
            5570 5580 5590 5600
KSAEMTVTTQ TTTPGATSQG ILPWDTSTTL FQGGTHSTVS
            5610 5620 5630 5640
QGFPHSEITT LRSRTPGDVS WMTTPPVEET SSGFSLMSPS
            5650 5660 5670 5680
MTSPSPVSST SPESIPSSPL PVTALLTSVL VTTTNVLGTT
            5690 5700 5710 5720
SPETVTSSPP NLSSPTQERL TTYKDTAHTE AMHASMHTNT
            5730 5740 5750 5760
AVANVGTSIS GHESQSSVPA DSHTSKATSP MGITFAMGDT
            5770 5780 5790 5800
SVSTSTPAFF ETRIQTESTS SLIPGLRDTR TSEEINTVTE
            5810 5820 5830 5840
TSTVLSEVPT TTTTEVSRTE VITSSRTTIS GPDHSKMSPY
            5850 5860 5870 5880
ISTETITRLS TFPFVTGSTE MAITNQTGPI GTISQATLTL
            5890 5900 5910 5920
DTSSTASWEG THSPVTQRFP HSEETTTMSR STKGVSWQSP
            5930 5940 5950 5960
PSVEETSSPS SPVPLPAITS HSSLYSAVSG SSPTSALPVT
            5970 5980 5990 6000
SLLTSGRRKT IDMLDTHSEL VTSSLPSASS FSGEILTSEA
            6010 6020 6030 6040
STNTETIHFS ENTAETNMGT TNSMHKLHSS VSIHSQPSGH
            6050 6060 6070 6080
TPPKVTGSMM EDAIVSTSTP GSPETKNVDR DSTSPLTPEL
            6090 6100 6110 6120
KEDSTALVMN STTESNTVFS SVSLDAATEV SRAEVTYYDP
            6130 6140 6150 6160
TFMPASAQST KSPDISPEAS SSHSNSPPLT ISTHKTIATQ
            6170 6180 6190 6200
TGPSGVTSLG QLTLDTSTIA TSAGTPSART QDFVDSETTS
            6210 6220 6230 6240
VMNNDLNDVL KTSPFSAEEA NSLSSQAPLL VTTSPSPVTS
            6250 6260 6270 6280
TLQEHSTSSL VSVTSVPTPT LAKITDMDTN LEPVTRSPQN
            6290 6300 6310 6320
LRNTLATSEA TTDTHTMHPS INTAMANVGT TSSPNEFYFT
            6330 6340 6350 6360
VSPDSDPYKA TSAVVITSTS GDSIVSTSMP RSSAMKKIES
            6370 6380 6390 6400
ETTFSLIFRL RETSTSQKIG SSSDTSTVFD KAFTAATTEV
            6410 6420 6430 6440
SRTELTSSSR TSIQGTEKPT MSPDTSTRSV TMLSTFAGLT
            6450 6460 6470 6480
KSEERTIATQ TGPHRATSQG TLTWDTSITT SQAGTHSAMT
            6490 6500 6510 6520
HGFSQLDLST LTSRVPEYIS GTSPPSVEKT SSSSSLLSLP
            6530 6540 6550 6560
AITSPSPVPT TLPESRPSSP VHLTSLPTSG LVKTTDMLAS
            6570 6580 6590 6600
VASLPPNLGS TSHKIPTTSE DIKDTEKMYP STNIAVTNVG
            6610 6620 6630 6640
TTTSEKESYS SVPAYSEPPK VTSPMVTSFN IRDTIVSTSM
            6650 6660 6670 6680
PGSSEITRIE MESTFSVAHG LKGTSTSQDP IVSTEKSAVL
            6690 6700 6710 6720
HKLTTGATET SRTEVASSRR TSIPGPDHST ESPDISTEVI
            6730 6740 6750 6760
PSLPISLGIT ESSNMTIITR TGPPLGSTSQ GTFTLDTPTT
            6770 6780 6790 6800
SSRAGTHSMA TQEFPHSEMT TVMNKDPEIL SWTIPPSIEK
            6810 6820 6830 6840
TSFSSSLMPS PAMTSPPVSS TLPKTIHTTP SPMTSLLTPS
            6850 6860 6870 6880
LVMTTDTLGT SPEPTTSSPP NLSSTSHVIL TTDEDTTAIE
            6890 6900 6910 6920
AMHPSTSTAA TNVETTCSGH GSQSSVLTDS EKTKATAPMD
            6930 6940 6950 6960
TTSTMGHTTV STSMSVSSET TKIKRESTYS LTPGLRETSI
            6970 6980 6990 7000
SQNASFSTDT SIVLSEVPTG TTAEVSRTEV TSSGRTSIPG
            7010 7020 7030 7040
PSQSTVLPEI STRTMTRLFA SPTMTESAEM TIPTQTGPSG
            7050 7060 7070 7080
STSQDTLTLD TSTTKSQAKT HSTLTQRFPH SEMTTLMSRG
            7090 7100 7110 7120
PGDMSWQSSP SLENPSSLPS LLSLPATTSP PPISSTLPVT
            7130 7140 7150 7160
ISSSPLPVTS LLTSSPVTTT DMLHTSPELV TSSPPKLSHT
            7170 7180 7190 7200
SDERLTTGKD TTNTEAVHPS TNTAASNVEI PSFGHESPSS
            7210 7220 7230 7240
ALADSETSKA TSPMFITSTQ EDTTVAISTP HFLETSRIQK
            7250 7260 7270 7280
ESISSLSPKL RETGSSVETS SAIETSAVLS EVSIGATTEI
            7290 7300 7310 7320
SRTEVTSSSR TSISGSAEST MLPEISTTRK IIKFPTSPIL
            7330 7340 7350 7360
AESSEMTIKT QTSPPGSTSE STFTLDTSTT PSLVITHSTM
            7370 7380 7390 7400
TQRLPHSEIT TLVSRGAGDV PRPSSLPVEE TSPPSSQLSL
            7410 7420 7430 7440
SAMISPSPVS STLPASSHSS SASVTSPLTP GQVKTTEVLD
            7450 7460 7470 7480
ASAEPETSSP PSLSSTSVEI LATSEVTTDT EKIHPFPNTA
            7490 7500 7510 7520
VTKVGTSSSG HESPSSVLPD SETTKATSAM GTISIMGDTS
            7530 7540 7550 7560
VSTLTPALSN TRKIQSEPAS SLTTRLRETS TSEETSLATE
            7570 7580 7590 7600
ANTVLSKVST GATTEVSRTE AISFSRTSMS GPEQSTMSQD
            7610 7620 7630 7640
ISIGTIPRIS ASSVLTESAK MTITTQTGPS ESTLESTLNL
            7650 7660 7670 7680
NTATTPSWVE THSIVIQGFP HPEMTTSMGR GPGGVSWPSP
            7690 7700 7710 7720
PFVKETSPPS SPLSLPAVTS PHPVSTTFLA HIPPSPLPVT
            7730 7740 7750 7760
SLLTSGPATT TDILGTSTEP GTSSSSSLST TSHERLTTYK
            7770 7780 7790 7800
DTAHTEAVHP STNTGGTNVA TTSSGYKSQS SVLADSSPMC
            7810 7820 7830 7840
TTSTMGDTSV LTSTPAFLET RRIQTELASS LTPGLRESSG
            7850 7860 7870 7880
SEGTSSGTKM STVLSKVPTG ATTEISKEDV TSIPGPAQST
            7890 7900 7910 7920
ISPDISTRTV SWFSTSPVMT ESAEITMNTH TSPLGATTQG
            7930 7940 7950 7960
TSTLATSSTT SLTMTHSTIS QGFSHSQMST LMRRGPEDVS
            7970 7980 7990 8000
WMSPPLLEKT RPSFSLMSSP ATTSPSPVSS TLPESISSSP
            8010 8020 8030 8040
LPVTSLLTSG LAKTTDMLHK SSEPVTNSPA NLSSTSVEIL
            8050 8060 8070 8080
ATSEVTTDTE KTHPSSNRTV TDVGTSSSGH ESTSFVLADS
            8090 8100 8110 8120
QTSKVTSPMV ITSTMEDTSV STSTPGFFET SRIQTEPTSS
            8130 8140 8150 8160
LTLGLRKTSS SEGTSLATEM STVLSGVPTG ATAEVSRTEV
            8170 8180 8190 8200
TSSSRTSISG FAQLTVSPET STETITRLPT SSIMTESAEM
            8210 8220 8230 8240
MIKTQTDPPG STPESTHTVD ISTTPNWVET HSTVTQRFSH
            8250 8260 8270 8280
SEMTTLVSRS PGDMLWPSQS SVEETSSASS LLSLPATTSP
            8290 8300 8310 8320
SPVSSTLVED FPSASLPVTS LLTPGLVITT DRMGISREPG
            8330 8340 8350 8360
TSSTSNLSST SHERLTTLED TVDTEDMQPS THTAVTNVRT
            8370 8380 8390 8400
SISGHESQSS VLSDSETPKA TSPMGTTYTM GETSVSISTS
            8410 8420 8430 8440
DFFETSRIQI EPTSSLTSGL RETSSSERIS SATEGSTVLS
            8450 8460 8470 8480
EVPSGATTEV SRTEVISSRG TSMSGPDQFT ISPDISTEAI
            8490 8500 8510 8520
TRLSTSPIMT ESAESAITIE TGSPGATSEG TLTLDTSTTT
            8530 8540 8550 8560
FWSGTHSTAS PGFSHSEMTT LMSRTPGDVP WPSLPSVEEA
            8570 8580 8590 8600
SSVSSSLSSP AMTSTSFFSA LPESISSSPH PVTALLTLGP
            8610 8620 8630 8640
VKTTDMLRTS SEPETSSPPN LSSTSAEILA TSEVTKDREK
            8650 8660 8670 8680
IHPSSNTPVV NVGTVIYKHL SPSSVLADLV TTKPTSPMAT
            8690 8700 8710 8720
TSTLGNTSVS TSTPAFPETM MTQPTSSLTS GLREISTSQE
            8730 8740 8750 8760
TSSATERSAS LSGMPTGATT KVSRTEALSL GRTSTPGPAQ
            8770 8780 8790 8800
STISPEISTE TITRISTPLT TTGSAEMTIT PKTGHSGASS
            8810 8820 8830 8840
QGTFTLDTSS RASWPGTHSA ATHRSPHSGM TTPMSRGPED
            8850 8860 8870 8880
VSWPSRPSVE KTSPPSSLVS LSAVTSPSPL YSTPSESSHS
            8890 8900 8910 8920
SPLRVTSLFT PVMMKTTDML DTSLEPVTTS PPSMNITSDE
            8930 8940 8950 8960
SLATSKATME TEAIQLSENT AVTQMGTISA RQEFYSSYPG
            8970 8980 8990 9000
LPEPSKVTSP VVTSSTIKDI VSTTIPASSE ITRIEMESTS
            9010 9020 9030 9040
TLTPTPRETS TSQEIHSATK PSTVPYKALT SATIEDSMTQ
            9050 9060 9070 9080
VMSSSRGPSP DQSTMSQDIS SEVITRLSTS PIKAESTEMT
            9090 9100 9110 9120
ITTQTGSPGA TSRGTLTLDT STTFMSGTHS TASQGFSHSQ
            9130 9140 9150 9160
MTALMSRTPG DVPWLSHPSV EEASSASFSL SSPVMTSSSP
            9170 9180 9190 9200
VSSTLPDSIH SSSLPVTSLL TSGLVKTTEL LGTSSEPETS
            9210 9220 9230 9240
SPPNLSSTSA EILATTEVTT DTEKLEMTNV VTSGYTHESP
            9250 9260 9270 9280
SSVLADSVTT KATSSMGITY PTGDTNVLTS TPAFSDTSRI
            9290 9300 9310 9320
QTKSKLSLTP GLMETSISEE TSSATEKSTV LSSVPTGATT
            9330 9340 9350 9360
EVSRTEAISS SRTSIPGPAQ STMSSDTSME TITRISTPLT
            9370 9380 9390 9400
RKESTDMAIT PKTGPSGATS QGTFTLDSSS TASWPGTHSA
            9410 9420 9430 9440
TTQRFPQSVV TTPMSRGPED VSWPSPLSVE KNSPPSSLVS
            9450 9460 9470 9480
SSSVTSPSPL YSTPSGSSHS SPVPVTSLFT SIMMKATDML
            9490 9500 9510 9520
DASLEPETTS APNMNITSDE SLATSKATTE TEAIHVFENT
            9530 9540 9550 9560
AASHVETTSA TEELYSSSPG FSEPTKVISP VVTSSSIRDN
            9570 9580 9590 9600
MVSTTMPGSS GITRIEIESM SSLTPGLRET RTSQDITSST
            9610 9620 9630 9640
ETSTVLYKMS SGATPEVSRT EVMPSSRTSI PGPAQSTMSL
            9650 9660 9670 9680
DISDEVVTRL STSPIMTESA EITITTQTGY SLATSQVTLP
            9690 9700 9710 9720
LGTSMTFLSG THSTMSQGLS HSEMTNLMSR GPESLSWTSP
            9730 9740 9750 9760
RFVETTRSSS SLTSLPLTTS LSPVSSTLLD SSPSSPLPVT
            9770 9780 9790 9800
SLILPGLVKT TEVLDTSSEP KTSSSPNLSS TSVEIPATSE
            9810 9820 9830 9840
IMTDTEKIHP SSNTAVAKVR TSSSVHESHS SVLADSETTI
            9850 9860 9870 9880
TIPSMGITSA VDDTTVFTSN PAFSETRRIP TEPTFSLTPG
            9890 9900 9910 9920
FRETSTSEET TSITETSAVL YGVPTSATTE VSMTEIMSSN
            9930 9940 9950 9960
RTHIPDSDQS TMSPDIITEV ITRLSSSSMM SESTQMTITT
            9970 9980 9990 10000
QKSSPGATAQ STLTLATTTA PLARTHSTVP PRFLHSEMTT
          10010 10020 10030 10040
LMSRSPENPS WKSSPFVEKT SSSSSLLSLP VTTSPSVSST
          10050 10060 10070 10080
LPQSIPSSSF SVTSLLTPGM VKTTDTSTEP GTSLSPNLSG
          10090 10100 10110 10120
TSVEILAASE VTTDTEKIHP SSSMAVTNVG TTSSGHELYS
          10130 10140 10150 10160
SVSIHSEPSK ATYPVGTPSS MAETSISTSM PANFETTGFE
          10170 10180 10190 10200
AEPFSHLTSG FRKTNMSLDT SSVTPTNTPS SPGSTHLLQS
          10210 10220 10230 10240
SKTDFTSSAK TSSPDWPPAS QYTEIPVDII TPFNASPSIT
          10250 10260 10270 10280
ESTGITSFPE SRFTMSVTES THHLSTDLLP SAETISTGTV
          10290 10300 10310 10320
MPSLSEAMTS FATTGVPRAI SGSGSPFSRT ESGPGDATLS
          10330 10340 10350 10360
TIAESLPSST PVPFSSSTFT TTDSSTIPAL HEITSSSATP
          10370 10380 10390 10400
YRVDTSLGTE SSTTEGRLVM VSTLDTSSQP GRTSSTPILD
          10410 10420 10430 10440
TRMTESVELG TVTSAYQVPS LSTRLTRTDG IMEHITKIPN
          10450 10460 10470 10480
EAAHRGTIRP VKGPQTSTSP ASPKGLHTGG TKRMETTTTA
          10490 10500 10510 10520
LKTTTTALKT TSRATLTTSV YTPTLGTLTP LNASRQMAST
          10530 10540 10550 10560
ILTEMMITTP YVFPDVPETT SSLATSLGAE TSTALPRTTP
          10570 10580 10590 10600
SVLNRESETT ASLVSRSGAE RSPVIQTLDV SSSEPDTTAS
          10610 10620 10630 10640
WVIHPAETIP TVSKTTPNFF HSELDTVSST ATSHGADVSS
          10650 10660 10670 10680
AIPTNISPSE LDALTPLVTI SGTDTSTTFP TLTKSPHETE
          10690 10700 10710 10720
TRTTWLTHPA ETSSTIPRTI PNFSHHESDA TPSIATSPGA
          10730 10740 10750 10760
ETSSAIPIMT VSPGAEDLVT SQVTSSGTDR NMTIPTLTLS
          10770 10780 10790 10800
PGEPKTIASL VTHPEAQTSS AIPTSTISPA VSRLVTSMVT
          10810 10820 10830 10840
SLAAKTSTTN RALTNSPGEP ATTVSLVTHP AQTSPTVPWT
          10850 10860 10870 10880
TSIFFHSKSD TTPSMTTSHG AESSSAVPTP TVSTEVPGVV
          10890 10900 10910 10920
TPLVTSSRAV ISTTIPILTL SPGEPETTPS MATSHGEEAS
          10930 10940 10950 10960
SAIPTPTVSP GVPGVVTSLV TSSRAVTSTT IPILTFSLGE
          10970 10980 10990 11000
PETTPSMATS HGTEAGSAVP TVLPEVPGMV TSLVASSRAV
          11010 11020 11030 11040
TSTTLPTLTL SPGEPETTPS MATSHGAEAS STVPTVSPEV
          11050 11060 11070 11080
PGVVTSLVTS SSGVNSTSIP TLILSPGELE TTPSMATSHG
          11090 11100 11110 11120
AEASSAVPTP TVSPGVSGVV TPLVTSSRAV TSTTIPILTL
          11130 11140 11150 11160
SSSEPETTPS MATSHGVEAS SAVLTVSPEV PGMVTSLVTS
          11170 11180 11190 11200
SRAVTSTTIP TLTISSDEPE TTTSLVTHSE AKMISAIPTL
          11210 11220 11230 11240
AVSPTVQGLV TSLVTSSGSE TSAFSNLTVA SSQPETIDSW
          11250 11260 11270 11280
VAHPGTEASS VVPTLTVSTG EPFTNISLVT HPAESSSTLP
          11290 11300 11310 11320
RTTSRFSHSE LDTMPSTVTS PEAESSSAIS TTISPGIPGV
          11330 11340 11350 11360
LTSLVTSSGR DISATFPTVP ESPHESEATA SWVTHPAVTS
          11370 11380 11390 11400
TTVPRTTPNY SHSEPDTTPS IATSPGAEAT SDFPTITVSP
          11410 11420 11430 11440
DVPDMVTSQV TSSGTDTSIT IPTLTLSSGE PETTTSFITY
          11450 11460 11470 11480
SETHTSSAIP TLPVSPGASK MLTSLVISSG TDSTTTFPTL
          11490 11500 11510 11520
TETPYEPETT AIQLIHPAET NTMVPKTTPK FSHSKSDTTL
          11530 11540 11550 11560
PVAITSPGPE ASSAVSTTTI SPDMSDLVTS LVPSSGTDTS
          11570 11580 11590 11600
TTFPTLSETP YEPETTVTWL THPAETSTTV SGTIPNFSHR
          11610 11620 11630 11640
GSDTAPSMVT SPGVDTRSGV PTTTIPPSIP GVVTSQVTSS
          11650 11660 11670 11680
ATDTSTAIPT LTPSPGEPET TASSATHPGT QTGFTVPIRT
          11690 11700 11710 11720
VPSSEPDTMA SWVTHPPQTS TPVSRTTSSF SHSSPDATPV
          11730 11740 11750 11760
MATSPRTEAS SAVLTTISPG APEMVTSQIT SSGAATSTTV
          11770 11780 11790 11800
PTLTHSPGMP ETTALLSTHP RTGTSKTFPA STVFPQVSET
          11810 11820 11830 11840
TASLTIRPGA ETSTALPTQT TSSLFTLLVT GTSRVDLSPT
          11850 11860 11870 11880
ASPGVSAKTA PLSTHPGTET STMIPTSTLS LGLLETTGLL
        11890 11900 11910 11920
ATSSSAETST STLTLTVSPA VSGLSSASIT TDKPQTVTSW
          11930 11940 11950 11960
NTETSPSVTS VGPPEFSRTV TGTTMTLIPS EMPTPPKTSH
          11970 11980 11990 12000
GEGVSPTTIL RTTMVEATNL ATTGSSPTVA KTTTTFNTLA
          12010 12020 12030 12040
GSLFTPLTTP GMSTLASESV TSRTSYNHRS WISTTSSYNR
          12050 12060 12070 12080
RYWTPATSTP VTSTFSPGIS TSSIPSSTAA TVPFMVPFTL
          12090 12100 12110 12120
NFTITNLQYE EDMRHPGSRK FNATERELQG LLKPLFRNSS
          12130 12140 12150 12160
LEYLYSGCRL ASLRPEKDSS AMAVDAICTH RPDPEDLGLD
          12170 12180 12190 12200
RERLYWELSN LTNGIQELGP YTLDRNSLYV NGFTHRSSMP
          12210 12220 12230 12240
TTSTPGTSTV DVGTSGTPSS SPSPTAAGPL LMPFTLNFTI
          12250 12260 12270 12280
TNLQYEEDMR RTGSRKFNTM ESVLQGLLKP LFKNTSVGPL
          12290 12300 12310 12320
YSGCRLTLLR PEKDGAATGV DAICTHRLDP KSPGLNREQL
          12330 12340 12350 12360
YWELSKLTND IEELGPYTLD RNSLYVNGFT HQSSVSTTST
          12370 12380 12390 12400
PGTSTVDLRT SGTPSSLSSP TIMAAGPLLV PFTLNFTITN
          12410 12420 12430 12440
LQYGEDMGHP GSRKFNTTER VLQGLLGPIF KNTSVGPLYS
          12450 12460 12470 12480
GCRLTSLRSE KDGAATGVDA ICIHHLDPKS PGLNRERLYW
          12490 12500 12510 12520
ELSQLTNGIK ELGPYTLDRN SLYVNGFTHR TSVPTTSTPG
          12530 12540 12550 12560
TSTVDLGTSG TPFSLPSPAT AGPLLVLFTL NFTITNLKYE
          12570 12580 12590 12600
EDMHRPGSRK FNTTERVLQT LLGPMFKNTS VGLLYSGCRL
          12610 12620 12630 12640
TLLRSEKDGA ATGVDAICTH RLDPKSPGLD REQLYWELSQ
          12650 12660 12670 12680
LTNGIKELGP YTLDRNSLYV NGFTHWIPVP TSSTPGTSTV
          12690 12700 12710 12720
DLGSGTPSSL PSPTAAGPLL VPFTLNFTIT NLQYEEDMHH
          12730 12740 12750 12760
PGSRKFNTTE RVLQGLLGPM FKNTSVGLLY SGCRLTLLRS
          12770 12780 12790 12800
EKDGAATGVD AICTHRLDPK SPGVDREQLY WELSQLTNGI
          12810 12820 12830 12840
KELGPYTLDR NSLYVNGFTH QTSAPNTSTP GTSTVDLGTS
          12850 12860 12870 12880
GTPSSLPSPT SAGPLLVPFT LNFTITNLQY EEDMRHPGSR
          12890 12900 12910 12920
KFNTTERVLQ GLLKPLFKST SVGPLYSGCR LTLLRSEKDG
          12930 12940 12950 12960
AATGVDAICT HRLDPKSPGV DREQLYWELS QLTNGIKELG
          12970 12980 12990 13000
PYTLDRNSLY VNGFTHQTSA PNTSTPGTST VDLGTSGTPS
          13010 13020 13030 13040
SLPSPTSAGP LLVPFTLNFT ITNLQYEEDM HHPGSRKFNT
          13050 13060 13070 13080
TERVLQGLLG PMFKNTSVGL LYSGCRLTLL RPEKNGAATG
          13090 13100 13110 13120
MDAICSHRLD PKSPGLNREQ LYWELSQLTH GIKELGPYTL
          13130 13140 13150 13160
DRNSLYVNGF THRSSVAPTS TPGTSTVDLG TSGTPSSLPS
          13170 13180 13190 13200
PTTAVPLLVP FTLNFTITNL QYGEDMRHPG SRKFNTTERV
          13210 13220 13230 13240
LQGLLGPLFK NSSVGPLYSG CRLISLRSEK DGAATGVDAI
          13250 13260 13270 13280
CTHHLNPQSP GLDREQLYWQ LSQMTNGIKE LGPYTLDRNS
          13290 13300 13310 13320
LYVNGFTHRS SGLTTSTPWT STVDLGTSGT PSPVPSPTTA
          13330 13340 13350 13360
GPLLVPFTLN FTITNLQYEE DMHRPGSRKF NTTERVLQGL
          13370 13380 13390 13400
LSPIFKNSSV GPLYSGCRLT SLRPEKDGAA TGMDAVCLYH
          13410 13420 13430 13440
PNPKRPGLDR EQLYWELSQL THNITELGPY SLDRDSLYVN
          13450 13460 13470 13480
GFTHQNSVPT TSTPGTSTVY WATTGTPSSF PGHTEPGPLL
          13490 13500 13510 13520
IPFTFNFTIT NLHYEENMQH PGSRKFNTTE RVLQGLLKPL
          13530 13540 13550 13560
FKNTSVGPLY SGCRLTSLRP EKDGAATGMD AVCLYHPNPK
          13570 13580 13590 13600
RPGLDREQLY WELSQLTHNI TELGPYSLDR DSLYVNGFTH
          13610 13620 13630 13640
QNSVPTTSTP GTSTVYWATT GTPSSFPGHT EPGPLLIPFT
          13650 13660 13670 13680
FNFTITNLHY EENMQHPGSR KFNTTERVLQ GLLKPLFKNT
          13690 13700 13710 13720
SVGPLYSGCR LTLLRPEKHE AATGVDTICT HRVDPIGPGL
          13730 13740 13750 13760
DRERLYWELS QLTNSITELG PYTLDRDSLY VNGFNPRSSV
          13770 13780 13790 13800
PTTSTPGTST VHLATSGTPS SLPGHTAPVP LLIPFTLNFT
          13810 13820 13830 13840
ITNLHYEENM QHPGSRKFNT TERVLQGLLK PLFKNTSVGP
          13850 13860 13870 13880
LYSGCRLTLL RPEKHEAATG VDTICTHRVD PIGPGLXXEX
          13890 13900 13910 13920
LYWELSXLTX XIXELGPYTL DRXSLYVNGF THXXSXPTTS
          13930 13940 13950 13960
TPGTSTVXXG TSGTPSSXPX XTSAGPLLVP FTLNFTITNL
          13970 13980 13990 14000
QYEEDMHHPG SRKFNTTERV LQGLLGPMFK NTSVGLLYSG
          14010 14020 14030 14040
CRLTLLRPEK NGAATGMDAI CSHRLDPKSP GLDREQLYWE
          14050 14060 14070 14080
LSQLTHGIKE LGPYTLDRNS LYVNGFTHRS SVAPTSTPGT
          14090 14100 14110 14120
STVDLGTSGT PSSLPSPTTA VPLLVPFTLN FTITNLQYGE
          14130 14140 14150 14160
DMRHPGSRKF NTTERVLQGL LGPLFKNSSV GPLYSGCRLI
          14170 14180 14190 14200
SLRSEKDGAA TGVDAICTHH LNPQSPGLDR EQLYWQLSQM
          14210 14220 14230 14240
TNGIKELGPY TLDRNSLYVN GFTHRSSGLT TSTPWTSTVD
          14250 14260 14270 14280
LGTSGTPSPV PSPTTAGPLL VPFTLNFTIT NLQYEEDMHR
          14290 14300 14310 14320
PGSRKFNATE RVLQGLLSPI FKNSSVGPLY SGCRLTSLRP
          14330 14340 14350 14360
EKDGAATGMD AVCLYHPNPK RPGLDREQLY WELSQLTHNI
          14370 14380 14390 14400
TELGPYSLDR DSLYVNGFTH QSSMTTTRTP DTSTMHLATS
          14410 14420 14430 14440
RTPASLSGPT TASPLLVLFT INCTITNLQY EEDMRRTGSR
          14450 14460 14470 14480
KFNTMESVLQ GLLKPLFKNT SVGPLYSGCR LTLLRPKKDG
          14490 14500 14510 14520
AATGVDAICT HRLDPKSPGL NREQLYWELS KLTNDIEELG
          14530 14540 14550 14560
PYTLDRNSLY VNGFTHQSSV STTSTPGTST VDLRTSGTPS
          14570 14580 14590 14600
SLSSPTIMXX XPLLXPFTXN XTITNLXXXX XMXXPGSRKF
          14610 14620 14630 14640
NTTERVLQGL LRPLFKNTSV SSLYSGCRLT LLRPEKDGAA
          14650 14660 14670 14680
TRVDAACTYR PDPKSPGLDR EQLYWELSQL THSITELGPY
          14690 14700 14710 14720
TLDRVSLYVN GFNPRSSVPT TSTPGTSTVH LATSGTPSSL
          14730 14740 14750 14760
PGHTXXXPLL XPFTXNXTIT NLXXXXXMXX PGSRKFNTTE
          14770 14780 14790 14800
RVLQGLLKPL FRNSSLEYLY SGCRLASLRP EKDSSAMAVD
          14810 14820 14830 14840
AICTHRPDPE DLGLDRERLY WELSNLTNGI QELGPYTLDR
          14850 14860 14870 14880
NSLYVNGFTH RSSGLTTSTP WTSTVDLGTS GTPSPVPSPT
          14890 14900 14910 14920
TAGPLLVPFT LNFTITNLQY EEDMHRPGSR RFNTTERVLQ
          14930 14940 14950 14960
GLLTPLFKNT SVGPLYSGCR LTLLRPEKQE AATGVDTICT
          14970 14980 14990 15000
HRVDPIGPGL DRERLYWELS QLTNSITELG PYTLDRDSLY
          15010 15020 15030 15040
VNGFNPWSSV PTTSTPGTST VHLATSGTPS SLPGHTAPVP
          15050 15060 15070 15080
LLIPFTLNFT ITDLHYEENM QHPGSRKFNT TERVLQGLLK
          15090 15100 15110 15120
PLFKSTSVGP LYSGCRLTLL RPEKHGAATG VDAICTLRLD
          15130 15140 15150 15160
PTGPGLDRER LYWELSQLTN SVTELGPYTL DRDSLYVNGF
          15170 15180 15190 15200
THRSSVPTTS IPGTSAVHLE TSGTPASLPG HTAPGPLLVP
          15210 15220 15230 15240
FTLNFTITNL QYEEDMRHPG SRKFSTTERV LQGLLKPLFK
          15250 15260 15270 15280
NTSVSSLYSG CRLTLLRPEK DGAATRVDAV CTHRPDPKSP
          15290 15300 15310 15320
GLDRERLYWK LSQLTHGITE LGPYTLDRHS LYVNGFTHQS
          15330 15340 15350 15360
SMTTTRTPDT STMHLATSRT PASLSGPTTA SPLLVLFTIN
          15370 15380 15390 15400
FTITNLRYEE NMHHPGSRKF NTTERVLQGL LRPVFKNTSV
          15410 15420 15430 15440
GPLYSGCRLT TLRPKKDGAA TKVDAICTYR PDPKSPGLDR
          15450 15460 15470 15480
EQLYWELSQL THSITELGPY TQDRDSLYVN GFTHRSSVPT
          15490 15500 15510 15520
TSIPGTSAVH LETSGTPASL PGHTAPGPLL VPFTLNFTIT
          15530 15540 15550 15560
NLQYEEDMRH PGSRKFNTTE RVLQGLLKPL FKSTSVGPLY
          15570 15580 15590 15600
SGCRLTLLRP EKRGAATGVD TICTHRLDPL NPGLDREQLY
          15610 15620 15630 15640
WELSKLTRGI IELGPYLLDR GSLYVNGFTH RTSVPTTSTP
          15650 15660 15670 15680
GTSTVDLGTS GTPFSLPSPA XXXPLLXPFT XNXTITNLXX
          15690 15700 15710 15720
XXXMXXPGSR KFNTTERVLQ TLLGPMFKNT SVGLLYSGCR
          15730 15740 15750 15760
LTLLRSEKDG AATGVDAICT HRLDPKSPGV DREQLYWELS
          15770 15780 15790 15800
QLTNGIKELG PYTLDRNSLY VNGFTHWIPV PTSSTPGTST
          15810 15820 15830 15840
VDLGSGTPSS LPSPTTAGPL LVPFTLNFTI TNLKYEEDMH
          15850 15860 15870 15880
CPGSRKFNTT ERVLQSLLGP MFKNTSVGPL YSGCRLTLLR
          15890 15900 15910 15920
SEKDGAATGV DAICTHRLDP KSPGVDREQL YWELSQLTNG
          15930 15940 15950 15960
IKELGPYTLD RNSLYVNGFT HQTSAPNTST PGTSTVDLGT
          15970 15980 15990 16000
SGTPSSLPSP TXXXPLLXPF TXNXTITNLX XXXXMXXPGS
          16010 16020 16030 16040
RKFNTTEXVL QGLLXPXFKN XSVGXLYSGC RLTXLRXEKX
          16050 16060 16070 16080
GAATGXDAIC XHXXXPKXPG LXXEXLYWEL SXLTXXIXEL
          16090 16100 16110 16120
GPYTLDRXSL YVNGFTHWIP VPTSSTPGTS TVDLGSGTPS
          16130 16140 16150 16160
SLPSPTTAGP LLVPFTLNFT ITNLKYEEDM HCPGSRKFNT
          16170 16180 16190 16200
TERVLQSLLG PMFKNTSVGP LYSGCRLTSL RSEKDGAATG
          16210 16220 16230 16240
VDAICTHRVD PKSPGVDREQ LYWELSQLTN GIKELGPYTL
          16250 16260 16270 16280
DRNSLYVNGF THQTSAPNTS TPGTSTVXXG TSGTPSSXPX
          16290 16300 16310 16320
XTSAGPLLVP FTLNFTITNL QYEEDMHHPG SRKFNTTERV
          16330 16340 16350 16360
LQGLLGPMFK NTSVGLLYSG CRLTLLRPEK NGATTGMDAI
          16370 16380 16390 16400
CTHRLDPKSP GLXXEXLYWE LSXLTXXIXE LGPYTLDRXS
          16410 16420 16430 16440
LYVNGFTHXX SXPTTSTPGT STVXXGTSGT PSSXPXXTXX
          16450 16460 16470 16480
XPLLXPFTXN XTITNLXXXX XMXXPGSRKF NTTERVLQGL
          16490 16500 16510 16520
LKPLFRNSSL EYLYSGCRLA SLRPEKDSSA MAVDAICTHR
          16530 16540 16550 16560
PDPEDLGLDR ERLYWELSNL TNGIQELGPY TLDRNSLYVN
          16570 16580 16590 16600
GFTHRSSMPT TSTPGTSTVD VGTSGTPSSS PSPTTAGPLL
          16610 16620 16630 16640
IPFTLNFTIT NLQYGEDMGH PGSRKFNTTE RVLQGLLGPI
          16650 16660 16670 16680
FKNTSVGPLY SGCRLTSLRS EKDGAATGVD AICIHHLDPK
          16690 16700 16710 16720
SPGLNRERLY WELSQLTNGI KELGPYTLDR NSLYVNGFTH
          16730 16740 16750 16760
RTSVPTTSTP GTSTVDLGTS GTPFSLPSPA TAGPLLVLFT
          16770 16780 16790 16800
LNFTITNLKY EEDMHRPGSR KFNTTERVLQ TLLGPMFKNT
          16810 16820 16830 16840
SVGLLYSGCR LTLLRSEKDG AATGVDAICT HRLDPKSPGL
          16850 16860 16870 16880
XXEXLYWELS XLTXXIXELG PYTLDRXSLY VNGFTHXXSX
          16890 16900 16910 16920
PTTSTPGTST VXXGTSGTPS SXPXXTXXXP LLXPFTXNXT
          16930 16940 16950 16960
ITNLXXXXXM XXPGSRKFNT TERVLQGLLR PVFKNTSVGP
          16970 16980 16990 17000
LYSGCRLTLL RPKKDGAATK VDAICTYRPD PKSPGLDREQ
          17010 17020 17030 17040
LYWELSQLTH SITELGPYTQ DRDSLYVNGF THRSSVPTTS
          17050 17060 17070 17080
IPGTSAVHLE TTGTPSSFPG HTEPGPLLIP FTFNFTITNL
          17090 17100 17110 17120
RYEENMQHPG SRKFNTTERV LQGLLTPLFK NTSVGPLYSG
          17130 17140 17150 17160
CRLTLLRPEK QEAATGVDTI CTHRVDPIGP GLDRERLYWE
          17170 17180 17190 17200
LSQLTNSITE LGPYTLDRDS LYVDGFNPWS SVPTTSTPGT
          17210 17220 17230 17240
STVHLATSGT PSPLPGHTAP VPLLIPFTLN FTITDLHYEE
          17250 17260 17270 17280
NMQHPGSRKF NTTERVLQGL LKPLFKSTSV GPLYSGCRLT
          17290 17300 17310 17320
LLRPEKHGAA TGVDAICTLR LDPTGPGLDR ERLYWELSQL
          17330 17340 17350 17360
TNSITELGPY TLDRDSLYVN GFNPWSSVPT TSTPGTSTVH
          17370 17380 17390 17400
LATSGTPSSL PGHTTAGPLL VPFTLNFTIT NLKYEEDMHC
          17410 17420 17430 17440
PGSRKFNTTE RVLQSLHGPM FKNTSVGPLY SGCRLTLLRS
          17450 17460 17470 17480
EKDGAATGVD AICTHRLDPK SPGLXXEXLY WELSXLTXXI
          17490 17500 17510 17520
XELGPYTLDR XSLYVNGFTH XXSXPTTSTP GTSTVXXGTS
          17530 17540 17550 17560
GTPSSXPXXT XXXPLLXPFT XNXTITNLXX XXXMXXPGSR
          17570 17580 17590 17600
KFNTTEXVLQ GLLXPXFKNX SVGXLYSGCR LTXLRXEKXG
          17610 17620 17630 17640
AATGXDAICX HXXXPKXPGL XXEXLYWELS XLTNSITELG
          17650 17660 17670 17680
PYTLDRDSLY VNGFTHRSSM PTTSIPGTSA VHLETSGTPA
          17690 17700 17710 17720
SLPGHTAPGP LLVPFTLNFT ITNLQYEEDM RHPGSRKFNT
          17730 17740 17750 17760
TERVLQGLLK PLFKSTSVGP LYSGCRLTLL RPEKRGAATG
          17770 17780 17790 17800
VDTICTHRLD PLNPGLXXEX LYWELSXLTX XIXELGPYTL
          17810 17820 17830 17840
DRXSLYVNGF THXXSXPTTS TPGTSTVXXG TSGTPSSXPX
          17850 17860 17870 17880
XTXXXPLLXP FTXNXTITNL XXXXXMXXPG SRKFNTTEXV
          17890 17900 17910 17920
LQGLLXPXFK NXSVGXLYSG CRLTXLRXEK XGAATGXDAI
          17930 17940 17950 17960
CXHXXXPKXP GLXXEXLYWE LSXLTXXIXE LGPYTLDRXS
          17970 17980 17990 18000
LYVNGFHPRS SVPTTSTPGT STVHLATSGT PSSLPGHTAP
          18010 18020 18030 18040
VPLLIPFTLN FTITNLHYEE NMQHPGSRKF NTTERVLQGL
          18050 18060 18070 18080
LGPMFKNTSV GLLYSGCRLT LLRPEKNGAA TGMDAICSHR
          18090 18100 18110 18120
LDPKSPGLXX EXLYWELSXL TXXIXELGPY TLDRXSLYVN
          18130 18140 18150 18160
GFTHXXSXPT TSTPGTSTVX XGTSGTPSSX PXXTXXXPLL
          18170 18180 18190 18200
XPFTXNXTIT NLXXXXXMXX PGSRKFNTTE XVLQGLLXPX
          18210 18220 18230 18240
FKNXSVGXLY SGCRLTXLRX EKXGAATGXD AICXHXXXPK
          18250 18260 18270 18280
XPGLXXEXLY WELSXLTXXI XELGPYTLDR XSLYVNGFTH
          18290 18300 18310 18320
QNSVPTTSTP GTSTVYWATT GTPSSFPGHT EPGPLLIPFT
          18330 18340 18350 18360
FNFTITNLHY EENMQHPGSR KFNTTERVLQ GLLTPLFKNT
          18370 18380 18390 18400
SVGPLYSGCR LTLLRPEKQE AATGVDTICT HRVDPIGPGL
          18410 18420 18430 18440
XXEXLYWELS XLTXXIXELG PYTLDRXSLY VNGFTHXXSX
          18450 18460 18470 18480
PTTSTPGTST VXXGTSGTPS SXPXXTXXXP LLXPFTXNXT
          18490 18500 18510 18520
ITNLXXXXXM XXPGSRKFNT TEXVLQGLLX PXFKNXSVGX
          18530 18540 18550 18560
LYSGCRLTXL RXEKXGAATG XDAICXHXXX PKXPGLXXEX
          18570 18580 18590 18600
LYWELSXLTX XIXELGPYTL DRXSLYVNGF THRSSVPTTS
          18610 18620 18630 18640
SPGTSTVHLA TSGTPSSLPG HTAPVPLLIP FTLNFTITNL
          18650 18660 18670 18680
HYEENMQHPG SRKFNTTERV LQGLLKPLFK STSVGPLYSG
          18690 18700 18710 18720
CRLTLLRPEK HGAATGVDAI CTLRLDPTGP GLXXEXLYWE
          18730 18740 18750 18760
LSXLTXXIXE LGPYTLDRXS LYVNGFTHXX SXPTTSTPGT
          18770 18780 18790 18800
STVXXGTSGT PSSXPXXTXX XPLLXPFTXN XTITNLXXXX
          18810 18820 18830 18840
XMXXPGSRKF NTTEXVLQGL LXPXFKNXSV GXLYSGCRLT
          18850 18860 18870 18880
XLRXEKXGAA TGXDAICXHX XXPKXPGLXX EXLYWELSXL
          18890 18900 18910 18920
TXXIXELGPY TLDRXSLYVN GFTHRTSVPT TSTPGTSTVH
          18930 18940 18950 18960
LATSGTPSSL PGHTAPVPLL IPFTLNFTIT NLQYEEDMHR
          18970 18980 18990 19000
PGSRKFNTTE RVLQGLLSPI FKNSSVGPLY SGCRLTSLRP
          19010 19020 19030 19040
EKDGAATGMD AVCLYHPNPK RPGLDREQLY CELSQLTHNI
          19050 19060 19070 19080
TELGPYSLDR DSLYVNGFTH QNSVPTTSTP GTSTVYWATT
          19090 19100 19110 19120
GTPSSFPGHT XXXPLLXPFT XNXTITNLXX XXXMXXPGSR
          19130 19140 19150 19160
KFNTTEXVLQ GLLXPXFKNX SVGXLYSGCR LTXLRXEKXG
          19170 19180 19190 19200
AATGXDAICX HXXXPKXPGL XXEXLYWELS XLTXXIXELG
          19210 19220 19230 19240
PYTLDRXSLY VNGFTHWSSG LTTSTPWTST VDLGTSGTPS
          19250 19260 19270 19280
PVPSPTTAGP LLVPFTLNFT ITNLQYEEDM HRPGSRKFNA
          19290 19300 19310 19320
TERVLQGLLS PIFKNTSVGP LYSGCRLTLL RPEKQEAATG
          19330 19340 19350 19360
VDTICTHRVD PIGPGLXXEX LYWELSXLTX XIXELGPYTL
          19370 19380 19390 19400
DRXSLYVNGF THXXSXPTTS TPGTSTVXXG TSGTPSSXPX
          19410 19420 19430 19440
XTXXXPLLXP FTXNXTITNL XXXXXMXXPG SRKFNTTEXV
          19450 19460 19470 19480
LQGLLXPXFK NXSVGXLYSG CRLTXLRXEK XGAATGXDAI
          19490 19500 19510 19520
CXHXXXPKXP GLXXEXLYWE LSXLTXXIXE LGPYTLDRXS
          19530 19540 19550 19560
LYVNGFTHRS FGLTTSTPWT STVDLGTSGT PSPVPSPTTA
          19570 19580 19590 19600
GPLLVPFTLN FTITNLQYEE DMHRPGSRKF NTTERVLQGL
          19610 19620 19630 19640
LTPLFRNTSV SSLYSGCRLT LLRPEKDGAA TRVDAVCTHR
          19650 19660 19670 19680
PDPKSPGLXX EXLYWELSXL TXXIXELGPY TLDRXSLYVN
          19690 19700 19710 19720
GFTHXXSXPT TSTPGTSTVX XGTSGTPSSX PXXTXXXPLL
          19730 19740 19750 19760
XPFTXNXTIT NLXXXXXMXX PGSRKFNTTE XVLQGLLXPX
          19770 19780 19790 19800
FKNXSVGXLY SGCRLTXLRX EKXGAATGXD AICXHXXXPK
          19810 19820 19830 19840
XPGLXXEXLY WELSXLTXXI XELGPYTLDR XSLYVNGFTH
          19850 19860 19870 19880
WIPVPTSSTP GTSTVDLGSG TPSSLPSPTT AGPLLVPFTL
          19890 19900 19910 19920
NFTITNLQYG EDMGHPGSRK FNTTERVLQG LLGPIFKNTS
          19930 19940 19950 19960
VGPLYSGCRL TSLRSEKDGA ATGVDAICIH HLDPKSPGLX
          19970 19980 19990 20000
XEXLYWELSX LTXXIXELGP YTLDRXSLYV NGFTHXXSXP
          20010 20020 20030 20040
TTSTPGTSTV XXGTSGTPSS XPXXTXXXPL LXPFTXNXTI
          20050 20060 20070 20080
TNLXXXXXMX XPGSRKFNTT EXVLQGLLXP XFKNXSVGXL
          20090 20100 20110 20120
YSGCRLTXLR XEKXGAATGX DAICXHXXXP KXPGLXXEXL
          20130 20140 20150 20160
YWELSXLTXX IXELGPYTLD RXSLYVNGFT HQTFAPNTST
          20170 20180 20190 20200
PGTSTVDLGT SGTPSSLPSP TSAGPLLVPF TLNFTITNLQ
          20210 20220 20230 20240
YEEDMHHPGS RKFNTTERVL QGLLGPMFKN TSVGLLYSGC
          20250 20260 20270 20280
RLTLLRPEKN GAATRVDAVC THRPDPKSPG LXXEXLYWEL
          20290 20300 20310 20320
SXLTXXIXEL GPYTLDRXSL YVNGFTHXXS XPTTSTPGTS
          20330 20340 20350 20360
TVXXGTSGTP SSXPXXTAPV PLLIPFTLNF TITNLHYEEN
          20370 20380 20390 20400
MQHPGSRKFN TTERVLQGLL KPLFKSTSVG PLYSGCRLTL
          20410 20420 20430 20440
LRPEKHGAAT GVDAICTLRL DPTGPGLDRE RLYWELSQLT
          20450 20460 20470 20480
NSVTELGPYT LDRDSLYVNG FTQRSSVPTT SIPGTSAVHL
          20490 20500 20510 20520
ETSGTPASLP GHTAPGPLLV PFTLNFTITN LQYEVDMRHP
          20530 20540 20550 20560
GSRKFNTTER VLQGLLKPLF KSTSVGPLYS GCRLTLLRPE
          20570 20580 20590 20600
KRGAATGVDT ICTHRLDPLN PGLDREQLYW ELSKLTRGII
          20610 20620 20630 20640
ELGPYLLDRG SLYVNGFTHR NFVPITSTPG TSTVHLGTSE
          20650 20660 20670 20680
TPSSLPRPIV PGPLLVPFTL NFTITNLQYE EAMRHPGSRK
          20690 20700 20710 20720
FNTTERVLQG LLRPLFKNTS IGPLYSSCRL TLLRPEKDKA
          20730 20740 20750 20760
ATRVDAICTH HPDPQSPGLN REQLYWELSQ LTHGITELGP
          20770 20780 20790 20800
YTLDRDSLYV DGFTHWSPIP TTSTPGTSIV NLGTSGIPPS
          20810 20820 20830 20840
LPETTXXXPL LXPFTXNXTI TNLXXXXXMX XPGSRKFNTT
          20850 20860 20870 20880
ERVLQGLLKP LFKSTSVGPL YSGCRLTLLR PEKDGVATRV
          20890 20900 20910 20920
DAICTHRPDP KIPGLDRQQL YWELSQLTHS ITELGPYTLD
          20930 20940 20950 20960
RDSLYVNGFT QRSSVPTTST PGTFTVQPET SETPSSLPGP
          20970 20980 20990 21000
TATGPVLLPF TLNFTITNLQ YEEDMHRPGS RKFNTTERVL
          21010 21020 21030 21040
QGLLMPLFKN TSVSSLYSGC RLTLLRPEKD GAATRVDAVC
          21050 21060 21070 21080
THRPDPKSPG LDRERLYWKL SQLTHGITEL GPYTLDRHSL
          21090 21100 21110 21120
YVNGFTHQSS MTTTRTPDTS TMHLATSRTP ASLSGPTTAS
          21130 21140 21150 21160
PLLVLFTINF TITNLRYEEN MHHPGSRKFN TTERVLQGLL
          21170 21180 21190 21200
RPVFKNTSVG PLYSGCRLTL LRPKKDGAAT KVDAICTYRP
          21210 21220 21230 21240
DPKSPGLDRE QLYWELSQLT HSITELGPYT LDRDSLYVNG
          21250 21260 21270 21280
FTQRSSVPTT SIPGTPTVDL GTSGTPVSKP GPSAASPLLV
          21290 21300 21310 21320
LFTLNFTITN LRYEENMQHP GSRKFNTTER VLQGLLRSLF
          21330 21340 21350 21360
KSTSVGPLYS GCRLTLLRPE KDGTATGVDA ICTHHPDPKS
          21370 21380 21390 21400
PRLDREQLYW ELSQLTHNIT ELGHYALDND SLFVNGFTHR
          21410 21420 21430 21440
SSVSTTSTPG TPTVYLGASK TPASIFGPSA ASHLLILFTL
          21450 21460 21470 21480
NFTITNLRYE ENMWPGSRKF NTTERVLQGL LRPLFKNTSV
          21490 21500 21510 21520
GPLYSGSRLT LLRPEKDGEA TGVDAICTHR PDPTGPGLDR
          21530 21540 21550 21560
EQLYLELSQL THSITELGPY TLDRDSLYVN GFTHRSSVPT
          21570 21580 21590 21600
TSTGVVSEEP FTLNFTINNL RYMADMGQPG SLKFNITDNV
          21610 21620 21630 21640
MKHLLSPLFQ RSSLGARYTG CRVIALRSVK NGAETRVDLL
          21650 21660 21670 21680
CTYLQPLSGP GLPIKQVFHE LSQQTHGITR LGPYSLDKDS
          21690 21700 21710 21720
LYLNGYNEPG LDEPPTTPKP ATTFLPPLSE ATTAMGYHLK
          21730 21740 21750 21760
TLTLNFTISN LQYSPDMGKG SATFNSTEGV LQHLLRPLFQ
          21770 21780 21790 21800
KSSMGPFYLG CQLISLRPEK DGAATGVDTT CTYHPDPVGP
          21810 21820 21830 21840
GLDIQQLYWE LSQLTHGVTQ LGFYVLDRDS LFINGYAPQN
          21850 21860 21870 21880
LSIRGEYQIN FHIVNWNLSN PDPTSSEYIT LLRDIQDKVT
          21890 21900 21910 21920
TLYKGSQLHD TFRFCLVTNL TMDSVLVTVK ALFSSNLDPS
          21930 21940 21950 21960
LVEQVFLDKT LNASFHWLGS TYQLVDIHVT EMESSVYQPT
          21970 21980 21990 22000
SSSSTQHFYL NFTITNLPYS QDKAQPGTTN YQRNKRNIED
          22010 22020 22030 22040
ALNQLFRNSS IKSYFSDCQV STFRSVPNRH HTGVDSLCNF
          22050 22060 22070 22080
SPLARRVDRV AIYEEFLRMT RNGTQLQNFT LDRSSVLVDG
          22090 22100 22110 22120
YSPNRNEPLT GNSDLPFWAV ILIGLAGLLG LITCLICGVL
          22130 22140 22150
VTTRRRKKEG EYNVQQQCPG YYQSHLDLED LQ
```

Most preferably, the Mucin-16 assay detects one or more soluble forms of Mucin-16. Mucin-16 is a single-pass type I membrane protein having a large extracellular domain, most or all of which is present in soluble forms of Mucin-16 generated either through alternative splicing event which deletes all or a portion of the transmembrane domain, or by proteolysis of the membrane-bound form. In the case of an immunoassay, one or more antibodies that bind to epitopes within this extracellular domain may be used to detect these soluble form(s). The following domains have been identified in Mucin-16:

| Residues | Length | Domain ID |
|---|---|---|
| 1-22152 | 22152 | Mucin-16 |
| 1-22096 | 22906 | extracellular |
| 22907-22117 | 21 | transmembrane |
| 22128-22152 | 35 | cytoplasmic |

As used herein, the term “Carcinoembryonic antigen-related cell adhesion molecule 5” refers to one or polypeptides present in a biological sample that are derived from the Carcinoembryonic antigen-related cell adhesion molecule 5 precursor (Swiss-Prot P06731 (SEQ ID NO: 13)).

```
                10 20 30 40
MESPSAPPHR WCIPWQRLLL TASLLTFWNP PTTAKLTIES
                50 60 70 80
TPFNVAEGKE VLLLVHNLPQ HLFGYSWYKG ERVDGNRQII
                90 100 110 120
GYVIGTQQAT PGPAYSGREI IYPNASLLIQ NIIQNDTGFY
              130 140 150 160
TLHVIKSDLV NEEATGQFRV YPELPKPSIS SNNSKPVEDK
              170 180 190 200
DAVAFTCEPE TQDATYLWWV NNQSLPVSPR LQLSNGNRTL
              210 220 230 240
TLFNVTRNDT ASYKCETQNP VSARRSDSVI LNVLYGPDAP
              250 260 270 280
TISPLNTSYR SGENLNLSCH AASNPPAQYS WFVNGTFQQS
              290 300 310 320
TQELFIPNIT VNNSGSYTCQ AHNSDTGLNR TTVTTITVYA
              330 340 350 360
EPPKPFITSN NSNPVEDEDA VALTCEPEIQ NTTYLWWVNN
              370 380 390 400
QSLPVSPRLQ LSNDNRTLTL LSVTRNDVGP YECGIQNELS
              410 420 430 440
VDHSDPVILN VLYGPDDPTI SPSYTYYRPG VNLSLSCHAA
              450 460 470 480
SNPPAQYSWL IDGNIQQHTQ ELFISNITEK NSGLYTCQAN
              490 500 510 520
NSASGHSRTT VKTITVSAEL PKPSISSNNS KPVEDKDAVA
              530 540 550 560
FTCEPEAQNT TYLWWVNGQS LPVSPRLQLS NGNRTLTLFN
              570 580 590 600
VTRNDARAYV CGIQNSVSAN RSDPVTLDVL YGPDTPIISP
              610 620 630 640
PDSSYLSGAN LNLSCHSASN PSPQYSWRIN GIPQQHTQVL
              650 660 670 680
FIAKITPNNN GTYACFVSNL ATGRNNSIVK SITVSASGTS
              690 700
PGLSAGATVG IMIGVLVGVA LI
```

The following domains have been identified in Carcinoembryonic antigen-related cell adhesion molecule 5:

| Residues | Length | Domain ID |
|---|---|---|
| 1-34 | 34 | Signal sequence |
| 35-685 | 5 | Carcinoembryonic antigen-related |
| | | cell adhesion molecule 5 |
| 686-702 | 17 | Propeptide |

As used herein, the term “cellular tumor antigen p53” refers to one or more polypeptides present in a biological sample that are derived from the cellular tumor antigen p53 precursor (Swiss-Prot P04637 (SEQ ID NO: 14)).

```
                10 20 30 40
MEEPQSDPSV EPPLSQETFS DLWKLLPENN VLSPLPSQAM
                50 60 70 80
DDLMLSPDDI EQWFTEDPGP DEAPRMPEAA PRVAPAPAAP
                90 100 110 120
TPAAPAPAPS WPLSSSVPSQ KTYQGSYGFR LGFLHSGTAK
              130 140 150 160
SVTCTYSPAL NKMFCQLAKT CPVQLWVDST PPPGTRVRAM
              170 180 190 200
AIYKQSQHMT EVVRRCPHHE RCSDSDGLAP PQHLIRVEGN
              210 220 230 240
LRVEYLDDRN TFRHSVVVPY EPPEVGSDCT TIHYNYMCNS
              250 260 270 280
SCMGGMNRRP ILTIITLEDS SGNLLGRNSF EVRVCACPGR
              290 300 310 320
DRRTEEENLR KKGEPHHELP PGSTKRALPN NTSSSPQPKK
              330 340 350 360
KPLDGEYFTL QIRGRERFEM FRELNEALEL KDAQAGKEPG
              370 380 390
GSRAHSSHLK SKKGQSTSRH KKLMFKTEGP DSD
```

Isoform 2 of cellular tumor antigen p53 has the following changes from this isoform 1 sequence:

332-341:
IRGRERFEMF (SEQ ID NO: 15) →
DGTSFQKENC (SEQ ID NO: 16)
342-393:
Missing

As used herein, the term “relating a signal to the presence or amount” of an analyte reflects this understanding. Assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Because an antibody epitope is on the order of 8 amino acids, an immunoassay configured to detect a marker of interest will also detect polypeptides related to the marker sequence, so long as those polypeptides contain the epitope(s) necessary to bind to the antibody or antibodies used in the assay. The term “related marker” as used herein with regard to a biomarker such as one of the kidney injury markers described herein refers to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

In this regard, the skilled artisan will understand that the signals obtained from an immunoassay are a direct result of complexes formed between one or more antibodies and the target biomolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. Expression of biomarkers may also be determined by means other than immunoassays, including protein measurements (such as dot blots, western blots, chromatographic methods, mass spectrometry, etc.) and nucleic acid measurements (mRNA quatitation). This list is not meant to be limiting.

The term “positive going” marker as that term is used herein refer to a marker that is determined to be elevated in subjects suffering from a disease or condition, relative to subjects not suffering from that disease or condition. The term “negative going” marker as that term is used herein refer to a marker that is determined to be reduced in subjects suffering from a disease or condition, relative to subjects not suffering from that disease or condition.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample may be obtained from a subject, or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain embodiments, such a sample may be obtained for the purpose of determining the outcome of an ongoing condition or the effect of a treatment regimen on a condition. Preferred body fluid samples include blood, serum, plasma, cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, for a kidney injury marker of the present invention, optionally together with other clinical characteristics, to arrive at a diagnosis (that is, the occurrence or nonoccurrence) of an acute renal injury or ARF for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient.

Marker Assays

In general, immunoassays involve contacting a sample containing or suspected of containing a biomarker of interest with at least one antibody that specifically binds to the biomarker. A signal is then generated indicative of the presence or amount of complexes formed by the binding of polypeptides in the sample to the antibody or other binding species. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In certain aspects, the present invention provides kits for the analysis of the described kidney injury markers. The kit comprises reagents for the analysis of at least one test sample which comprise at least one antibody that a kidney injury marker. The kit can also include devices and instructions for performing one or more of the diagnostic and/or prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies used in the immunoassays described herein preferably specifically bind to a kidney injury marker of the present invention. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Numerous publications discuss the use of phage display technology to produce and screen libraries of polypeptides for binding to a selected analyte. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

While the present application describes antibody-based binding assays in detail, alternatives to antibodies as binding species in assays are well known in the art. These include receptors for a particular target, aptamers, etc. Aptamers are oligonucleic acid or peptide molecules that bind to a specific target molecule. Aptamers are usually created by selecting them from a large random sequence pool, but natural aptamers also exist. High-affinity aptamers containing modified nucleotides conferring improved characteristics on the ligand, such as improved in vivo stability or improved delivery characteristics. Examples of such modifications include chemical substitutions at the ribose and/or phosphate and/or base positions, and may include amino acid side chain functionalities.

Assay Correlations

The term “correlating” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and more risky, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5th percentile of the concentration seen in a normal population. Another method may be to look at serial samples from the same patient, where a prior “baseline” result is used to monitor for temporal changes in a biomarker level.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1−specificity, the ROC graph is sometimes called the sensitivity vs (1−specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

Additional clinical indicia may be combined with the kidney injury marker assay result(s) of the present invention. These include other biomarkers related to renal status. Examples include the following, which recite the common biomarker name, followed by the Swiss-Prot entry number for that biomarker or its parent: Actin (P68133); Adenosine deaminase binding protein (DPP4, P27487); Alpha-1-acid glycoprotein 1 (P02763); Alpha-1-microglobulin (P02760); Albumin (P02768); Angiotensinogenase (Renin, P00797); Annexin A2 (P07355); Beta-glucuronidase (P08236); B-2-microglobulin (P61679); Beta-galactosidase (P16278); BMP-7 (P18075); Brain natriuretic peptide (proBNP, BNP-32, NTproBNP; P16860); Calcium-binding protein Beta (S100-beta, P04271); Carbonic anhydrase (Q16790); Casein Kinase 2 (P68400); Ceruloplasmin (P00450); Clusterin (P10909); Complement C3 (P01024); Cysteine-rich protein (CYR61, O00622); Cytochrome C (P99999); Epidermal growth factor (EGF, P01133); Endothelin-1 (P05305); Exosomal Fetuin-A (P02765); Fatty acid-binding protein, heart (FABP3, P05413); Fatty acid-binding protein, liver (P07148); Ferritin (light chain, P02793; heavy chain P02794); Fructose-1,6-biphosphatase (P09467); GRO-alpha (CXCL1, (P09341); Growth Hormone (P01241); Hepatocyte growth factor (P14210); Insulin-like growth factor I (P01343); Immunoglobulin G; Immunoglobulin Light Chains (Kappa and Lambda); Interferon gamma (P01308); Lysozyme (P61626); Interleukin-1alpha (P01583); Interleukin-2 (P60568); Interleukin-4 (P60568); Interleukin-9 (P15248); Interleukin-12p40 (P29460); Interleukin-13 (P35225); Interleukin-16 (Q14005); L1 cell adhesion molecule (P32004); Lactate dehydrogenase (P00338); Leucine Aminopeptidase (P28838); Meprin A-alpha subunit (Q16819); Meprin A-beta subunit (Q16820); Midkine (P21741); MIP2-alpha (CXCL2, P19875); MMP-2 (P08253); MMP-9 (P14780); Netrin-1 (095631); Neutral endopeptidase (P08473); Osteopontin (P10451); Renal papillary antigen 1 (RPA1); Renal papillary antigen 2 (RPA2); Retinol binding protein (P09455); Ribonuclease; S100 calcium-binding protein A6 (P06703); Serum Amyloid P Component (P02743); Sodium/Hydrogen exchanger isoform (NHE3, P48764); Spermidine/spermine N1-acetyltransferase (P21673); TGF-Beta1 (P01137); Transferrin (P02787); Trefoil factor 3 (TFF3, Q07654); Toll-Like protein 4 (O00206); Total protein; Tubulointerstitial nephritis antigen (Q9UJW2); Uromodulin (Tamm-Horsfall protein, P07911).

For purposes of risk stratification, Adiponectin (Q15848); Alkaline phosphatase (P05186); Aminopeptidase N (P15144); CalbindinD28k (P05937); Cystatin C (P01034); 8 subunit of FIFO ATPase (P03928); Gamma-glutamyltransferase (P19440); GSTa (alpha-glutathione-S-transferase, P08263); GSTpi (Glutathione-S-transferase P; GST class-pi; P09211); IGFBP-1 (P08833); IGFBP-2 (P18065); IGFBP-6 (P24592); Integral membrane protein 1 (Itm1, P46977); Interleukin-6 (P05231); Interleukin-8 (P10145); Interleukin-18 (Q14116); IP-10 (10 kDa interferon-gamma-induced protein, P02778); IRPR (IFRD1, O00458); Isovaleryl-CoA dehydrogenase (IVD, P26440); I-TAC/CXCL11 (O14625); Keratin 19 (P08727); Kim-1 (Hepatitis A virus cellular receptor 1, 043656); L-arginine:glycine amidinotransferase (P50440); Leptin (P41159); Lipocalin2 (NGAL, P80188); MCP-1 (P13500); MIG (Gamma-interferon-induced monokine Q07325); MIP-1a (P10147); MIP-3a (P78556); MIP-1beta (P13236); MIP-1d (Q16663); NAG (N-acetyl-beta-D-glucosaminidase, P54802); Organic ion transporter (OCT2, O15244); Osteoprotegerin (O14788); P8 protein (O60356); Plasminogen activator inhibitor 1 (PAI-1, P05121); ProANP(1-98) (P01160); Protein phosphatase 1-beta (PPI-beta, P62140); Rab GDI-beta (P50395); Renal kallikrein (Q86U61); RT1.B-1 (alpha) chain of the integral membrane protein (Q5Y7A8); Soluble tumor necrosis factor receptor superfamily member 1A (sTNFR-I, P19438); Soluble tumor necrosis factor receptor superfamily member 1B (sTNFR-II, P20333); Tissue inhibitor of metalloproteinases 3 (TIMP-3, P35625); uPAR (Q03405) may be combined with the kidney injury marker assay result(s) of the present invention.

Other clinical indicia which may be combined with the kidney injury marker assay result(s) of the present invention includes demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a urine total protein measurement, a glomerular filtration rate, an estimated glomerular filtration rate, a urine production rate, a serum or plasma creatinine concentration, a renal papillary antigen 1 (RPA1) measurement; a renal papillary antigen 2 (RPA2) measurement; a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, and/or a renal failure index calculated as urine sodium/(urine creatinine/plasma creatinine). Other measures of renal function which may be combined with the kidney injury marker assay result(s) are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

Combining assay results/clinical indicia in this manner can comprise the use of multivariate logistical regression, loglinear modeling, neural network analysis, n-of-m analysis, decision tree analysis, etc. This list is not meant to be limiting.

Diagnosis of Acute Renal Failure

As noted above, the terms “acute renal (or kidney) injury” and “acute renal (or kidney) failure” as used herein are defined in part in terms of changes in serum creatinine from a baseline value. Most definitions of ARF have common elements, including the use of serum creatinine and, often, urine output. Patients may present with renal dysfunction without an available baseline measure of renal function for use in this comparison. In such an event, one may estimate a baseline serum creatinine value by assuming the patient initially had a normal GFR. Glomerular filtration rate (GFR) is the volume of fluid filtered from the renal (kidney) glomerular capillaries into the Bowman's capsule per unit time. Glomerular filtration rate (GFR) can be calculated by measuring any chemical that has a steady level in the blood, and is freely filtered but neither reabsorbed nor secreted by the kidneys. GFR is typically expressed in units of ml/min:

$$GFR = \frac{Urine Concentration \times Urine Flow}{Plasma Concentration}$$

By normalizing the GFR to the body surface area, a GFR of approximately 75-100 ml/min per 1.73 m² can be assumed. The rate therefore measured is the quantity of the substance in the urine that originated from a calculable volume of blood.

There are several different techniques used to calculate or estimate the glomerular filtration rate (GFR or eGFR). In clinical practice, however, creatinine clearance is used to measure GFR. Creatinine is produced naturally by the body (creatinine is a metabolite of creatine, which is found in muscle). It is freely filtered by the glomerulus, but also actively secreted by the renal tubules in very small amounts such that creatinine clearance overestimates actual GFR by 10-20%. This margin of error is acceptable considering the ease with which creatinine clearance is measured.

Creatinine clearance (CCr) can be calculated if values for creatinine's urine concentration (U_{Cr}), urine flow rate (V), and creatinine's plasma concentration (P_{Cr}) are known. Since the product of urine concentration and urine flow rate yields creatinine's excretion rate, creatinine clearance is also said to be its excretion rate (U_{Cr}×V) divided by its plasma concentration. This is commonly represented mathematically as:

$${C}_{Cr} = \frac{{U}_{Cr} \times V}{{P}_{Cr}}$$

Commonly a 24 hour urine collection is undertaken, from empty-bladder one morning to the contents of the bladder the following morning, with a comparative blood test then taken:

$${C}_{Cr} = \frac{{U}_{Cr} \times 24 \text{-} hour volume}{{P}_{Cr} \times 24 \times 60 mins}$$

To allow comparison of results between people of different sizes, the CCr is often corrected for the body surface area (BSA) and expressed compared to the average sized man as ml/min/1.73 m2. While most adults have a BSA that approaches 1.7 (1.6-1.9), extremely obese or slim patients should have their CCr corrected for their actual BSA:

$${C}_{Cr - corrected} = \frac{{C}_{Cr} \times 1.73}{BSA}$$

The accuracy of a creatinine clearance measurement (even when collection is complete) is limited because as glomerular filtration rate (GFR) falls creatinine secretion is increased, and thus the rise in serum creatinine is less. Thus, creatinine excretion is much greater than the filtered load, resulting in a potentially large overestimation of the GFR (as much as a twofold difference). However, for clinical purposes it is important to determine whether renal function is stable or getting worse or better. This is often determined by monitoring serum creatinine alone. Like creatinine clearance, the serum creatinine will not be an accurate reflection of GFR in the non-steady-state condition of ARF. Nonetheless, the degree to which serum creatinine changes from baseline will reflect the change in GFR. Serum creatinine is readily and easily measured and it is specific for renal function.

For purposes of determining urine output on a Urine output on a mL/kg/hr basis, hourly urine collection and measurement is adequate. In the case where, for example, only a cumulative 24-h output was available and no patient weights are provided, minor modifications of the RIFLE urine output criteria have been described. For example, Bagshaw et al., Nephrol. Dial. Transplant. 23: 1203-1210, 2008, assumes an average patient weight of 70 kg, and patients are assigned a RIFLE classification based on the following: <35 mL/h (Risk), <21 mL/h (Injury) or <4 mL/h (Failure).

Selecting a Treatment Regimen

Once a diagnosis is obtained, the clinician can readily select a treatment regimen that is compatible with the diagnosis, such as initiating renal replacement therapy, withdrawing delivery of compounds that are known to be damaging to the kidney, kidney transplantation, delaying or avoiding procedures that are known to be damaging to the kidney, modifying diuretic administration, initiating goal directed therapy, etc. The skilled artisan is aware of appropriate treatments for numerous diseases discussed in relation to the methods of diagnosis described herein. See, e.g., Merck Manual of Diagnosis and Therapy, 17th Ed. Merck Research Laboratories, Whitehouse Station, N.J., 1999. In addition, since the methods and compositions described herein provide prognostic information, the markers of the present invention may be used to monitor a course of treatment. For example, improved or worsened prognostic state may indicate that a particular treatment is or is not efficacious.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### Example 1

### Contrast-Induced Nephropathy Sample Collection

The objective of this sample collection study is to collect samples of plasma and urine and clinical data from patients before and after receiving intravascular contrast media. Approximately 250 adults undergoing radiographic/angiographic procedures involving intravascular administration of iodinated contrast media are enrolled. To be enrolled in the study, each patient must meet all of the following inclusion criteria and none of the following exclusion criteria:

### Inclusion Criteria

males and females 18 years of age or older;
undergoing a radiographic/angiographic procedure (such as a CT scan or coronary intervention) involving the intravascular administration of contrast media;
expected to be hospitalized for at least 48 hours after contrast administration.
able and willing to provide written informed consent for study participation and to comply with all study procedures.

### Exclusion Criteria

renal transplant recipients;
acutely worsening renal function prior to the contrast procedure;
already receiving dialysis (either acute or chronic) or in imminent need of dialysis at enrollment;
expected to undergo a major surgical procedure (such as involving cardiopulmonary bypass) or an additional imaging procedure with contrast media with significant risk for further renal insult within the 48 hrs following contrast administration;
participation in an interventional clinical study with an experimental therapy within the previous 30 days;
known infection with human immunodeficiency virus (HIV) or a hepatitis virus.

Immediately prior to the first contrast administration (and after any pre-procedure hydration), an EDTA anti-coagulated blood sample (10 mL) and a urine sample (10 mL) are collected from each patient. Blood and urine samples are then collected at 4 (±0.5), 8 (±1), 24 (±2) 48 (±2), and 72 (±2) hrs following the last administration of contrast media during the index contrast procedure. Blood is collected via direct venipuncture or via other available venous access, such as an existing femoral sheath, central venous line, peripheral intravenous line or hep-lock. These study blood samples are processed to plasma at the clinical site, frozen and shipped to Astute Medical, Inc., San Diego, Calif. The study urine samples are frozen and shipped to Astute Medical, Inc.

Serum creatinine is assessed at the site immediately prior to the first contrast administration (after any pre-procedure hydration) and at 4 (±0.5), 8 (±1), 24 (±2) and 48 (±2)), and 72 (±2) hours following the last administration of contrast (ideally at the same time as the study samples are obtained). In addition, each patient's status is evaluated through day 30 with regard to additional serum and urine creatinine measurements, a need for dialysis, hospitalization status, and adverse clinical outcomes (including mortality).

Prior to contrast administration, each patient is assigned a risk based on the following assessment: systolic blood pressure <80 mm Hg=5 points; intra-arterial balloon pump=5 points; congestive heart failure (Class III-IV or history of pulmonary edema)=5 points; age >75 yrs=4 points; hematocrit level <39% for men, <35% for women=3 points; diabetes=3 points; contrast media volume=1 point for each 100 mL; serum creatinine level >1.5 g/dL=4 points OR estimated GFR 40-60 mL/min/1.73 m²=2 points, 20-40 mL/min/1.73 m²=4 points, <20 mL/min/1.73 m²=6 points. The risks assigned are as follows: risk for CIN and dialysis: 5 or less total points=risk of CIN—7.5%, risk of dialysis—0.04%; 6-10 total points=risk of CIN—14%, risk of dialysis—0.12%; 11-16 total points=risk of CIN—26.1%, risk of dialysis—1.09%; >16 total points=risk of CIN—57.3%, risk of dialysis—12.8%.

### Example 2

### Cardiac Surgery Sample Collection

The objective of this sample collection study is to collect samples of plasma and urine and clinical data from patients before and after undergoing cardiovascular surgery, a procedure known to be potentially damaging to kidney function. Approximately 900 adults undergoing such surgery are enrolled. To be enrolled in the study, each patient must meet all of the following inclusion criteria and none of the following exclusion criteria:

### Inclusion Criteria

males and females 18 years of age or older;
undergoing cardiovascular surgery;
Toronto/Ottawa Predictive Risk Index for Renal Replacement risk score of at least 2 (Wijeysundera et al., JAMA 297: 1801-9, 2007); and
able and willing to provide written informed consent for study participation and to comply with all study procedures.

### Exclusion Criteria

known pregnancy;
previous renal transplantation;
acutely worsening renal function prior to enrollment (e.g., any category of RIFLE criteria);
already receiving dialysis (either acute or chronic) or in imminent need of dialysis at enrollment;
currently enrolled in another clinical study or expected to be enrolled in another clinical study within 7 days of cardiac surgery that involves drug infusion or a therapeutic intervention for AKI;
known infection with human immunodeficiency virus (HIV) or a hepatitis virus.

Within 3 hours prior to the first incision (and after any pre-procedure hydration), an EDTA anti-coagulated blood sample (10 mL), whole blood (3 mL), and a urine sample (35 mL) are collected from each patient. Blood and urine samples are then collected at 3 (±0.5), 6 (±0.5), 12 (±1), 24 (±2) and 48 (±2) hrs following the procedure and then daily on days 3 through 7 if the subject remains in the hospital. Blood is collected via direct venipuncture or via other available venous access, such as an existing femoral sheath, central venous line, peripheral intravenous line or hep-lock. These study blood samples are frozen and shipped to Astute Medical, Inc., San Diego, Calif. The study urine samples are frozen and shipped to Astute Medical, Inc.

### Example 3

### Acutely Ill Subject Sample Collection

The objective of this study is to collect samples from acutely ill patients. Approximately 900 adults expected to be in the ICU for at least 48 hours will be enrolled. To be enrolled in the study, each patient must meet all of the following inclusion criteria and none of the following exclusion criteria:

### Inclusion Criteria

males and females 18 years of age or older;
Study population 1: approximately 300 patients that have at least one of:
shock (SBP <90 mmHg and/or need for vasopressor support to maintain MAP >60 mmHg and/or documented drop in SBP of at least 40 mmHg); and sepsis;
Study population 2: approximately 300 patients that have at least one of:
IV antibiotics ordered in computerized physician order entry (CPOE) within 24 hours of enrollment;
contrast media exposure within 24 hours of enrollment;
increased Intra-Abdominal Pressure with acute decompensated heart failure; and
severe trauma as the primary reason for ICU admission and likely to be hospitalized in the ICU for 48 hours after enrollment;
Study population 3: approximately 300 patients expected to be hospitalized through acute care setting (ICU or ED) with a known risk factor for acute renal injury (e.g. sepsis, hypotension/shock (Shock=systolic BP<90 mmHg and/or the need for vasopressor support to maintain a MAP >60 mmHg and/or a documented drop in SBP >40 mmHg), major trauma, hemorrhage, or major surgery); and/or expected to be hospitalized to the ICU for at least 24 hours after enrollment.
Exclusion Criteria known pregnancy;
institutionalized individuals;
previous renal transplantation;
known acutely worsening renal function prior to enrollment (e.g., any category of RIFLE criteria);
received dialysis (either acute or chronic) within 5 days prior to enrollment or in imminent need of dialysis at the time of enrollment;
known infection with human immunodeficiency virus (HIV) or a hepatitis virus;
meets only the SBP <90 mmHg inclusion criterion set forth above, and does not have shock in the attending physician's or principal investigator's opinion.

After providing informed consent, an EDTA anti-coagulated blood sample (10 mL) and a urine sample (25-30 mL) are collected from each patient. Blood and urine samples are then collected at 4 (±0.5) and 8 (±1) hours after contrast administration (if applicable); at 12 (±1), 24 (±2), and 48 (±2) hours after enrollment, and thereafter daily up to day 7 to day 14 while the subject is hospitalized. Blood is collected via direct venipuncture or via other available venous access, such as an existing femoral sheath, central venous line, peripheral intravenous line or hep-lock. These study blood samples are processed to plasma at the clinical site, frozen and shipped to Astute Medical, Inc., San Diego, Calif. The study urine samples are frozen and shipped to Astute Medical, Inc.

### Example 4

### Immunoassay Format

Analytes are measured using standard sandwich enzyme immunoassay techniques. A first antibody which binds the analyte is immobilized in wells of a 96 well polystyrene microplate. Analyte standards and test samples are pipetted into the appropriate wells and any analyte present is bound by the immobilized antibody. After washing away any unbound substances, a horseradish peroxidase-conjugated second antibody which binds the analyte is added to the wells, thereby forming sandwich complexes with the analyte (if present) and the first antibody. Following a wash to remove any unbound antibody-enzyme reagent, a substrate solution comprising tetramethylbenzidine and hydrogen peroxide is added to the wells. Color develops in proportion to the amount of analyte present in the sample. The color development is stopped and the intensity of the color is measured at 540 nm or 570 nm. An analyte concentration is assigned to the test sample by comparison to a standard curve determined from the analyte standards.

### Example 5

### Apparently Healthy Donor and Chronic Disease Patient Samples

Human urine samples from donors with no known chronic or acute disease (“Apparently Healthy Donors”) were purchased from two vendors (Golden West Biologicals, Inc., 27625 Commerce Center Dr., Temecula, Calif. 92590 and Virginia Medical Research, Inc., 915 First Colonial Rd., Virginia Beach, Va. 23454). The urine samples were shipped and stored frozen at less than −20° C. The vendors supplied demographic information for the individual donors including gender, race (Black/White), smoking status and age.

Human urine samples from donors with various chronic diseases (“Chronic Disease Patients”) including congestive heart failure, coronary artery disease, chronic kidney disease, chronic obstructive pulmonary disease, diabetes mellitus and hypertension were purchased from Virginia Medical Research, Inc., 915 First Colonial Rd., Virginia Beach, Va. 23454. The urine samples were shipped and stored frozen at less than −20 degrees centigrade. The vendor provided a case report form for each individual donor with age, gender, race (Black/White), smoking status and alcohol use, height, weight, chronic disease(s) diagnosis, current medications and previous surgeries.

### Example 6

### Use of Kidney Injury Markers for Evaluating Renal Status in Patients

Patients from the intensive care unit (ICU) were enrolled in the following study. Each patient was classified by kidney status as non-injury (0), risk of injury (R), injury (I), and failure (F) according to the maximum stage reached within 7 days of enrollment as determined by the RIFLE criteria. EDTA anti-coagulated blood samples (10 mL) and a urine samples (25-30 mL) were collected from each patient at enrollment, 4 (±0.5) and 8 (±1) hours after contrast administration (if applicable); at 12 (±1), 24 (±2), and 48 (±2) hours after enrollment, and thereafter daily up to day 7 to day 14 while the subject is hospitalized. Tumor necrosis factor receptor superfamily member 10B, Cadherin-16, Caspase-9, Bcl2 antagonist of cell death, Caspase-1, Cadherin-1, Poly [ADP-ribose] polymerase 1, Cyclin-dependent kinase inhibitor 1, Cadherin-5, Myoglobin, Apolipoprotein A-II, Mucin-16, Carcinoembryonic antigen-related cell adhesion molecule 5, and Cellular tumor antigen p53 were each measured by standard immunoassay methods using commercially available assay reagents in the urine samples and the plasma component of the blood samples collected. Concentrations were reported as follows: Tumor necrosis factor receptor superfamily member 10B—ng/ml, Cadherin-16—ng/ml, Caspase-9—ng/ml, Bcl2 antagonist of cell death—absorbance units, Caspase-1—pg/ml, Cadherin-1—pg/ml, Poly [ADP-ribose] polymerase 1—ng/ml, Cyclin-dependent kinase inhibitor 1—pg/ml, Cadherin-5—ng/ml, Myoglobin—ng/ml, Apolipoprotein A-II—ng/ml, Mucin-16, Carcinoembryonic antigen-related cell adhesion molecule 5, and Cellular tumor antigen p53—U/ml, Carcinoembryonic antigen-related cell adhesion molecule 5—ng/ml, and Cellular tumor antigen p53—ng/ml.

Two cohorts were defined as described in the introduction to each of the following tables. In the following tables, the time “prior max stage” represents the time at which a sample is collected, relative to the time a particular patient reaches the lowest disease stage as defined for that cohort, binned into three groups which are +/−12 hours. For example, “24 hr prior” which uses 0 vs R, I, F as the two cohorts would mean 24 hr (+/−12 hours) prior to reaching stage R (or I if no sample at R, or F if no sample at R or I).

A receiver operating characteristic (ROC) curve was generated for each biomarker measured and the area under each ROC curve (AUC) was determined. Patients in Cohort 2 were also separated according to the reason for adjudication to cohort 2 as being based on serum creatinine measurements (sCr), being based on urine output (UO), or being based on either serum creatinine measurements or urine output. Using the same example discussed above (0 vs R, I, F), for those patients adjudicated to stage R, I, or F on the basis of serum creatinine measurements alone, the stage 0 cohort may have included patients adjudicated to stage R, I, or F on the basis of urine output; for those patients adjudicated to stage R, I, or F on the basis of urine output alone, the stage 0 cohort may have included patients adjudicated to stage R, I, or F on the basis of serum creatinine measurements; and for those patients adjudicated to stage R, I, or F on the basis of serum creatinine measurements or urine output, the stage 0 cohort contains only patients in stage 0 for both serum creatinine measurements and urine output. Also, in the data for patients adjudicated on the basis of serum creatinine measurements or urine output, the adjudication method which yielded the most severe RIFLE stage was used.

The ability to distinguish cohort 1 from Cohort 2 was determined using ROC analysis. SE is the standard error of the AUC, n is the number of sample or individual patients (“pts,” as indicated). Standard errors were calculated as described in Hanley, J. A., and McNeil, B. J., The meaning and use of the area under a receiver operating characteristic (ROC) curve. Radiology (1982) 143: 29-36; p values were calculated with a two-tailed Z-test, and are reported as p<0.05 in tables 1-6 and p<0.10 in tables 7-14. An AUC <0.5 is indicative of a negative going marker for the comparison, and an AUC >0.5 is indicative of a positive going marker for the comparison.

Various threshold (or “cutoff”) concentrations were selected, and the associated sensitivity and specificity for distinguishing cohort 1 from cohort 2 were determined. OR is the odds ratio calculated for the particular cutoff concentration, and 95% CI is the confidence interval for the odds ratio.

TABLE 1
Comparison of marker levels in urine samples collected from Cohort 1
(patients that did not progress beyond RIFLE stage 0) and in urine samples collected from
subjects at 0, 24 hours, and 48 hours prior to reaching stage R, I or F in Cohort 2.
Apolipoprotein A-II

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 71.6 | 85.6 | 71.6 | 72.2 | 71.6 | 75.2 |
| Average | 199 | 234 | 199 | 394 | 199 | 136 |
| Stdev | 723 | 763 | 723 | 2590 | 723 | 224 |
| p (t-test) | | 0.70 | | 0.21 | | 0.58 |
| Min | 2.08 | 4.67 | 2.08 | 9.26 | 2.08 | 1.00E−9 |
| Max | 6400 | 6400 | 6400 | 24300 | 6400 | 1420 |
| n (Samp) | 366 | 74 | 366 | 88 | 366 | 41 |
| n (Patient) | 196 | 74 | 196 | 88 | 196 | 41 |
| sCr only | | | | | | |
| Median | 75.8 | 85.0 | 75.8 | 85.9 | 75.8 | 75.2 |
| Average | 201 | 358 | 201 | 131 | 201 | 120 |
| Stdev | 1050 | 1220 | 1050 | 145 | 1050 | 127 |
| p (t-test) | | 0.45 | | 0.69 | | 0.71 |
| Min | 1.00E−9 | 4.67 | 1.00E−9 | 9.44 | 1.00E−9 | 5.58 |
| Max | 24300 | 6400 | 24300 | 677 | 24300 | 488 |
| n (Samp) | 750 | 27 | 750 | 37 | 750 | 23 |
| n (Patient) | 294 | 27 | 294 | 37 | 294 | 23 |
| UO only | | | | | | |
| Median | 72.9 | 95.4 | 72.9 | 82.5 | 72.9 | 117 |
| Average | 219 | 249 | 219 | 483 | 219 | 174 |
| Stdev | 774 | 808 | 774 | 2820 | 774 | 260 |
| p (t-test) | | 0.78 | | 0.16 | | 0.73 |
| Min | 2.08 | 18.9 | 2.08 | 9.26 | 2.08 | 1.00E−9 |
| Max | 6400 | 6400 | 6400 | 24300 | 6400 | 1420 |
| n (Samp) | 297 | 65 | 297 | 74 | 297 | 35 |
| n (Patient) | 132 | 65 | 132 | 74 | 132 | 35 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | 0.54 | 0.59 | 0.53 | 0.54 | 0.56 | 0.53 | 0.51 | 0.60 |
| SE | 0.038 | 0.058 | 0.040 | 0.035 | 0.050 | 0.038 | 0.048 | 0.062 | 0.053 |
| p | 0.066 | 0.54 | 0.019 | 0.47 | 0.45 | 0.11 | 0.57 | 0.82 | 0.068 |
| nCohort 1 | 366 | 750 | 297 | 366 | 750 | 297 | 366 | 750 | 297 |
| nCohort 2 | 74 | 27 | 65 | 88 | 37 | 74 | 41 | 23 | 35 |
| Cutoff 1 | 53.4 | 58.0 | 64.3 | 46.8 | 58.2 | 56.1 | 46.8 | 47.2 | 68.6 |
| Sens 1 | 70% | 70% | 71% | 70% | 70% | 70% | 71% | 74% | 71% |
| Spec 1 | 38% | 36% | 45% | 32% | 37% | 38% | 32% | 28% | 48% |
| Cutoff 2 | 36.3 | 34.3 | 48.8 | 40.5 | 33.2 | 44.5 | 28.5 | 41.3 | 37.4 |
| Sens 2 | 81% | 81% | 80% | 81% | 81% | 81% | 80% | 83% | 80% |
| Spec 2 | 20% | 16% | 31% | 24% | 15% | 25% | 12% | 23% | 19% |
| Cutoff 3 | 33.5 | 26.3 | 36.2 | 28.5 | 26.3 | 33.2 | 21.0 | 27.1 | 23.1 |
| Sens 3 | 91% | 93% | 91% | 91% | 92% | 91% | 90% | 91% | 91% |
| Spec 3 | 17% | 9% | 16% | 12% | 9% | 14% | 7% | 10% | 8% |
| Cutoff 4 | 107 | 114 | 113 | 107 | 114 | 113 | 107 | 114 | 113 |
| Sens 4 | 39% | 41% | 42% | 34% | 41% | 41% | 39% | 35% | 54% |
| Spec 4 | 70% | 70% | 70% | 70% | 70% | 70% | 70% | 70% | 70% |
| Cutoff 5 | 135 | 148 | 144 | 135 | 148 | 144 | 135 | 148 | 144 |
| Sens 5 | 32% | 26% | 34% | 26% | 30% | 31% | 32% | 17% | 46% |
| Spec 5 | 80% | 80% | 80% | 80% | 80% | 80% | 80% | 80% | 80% |
| Cutoff 6 | 226 | 227 | 292 | 226 | 227 | 292 | 226 | 227 | 292 |
| Sens 6 | 18% | 15% | 14% | 10% | 11% | 9% | 12% | 13% | 9% |
| Spec 6 | 90% | 90% | 90% | 90% | 90% | 90% | 90% | 90% | 90% |
| OR Quart 2 | 0.67 | 0.42 | 1.2 | 1.4 | 0.58 | 1.1 | 0.46 | 1.4 | 0.60 |
| p Value | 0.32 | 0.21 | 0.68 | 0.33 | 0.31 | 0.87 | 0.14 | 0.56 | 0.39 |
| 95% CI of | 0.30 | 0.11 | 0.51 | 0.72 | 0.21 | 0.50 | 0.17 | 0.44 | 0.19 |
| OR Quart 2 | 1.5 | 1.6 | 2.8 | 2.7 | 1.6 | 2.3 | 1.3 | 4.5 | 1.9 |
| OR Quart 3 | 1.2 | 1.1 | 1.7 | 0.88 | 0.79 | 0.99 | 0.72 | 0.80 | 0.60 |
| p Value | 0.59 | 0.79 | 0.22 | 0.72 | 0.62 | 0.97 | 0.48 | 0.74 | 0.39 |
| 95% CI of | 0.60 | 0.41 | 0.73 | 0.43 | 0.30 | 0.46 | 0.29 | 0.21 | 0.19 |
| OR Quart 3 | 2.5 | 3.2 | 3.8 | 1.8 | 2.0 | 2.1 | 1.8 | 3.0 | 1.9 |
| OR Quart 4 | 1.6 | 1.3 | 2.6 | 1.5 | 1.3 | 1.7 | 1.2 | 1.4 | 2.4 |
| p Value | 0.17 | 0.62 | 0.018 | 0.20 | 0.53 | 0.12 | 0.69 | 0.57 | 0.056 |
| 95% CI of | 0.81 | 0.47 | 1.2 | 0.80 | 0.56 | 0.86 | 0.52 | 0.44 | 0.98 |
| OR Quart 4 | 3.2 | 3.5 | 5.6 | 3.0 | 3.1 | 3.5 | 2.7 | 4.5 | 6.0 |

Bcl2 antagonist of cell death

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.00173 | 0.00173 | nd | nd | nd | nd |
| Average | 0.0412 | 0.00246 | nd | nd | nd | nd |
| Stdev | 0.150 | 0.00254 | nd | nd | nd | nd |
| p (t-test) | | 0.38 | nd | nd | nd | nd |
| Min | 0.00173 | 0.00173 | nd | nd | nd | nd |
| Max | 0.833 | 0.0105 | nd | nd | nd | nd |
| n (Samp) | 54 | 12 | nd | nd | nd | nd |
| n (Patient) | 36 | 12 | nd | nd | nd | nd |
| sCr only | | | | | | |
| Median | 0.00173 | 0.00173 | nd | nd | nd | nd |
| Average | 0.0279 | 0.00319 | nd | nd | nd | nd |
| Stdev | 0.122 | 0.00359 | nd | nd | nd | nd |
| p (t-test) | | 0.62 | nd | nd | nd | nd |
| Min | 0.00173 | 0.00173 | nd | nd | nd | nd |
| Max | 0.833 | 0.0105 | nd | nd | nd | nd |
| n (Samp) | 84 | 6 | nd | nd | nd | nd |
| n (Patient) | 59 | 6 | nd | nd | nd | nd |
| UO only | | | | | | |
| Median | 0.00173 | 0.00173 | nd | nd | nd | nd |
| Average | 0.0457 | 0.00173 | nd | nd | nd | nd |
| Stdev | 0.172 | 0 | nd | nd | nd | nd |
| p (t-test) | | 0.38 | nd | nd | nd | nd |
| Min | 0.00173 | 0.00173 | nd | nd | nd | nd |
| Max | 0.833 | 0.00173 | nd | nd | nd | nd |
| n (Samp) | 40 | 12 | nd | nd | nd | nd |
| n (Patient) | 26 | 12 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.38 | 0.43 | 0.39 | nd | nd | nd | nd | nd | nd |
| SE | 0.094 | 0.13 | 0.097 | nd | nd | nd | nd | nd | nd |
| p | 0.20 | 0.60 | 0.24 | nd | nd | nd | nd | nd | nd |
| nCohort 1 | 54 | 84 | 40 | nd | nd | nd | nd | nd | nd |
| nCohort 2 | 12 | 6 | 12 | nd | nd | nd | nd | nd | nd |
| Cutoff 1 | 0 | 0 | 0 | nd | nd | nd | nd | nd | nd |
| Sens 1 | 100% | 100% | 100% | nd | nd | nd | nd | nd | nd |
| Spec 1 | 0% | 0% | 0% | nd | nd | nd | nd | nd | nd |
| Cutoff 2 | 0 | 0 | 0 | nd | nd | nd | nd | nd | nd |
| Sens 2 | 100% | 100% | 100% | nd | nd | nd | nd | nd | nd |
| Spec 2 | 0% | 0% | 0% | nd | nd | nd | nd | nd | nd |
| Cutoff 3 | 0 | 0 | 0 | nd | nd | nd | nd | nd | nd |
| Sens 3 | 100% | 100% | 100% | nd | nd | nd | nd | nd | nd |
| Spec 3 | 0% | 0% | 0% | nd | nd | nd | nd | nd | nd |
| Cutoff 4 | 0.00509 | 0.00173 | 0.00173 | nd | nd | nd | nd | nd | nd |
| Sens 4 | 8% | 17% | 0% | nd | nd | nd | nd | nd | nd |
| Spec 4 | 70% | 71% | 8% | nd | nd | nd | nd | nd | nd |
| Cutoff 5 | 0.0148 | 0.00943 | 0.00509 | nd | nd | nd | nd | nd | nd |
| Sens 5 | 0% | 17% | 0% | nd | nd | nd | nd | nd | nd |
| Spec 5 | 83% | 82% | 80% | nd | nd | nd | nd | nd | nd |
| Cutoff 6 | 0.0354 | 0.0235 | 0.0148 | nd | nd | nd | nd | nd | nd |
| Sens 6 | 0% | 0% | 0% | nd | nd | nd | nd | nd | nd |
| Spec 6 | 91% | 92% | 90% | nd | nd | nd | nd | nd | nd |
| OR Quart 2 | 9.6 | 4.9 | >0 | nd | nd | nd | nd | nd | nd |
| p Value | 0.050 | 0.17 | <na | nd | nd | nd | nd | nd | nd |
| 95% CI of | 1.0 | 0.50 | >na | nd | nd | nd | nd | nd | nd |
| OR Quart 2 | 92 | 48 | na | nd | nd | nd | nd | nd | nd |
| OR Quart 3 | 3.4 | 1.0 | >160 | nd | nd | nd | nd | nd | nd |
| p Value | 0.31 | 1.0 | <5.9E−4 | nd | nd | nd | nd | nd | nd |
| 95% CI of | 0.32 | 0.059 | >8.8 | nd | nd | nd | nd | nd | nd |
| OR Quart 3 | 37 | 17 | na | nd | nd | nd | nd | nd | nd |
| OR Quart 4 | 2.3 | 0 | >0 | nd | nd | nd | nd | nd | nd |
| p Value | 0.52 | na | <na | nd | nd | nd | nd | nd | nd |
| 95% CI of | 0.19 | na | >na | nd | nd | nd | nd | nd | nd |
| OR Quart 4 | 28 | na | na | nd | nd | nd | nd | nd | nd |

Caspase-9

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.469 | 0.955 | nd | nd | nd | nd |
| Average | 1.11 | 0.937 | nd | nd | nd | nd |
| Stdev | 2.89 | 1.01 | nd | nd | nd | nd |
| p (t-test) | | 0.84 | nd | nd | nd | nd |
| Min | 0.0360 | 0.0360 | nd | nd | nd | nd |
| Max | 19.5 | 3.69 | nd | nd | nd | nd |
| n (Samp) | 54 | 12 | nd | nd | nd | nd |
| n (Patient) | 36 | 12 | nd | nd | nd | nd |
| sCr only | | | | | | |
| Median | 0.469 | 1.05 | nd | nd | nd | nd |
| Average | 1.08 | 1.44 | nd | nd | nd | nd |
| Stdev | 2.44 | 1.42 | nd | nd | nd | nd |
| p (t-test) | | 0.73 | nd | nd | nd | nd |
| Min | 0.0360 | 0.0360 | nd | nd | nd | nd |
| Max | 19.5 | 3.92 | nd | nd | nd | nd |
| n (Samp) | 86 | 6 | nd | nd | nd | nd |
| n (Patient) | 60 | 6 | nd | nd | nd | nd |
| UO only | | | | | | |
| Median | 0.586 | 0.790 | nd | nd | nd | nd |
| Average | 1.35 | 0.929 | nd | nd | nd | nd |
| Stdev | 3.31 | 0.998 | nd | nd | nd | nd |
| p (t-test) | | 0.67 | nd | nd | nd | nd |
| Min | 0.0360 | 0.0360 | nd | nd | nd | nd |
| Max | 19.5 | 3.69 | nd | nd | nd | nd |
| n (Samp) | 40 | 12 | nd | nd | nd | nd |
| n (Patient) | 26 | 12 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | 0.66 | 0.56 | nd | nd | nd | nd | nd | nd |
| SE | 0.094 | 0.13 | 0.097 | nd | nd | nd | nd | nd | nd |
| p | 0.32 | 0.20 | 0.52 | nd | nd | nd | nd | nd | nd |
| nCohort 1 | 54 | 86 | 40 | nd | nd | nd | nd | nd | nd |
| nCohort 2 | 12 | 6 | 12 | nd | nd | nd | nd | nd | nd |
| Cutoff 1 | 0.208 | 0.330 | 0.208 | nd | nd | nd | nd | nd | nd |
| Sens 1 | 75% | 83% | 83% | nd | nd | nd | nd | nd | nd |
| Spec 1 | 37% | 38% | 28% | nd | nd | nd | nd | nd | nd |
| Cutoff 2 | 0 | 0.330 | 0.208 | nd | nd | nd | nd | nd | nd |
| Sens 2 | 100% | 83% | 83% | nd | nd | nd | nd | nd | nd |
| Spec 2 | 0% | 38% | 28% | nd | nd | nd | nd | nd | nd |
| Cutoff 3 | 0 | 0 | 0 | nd | nd | nd | nd | nd | nd |
| Sens 3 | 100% | 100% | 100% | nd | nd | nd | nd | nd | nd |
| Spec 3 | 0% | 0% | 0% | nd | nd | nd | nd | nd | nd |
| Cutoff 4 | 0.703 | 0.811 | 0.781 | nd | nd | nd | nd | nd | nd |
| Sens 4 | 58% | 67% | 50% | nd | nd | nd | nd | nd | nd |
| Spec 4 | 70% | 71% | 72% | nd | nd | nd | nd | nd | nd |
| Cutoff 5 | 1.04 | 1.04 | 0.868 | nd | nd | nd | nd | nd | nd |
| Sens 5 | 33% | 50% | 50% | nd | nd | nd | nd | nd | nd |
| Spec 5 | 81% | 80% | 80% | nd | nd | nd | nd | nd | nd |
| Cutoff 6 | 1.76 | 2.17 | 2.17 | nd | nd | nd | nd | nd | nd |
| Sens 6 | 8% | 17% | 8% | nd | nd | nd | nd | nd | nd |
| Spec 6 | 91% | 91% | 90% | nd | nd | nd | nd | nd | nd |
| OR Quart 2 | 0.58 | 1.0 | 1.6 | nd | nd | nd | nd | nd | nd |
| p Value | 0.58 | 1.0 | 0.62 | nd | nd | nd | nd | nd | nd |
| 95% CI of | 0.083 | 0.059 | 0.23 | nd | nd | nd | nd | nd | nd |
| OR Quart 2 | 4.0 | 17 | 12 | nd | nd | nd | nd | nd | nd |
| OR Quart 3 | 0.29 | 1.0 | 1.0 | nd | nd | nd | nd | nd | nd |
| p Value | 0.31 | 1.0 | 1.0 | nd | nd | nd | nd | nd | nd |
| 95% CI of | 0.027 | 0.059 | 0.12 | nd | nd | nd | nd | nd | nd |
| OR Quart 3 | 3.1 | 17 | 8.4 | nd | nd | nd | nd | nd | nd |
| OR Quart 4 | 2.4 | 3.3 | 3.4 | nd | nd | nd | nd | nd | nd |
| p Value | 0.29 | 0.32 | 0.20 | nd | nd | nd | nd | nd | nd |
| 95% CI of | 0.48 | 0.32 | 0.53 | nd | nd | nd | nd | nd | nd |
| OR Quart 4 | 12 | 34 | 22 | nd | nd | nd | nd | nd | nd |

Cadherin-1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 48800 | 32700 | 48800 | 111000 | nd | nd |
| Average | 84900 | 79300 | 84900 | 143000 | nd | nd |
| Stdev | 121000 | 100000 | 121000 | 147000 | nd | nd |
| p (t-test) | | 0.86 | | 0.091 | nd | nd |
| Min | 160 | 1620 | 160 | 1660 | nd | nd |
| Max | 744000 | 363000 | 744000 | 543000 | nd | nd |
| n (Samp) | 52 | 20 | 52 | 20 | nd | nd |
| n (Patient) | 41 | 20 | 41 | 20 | nd | nd |
| UO only | | | | | | |
| Median | 37500 | 50200 | 37500 | 111000 | nd | nd |
| Average | 75900 | 91900 | 75900 | 146000 | nd | nd |
| Stdev | 124000 | 107000 | 124000 | 140000 | nd | nd |
| p (t-test) | | 0.65 | | 0.046 | nd | nd |
| Min | 160 | 1620 | 160 | 2220 | nd | nd |
| Max | 744000 | 363000 | 744000 | 543000 | nd | nd |
| n (Samp) | 42 | 16 | 42 | 21 | nd | nd |
| n (Patient) | 33 | 16 | 33 | 21 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.45 | nd | 0.55 | 0.64 | nd | 0.71 | nd | nd | nd |
| SE | 0.077 | nd | 0.086 | 0.076 | nd | 0.073 | nd | nd | nd |
| p | 0.55 | nd | 0.58 | 0.064 | nd | 0.0041 | nd | nd | nd |
| nCohort 1 | 52 | nd | 42 | 52 | nd | 42 | nd | nd | nd |
| nCohort 2 | 20 | nd | 16 | 20 | nd | 21 | nd | nd | nd |
| Cutoff 1 | 10500 | nd | 21400 | 51100 | nd | 54200 | nd | nd | nd |
| Sens 1 | 70% | nd | 75% | 70% | nd | 71% | nd | nd | nd |
| Spec 1 | 15% | nd | 36% | 54% | nd | 60% | nd | nd | nd |
| Cutoff 2 | 9800 | nd | 10400 | 28500 | nd | 36500 | nd | nd | nd |
| Sens 2 | 80% | nd | 81% | 80% | nd | 81% | nd | nd | nd |
| Spec 2 | 13% | nd | 24% | 38% | nd | 50% | nd | nd | nd |
| Cutoff 3 | 4310 | nd | 3350 | 8380 | nd | 11700 | nd | nd | nd |
| Sens 3 | 90% | nd | 94% | 90% | nd | 90% | nd | nd | nd |
| Spec 3 | 8% | nd | 10% | 12% | nd | 29% | nd | nd | nd |
| Cutoff 4 | 94000 | nd | 80700 | 94000 | nd | 80700 | nd | nd | nd |
| Sens 4 | 25% | nd | 38% | 55% | nd | 62% | nd | nd | nd |
| Spec 4 | 71% | nd | 71% | 71% | nd | 71% | nd | nd | nd |
| Cutoff 5 | 134000 | nd | 122000 | 134000 | nd | 122000 | nd | nd | nd |
| Sens 5 | 20% | nd | 31% | 40% | nd | 48% | nd | nd | nd |
| Spec 5 | 81% | nd | 81% | 81% | nd | 81% | nd | nd | nd |
| Cutoff 6 | 152000 | nd | 144000 | 152000 | nd | 144000 | nd | nd | nd |
| Sens 6 | 15% | nd | 19% | 35% | nd | 33% | nd | nd | nd |
| Spec 6 | 90% | nd | 90% | 90% | nd | 90% | nd | nd | nd |
| OR Quart 2 | 0.74 | nd | 0.62 | 1.4 | nd | 0.92 | nd | nd | nd |
| p Value | 0.70 | nd | 0.59 | 0.67 | nd | 0.93 | nd | nd | nd |
| 95% CI of | 0.16 | nd | 0.11 | 0.27 | nd | 0.16 | nd | nd | nd |
| OR Quart 2 | 3.4 | nd | 3.5 | 7.5 | nd | 5.5 | nd | nd | nd |
| OR Quart 3 | 0.74 | nd | 0.68 | 1.9 | nd | 1.8 | nd | nd | nd |
| p Value | 0.70 | nd | 0.66 | 0.43 | nd | 0.48 | nd | nd | nd |
| 95% CI of | 0.16 | nd | 0.12 | 0.38 | nd | 0.35 | nd | nd | nd |
| OR Quart 3 | 3.4 | nd | 3.8 | 9.6 | nd | 9.5 | nd | nd | nd |
| OR Quart 4 | 1.7 | nd | 1.7 | 4.0 | nd | 6.7 | nd | nd | nd |
| p Value | 0.48 | nd | 0.52 | 0.080 | nd | 0.022 | nd | nd | nd |
| 95% CI of | 0.41 | nd | 0.35 | 0.85 | nd | 1.3 | nd | nd | nd |
| OR Quart 4 | 6.7 | nd | 7.9 | 19 | nd | 34 | nd | nd | nd |

Cadherin-5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.00224 | 0.00224 | nd | nd | nd | nd |
| Average | 0.0957 | 0.0142 | nd | nd | nd | nd |
| Stdev | 0.210 | 0.0183 | nd | nd | nd | nd |
| p (t-test) | | 0.28 | nd | nd | nd | nd |
| Min | 0.00224 | 0.00224 | nd | nd | nd | nd |
| Max | 1.10 | 0.0502 | nd | nd | nd | nd |
| n (Samp) | 47 | 8 | nd | nd | nd | nd |
| n (Patient) | 30 | 8 | nd | nd | nd | nd |
| UO only | | | | | | |
| Median | 0.00224 | 0.00224 | nd | nd | nd | nd |
| Average | 0.0806 | 0.00753 | nd | nd | nd | nd |
| Stdev | 0.218 | 0.0108 | nd | nd | nd | nd |
| p (t-test) | | 0.32 | nd | nd | nd | nd |
| Min | 0.00224 | 0.00224 | nd | nd | nd | nd |
| Max | 1.10 | 0.0309 | nd | nd | nd | nd |
| n (Samp) | 34 | 9 | nd | nd | nd | nd |
| n (Patient) | 20 | 9 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.46 | nd | 0.44 | nd | nd | nd | nd | nd | nd |
| SE | 0.11 | nd | 0.11 | nd | nd | nd | nd | nd | nd |
| p | 0.72 | nd | 0.62 | nd | nd | nd | nd | nd | nd |
| nCohort 1 | 47 | nd | 34 | nd | nd | nd | nd | nd | nd |
| nCohort 2 | 8 | nd | 9 | nd | nd | nd | nd | nd | nd |
| Cutoff 1 | 0 | nd | 0 | nd | nd | nd | nd | nd | nd |
| Sens 1 | 100% | nd | 100% | nd | nd | nd | nd | nd | nd |
| Spec 1 | 0% | nd | 0% | nd | nd | nd | nd | nd | nd |
| Cutoff 2 | 0 | nd | 0 | nd | nd | nd | nd | nd | nd |
| Sens 2 | 100% | nd | 100% | nd | nd | nd | nd | nd | nd |
| Spec 2 | 0% | nd | 0% | nd | nd | nd | nd | nd | nd |
| Cutoff 3 | 0 | nd | 0 | nd | nd | nd | nd | nd | nd |
| Sens 3 | 100% | nd | 100% | nd | nd | nd | nd | nd | nd |
| Spec 3 | 0% | nd | 0% | nd | nd | nd | nd | nd | nd |
| Cutoff 4 | 0.0405 | nd | 0.00224 | nd | nd | nd | nd | nd | nd |
| Sens 4 | 12% | nd | 22% | nd | nd | nd | nd | nd | nd |
| Spec 4 | 72% | nd | 71% | nd | nd | nd | nd | nd | nd |
| Cutoff 5 | 0.137 | nd | 0.127 | nd | nd | nd | nd | nd | nd |
| Sens 5 | 0% | nd | 0% | nd | nd | nd | nd | nd | nd |
| Spec 5 | 81% | nd | 82% | nd | nd | nd | nd | nd | nd |
| Cutoff 6 | 0.329 | nd | 0.239 | nd | nd | nd | nd | nd | nd |
| Sens 6 | 0% | nd | 0% | nd | nd | nd | nd | nd | nd |
| Spec 6 | 91% | nd | 91% | nd | nd | nd | nd | nd | nd |
| OR Quart 2 | 2.2 | nd | 0 | nd | nd | nd | nd | nd | nd |
| p Value | 0.55 | nd | na | nd | nd | nd | nd | nd | nd |
| 95% CI of | 0.17 | nd | na | nd | nd | nd | nd | nd | nd |
| OR Quart 2 | 27 | nd | na | nd | nd | nd | nd | nd | nd |
| OR Quart 3 | 3.5 | nd | 5.4 | nd | nd | nd | nd | nd | nd |
| p Value | 0.30 | nd | 0.088 | nd | nd | nd | nd | nd | nd |
| 95% CI of | 0.32 | nd | 0.78 | nd | nd | nd | nd | nd | nd |
| OR Quart 3 | 39 | nd | 38 | nd | nd | nd | nd | nd | nd |
| OR Quart 4 | 2.4 | nd | 0.50 | nd | nd | nd | nd | nd | nd |
| p Value | 0.51 | nd | 0.60 | nd | nd | nd | nd | nd | nd |
| 95% CI of | 0.19 | nd | 0.038 | nd | nd | nd | nd | nd | nd |
| OR Quart 4 | 30 | nd | 6.5 | nd | nd | nd | nd | nd | nd |

Cyclin-dependent kinase inhibitor 1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.713 | 0.116 | 0.713 | 1.98 | 0.713 | 1.09 |
| Average | 103 | 8.88 | 103 | 19.5 | 103 | 5.00 |
| Stdev | 670 | 19.5 | 670 | 49.0 | 670 | 9.16 |
| p (t-test) | | 0.33 | | 0.35 | | 0.47 |
| Min | 1.14E−14 | 1.70E−14 | 1.14E−14 | 1.70E−14 | 1.14E−14 | 1.70E−14 |
| Max | 6950 | 112 | 6950 | 327 | 6950 | 42.0 |
| n (Samp) | 165 | 48 | 165 | 56 | 165 | 25 |
| n (Patient) | 102 | 48 | 102 | 56 | 102 | 25 |
| sCr only | | | | | | |
| Median | 1.09 | 5.28 | 1.09 | 10.7 | 1.09 | 2.45 |
| Average | 62.4 | 15.7 | 62.4 | 19.5 | 62.4 | 6.76 |
| Stdev | 482 | 28.6 | 482 | 21.2 | 482 | 12.3 |
| p (t-test) | | 0.70 | | 0.69 | | 0.68 |
| Min | 1.14E−14 | 0.116 | 1.14E−14 | 1.70E−14 | 1.14E−14 | 1.70E−14 |
| Max | 6950 | 112 | 6950 | 71.0 | 6950 | 44.1 |
| n (Samp) | 325 | 16 | 325 | 20 | 325 | 13 |
| n (Patient) | 168 | 16 | 168 | 20 | 168 | 13 |
| UO only | | | | | | |
| Median | 0.850 | 0.116 | 0.850 | 2.45 | 0.850 | 1.09 |
| Average | 119 | 24.9 | 119 | 23.7 | 119 | 10.5 |
| Stdev | 729 | 97.5 | 729 | 60.2 | 729 | 24.6 |
| p (t-test) | | 0.39 | | 0.35 | | 0.48 |
| Min | 1.14E−14 | 1.70E−14 | 1.14E−14 | 0.116 | 1.14E−14 | 0.116 |
| Max | 6950 | 630 | 6950 | 327 | 6950 | 112 |
| n (Samp) | 139 | 46 | 139 | 51 | 139 | 23 |
| n (Patient) | 85 | 46 | 85 | 51 | 85 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.45 | 0.61 | 0.45 | 0.53 | 0.62 | 0.54 | 0.46 | 0.46 | 0.49 |
| SE | 0.048 | 0.077 | 0.050 | 0.045 | 0.069 | 0.048 | 0.063 | 0.084 | 0.065 |
| p | 0.35 | 0.16 | 0.28 | 0.51 | 0.088 | 0.44 | 0.58 | 0.60 | 0.89 |
| nCohort 1 | 165 | 325 | 139 | 165 | 325 | 139 | 165 | 325 | 139 |
| nCohort 2 | 48 | 16 | 46 | 56 | 20 | 51 | 25 | 13 | 23 |
| Cutoff 1 | 1.70E−14 | 0.850 | 1.70E−14 | 1.70E−14 | 0.850 | 1.14E−14 | 1.70E−14 | 1.70E−14 | 1.14E−14 |
| Sens 1 | 98% | 75% | 98% | 96% | 75% | 100% | 96% | 92% | 100% |
| Spec 1 | 2% | 50% | 2% | 2% | 50% | 2% | 2% | 2% | 2% |
| Cutoff 2 | 1.70E−14 | 0.116 | 1.70E−14 | 1.70E−14 | 1.70E−14 | 1.14E−14 | 1.70E−14 | 1.70E−14 | 1.14E−14 |
| Sens 2 | 98% | 81% | 98% | 96% | 90% | 100% | 96% | 92% | 100% |
| Spec 2 | 2% | 43% | 2% | 2% | 2% | 2% | 2% | 2% | 2% |
| Cutoff 3 | 1.70E−14 | 1.14E−14 | 1.70E−14 | 1.70E−14 | 1.70E−14 | 1.14E−14 | 1.70E−14 | 1.70E−14 | 1.14E−14 |
| Sens 3 | 98% | 100% | 98% | 96% | 90% | 100% | 96% | 92% | 100% |
| Spec 3 | 2% | 2% | 2% | 2% | 2% | 2% | 2% | 2% | 2% |
| Cutoff 4 | 7.84 | 9.28 | 9.28 | 7.84 | 9.28 | 9.28 | 7.84 | 9.28 | 9.28 |
| Sens 4 | 23% | 25% | 26% | 38% | 50% | 35% | 24% | 15% | 22% |
| Spec 4 | 70% | 71% | 71% | 70% | 71% | 71% | 70% | 71% | 71% |
| Cutoff 5 | 18.3 | 15.5 | 22.7 | 18.3 | 15.5 | 22.7 | 18.3 | 15.5 | 22.7 |
| Sens 5 | 17% | 25% | 15% | 25% | 45% | 20% | 4% | 15% | 13% |
| Spec 5 | 80% | 80% | 81% | 80% | 80% | 81% | 80% | 80% | 81% |
| Cutoff 6 | 60.3 | 44.8 | 73.1 | 60.3 | 44.8 | 73.1 | 60.3 | 44.8 | 73.1 |
| Sens 6 | 2% | 6% | 4% | 7% | 10% | 6% | 0% | 0% | 4% |
| Spec 6 | 90% | 90% | 91% | 90% | 90% | 91% | 90% | 90% | 91% |
| OR Quart 2 | 1.3 | 0.33 | 1.0 | 0.25 | 0.19 | 0.60 | 4.6 | 2.6 | 2.7 |
| p Value | 0.60 | 0.34 | 0.96 | 0.0079 | 0.13 | 0.31 | 0.027 | 0.26 | 0.13 |
| 95% CI of | 0.50 | 0.033 | 0.38 | 0.091 | 0.022 | 0.23 | 1.2 | 0.50 | 0.75 |
| OR Quart 2 | 3.3 | 3.2 | 2.8 | 0.70 | 1.7 | 1.6 | 18 | 14 | 9.6 |
| OR Quart 3 | 0.78 | 2.8 | 1.2 | 0.77 | 0.79 | 1.1 | 0 | 0 | 0.23 |
| p Value | 0.64 | 0.13 | 0.76 | 0.53 | 0.73 | 0.82 | na | na | 0.20 |
| 95% CI of | 0.28 | 0.73 | 0.44 | 0.34 | 0.20 | 0.45 | na | na | 0.025 |
| OR Quart 3 | 2.2 | 11 | 3.1 | 1.7 | 3.0 | 2.7 | na | na | 2.2 |
| OR Quart 4 | 2.3 | 1.3 | 1.8 | 0.90 | 2.1 | 1.2 | 4.6 | 3.2 | 2.7 |
| p Value | 0.073 | 0.71 | 0.22 | 0.79 | 0.19 | 0.70 | 0.027 | 0.16 | 0.13 |
| 95% CI of | 0.93 | 0.29 | 0.71 | 0.40 | 0.69 | 0.49 | 1.2 | 0.63 | 0.75 |
| OR Quart 4 | 5.5 | 6.1 | 4.5 | 2.0 | 6.4 | 2.9 | 18 | 16 | 9.6 |

Carcinoembryonic antigen-related cell adhesion molecule 5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.646 | 1.40 | 0.646 | 1.30 | 0.646 | 1.38 |
| Average | 2.04 | 4.66 | 2.04 | 4.98 | 2.04 | 4.81 |
| Stdev | 4.06 | 7.77 | 4.06 | 10.7 | 4.06 | 9.92 |
| p (t-test) | | 6.7E−4 | | 7.2E−4 | | 0.0064 |
| Min | 0.00336 | 0.00336 | 0.00336 | 0.0411 | 0.00336 | 0.0844 |
| Max | 43.4 | 29.4 | 43.4 | 54.4 | 43.4 | 47.4 |
| n (Samp) | 253 | 48 | 253 | 57 | 253 | 26 |
| n (Patient) | 102 | 48 | 102 | 57 | 102 | 26 |
| sCr only | | | | | | |
| Median | 0.947 | 1.48 | 0.947 | 1.66 | 0.947 | 2.29 |
| Average | 12.1 | 3.83 | 12.1 | 7.05 | 12.1 | 3.90 |
| Stdev | 192 | 5.63 | 192 | 12.5 | 192 | 4.53 |
| p (t-test) | | 0.86 | | 0.90 | | 0.88 |
| Min | 0.00336 | 0.00336 | 0.00336 | 0.0411 | 0.00336 | 0.173 |
| Max | 4070 | 21.1 | 4070 | 51.3 | 4070 | 15.3 |
| n (Samp) | 447 | 16 | 447 | 21 | 447 | 13 |
| n (Patient) | 170 | 16 | 170 | 21 | 170 | 13 |
| UO only | | | | | | |
| Median | 0.625 | 1.44 | 0.625 | 1.25 | 0.625 | 1.35 |
| Average | 2.35 | 4.89 | 2.35 | 4.76 | 2.35 | 4.02 |
| Stdev | 4.80 | 7.57 | 4.80 | 10.6 | 4.80 | 9.89 |
| p (t-test) | | 0.0038 | | 0.015 | | 0.16 |
| Min | 0.00336 | 0.00336 | 0.00336 | 0.0946 | 0.00336 | 0.00336 |
| Max | 43.4 | 29.4 | 43.4 | 54.4 | 43.4 | 47.4 |
| n (Samp) | 212 | 47 | 212 | 52 | 212 | 25 |
| n (Patient) | 85 | 47 | 85 | 52 | 85 | 25 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.56 | 0.64 | 0.61 | 0.62 | 0.61 | 0.60 | 0.65 | 0.55 |
| SE | 0.046 | 0.076 | 0.047 | 0.043 | 0.067 | 0.045 | 0.061 | 0.084 | 0.062 |
| p | 0.012 | 0.44 | 0.0036 | 0.0089 | 0.081 | 0.014 | 0.11 | 0.068 | 0.41 |
| nCohort 1 | 253 | 447 | 212 | 253 | 447 | 212 | 253 | 447 | 212 |
| nCohort 2 | 48 | 16 | 47 | 57 | 21 | 52 | 26 | 13 | 25 |
| Cutoff 1 | 0.540 | 0.414 | 0.550 | 0.607 | 1.10 | 0.607 | 0.456 | 1.06 | 0.456 |
| Sens 1 | 71% | 75% | 70% | 70% | 71% | 71% | 73% | 77% | 72% |
| Spec 1 | 45% | 32% | 46% | 48% | 53% | 49% | 42% | 53% | 43% |
| Cutoff 2 | 0.383 | 0.271 | 0.485 | 0.394 | 0.394 | 0.444 | 0.303 | 0.849 | 0.303 |
| Sens 2 | 81% | 81% | 81% | 81% | 81% | 81% | 81% | 85% | 80% |
| Spec 2 | 37% | 23% | 43% | 38% | 31% | 42% | 31% | 47% | 32% |
| Cutoff 3 | 0.146 | 0.102 | 0.148 | 0.173 | 0.0990 | 0.211 | 0.146 | 0.211 | 0.0990 |
| Sens 3 | 92% | 94% | 91% | 91% | 90% | 90% | 92% | 92% | 92% |
| Spec 3 | 13% | 7% | 12% | 16% | 7% | 20% | 13% | 17% | 8% |
| Cutoff 4 | 1.54 | 1.91 | 1.54 | 1.54 | 1.91 | 1.54 | 1.54 | 1.91 | 1.54 |
| Sens 4 | 38% | 44% | 43% | 44% | 48% | 42% | 42% | 54% | 32% |
| Spec 4 | 70% | 70% | 70% | 70% | 70% | 70% | 70% | 70% | 70% |
| Cutoff 5 | 2.53 | 3.14 | 2.77 | 2.53 | 3.14 | 2.77 | 2.53 | 3.14 | 2.77 |
| Sens 5 | 35% | 44% | 34% | 28% | 33% | 25% | 27% | 31% | 16% |
| Spec 5 | 80% | 80% | 80% | 80% | 80% | 80% | 80% | 80% | 80% |
| Cutoff 6 | 5.50 | 8.51 | 6.40 | 5.50 | 8.51 | 6.40 | 5.50 | 8.51 | 6.40 |
| Sens 6 | 21% | 12% | 23% | 18% | 19% | 17% | 19% | 15% | 12% |
| Spec 6 | 90% | 90% | 90% | 90% | 90% | 90% | 90% | 90% | 90% |
| OR Quart 2 | 2.2 | 0.74 | 1.8 | 1.6 | 0.66 | 1.3 | 0.57 | 0.50 | 0.58 |
| p Value | 0.14 | 0.69 | 0.30 | 0.36 | 0.65 | 0.61 | 0.46 | 0.57 | 0.47 |
| 95% CI of | 0.78 | 0.16 | 0.60 | 0.60 | 0.11 | 0.48 | 0.13 | 0.044 | 0.13 |
| OR Quart 2 | 6.2 | 3.4 | 5.2 | 4.1 | 4.0 | 3.5 | 2.5 | 5.5 | 2.5 |
| OR Quart 3 | 2.4 | 0.49 | 2.2 | 3.0 | 2.8 | 3.2 | 2.1 | 2.6 | 2.8 |
| p Value | 0.093 | 0.41 | 0.14 | 0.015 | 0.14 | 0.013 | 0.19 | 0.27 | 0.074 |
| 95% CI of | 0.86 | 0.087 | 0.77 | 1.2 | 0.72 | 1.3 | 0.69 | 0.49 | 0.90 |
| OR Quart 3 | 6.7 | 2.7 | 6.2 | 7.4 | 11 | 7.8 | 6.6 | 14 | 8.4 |
| OR Quart 4 | 3.3 | 1.8 | 4.0 | 2.4 | 2.8 | 2.0 | 1.7 | 2.6 | 0.98 |
| p Value | 0.018 | 0.37 | 0.0064 | 0.058 | 0.14 | 0.17 | 0.40 | 0.27 | 0.98 |
| 95% CI of | 1.2 | 0.51 | 1.5 | 0.97 | 0.72 | 0.76 | 0.51 | 0.49 | 0.27 |
| OR Quart 4 | 8.9 | 6.3 | 11 | 6.0 | 11 | 5.0 | 5.3 | 14 | 3.6 |

Myoglobin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.21 | 0.965 | 1.21 | 0.546 | 1.21 | 0.330 |
| Average | 26.0 | 38.3 | 26.0 | 70.8 | 26.0 | 47.2 |
| Stdev | 82.5 | 97.7 | 82.5 | 151 | 82.5 | 131 |
| p (t-test) | | 0.36 | | 0.0021 | | 0.24 |
| Min | 0.000105 | 0.000105 | 0.000105 | 0.0276 | 0.000105 | 0.0254 |
| Max | 618 | 469 | 618 | 618 | 618 | 469 |
| n (Samp) | 253 | 48 | 253 | 57 | 253 | 26 |
| n (Patient) | 102 | 48 | 102 | 57 | 102 | 26 |
| sCr only | | | | | | |
| Median | 0.608 | 0.186 | 0.608 | 0.375 | 0.608 | 0.349 |
| Average | 32.4 | 66.7 | 32.4 | 58.7 | 32.4 | 62.5 |
| Stdev | 99.0 | 156 | 99.0 | 131 | 99.0 | 173 |
| p (t-test) | | 0.18 | | 0.24 | | 0.29 |
| Min | 0.000105 | 0.000105 | 0.000105 | 0.0375 | 0.000105 | 0.0567 |
| Max | 618 | 469 | 618 | 442 | 618 | 618 |
| n (Samp) | 447 | 16 | 447 | 21 | 447 | 13 |
| n (Patient) | 170 | 16 | 170 | 21 | 170 | 13 |
| UO only | | | | | | |
| Median | 1.23 | 1.00 | 1.23 | 0.855 | 1.23 | 0.366 |
| Average | 29.6 | 29.5 | 29.6 | 76.5 | 29.6 | 68.8 |
| Stdev | 90.0 | 76.4 | 90.0 | 160 | 90.0 | 155 |
| p (t-test) | | 0.99 | | 0.0051 | | 0.062 |
| Min | 0.00616 | 0.0266 | 0.00616 | 0.000105 | 0.00616 | 0.0254 |
| Max | 618 | 469 | 618 | 618 | 618 | 469 |
| n (Samp) | 212 | 47 | 212 | 52 | 212 | 25 |
| n (Patient) | 85 | 47 | 85 | 52 | 85 | 25 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.51 | 0.48 | 0.51 | 0.50 | 0.53 | 0.51 | 0.45 | 0.52 | 0.50 |
| SE | 0.046 | 0.074 | 0.047 | 0.042 | 0.066 | 0.045 | 0.061 | 0.082 | 0.061 |
| p | 0.85 | 0.84 | 0.87 | 0.94 | 0.66 | 0.84 | 0.44 | 0.76 | 0.97 |
| nCohort 1 | 253 | 447 | 212 | 253 | 447 | 212 | 253 | 447 | 212 |
| nCohort 2 | 48 | 16 | 47 | 57 | 21 | 52 | 26 | 13 | 25 |
| Cutoff 1 | 0.0819 | 0.0422 | 0.0832 | 0.0855 | 0.0849 | 0.0855 | 0.0877 | 0.132 | 0.118 |
| Sens 1 | 71% | 75% | 70% | 70% | 71% | 71% | 73% | 77% | 72% |
| Spec 1 | 19% | 16% | 19% | 19% | 26% | 19% | 19% | 33% | 24% |
| Cutoff 2 | 0.0394 | 0.0333 | 0.0485 | 0.0746 | 0.0556 | 0.0767 | 0.0667 | 0.0667 | 0.0832 |
| Sens 2 | 81% | 81% | 81% | 81% | 81% | 81% | 81% | 85% | 80% |
| Spec 2 | 11% | 12% | 11% | 18% | 21% | 18% | 17% | 23% | 19% |
| Cutoff 3 | 0.0329 | 0 | 0.0333 | 0.0337 | 0.0399 | 0.0333 | 0.0296 | 0.0584 | 0.0296 |
| Sens 3 | 92% | 100% | 91% | 91% | 90% | 90% | 92% | 92% | 92% |
| Spec 3 | 8% | 0% | 7% | 8% | 15% | 7% | 6% | 22% | 5% |
| Cutoff 4 | 6.79 | 5.77 | 7.09 | 6.79 | 5.77 | 7.09 | 6.79 | 5.77 | 7.09 |
| Sens 4 | 44% | 44% | 43% | 35% | 38% | 37% | 23% | 31% | 32% |
| Spec 4 | 70% | 70% | 70% | 70% | 70% | 70% | 70% | 70% | 70% |
| Cutoff 5 | 16.7 | 16.8 | 18.6 | 16.7 | 16.8 | 18.6 | 16.7 | 16.8 | 18.6 |
| Sens 5 | 31% | 38% | 26% | 32% | 29% | 31% | 19% | 15% | 28% |
| Spec 5 | 80% | 80% | 80% | 80% | 80% | 80% | 80% | 80% | 80% |
| Cutoff 6 | 45.5 | 63.4 | 54.1 | 45.5 | 63.4 | 54.1 | 45.5 | 63.4 | 54.1 |
| Sens 6 | 19% | 12% | 13% | 21% | 19% | 23% | 12% | 15% | 16% |
| Spec 6 | 90% | 90% | 90% | 90% | 90% | 90% | 90% | 90% | 90% |
| OR Quart 2 | 0.33 | 0 | 0.33 | 0.47 | 1.0 | 0.42 | 0.65 | 1.7 | 0.47 |
| p Value | 0.020 | na | 0.025 | 0.068 | 1.0 | 0.056 | 0.51 | 0.48 | 0.24 |
| 95% CI of | 0.13 | na | 0.13 | 0.21 | 0.31 | 0.17 | 0.17 | 0.40 | 0.13 |
| OR Quart 2 | 0.84 | na | 0.87 | 1.1 | 3.2 | 1.0 | 2.4 | 7.3 | 1.7 |
| OR Quart 3 | 0.33 | 0.41 | 0.39 | 0.33 | 0.32 | 0.37 | 1.4 | 0.66 | 0.60 |
| p Value | 0.020 | 0.21 | 0.045 | 0.015 | 0.17 | 0.032 | 0.57 | 0.65 | 0.40 |
| 95% CI of | 0.13 | 0.10 | 0.15 | 0.14 | 0.064 | 0.15 | 0.45 | 0.11 | 0.18 |
| OR Quart 3 | 0.84 | 1.6 | 0.98 | 0.81 | 1.6 | 0.92 | 4.2 | 4.0 | 2.0 |
| OR Quart 4 | 0.84 | 0.86 | 0.83 | 0.86 | 1.2 | 0.93 | 1.4 | 1.0 | 1.0 |
| p Value | 0.66 | 0.79 | 0.65 | 0.68 | 0.78 | 0.84 | 0.56 | 1.0 | 0.97 |
| 95% CI of | 0.39 | 0.28 | 0.37 | 0.41 | 0.38 | 0.43 | 0.46 | 0.20 | 0.36 |
| OR Quart 4 | 1.8 | 2.6 | 1.8 | 1.8 | 3.6 | 2.0 | 4.3 | 5.1 | 2.9 |

Mucin-16

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.619 | 0.753 | 0.619 | 0.547 | nd | nd |
| Average | 1.06 | 1.12 | 1.06 | 23.1 | nd | nd |
| Stdev | 1.28 | 0.983 | 1.28 | 96.4 | nd | nd |
| p (t-test) | | 0.85 | | 0.10 | nd | nd |
| Min | 0.141 | 1.00E−9 | 0.141 | 0.223 | nd | nd |
| Max | 6.37 | 3.40 | 6.37 | 433 | nd | nd |
| n (Samp) | 52 | 20 | 52 | 20 | nd | nd |
| n (Patient) | 41 | 20 | 41 | 20 | nd | nd |
| UO only | | | | | | |
| Median | 0.547 | 0.654 | 0.547 | 0.684 | nd | nd |
| Average | 0.951 | 1.00 | 0.951 | 22.0 | nd | nd |
| Stdev | 1.18 | 0.898 | 1.18 | 94.1 | nd | nd |
| p (t-test) | | 0.87 | | 0.15 | nd | nd |
| Min | 0.0565 | 1.00E−9 | 0.0565 | 0.223 | nd | nd |
| Max | 6.37 | 2.82 | 6.37 | 433 | nd | nd |
| n (Samp) | 42 | 16 | 42 | 21 | nd | nd |
| n (Patient) | 33 | 16 | 33 | 21 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.54 | nd | 0.54 | 0.49 | nd | 0.56 | nd | nd | nd |
| SE | 0.077 | nd | 0.086 | 0.077 | nd | 0.078 | nd | nd | nd |
| p | 0.61 | nd | 0.67 | 0.87 | nd | 0.48 | nd | nd | nd |
| nCohort 1 | 52 | nd | 42 | 52 | nd | 42 | nd | nd | nd |
| nCohort 2 | 20 | nd | 16 | 20 | nd | 21 | nd | nd | nd |
| Cutoff 1 | 0.479 | nd | 0.412 | 0.344 | nd | 0.384 | nd | nd | nd |
| Sens 1 | 70% | nd | 75% | 70% | nd | 71% | nd | nd | nd |
| Spec 1 | 37% | nd | 36% | 17% | nd | 29% | nd | nd | nd |
| Cutoff 2 | 0.223 | nd | 0.223 | 0.278 | nd | 0.278 | nd | nd | nd |
| Sens 2 | 85% | nd | 81% | 90% | nd | 90% | nd | nd | nd |
| Spec 2 | 4% | nd | 7% | 10% | nd | 12% | nd | nd | nd |
| Cutoff 3 | 0.146 | nd | 0.0565 | 0.278 | nd | 0.278 | nd | nd | nd |
| Sens 3 | 90% | nd | 94% | 90% | nd | 90% | nd | nd | nd |
| Spec 3 | 4% | nd | 2% | 10% | nd | 12% | nd | nd | nd |
| Cutoff 4 | 0.937 | nd | 0.859 | 0.937 | nd | 0.859 | nd | nd | nd |
| Sens 4 | 45% | nd | 38% | 30% | nd | 43% | nd | nd | nd |
| Spec 4 | 71% | nd | 71% | 71% | nd | 71% | nd | nd | nd |
| Cutoff 5 | 1.24 | nd | 1.09 | 1.24 | nd | 1.09 | nd | nd | nd |
| Sens 5 | 40% | nd | 38% | 30% | nd | 33% | nd | nd | nd |
| Spec 5 | 81% | nd | 81% | 81% | nd | 81% | nd | nd | nd |
| Cutoff 6 | 1.87 | nd | 1.68 | 1.87 | nd | 1.68 | nd | nd | nd |
| Sens 6 | 20% | nd | 19% | 25% | nd | 24% | nd | nd | nd |
| Spec 6 | 90% | nd | 90% | 90% | nd | 90% | nd | nd | nd |
| OR Quart 2 | 0.74 | nd | 0.62 | 0.40 | nd | 0.50 | nd | nd | nd |
| p Value | 0.70 | nd | 0.59 | 0.26 | nd | 0.38 | nd | nd | nd |
| 95% CI of | 0.16 | nd | 0.11 | 0.082 | nd | 0.11 | nd | nd | nd |
| OR Quart 2 | 3.4 | nd | 3.5 | 1.9 | nd | 2.3 | nd | nd | nd |
| OR Quart 3 | 0.52 | nd | 0.68 | 0.77 | nd | 0.50 | nd | nd | nd |
| p Value | 0.43 | nd | 0.66 | 0.72 | nd | 0.38 | nd | nd | nd |
| 95% CI of | 0.10 | nd | 0.12 | 0.19 | nd | 0.11 | nd | nd | nd |
| OR Quart 3 | 2.6 | nd | 3.8 | 3.2 | nd | 2.3 | nd | nd | nd |
| OR Quart 4 | 2.1 | nd | 1.7 | 1.0 | nd | 1.2 | nd | nd | nd |
| p Value | 0.30 | nd | 0.52 | 1.0 | nd | 0.83 | nd | nd | nd |
| 95% CI of | 0.52 | nd | 0.35 | 0.25 | nd | 0.28 | nd | nd | nd |
| OR Quart 4 | 8.3 | nd | 7.9 | 4.0 | nd | 4.9 | nd | nd | nd |

Poly [ADP-ribose] polymerase 1 (cleaved)

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.00E−9 | 1.00E−9 | 1.00E−9 | 1.00E−9 | 1.00E−9 | 0.00439 |
| Average | 0.00474 | 0.00294 | 0.00474 | 0.00432 | 0.00474 | 0.00271 |
| Stdev | 0.00688 | 0.00538 | 0.00688 | 0.00586 | 0.00688 | 0.00261 |
| p (t-test) | | 0.11 | | 0.70 | | 0.16 |
| Min | 1.00E−9 | 1.00E−9 | 1.00E−9 | 1.00E−9 | 1.00E−9 | 1.00E−9 |
| Max | 0.0357 | 0.0144 | 0.0357 | 0.0183 | 0.0357 | 0.00723 |
| n (Samp) | 118 | 47 | 118 | 54 | 118 | 24 |
| n (Patient) | 97 | 47 | 97 | 54 | 97 | 24 |
| sCr only | | | | | | |
| Median | 1.00E−9 | 1.00E−9 | 1.00E−9 | 1.00E−9 | 1.00E−9 | 1.00E−9 |
| Average | 0.00375 | 0.00338 | 0.00375 | 0.00301 | 0.00375 | 0.00350 |
| Stdev | 0.00582 | 0.00600 | 0.00582 | 0.00648 | 0.00582 | 0.00540 |
| p (t-test) | | 0.82 | | 0.60 | | 0.89 |
| Min | 1.00E−9 | 1.00E−9 | 1.00E−9 | 1.00E−9 | 1.00E−9 | 1.00E−9 |
| Max | 0.0357 | 0.0144 | 0.0357 | 0.0253 | 0.0357 | 0.0144 |
| n (Samp) | 263 | 14 | 263 | 19 | 263 | 12 |
| n (Patient) | 159 | 14 | 159 | 19 | 159 | 12 |
| UO only | | | | | | |
| Median | 1.00E−9 | 1.00E−9 | 1.00E−9 | 0.00340 | 1.00E−9 | 0.00471 |
| Average | 0.00356 | 0.00258 | 0.00356 | 0.00527 | 0.00356 | 0.00448 |
| Stdev | 0.00628 | 0.00494 | 0.00628 | 0.00622 | 0.00628 | 0.00455 |
| p (t-test) | | 0.36 | | 0.12 | | 0.51 |
| Min | 1.00E−9 | 1.00E−9 | 1.00E−9 | 1.00E−9 | 1.00E−9 | 1.00E−9 |
| Max | 0.0357 | 0.0144 | 0.0357 | 0.0183 | 0.0357 | 0.0144 |
| n (Samp) | 105 | 45 | 105 | 49 | 105 | 23 |
| n (Patient) | 84 | 45 | 84 | 49 | 84 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.43 | 0.46 | 0.47 | 0.51 | 0.45 | 0.59 | 0.50 | 0.50 | 0.62 |
| SE | 0.050 | 0.081 | 0.052 | 0.048 | 0.070 | 0.050 | 0.065 | 0.086 | 0.068 |
| p | 0.18 | 0.63 | 0.51 | 0.85 | 0.52 | 0.060 | 0.97 | 0.97 | 0.071 |
| nCohort 1 | 118 | 263 | 105 | 118 | 263 | 105 | 118 | 263 | 105 |
| nCohort 2 | 47 | 14 | 45 | 54 | 19 | 49 | 24 | 12 | 23 |
| Cutoff 1 | 0 | 0 | 0 | 0 | 0 | 0 | 0 | 0 | 0 |
| Sens 1 | 100% | 100% | 100% | 100% | 100% | 100% | 100% | 100% | 100% |
| Spec 1 | 0% | 0% | 0% | 0% | 0% | 0% | 0% | 0% | 0% |
| Cutoff 2 | 0 | 0 | 0 | 0 | 0 | 0 | 0 | 0 | 0 |
| Sens 2 | 100% | 100% | 100% | 100% | 100% | 100% | 100% | 100% | 100% |
| Spec 2 | 0% | 0% | 0% | 0% | 0% | 0% | 0% | 0% | 0% |
| Cutoff 3 | 0 | 0 | 0 | 0 | 0 | 0 | 0 | 0 | 0 |
| Sens 3 | 100% | 100% | 100% | 100% | 100% | 100% | 100% | 100% | 100% |
| Spec 3 | 0% | 0% | 0% | 0% | 0% | 0% | 0% | 0% | 0% |
| Cutoff 4 | 0.00598 | 0.00471 | 0.00406 | 0.00598 | 0.00471 | 0.00406 | 0.00598 | 0.00471 | 0.00406 |
| Sens 4 | 17% | 21% | 24% | 24% | 16% | 47% | 4% | 17% | 61% |
| Spec 4 | 70% | 74% | 70% | 70% | 74% | 70% | 70% | 74% | 70% |
| Cutoff 5 | 0.0141 | 0.00723 | 0.00681 | 0.0141 | 0.00723 | 0.00681 | 0.0141 | 0.00723 | 0.00681 |
| Sens 5 | 11% | 21% | 13% | 19% | 11% | 31% | 0% | 17% | 17% |
| Spec 5 | 81% | 81% | 80% | 81% | 81% | 80% | 81% | 81% | 80% |
| Cutoff 6 | 0.0144 | 0.0144 | 0.0144 | 0.0144 | 0.0144 | 0.0144 | 0.0144 | 0.0144 | 0.0144 |
| Sens 6 | 0% | 0% | 0% | 2% | 5% | 2% | 0% | 0% | 0% |
| Spec 6 | 95% | 97% | 99% | 95% | 97% | 99% | 95% | 97% | 99% |
| OR Quart 2 | 0.59 | 0.33 | 1.3 | 2.3 | 1.0 | 1.3 | 18 | 1.5 | 1.0 |
| p Value | 0.39 | 0.34 | 0.56 | 0.073 | 0.99 | 0.63 | 0.0069 | 0.65 | 1.0 |
| 95% CI of | 0.18 | 0.033 | 0.50 | 0.93 | 0.20 | 0.45 | 2.2 | 0.25 | 0.23 |
| OR Quart 2 | 2.0 | 3.2 | 3.6 | 5.7 | 5.2 | 3.7 | 150 | 9.4 | 4.4 |
| OR Quart 3 | 7.4 | 3.4 | 1.0 | 1.3 | 4.6 | 2.2 | 5.6 | 3.8 | 2.3 |
| p Value | 8.9E−5 | 0.080 | 1.0 | 0.63 | 0.022 | 0.13 | 0.12 | 0.11 | 0.21 |
| 95% CI of | 2.7 | 0.87 | 0.36 | 0.49 | 1.2 | 0.79 | 0.62 | 0.76 | 0.62 |
| OR Quar 3 | 20 | 13 | 2.8 | 3.2 | 17 | 6.1 | 51 | 19 | 8.7 |
| OR Quart 4 | 1.0 | 0.33 | 1.5 | 1.0 | 0.33 | 2.9 | 7.2 | 0 | 2.0 |
| p Value | 0.96 | 0.34 | 0.41 | 1.0 | 0.34 | 0.038 | 0.074 | na | 0.33 |
| 95% CI of | 0.35 | 0.033 | 0.56 | 0.38 | 0.033 | 1.1 | 0.82 | na | 0.51 |
| OR Quart 4 | 3.1 | 3.2 | 4.1 | 2.6 | 3.2 | 7.9 | 64 | na | 7.5 |

KSP-Cadherin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.03 | 1.24 | 1.03 | 1.15 | nd | nd |
| Average | 1.47 | 1.58 | 1.47 | 1.90 | nd | nd |
| Stdev | 1.59 | 1.15 | 1.59 | 1.74 | nd | nd |
| p (t-test) | | 0.71 | | 0.25 | nd | nd |
| Min | 0.00263 | 0.0646 | 0.00263 | 0.291 | nd | nd |
| Max | 11.9 | 4.63 | 11.9 | 7.51 | nd | nd |
| n (Samp) | 85 | 32 | 85 | 25 | nd | nd |
| n (Patient) | 68 | 32 | 68 | 25 | nd | nd |
| sCr only | | | | | | |
| Median | 1.10 | 0.683 | 1.10 | 1.04 | nd | nd |
| Average | 1.58 | 1.36 | 1.58 | 1.60 | nd | nd |
| Stdev | 1.55 | 1.28 | 1.55 | 1.45 | nd | nd |
| p (t-test) | | 0.66 | | 0.98 | nd | nd |
| Min | 0.00263 | 0.0646 | 0.00263 | 0.291 | nd | nd |
| Max | 11.9 | 3.49 | 11.9 | 4.08 | nd | nd |
| n (Samp) | 152 | 10 | 152 | 6 | nd | nd |
| n (Patient) | 114 | 10 | 114 | 6 | nd | nd |
| UO only | | | | | | |
| Median | 1.02 | 1.40 | 1.02 | 1.61 | nd | nd |
| Average | 1.55 | 1.73 | 1.55 | 2.07 | nd | nd |
| Stdev | 1.72 | 1.11 | 1.72 | 1.77 | nd | nd |
| p (t-test) | | 0.61 | | 0.19 | nd | nd |
| Min | 0.00263 | 0.557 | 0.00263 | 0.371 | nd | nd |
| Max | 11.9 | 4.63 | 11.9 | 7.51 | nd | nd |
| n (Samp) | 73 | 27 | 73 | 25 | nd | nd |
| n (Patient) | 59 | 27 | 59 | 25 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | 0.44 | 0.62 | 0.58 | 0.50 | 0.60 | nd | nd | nd |
| SE | 0.061 | 0.097 | 0.065 | 0.067 | 0.12 | 0.068 | nd | nd | nd |
| p | 0.25 | 0.53 | 0.068 | 0.26 | 1.00 | 0.12 | nd | nd | nd |
| nCohort 1 | 85 | 152 | 73 | 85 | 152 | 73 | nd | nd | nd |
| nCohort 2 | 32 | 10 | 27 | 25 | 6 | 25 | nd | nd | nd |
| Cutoff 1 | 0.946 | 0.622 | 0.996 | 0.845 | 0.569 | 0.845 | nd | nd | nd |
| Sens 1 | 72% | 70% | 70% | 72% | 83% | 72% | nd | nd | nd |
| Spec 1 | 46% | 25% | 48% | 40% | 22% | 41% | nd | nd | nd |
| Cutoff 2 | 0.734 | 0.380 | 0.935 | 0.569 | 0.569 | 0.723 | nd | nd | nd |
| Sens 2 | 81% | 80% | 81% | 80% | 83% | 80% | nd | nd | nd |
| Spec 2 | 35% | 14% | 47% | 22% | 22% | 34% | nd | nd | nd |
| Cutoff 3 | 0.555 | 0.0646 | 0.739 | 0.398 | 0.252 | 0.426 | nd | nd | nd |
| Sens 3 | 91% | 90% | 93% | 92% | 100% | 92% | nd | nd | nd |
| Spec 3 | 21% | 3% | 36% | 15% | 8% | 16% | nd | nd | nd |
| Cutoff 4 | 1.66 | 1.69 | 1.71 | 1.66 | 1.69 | 1.71 | nd | nd | nd |
| Sens 4 | 28% | 40% | 30% | 40% | 33% | 40% | nd | nd | nd |
| Spec 4 | 71% | 70% | 71% | 71% | 70% | 71% | nd | nd | nd |
| Cutoff 5 | 2.10 | 2.45 | 2.19 | 2.10 | 2.45 | 2.19 | nd | nd | nd |
| Sens 5 | 25% | 30% | 26% | 36% | 33% | 36% | nd | nd | nd |
| Spec 5 | 80% | 80% | 81% | 80% | 80% | 81% | nd | nd | nd |
| Cutoff 6 | 2.74 | 3.39 | 3.17 | 2.74 | 3.39 | 3.17 | nd | nd | nd |
| Sens 6 | 16% | 10% | 11% | 16% | 17% | 20% | nd | nd | nd |
| Spec 6 | 91% | 90% | 90% | 91% | 90% | 90% | nd | nd | nd |
| OR Quart 2 | 2.2 | 0.32 | 16 | 0.76 | 0.47 | 0.95 | nd | nd | nd |
| p Value | 0.22 | 0.34 | 0.012 | 0.69 | 0.55 | 0.94 | nd | nd | nd |
| 95% CI of | 0.62 | 0.032 | 1.9 | 0.20 | 0.041 | 0.24 | nd | nd | nd |
| OR Quart 2 | 7.5 | 3.3 | 140 | 2.9 | 5.5 | 3.8 | nd | nd | nd |
| OR Quart 3 | 2.5 | 1.0 | 14 | 0.80 | 0.49 | 1.3 | nd | nd | nd |
| p Value | 0.14 | 1.0 | 0.018 | 0.74 | 0.56 | 0.73 | nd | nd | nd |
| 95% CI of | 0.74 | 0.19 | 1.6 | 0.21 | 0.042 | 0.33 | nd | nd | nd |
| OR Quart 3 | 8.6 | 5.3 | 120 | 3.0 | 5.6 | 4.9 | nd | nd | nd |
| OR Quart 4 | 1.7 | 1.0 | 9.3 | 1.7 | 0.97 | 2.1 | nd | nd | nd |
| p Value | 0.39 | 0.97 | 0.045 | 0.41 | 0.98 | 0.24 | nd | nd | nd |
| 95% CI of | 0.50 | 0.19 | 1.1 | 0.50 | 0.13 | 0.59 | nd | nd | nd |
| OR Quart 4 | 6.1 | 5.4 | 83 | 5.5 | 7.3 | 7.7 | nd | nd | nd |

Tumor necrosis factor receptor superfamily member 10B

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.124 | 0.573 | 0.124 | 0.395 | nd | nd |
| Average | 0.891 | 1.21 | 0.891 | 1.09 | nd | nd |
| Stdev | 1.49 | 1.55 | 1.49 | 1.82 | nd | nd |
| p (t-test) | | 0.43 | | 0.64 | nd | nd |
| Min | 0.00360 | 0.0182 | 0.00360 | 0.00464 | nd | nd |
| Max | 6.71 | 4.65 | 6.71 | 6.48 | nd | nd |
| n (Samp) | 52 | 20 | 52 | 20 | nd | nd |
| n (Patient) | 41 | 20 | 41 | 20 | nd | nd |
| UO only | | | | | | |
| Median | 0.106 | 0.709 | 0.106 | 0.464 | nd | nd |
| Average | 0.595 | 1.40 | 0.595 | 1.25 | nd | nd |
| Stdev | 1.03 | 1.68 | 1.03 | 1.96 | nd | nd |
| p (t-test) | | 0.031 | | 0.087 | nd | nd |
| Min | 0.00360 | 0.0573 | 0.00360 | 0.0172 | nd | nd |
| Max | 4.38 | 4.65 | 4.38 | 6.48 | nd | nd |
| n (Samp) | 42 | 16 | 42 | 21 | nd | nd |
| n (Patient) | 33 | 16 | 33 | 21 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.64 | nd | 0.71 | 0.62 | nd | 0.68 | nd | nd | nd |
| SE | 0.076 | nd | 0.081 | 0.076 | nd | 0.074 | nd | nd | nd |
| p | 0.064 | nd | 0.0099 | 0.12 | nd | 0.014 | nd | nd | nd |
| nCohort 1 | 52 | nd | 42 | 52 | nd | 42 | nd | nd | nd |
| nCohort 2 | 20 | nd | 16 | 20 | nd | 21 | nd | nd | nd |
| Cutoff 1 | 0.182 | nd | 0.107 | 0.154 | nd | 0.154 | nd | nd | nd |
| Sens 1 | 70% | nd | 75% | 70% | nd | 71% | nd | nd | nd |
| Spec 1 | 54% | nd | 52% | 52% | nd | 55% | nd | nd | nd |
| Cutoff 2 | 0.0761 | nd | 0.0761 | 0.141 | nd | 0.141 | nd | nd | nd |
| Sens 2 | 80% | nd | 81% | 80% | nd | 81% | nd | nd | nd |
| Spec 2 | 40% | nd | 40% | 52% | nd | 55% | nd | nd | nd |
| Cutoff 3 | 0.0591 | nd | 0.0591 | 0.0796 | nd | 0.101 | nd | nd | nd |
| Sens 3 | 90% | nd | 94% | 90% | nd | 90% | nd | nd | nd |
| Spec 3 | 38% | nd | 38% | 44% | nd | 48% | nd | nd | nd |
| Cutoff 4 | 0.786 | nd | 0.398 | 0.786 | nd | 0.398 | nd | nd | nd |
| Sens 4 | 45% | nd | 56% | 30% | nd | 52% | nd | nd | nd |
| Spec 4 | 71% | nd | 71% | 71% | nd | 71% | nd | nd | nd |
| Cutoff 5 | 1.56 | nd | 0.819 | 1.56 | nd | 0.819 | nd | nd | nd |
| Sens 5 | 25% | nd | 50% | 15% | nd | 33% | nd | nd | nd |
| Spec 5 | 81% | nd | 81% | 81% | nd | 81% | nd | nd | nd |
| Cutoff 6 | 3.11 | nd | 2.19 | 3.11 | nd | 2.19 | nd | nd | nd |
| Sens 6 | 15% | nd | 25% | 10% | nd | 14% | nd | nd | nd |
| Spec 6 | 90% | nd | 90% | 90% | nd | 90% | nd | nd | nd |
| OR Quart 2 | 8.5 | nd | >7.0 | 11 | nd | 11 | nd | nd | nd |
| p Value | 0.061 | nd | <0.097 | 0.036 | nd | 0.038 | nd | nd | nd |
| 95% CI of | 0.90 | nd | >0.71 | 1.2 | nd | 1.1 | nd | nd | nd |
| OR Quart 2 | 80 | nd | na | 100 | nd | 100 | nd | nd | nd |
| OR Quart 3 | 11 | nd | >3.8 | 11 | nd | 8.4 | nd | nd | nd |
| p Value | 0.036 | nd | <0.27 | 0.036 | nd | 0.066 | nd | nd | nd |
| 95% CI of | 1.2 | nd | >0.35 | 1.2 | nd | 0.87 | nd | nd | nd |
| OR Quart 3 | 100 | nd | na | 100 | nd | 81 | nd | nd | nd |
| OR Quart 4 | 8.5 | nd | >16 | 6.5 | nd | 11 | nd | nd | nd |
| p Value | 0.061 | nd | <0.017 | 0.10 | nd | 0.038 | nd | nd | nd |
| 95% CI of | 0.90 | nd | >1.7 | 0.68 | nd | 1.1 | nd | nd | nd |
| OR Quart 4 | 80 | nd | na | 63 | nd | 100 | nd | nd | nd |

TABLE 2
Comparison of marker levels in urine samples collected from Cohort 1
(patients that did not progress beyond RIFLE stage 0 or R) and in urine samples collected from
subjects at 0, 24 hours, and 48 hours prior to reaching stage I or F in Cohort 2.
Apolipoprotein A-II

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 73.2 | 102 | 73.2 | 116 | 73.2 | 81.7 |
| Average | 185 | 272 | 185 | 697 | 185 | 87.4 |
| Stdev | 645 | 1020 | 645 | 3530 | 645 | 69.4 |
| p (t-test) | | 0.44 | | 0.0018 | | 0.44 |
| Min | 2.08 | 8.69 | 2.08 | 10.8 | 2.08 | 1.00E−9 |
| Max | 6400 | 6400 | 6400 | 24300 | 6400 | 301 |
| n (Samp) | 685 | 38 | 685 | 47 | 685 | 26 |
| n (Patient) | 283 | 38 | 283 | 47 | 283 | 26 |
| sCr only | | | | | | |
| Median | 76.6 | 106 | 76.6 | 127 | 76.6 | 98.8 |
| Average | 198 | 891 | 198 | 176 | 198 | 108 |
| Stdev | 990 | 2230 | 990 | 158 | 990 | 84.1 |
| p (t-test) | | 0.053 | | 0.93 | | 0.74 |
| Min | 1.00E−9 | 34.5 | 1.00E−9 | 24.0 | 1.00E−9 | 6.18 |
| Max | 24300 | 6400 | 24300 | 613 | 24300 | 288 |
| n (Samp) | 891 | 8 | 891 | 13 | 891 | 13 |
| n (Patient) | 334 | 8 | 334 | 13 | 334 | 13 |
| UO only | | | | | | |
| Median | 76.6 | 107 | 76.6 | 131 | 76.6 | 98.7 |
| Average | 195 | 297 | 195 | 789 | 195 | 105 |
| Stdev | 649 | 1060 | 649 | 3780 | 649 | 74.2 |
| p (t-test) | | 0.39 | | 0.0017 | | 0.52 |
| Min | 2.08 | 8.69 | 2.08 | 10.8 | 2.08 | 1.00E−9 |
| Max | 6400 | 6400 | 6400 | 24300 | 6400 | 301 |
| n (Samp) | 553 | 35 | 553 | 41 | 553 | 22 |
| n (Patient) | 202 | 35 | 202 | 41 | 202 | 22 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.59 | 0.60 | 0.60 | 0.66 | 0.66 | 0.67 | 0.47 | 0.51 | 0.53 |
| SE | 0.050 | 0.11 | 0.052 | 0.045 | 0.083 | 0.048 | 0.059 | 0.081 | 0.064 |
| p | 0.069 | 0.36 | 0.053 | 3.3E−4 | 0.055 | 2.3E−4 | 0.60 | 0.87 | 0.61 |
| nCohort 1 | 685 | 891 | 553 | 685 | 891 | 553 | 685 | 891 | 553 |
| nCohort 2 | 38 | 8 | 35 | 47 | 13 | 41 | 26 | 13 | 22 |
| Cutoff 1 | 70.0 | 59.9 | 76.9 | 85.8 | 85.8 | 99.4 | 39.3 | 39.3 | 56.5 |
| Sens 1 | 71% | 75% | 71% | 70% | 77% | 71% | 73% | 77% | 73% |
| Spec 1 | 48% | 37% | 50% | 59% | 56% | 63% | 22% | 21% | 35% |
| Cutoff 2 | 56.5 | 49.4 | 69.8 | 66.5 | 66.5 | 72.6 | 31.3 | 31.3 | 43.2 |
| Sens 2 | 82% | 88% | 80% | 81% | 85% | 80% | 81% | 85% | 82% |
| Spec 2 | 38% | 30% | 45% | 46% | 43% | 47% | 14% | 13% | 22% |
| Cutoff 3 | 33.5 | 34.3 | 33.5 | 43.2 | 43.2 | 44.8 | 11.0 | 16.2 | 27.1 |
| Sens 3 | 92% | 100% | 91% | 91% | 92% | 90% | 92% | 92% | 91% |
| Spec 3 | 16% | 16% | 14% | 26% | 24% | 24% | 2% | 3% | 9% |
| Cutoff 4 | 110 | 122 | 118 | 110 | 122 | 118 | 110 | 122 | 118 |
| Sens 4 | 42% | 50% | 46% | 53% | 62% | 54% | 31% | 38% | 36% |
| Spec 4 | 70% | 70% | 70% | 70% | 70% | 70% | 70% | 70% | 70% |
| Cutoff 5 | 148 | 154 | 154 | 148 | 154 | 154 | 148 | 154 | 154 |
| Sens 5 | 18% | 38% | 20% | 38% | 31% | 41% | 19% | 23% | 14% |
| Spec 5 | 80% | 80% | 80% | 80% | 80% | 80% | 80% | 80% | 80% |
| Cutoff 6 | 226 | 229 | 248 | 226 | 229 | 248 | 226 | 229 | 248 |
| Sens 6 | 11% | 12% | 11% | 21% | 31% | 24% | 4% | 8% | 5% |
| Spec 6 | 90% | 90% | 90% | 90% | 90% | 90% | 90% | 90% | 90% |
| OR Quart 2 | 1.2 | 2.0 | 1.2 | 1.4 | 0.50 | 0.49 | 1.3 | 0 | 0.79 |
| p Value | 0.79 | 0.57 | 0.76 | 0.56 | 0.57 | 0.31 | 0.59 | na | 0.73 |
| 95% CI of | 0.38 | 0.18 | 0.36 | 0.44 | 0.045 | 0.12 | 0.46 | na | 0.21 |
| OR Quart 2 | 3.5 | 22 | 4.1 | 4.5 | 5.5 | 2.0 | 4.0 | na | 3.0 |
| OR Quart 3 | 2.2 | 2.0 | 2.5 | 3.2 | 2.5 | 2.3 | 0.49 | 0.59 | 1.2 |
| p Value | 0.11 | 0.57 | 0.090 | 0.028 | 0.27 | 0.10 | 0.32 | 0.48 | 0.77 |
| 95% CI of | 0.83 | 0.18 | 0.87 | 1.1 | 0.49 | 0.84 | 0.12 | 0.14 | 0.36 |
| OR Quart 3 | 6.0 | 22 | 7.4 | 8.9 | 13 | 6.2 | 2.0 | 2.5 | 4.0 |
| OR Quart 4 | 2.1 | 3.0 | 2.5 | 4.4 | 2.5 | 3.5 | 1.5 | 1.0 | 1.4 |
| p Value | 0.16 | 0.34 | 0.090 | 0.0040 | 0.27 | 0.010 | 0.43 | 1.0 | 0.57 |
| 95% CI of | 0.76 | 0.31 | 0.87 | 1.6 | 0.49 | 1.3 | 0.53 | 0.29 | 0.44 |
| OR Quart 4 | 5.6 | 29 | 7.4 | 12 | 13 | 8.9 | 4.4 | 3.5 | 4.6 |

Caspase-1

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.02 | 0.826 | nd | nd | nd | nd |
| Average | 1.20 | 0.896 | nd | nd | nd | nd |
| Stdev | 1.22 | 0.687 | nd | nd | nd | nd |
| p (t-test) | | 0.49 | nd | nd | nd | nd |
| Min | 0.0223 | 0.0223 | nd | nd | nd | nd |
| Max | 4.68 | 2.33 | nd | nd | nd | nd |
| n (Samp) | 55 | 8 | nd | nd | nd | nd |
| n (Patient) | 35 | 8 | nd | nd | nd | nd |
| UO only | | | | | | |
| Median | 1.09 | 0.826 | nd | nd | nd | nd |
| Average | 1.20 | 0.896 | nd | nd | nd | nd |
| Stdev | 1.19 | 0.687 | nd | nd | nd | nd |
| p (t-test) | | 0.50 | nd | nd | nd | nd |
| Min | 0.0223 | 0.0223 | nd | nd | nd | nd |
| Max | 4.46 | 2.33 | nd | nd | nd | nd |
| n (Samp) | 41 | 8 | nd | nd | nd | nd |
| n (Patient) | 24 | 8 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.46 | nd | 0.46 | nd | nd | nd | nd | nd | nd |
| SE | 0.11 | nd | 0.11 | nd | nd | nd | nd | nd | nd |
| p | 0.74 | nd | 0.76 | nd | nd | nd | nd | nd | nd |
| nCohort 1 | 55 | nd | 41 | nd | nd | nd | nd | nd | nd |
| nCohort 2 | 8 | nd | 8 | nd | nd | nd | nd | nd | nd |
| Cutoff 1 | 0.496 | nd | 0.496 | nd | nd | nd | nd | nd | nd |
| Sens 1 | 75% | nd | 75% | nd | nd | nd | nd | nd | nd |
| Spec 1 | 40% | nd | 41% | nd | nd | nd | nd | nd | nd |
| Cutoff 2 | 0.298 | nd | 0.298 | nd | nd | nd | nd | nd | nd |
| Sens 2 | 88% | nd | 88% | nd | nd | nd | nd | nd | nd |
| Spec 2 | 36% | nd | 37% | nd | nd | nd | nd | nd | nd |
| Cutoff 3 | 0 | nd | 0 | nd | nd | nd | nd | nd | nd |
| Sens 3 | 100% | nd | 100% | nd | nd | nd | nd | nd | nd |
| Spec 3 | 0% | nd | 0% | nd | nd | nd | nd | nd | nd |
| Cutoff 4 | 1.54 | nd | 1.54 | nd | nd | nd | nd | nd | nd |
| Sens 4 | 12% | nd | 12% | nd | nd | nd | nd | nd | nd |
| Spec 4 | 71% | nd | 71% | nd | nd | nd | nd | nd | nd |
| Cutoff 5 | 2.07 | nd | 2.07 | nd | nd | nd | nd | nd | nd |
| Sens 5 | 12% | nd | 12% | nd | nd | nd | nd | nd | nd |
| Spec 5 | 82% | nd | 85% | nd | nd | nd | nd | nd | nd |
| Cutoff 6 | 2.59 | nd | 2.59 | nd | nd | nd | nd | nd | nd |
| Sens 6 | 0% | nd | 0% | nd | nd | nd | nd | nd | nd |
| Spec 6 | 91% | nd | 90% | nd | nd | nd | nd | nd | nd |
| OR Quart 2 | 1.0 | nd | 2.4 | nd | nd | nd | nd | nd | nd |
| p Value | 1.0 | nd | 0.50 | nd | nd | nd | nd | nd | nd |
| 95% CI of | 0.057 | nd | 0.19 | nd | nd | nd | nd | nd | nd |
| OR Quart 2 | 18 | nd | 31 | nd | nd | nd | nd | nd | nd |
| OR Quart 3 | 6.8 | nd | 6.0 | nd | nd | nd | nd | nd | nd |
| p Value | 0.099 | nd | 0.14 | nd | nd | nd | nd | nd | nd |
| 95% CI of | 0.69 | nd | 0.56 | nd | nd | nd | nd | nd | nd |
| OR Quart 3 | 67 | nd | 64 | nd | nd | nd | nd | nd | nd |
| OR Quart 4 | 1.1 | nd | 1.1 | nd | nd | nd | nd | nd | nd |
| p Value | 0.96 | nd | 0.95 | nd | nd | nd | nd | nd | nd |
| 95% CI of | 0.061 | nd | 0.061 | nd | nd | nd | nd | nd | nd |
| OR Quart 4 | 19 | nd | 20 | nd | nd | nd | nd | nd | nd |

Caspase-9

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.469 | 0.547 | nd | nd | nd | nd |
| Average | 0.965 | 0.998 | nd | nd | nd | nd |
| Stdev | 2.44 | 1.52 | nd | nd | nd | nd |
| p (t-test) | | 0.97 | nd | nd | nd | nd |
| Min | 0.0360 | 0.0360 | nd | nd | nd | nd |
| Max | 19.5 | 4.64 | nd | nd | nd | nd |
| n (Samp) | 78 | 8 | nd | nd | nd | nd |
| n (Patient) | 56 | 8 | nd | nd | nd | nd |
| UO only | | | | | | |
| Median | 0.469 | 0.547 | nd | nd | nd | nd |
| Average | 1.12 | 0.998 | nd | nd | nd | nd |
| Stdev | 2.73 | 1.52 | nd | nd | nd | nd |
| p (t-test) | | 0.90 | nd | nd | nd | nd |
| Min | 0.0360 | 0.0360 | nd | nd | nd | nd |
| Max | 19.5 | 4.64 | nd | nd | nd | nd |
| n (Samp) | 61 | 8 | nd | nd | nd | nd |
| n (Patient) | 43 | 8 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.54 | nd | 0.50 | nd | nd | nd | nd | nd | nd |
| SE | 0.11 | nd | 0.11 | nd | nd | nd | nd | nd | nd |
| p | 0.73 | nd | 0.98 | nd | nd | nd | nd | nd | nd |
| nCohort 1 | 78 | nd | 61 | nd | nd | nd | nd | nd | nd |
| nCohort 2 | 8 | nd | 8 | nd | nd | nd | nd | nd | nd |
| Cutoff 1 | 0.208 | nd | 0.208 | nd | nd | nd | nd | nd | nd |
| Sens 1 | 75% | nd | 75% | nd | nd | nd | nd | nd | nd |
| Spec 1 | 36% | nd | 28% | nd | nd | nd | nd | nd | nd |
| Cutoff 2 | 0 | nd | 0 | nd | nd | nd | nd | nd | nd |
| Sens 2 | 100% | nd | 100% | nd | nd | nd | nd | nd | nd |
| Spec 2 | 0% | nd | 0% | nd | nd | nd | nd | nd | nd |
| Cutoff 3 | 0 | nd | 0 | nd | nd | nd | nd | nd | nd |
| Sens 3 | 100% | nd | 100% | nd | nd | nd | nd | nd | nd |
| Spec 3 | 0% | nd | 0% | nd | nd | nd | nd | nd | nd |
| Cutoff 4 | 0.781 | nd | 0.781 | nd | nd | nd | nd | nd | nd |
| Sens 4 | 38% | nd | 38% | nd | nd | nd | nd | nd | nd |
| Spec 4 | 72% | nd | 70% | nd | nd | nd | nd | nd | nd |
| Cutoff 5 | 0.955 | nd | 0.955 | nd | nd | nd | nd | nd | nd |
| Sens 5 | 25% | nd | 25% | nd | nd | nd | nd | nd | nd |
| Spec 5 | 81% | nd | 80% | nd | nd | nd | nd | nd | nd |
| Cutoff 6 | 1.66 | nd | 1.49 | nd | nd | nd | nd | nd | nd |
| Sens 6 | 12% | nd | 12% | nd | nd | nd | nd | nd | nd |
| Spec 6 | 91% | nd | 90% | nd | nd | nd | nd | nd | nd |
| OR Quart 2 | 0.95 | nd | 0.47 | nd | nd | nd | nd | nd | nd |
| p Value | 0.96 | nd | 0.55 | nd | nd | nd | nd | nd | nd |
| 95% CI of | 0.12 | nd | 0.038 | nd | nd | nd | nd | nd | nd |
| OR Quart 2 | 7.4 | nd | 5.7 | nd | nd | nd | nd | nd | nd |
| OR Quart 3 | 1.0 | nd | 1.0 | nd | nd | nd | nd | nd | nd |
| p Value | 1.0 | nd | 1.0 | nd | nd | nd | nd | nd | nd |
| 95% CI of | 0.13 | nd | 0.12 | nd | nd | nd | nd | nd | nd |
| OR Quart 3 | 7.9 | nd | 8.1 | nd | nd | nd | nd | nd | nd |
| OR Quart 4 | 0.95 | nd | 1.5 | nd | nd | nd | nd | nd | nd |
| p Value | 0.96 | nd | 0.68 | nd | nd | nd | nd | nd | nd |
| 95% CI of | 0.12 | nd | 0.22 | nd | nd | nd | nd | nd | nd |
| OR Quart 4 | 7.4 | nd | 10 | nd | nd | nd | nd | nd | nd |

Cadherin-1

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | nd | nd | 57300 | 62400 | nd | nd |
| Average | nd | nd | 102000 | 78500 | nd | nd |
| Stdev | nd | nd | 128000 | 81700 | nd | nd |
| p (t-test) | nd | nd | | 0.47 | nd | nd |
| Min | nd | nd | 160 | 1620 | nd | nd |
| Max | nd | nd | 744000 | 260000 | nd | nd |
| n (Samp) | nd | nd | 96 | 16 | nd | nd |
| n (Patient) | nd | nd | 73 | 16 | nd | nd |
| UO only | | | | | | |
| Median | nd | nd | 58600 | 59500 | nd | nd |
| Average | nd | nd | 104000 | 82000 | nd | nd |
| Stdev | nd | nd | 134000 | 82800 | nd | nd |
| p (t-test) | nd | nd | | 0.54 | nd | nd |
| Min | nd | nd | 160 | 1620 | nd | nd |
| Max | nd | nd | 744000 | 260000 | nd | nd |
| n (Samp) | nd | nd | 81 | 15 | nd | nd |
| n (Patient) | nd | nd | 61 | 15 | nd | nd |

| | | | | | | | 48 hr prior to AKI stage | | |
|---|---|---|---|---|---|---|---|---|---|
| | 0 hr prior to AKI stage | | | 24 hr prior to AKI stage | | | | | UO |
| | sCr or UO | sCr only | UO only | sCr or UO | sCr only | UO only | sCr or UO | sCr only | only |
| AUC | nd | nd | nd | 0.44 | nd | 0.47 | nd | nd | nd |
| SE | nd | nd | nd | 0.080 | nd | 0.082 | nd | nd | nd |
| p | nd | nd | nd | 0.49 | nd | 0.72 | nd | nd | nd |
| nCohort 1 | nd | nd | nd | 96 | nd | 81 | nd | nd | nd |
| nCohort 2 | nd | nd | nd | 16 | nd | 15 | nd | nd | nd |
| Cutoff 1 | nd | nd | nd | 12500 | nd | 12900 | nd | nd | nd |
| Sens 1 | nd | nd | nd | 75% | nd | 73% | nd | nd | nd |
| Spec 1 | nd | nd | nd | 19% | nd | 22% | nd | nd | nd |
| Cutoff 2 | nd | nd | nd | 9460 | nd | 11700 | nd | nd | nd |
| Sens 2 | nd | nd | nd | 81% | nd | 80% | nd | nd | nd |
| Spec 2 | nd | nd | nd | 11% | nd | 22% | nd | nd | nd |
| Cutoff 3 | nd | nd | nd | 1660 | nd | 1660 | nd | nd | nd |
| Sens 3 | nd | nd | nd | 94% | nd | 93% | nd | nd | nd |
| Spec 3 | nd | nd | nd | 2% | nd | 2% | nd | nd | nd |
| Cutoff 4 | nd | nd | nd | 128000 | nd | 125000 | nd | nd | nd |
| Sens 4 | nd | nd | nd | 25% | nd | 27% | nd | nd | nd |
| Spec 4 | nd | nd | nd | 71% | nd | 70% | nd | nd | nd |
| Cutoff 5 | nd | nd | nd | 141000 | nd | 144000 | nd | nd | nd |
| Sens 5 | nd | nd | nd | 25% | nd | 27% | nd | nd | nd |
| Spec 5 | nd | nd | nd | 80% | nd | 80% | nd | nd | nd |
| Cutoff 6 | nd | nd | nd | 219000 | nd | 219000 | nd | nd | nd |
| Sens 6 | nd | nd | nd | 6% | nd | 7% | nd | nd | nd |
| Spec 6 | nd | nd | nd | 91% | nd | 90% | nd | nd | nd |
| OR Quart 2 | nd | nd | nd | 1.3 | nd | 1.0 | nd | nd | nd |
| p Value | nd | nd | nd | 0.72 | nd | 1.0 | nd | nd | nd |
| 95% CI of | nd | nd | nd | 0.31 | nd | 0.22 | nd | nd | nd |
| OR Quart 2 | nd | nd | nd | 5.5 | nd | 4.6 | nd | nd | nd |
| OR Quart 3 | nd | nd | nd | 0 | nd | 0.45 | nd | nd | nd |
| p Value | nd | nd | nd | na | nd | 0.39 | nd | nd | nd |
| 95% CI of | nd | nd | nd | na | nd | 0.075 | nd | nd | nd |
| OR Quart 3 | nd | nd | nd | na | nd | 2.8 | nd | nd | nd |
| OR Quart 4 | nd | nd | nd | 2.0 | nd | 1.3 | nd | nd | nd |
| p Value | nd | nd | nd | 0.32 | nd | 0.71 | nd | nd | nd |
| 95% CI of | nd | nd | nd | 0.51 | nd | 0.31 | nd | nd | nd |
| OR Quart 4 | nd | nd | nd | 7.8 | nd | 5.6 | nd | nd | nd |

Cyclin-dependent kinase inhibitor 1

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.09 | 3.35 | 1.09 | 4.76 | 1.09 | 2.45 |
| Average | 62.1 | 42.8 | 62.1 | 57.1 | 62.1 | 13.0 |
| Stdev | 497 | 133 | 497 | 173 | 497 | 18.2 |
| p (t-test) | | 0.85 | | 0.95 | | 0.68 |
| Min | 1.14E−14 | 1.70E−14 | 1.14E−14 | 0.116 | 1.14E−14 | 0.116 |
| Max | 6950 | 630 | 6950 | 907 | 6950 | 52.9 |
| n (Samp) | 302 | 24 | 302 | 35 | 302 | 17 |
| n (Patient) | 164 | 24 | 164 | 35 | 164 | 17 |
| sCr only | | | | | | |
| Median | nd | nd | 1.23 | 7.84 | 1.23 | 9.80 |
| Average | nd | nd | 58.3 | 15.2 | 58.3 | 15.4 |
| Stdev | nd | nd | 447 | 17.6 | 447 | 16.5 |
| p (t-test) | nd | nd | | 0.80 | | 0.80 |
| Min | nd | nd | 1.14E−14 | 0.116 | 1.14E−14 | 0.116 |
| Max | nd | nd | 6950 | 42.1 | 6950 | 39.2 |
| n (Samp) | nd | nd | 380 | 7 | 380 | 7 |
| n (Patient) | nd | nd | 196 | 7 | 196 | 7 |
| UO only | | | | | | |
| Median | 0.850 | 3.35 | 0.850 | 2.87 | 0.850 | 2.66 |
| Average | 70.1 | 42.8 | 70.1 | 62.4 | 70.1 | 12.3 |
| Stdev | 538 | 133 | 538 | 183 | 538 | 17.6 |
| p (t-test) | | 0.80 | | 0.94 | | 0.67 |
| Min | 1.14E−14 | 1.70E−14 | 1.14E−14 | 0.116 | 1.14E−14 | 0.116 |
| Max | 6950 | 630 | 6950 | 907 | 6950 | 52.9 |
| n (Samp) | 257 | 24 | 257 | 31 | 257 | 16 |
| n (Patient) | 134 | 24 | 134 | 31 | 134 | 16 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.55 | nd | 0.54 | 0.59 | 0.64 | 0.58 | 0.55 | 0.60 | 0.59 |
| SE | 0.063 | nd | 0.063 | 0.053 | 0.11 | 0.056 | 0.074 | 0.11 | 0.077 |
| p | 0.46 | nd | 0.50 | 0.080 | 0.21 | 0.16 | 0.49 | 0.36 | 0.24 |
| nCohort 1 | 302 | nd | 257 | 302 | 380 | 257 | 302 | 380 | 257 |
| nCohort 2 | 24 | nd | 24 | 35 | 7 | 31 | 17 | 7 | 16 |
| Cutoff 1 | 1.70E−14 | nd | 1.70E−14 | 0.713 | 6.55 | 1.70E−14 | 1.70E−14 | 2.02 | 0.116 |
| Sens 1 | 96% | nd | 96% | 71% | 71% | 100% | 100% | 71% | 75% |
| Spec 1 | 2% | nd | 2% | 47% | 65% | 2% | 2% | 52% | 45% |
| Cutoff 2 | 1.70E−14 | nd | 1.70E−14 | 1.70E−14 | 0.850 | 1.70E−14 | 1.70E−14 | 1.70E−14 | 1.70E−14 |
| Sens 2 | 96% | nd | 96% | 100% | 86% | 100% | 100% | 100% | 100% |
| Spec 2 | 2% | nd | 2% | 2% | 47% | 2% | 2% | 2% | 2% |
| Cutoff 3 | 1.70E−14 | nd | 1.70E−14 | 1.70E−14 | 1.70E−14 | 1.70E−14 | 1.70E−14 | 1.70E−14 | 1.70E−14 |
| Sens 3 | 96% | nd | 96% | 100% | 100% | 100% | 100% | 100% | 100% |
| Spec 3 | 2% | nd | 2% | 2% | 2% | 2% | 2% | 2% | 2% |
| Cutoff 4 | 7.84 | nd | 7.29 | 7.84 | 9.35 | 7.29 | 7.84 | 9.35 | 7.29 |
| Sens 4 | 38% | nd | 38% | 43% | 29% | 42% | 41% | 57% | 44% |
| Spec 4 | 70% | nd | 70% | 70% | 71% | 70% | 70% | 71% | 70% |
| Cutoff 5 | 16.1 | nd | 13.9 | 16.1 | 18.3 | 13.9 | 16.1 | 18.3 | 13.9 |
| Sens 5 | 21% | nd | 21% | 29% | 29% | 32% | 24% | 43% | 31% |
| Spec 5 | 80% | nd | 80% | 80% | 80% | 80% | 80% | 80% | 80% |
| Cutoff 6 | 43.4 | nd | 44.8 | 43.4 | 48.9 | 44.8 | 43.4 | 48.9 | 44.8 |
| Sens 6 | 12% | nd | 12% | 11% | 0% | 13% | 12% | 0% | 12% |
| Spec 6 | 90% | nd | 90% | 90% | 90% | 90% | 90% | 90% | 90% |
| OR Quart 2 | 0.23 | nd | 0 | 1.4 | 0.99 | 2.9 | >7.6 | 0 | 0.24 |
| p Value | 0.067 | nd | na | 0.55 | 0.99 | 0.13 | <0.061 | na | 0.21 |
| 95% CI of | 0.047 | nd | na | 0.44 | 0.061 | 0.73 | >0.91 | na | 0.026 |
| OR Quart 2 | 1.1 | nd | na | 4.7 | 16 | 11 | na | na | 2.2 |
| OR Quart 3 | 0.86 | nd | 0.87 | 2.6 | 3.0 | 3.3 | >3.1 | 0.99 | 1.3 |
| p Value | 0.79 | nd | 0.80 | 0.082 | 0.34 | 0.084 | <0.33 | 0.99 | 0.73 |
| 95% CI of | 0.30 | nd | 0.32 | 0.88 | 0.31 | 0.85 | >0.31 | 0.14 | 0.33 |
| OR Quart 3 | 2.5 | nd | 2.4 | 7.8 | 30 | 13 | na | 7.2 | 4.9 |
| OR Quart 4 | 0.85 | nd | 0.74 | 2.3 | 2.0 | 4.1 | >7.6 | 1.5 | 1.5 |
| p Value | 0.77 | nd | 0.58 | 0.13 | 0.57 | 0.035 | <0.061 | 0.66 | 0.53 |
| 95% CI of | 0.29 | nd | 0.26 | 0.78 | 0.18 | 1.1 | >0.91 | 0.25 | 0.41 |
| OR Quart 4 | 2.5 | nd | 2.1 | 7.1 | 22 | 16 | na | 9.2 | 5.7 |

Carcinoembryonic antigen-related cell adhesion molecule 5

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.852 | 1.70 | 0.852 | 2.56 | 0.852 | 1.46 |
| Average | 12.6 | 4.03 | 12.6 | 5.68 | 12.6 | 4.91 |
| Stdev | 199 | 4.99 | 199 | 9.51 | 199 | 11.3 |
| p (t-test) | | 0.83 | | 0.84 | | 0.87 |
| Min | 0.00336 | 0.00336 | 0.00336 | 0.0950 | 0.00336 | 0.148 |
| Max | 4070 | 19.5 | 4070 | 50.6 | 4070 | 47.4 |
| n (Samp) | 419 | 26 | 419 | 34 | 419 | 17 |
| n (Patient) | 164 | 26 | 164 | 34 | 164 | 17 |
| sCr only | | | | | | |
| Median | nd | nd | 0.978 | 3.62 | 0.978 | 5.87 |
| Average | nd | nd | 11.1 | 5.25 | 11.1 | 6.39 |
| Stdev | nd | nd | 180 | 7.06 | 180 | 4.92 |
| p (t-test) | nd | nd | | 0.93 | | 0.94 |
| Min | nd | nd | 0.00336 | 0.102 | 0.00336 | 0.173 |
| Max | nd | nd | 4070 | 22.0 | 4070 | 14.6 |
| n (Samp) | nd | nd | 511 | 8 | 511 | 7 |
| n (Patient) | nd | nd | 198 | 8 | 198 | 7 |
| UO only | | | | | | |
| Median | 0.852 | 1.48 | 0.852 | 2.42 | 0.852 | 1.12 |
| Average | 14.6 | 3.87 | 14.6 | 5.34 | 14.6 | 4.41 |
| Stdev | 216 | 5.02 | 216 | 9.63 | 216 | 11.3 |
| p (t-test) | | 0.80 | | 0.81 | | 0.85 |
| Min | 0.00336 | 0.0573 | 0.00336 | 0.00336 | 0.00336 | 0.148 |
| Max | 4070 | 19.5 | 4070 | 50.6 | 4070 | 47.4 |
| n (Samp) | 355 | 26 | 355 | 30 | 355 | 17 |
| n (Patient) | 134 | 26 | 134 | 30 | 134 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | nd | 0.61 | 0.67 | 0.65 | 0.66 | 0.57 | 0.76 | 0.54 |
| SE | 0.060 | nd | 0.061 | 0.052 | 0.11 | 0.056 | 0.074 | 0.11 | 0.073 |
| p | 0.029 | nd | 0.059 | 0.0014 | 0.16 | 0.0056 | 0.34 | 0.016 | 0.58 |
| nCohort 1 | 419 | nd | 355 | 419 | 511 | 355 | 419 | 511 | 355 |
| nCohort 2 | 26 | nd | 26 | 34 | 8 | 30 | 17 | 7 | 17 |
| Cutoff 1 | 0.969 | nd | 0.816 | 0.942 | 2.26 | 0.942 | 0.607 | 3.47 | 0.491 |
| Sens 1 | 73% | nd | 73% | 71% | 75% | 70% | 71% | 71% | 71% |
| Spec 1 | 53% | nd | 50% | 52% | 73% | 51% | 43% | 80% | 39% |
| Cutoff 2 | 0.590 | nd | 0.590 | 0.546 | 0.214 | 0.684 | 0.279 | 2.55 | 0.279 |
| Sens 2 | 81% | nd | 81% | 82% | 88% | 80% | 82% | 86% | 82% |
| Spec 2 | 42% | nd | 43% | 41% | 17% | 47% | 25% | 74% | 25% |
| Cutoff 3 | 0.150 | nd | 0.150 | 0.214 | 0.0990 | 0.444 | 0.150 | 0.172 | 0.150 |
| Sens 3 | 92% | nd | 92% | 91% | 100% | 90% | 94% | 100% | 94% |
| Spec 3 | 12% | nd | 12% | 18% | 7% | 37% | 12% | 14% | 12% |
| Cutoff 4 | 1.76 | nd | 1.84 | 1.76 | 1.97 | 1.84 | 1.76 | 1.97 | 1.84 |
| Sens 4 | 50% | nd | 46% | 59% | 75% | 57% | 41% | 86% | 29% |
| Spec 4 | 70% | nd | 70% | 70% | 70% | 70% | 70% | 70% | 70% |
| Cutoff 5 | 3.16 | nd | 3.25 | 3.16 | 3.51 | 3.25 | 3.16 | 3.51 | 3.25 |
| Sens 5 | 38% | nd | 35% | 44% | 50% | 40% | 24% | 57% | 18% |
| Spec 5 | 80% | nd | 80% | 80% | 80% | 80% | 80% | 80% | 80% |
| Cutoff 6 | 8.38 | nd | 8.86 | 8.38 | 8.51 | 8.86 | 8.38 | 8.51 | 8.86 |
| Sens 6 | 15% | nd | 12% | 15% | 12% | 13% | 12% | 29% | 6% |
| Spec 6 | 90% | nd | 90% | 90% | 90% | 90% | 90% | 90% | 90% |
| OR Quart 2 | 1.3 | nd | 1.7 | 1.5 | 0 | 2.4 | 0.74 | 0 | 1.7 |
| p Value | 0.70 | nd | 0.47 | 0.52 | na | 0.21 | 0.70 | na | 0.47 |
| 95% CI of | 0.29 | nd | 0.40 | 0.42 | na | 0.61 | 0.16 | na | 0.40 |
| OR Quart 2 | 6.2 | nd | 7.3 | 5.6 | na | 9.7 | 3.4 | na | 7.3 |
| OR Quart 3 | 3.2 | nd | 3.2 | 2.1 | 0.99 | 2.4 | 1.3 | 1.0 | 1.7 |
| p Value | 0.090 | nd | 0.088 | 0.24 | 0.99 | 0.21 | 0.73 | 1.0 | 0.47 |
| 95% CI of | 0.84 | nd | 0.84 | 0.61 | 0.14 | 0.61 | 0.33 | 0.062 | 0.40 |
| OR Quart 3 | 12 | nd | 12 | 7.1 | 7.2 | 9.7 | 4.8 | 16 | 7.3 |
| OR Quart 4 | 3.5 | nd | 3.2 | 4.4 | 2.0 | 4.8 | 1.3 | 5.1 | 1.3 |
| p Value | 0.061 | nd | 0.091 | 0.0096 | 0.42 | 0.017 | 0.73 | 0.14 | 0.70 |
| 95% CI of | 0.94 | nd | 0.83 | 1.4 | 0.36 | 1.3 | 0.33 | 0.59 | 0.29 |
| OR Quart 4 | 13 | nd | 12 | 14 | 11 | 17 | 4.8 | 44 | 6.2 |

Myoglobin

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.860 | 0.477 | 0.860 | 0.126 | 0.860 | 0.176 |
| Average | 35.5 | 18.1 | 35.5 | 44.2 | 35.5 | 6.85 |
| Stdev | 103 | 38.1 | 103 | 130 | 103 | 15.5 |
| p (t-test) | | 0.39 | | 0.64 | | 0.25 |
| Min | 0.000105 | 0.0151 | 0.000105 | 0.000105 | 0.000105 | 0.0254 |
| Max | 618 | 163 | 618 | 469 | 618 | 49.1 |
| n (Samp) | 419 | 26 | 419 | 34 | 419 | 17 |
| n (Patient) | 164 | 26 | 164 | 34 | 164 | 17 |
| sCr only | | | | | | |
| Median | nd | nd | 0.599 | 4.97 | 0.599 | 0.349 |
| Average | nd | nd | 37.0 | 95.0 | 37.0 | 19.7 |
| Stdev | nd | nd | 109 | 171 | 109 | 28.5 |
| p (t-test) | nd | nd | | 0.14 | | 0.68 |
| Min | nd | nd | 0.000105 | 0.0561 | 0.000105 | 0.0683 |
| Max | nd | nd | 618 | 420 | 618 | 71.6 |
| n (Samp) | nd | nd | 511 | 8 | 511 | 7 |
| n (Patient) | nd | nd | 198 | 8 | 198 | 7 |
| UO only | | | | | | |
| Median | 0.885 | 0.354 | 0.885 | 0.108 | 0.885 | 0.176 |
| Average | 40.5 | 17.6 | 40.5 | 36.1 | 40.5 | 5.29 |
| Stdev | 112 | 38.2 | 112 | 119 | 112 | 12.1 |
| p (t-test) | | 0.30 | | 0.84 | | 0.20 |
| Min | 0.000105 | 0.0151 | 0.000105 | 0.000105 | 0.000105 | 0.0254 |
| Max | 618 | 163 | 618 | 469 | 618 | 49.1 |
| n (Samp) | 355 | 26 | 355 | 30 | 355 | 17 |
| n (Patient) | 134 | 26 | 134 | 30 | 134 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.48 | nd | 0.45 | 0.39 | 0.57 | 0.38 | 0.40 | 0.55 | 0.39 |
| SE | 0.059 | nd | 0.060 | 0.053 | 0.11 | 0.057 | 0.074 | 0.11 | 0.074 |
| p | 0.72 | nd | 0.36 | 0.045 | 0.53 | 0.031 | 0.16 | 0.67 | 0.15 |
| nCohort 1 | 419 | nd | 355 | 419 | 511 | 355 | 419 | 511 | 355 |
| nCohort 2 | 26 | nd | 26 | 34 | 8 | 30 | 17 | 7 | 17 |
| Cutoff 1 | 0.0772 | nd | 0.0667 | 0.0584 | 0.118 | 0.0562 | 0.0849 | 0.132 | 0.0834 |
| Sens 1 | 73% | nd | 73% | 71% | 75% | 70% | 71% | 71% | 71% |
| Spec 1 | 22% | nd | 20% | 19% | 32% | 18% | 23% | 32% | 22% |
| Cutoff 2 | 0.0511 | nd | 0.0422 | 0.0375 | 0.0589 | 0.0366 | 0.0667 | 0.0849 | 0.0630 |
| Sens 2 | 81% | nd | 81% | 82% | 88% | 80% | 82% | 86% | 82% |
| Spec 2 | 18% | nd | 13% | 12% | 22% | 11% | 21% | 26% | 19% |
| Cutoff 3 | 0.0375 | nd | 0.0366 | 0.0331 | 0.0556 | 0.0331 | 0.0337 | 0.0667 | 0.0337 |
| Sens 3 | 92% | nd | 92% | 91% | 100% | 90% | 94% | 100% | 94% |
| Spec 3 | 12% | nd | 11% | 10% | 21% | 9% | 11% | 23% | 10% |
| Cutoff 4 | 7.09 | nd | 7.24 | 7.09 | 6.05 | 7.24 | 7.09 | 6.05 | 7.24 |
| Sens 4 | 31% | nd | 27% | 21% | 50% | 20% | 18% | 43% | 24% |
| Spec 4 | 70% | nd | 70% | 70% | 70% | 70% | 70% | 70% | 70% |
| Cutoff 5 | 19.0 | nd | 23.8 | 19.0 | 17.9 | 23.8 | 19.0 | 17.9 | 23.8 |
| Sens 5 | 19% | nd | 19% | 15% | 25% | 13% | 12% | 43% | 6% |
| Spec 5 | 80% | nd | 80% | 80% | 80% | 80% | 80% | 80% | 80% |
| Cutoff 6 | 66.4 | nd | 79.4 | 66.4 | 70.5 | 79.4 | 66.4 | 70.5 | 79.4 |
| Sens 6 | 12% | nd | 8% | 12% | 25% | 7% | 0% | 14% | 0% |
| Spec 6 | 90% | nd | 90% | 90% | 90% | 90% | 90% | 90% | 90% |
| OR Quart 2 | 0.83 | nd | 0.66 | 0.83 | 0.99 | 0.66 | 0.66 | 3.0 | 2.0 |
| p Value | 0.77 | nd | 0.53 | 0.77 | 0.99 | 0.53 | 0.65 | 0.34 | 0.42 |
| 95% CI of | 0.25 | nd | 0.18 | 0.25 | 0.14 | 0.18 | 0.11 | 0.31 | 0.37 |
| OR Quart 2 | 2.8 | nd | 2.4 | 2.8 | 7.2 | 2.4 | 4.0 | 29 | 11 |
| OR Quart 3 | 1.0 | nd | 1.0 | 1.2 | 0.49 | 0.83 | 2.1 | 0 | 2.0 |
| p Value | 0.99 | nd | 0.99 | 0.76 | 0.56 | 0.77 | 0.32 | na | 0.42 |
| 95% CI of | 0.32 | nd | 0.31 | 0.39 | 0.044 | 0.25 | 0.50 | na | 0.37 |
| OR Quart 3 | 3.2 | nd | 3.3 | 3.7 | 5.5 | 2.8 | 8.4 | na | 11 |
| OR Quart 4 | 1.6 | nd | 1.8 | 3.0 | 1.5 | 2.8 | 2.1 | 3.0 | 3.7 |
| p Value | 0.42 | nd | 0.29 | 0.029 | 0.66 | 0.042 | 0.32 | 0.34 | 0.11 |
| 95% CI of | 0.54 | nd | 0.61 | 1.1 | 0.25 | 1.0 | 0.50 | 0.31 | 0.75 |
| OR Quart 4 | 4.5 | nd | 5.1 | 7.9 | 9.1 | 7.6 | 8.4 | 29 | 18 |

Mucin-16

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | nd | nd | 0.684 | 0.822 | nd | nd |
| Average | nd | nd | 1.24 | 28.1 | nd | nd |
| Stdev | nd | nd | 1.59 | 108 | nd | nd |
| p (t-test) | nd | nd | | 0.014 | nd | nd |
| Min | nd | nd | 1.00E−9 | 0.304 | nd | nd |
| Max | nd | nd | 10.1 | 433 | nd | nd |
| n (Samp) | nd | nd | 96 | 16 | nd | nd |
| n (Patient) | nd | nd | 73 | 16 | nd | nd |
| UO only | | | | | | |
| Median | nd | nd | 0.623 | 0.822 | nd | nd |
| Average | nd | nd | 1.23 | 29.9 | nd | nd |
| Stdev | nd | nd | 1.64 | 111 | nd | nd |
| p (t-test) | nd | nd | | 0.020 | nd | nd |
| Min | nd | nd | 1.00E−9 | 0.304 | nd | nd |
| Max | nd | nd | 10.1 | 433 | nd | nd |
| n (Samp) | nd | nd | 81 | 15 | nd | nd |
| n (Patient) | nd | nd | 61 | 15 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.55 | nd | 0.56 | nd | nd | nd |
| SE | nd | nd | nd | 0.080 | nd | 0.083 | nd | nd | nd |
| p | nd | nd | nd | 0.49 | nd | 0.45 | nd | nd | nd |
| nCohort 1 | nd | nd | nd | 96 | nd | 81 | nd | nd | nd |
| nCohort 2 | nd | nd | nd | 16 | nd | 15 | nd | nd | nd |
| Cutoff 1 | nd | nd | nd | 0.412 | nd | 0.412 | nd | nd | nd |
| Sens 1 | nd | nd | nd | 75% | nd | 73% | nd | nd | nd |
| Spec 1 | nd | nd | nd | 32% | nd | 36% | nd | nd | nd |
| Cutoff 2 | nd | nd | nd | 0.344 | nd | 0.344 | nd | nd | nd |
| Sens 2 | nd | nd | nd | 88% | nd | 87% | nd | nd | nd |
| Spec 2 | nd | nd | nd | 21% | nd | 26% | nd | nd | nd |
| Cutoff 3 | nd | nd | nd | 0.278 | nd | 0.278 | nd | nd | nd |
| Sens 3 | nd | nd | nd | 100% | nd | 100% | nd | nd | nd |
| Spec 3 | nd | nd | nd | 11% | nd | 15% | nd | nd | nd |
| Cutoff 4 | nd | nd | nd | 1.17 | nd | 1.09 | nd | nd | nd |
| Sens 4 | nd | nd | nd | 25% | nd | 33% | nd | nd | nd |
| Spec 4 | nd | nd | nd | 71% | nd | 70% | nd | nd | nd |
| Cutoff 5 | nd | nd | nd | 1.41 | nd | 1.44 | nd | nd | nd |
| Sens 5 | nd | nd | nd | 19% | nd | 20% | nd | nd | nd |
| Spec 5 | nd | nd | nd | 80% | nd | 80% | nd | nd | nd |
| Cutoff 6 | nd | nd | nd | 3.40 | nd | 3.26 | nd | nd | nd |
| Sens 6 | nd | nd | nd | 12% | nd | 13% | nd | nd | nd |
| Spec 6 | nd | nd | nd | 91% | nd | 90% | nd | nd | nd |
| OR Quart 2 | nd | nd | nd | 0.72 | nd | 1.0 | nd | nd | nd |
| p Value | nd | nd | nd | 0.69 | nd | 1.0 | nd | nd | nd |
| 95% CI of | nd | nd | nd | 0.15 | nd | 0.18 | nd | nd | nd |
| OR Quart 2 | nd | nd | nd | 3.6 | nd | 5.5 | nd | nd | nd |
| OR Quart 3 | nd | nd | nd | 1.3 | nd | 1.8 | nd | nd | nd |
| p Value | nd | nd | nd | 0.72 | nd | 0.44 | nd | nd | nd |
| 95% CI of | nd | nd | nd | 0.31 | nd | 0.39 | nd | nd | nd |
| OR Quart 3 | nd | nd | nd | 5.5 | nd | 8.8 | nd | nd | nd |
| OR Quart 4 | nd | nd | nd | 1.0 | nd | 1.4 | nd | nd | nd |
| p Value | nd | nd | nd | 1.0 | nd | 0.68 | nd | nd | nd |
| 95% CI of | nd | nd | nd | 0.22 | nd | 0.28 | nd | nd | nd |
| OR Quart 4 | nd | nd | nd | 4.5 | nd | 7.1 | nd | nd | nd |

Tumor necrosis factor receptor superfamily member 10B

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | nd | nd | 0.344 | 0.306 | nd | nd |
| Average | nd | nd | 0.956 | 1.45 | nd | nd |
| Stdev | nd | nd | 1.45 | 1.97 | nd | nd |
| p (t-test) | nd | nd | | 0.24 | nd | nd |
| Min | nd | nd | 0.00360 | 0.0172 | nd | nd |
| Max | nd | nd | 6.71 | 5.86 | nd | nd |
| n (Samp) | nd | nd | 96 | 16 | nd | nd |
| n (Patient) | nd | nd | 73 | 16 | nd | nd |
| UO only | | | | | | |
| Median | nd | nd | 0.343 | 0.287 | nd | nd |
| Average | nd | nd | 0.805 | 1.42 | nd | nd |
| Stdev | nd | nd | 1.26 | 2.00 | nd | nd |
| p (t-test) | nd | nd | | 0.12 | nd | nd |
| Min | nd | nd | 0.00360 | 0.0172 | nd | nd |
| Max | nd | nd | 6.48 | 5.86 | nd | nd |
| n (Samp) | nd | nd | 81 | 15 | nd | nd |
| n (Patient) | nd | nd | 61 | 15 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.56 | nd | 0.55 | nd | nd | nd |
| SE | nd | nd | nd | 0.080 | nd | 0.083 | nd | nd | nd |
| p | nd | nd | nd | 0.46 | nd | 0.53 | nd | nd | nd |
| nCohort 1 | nd | nd | nd | 96 | nd | 81 | nd | nd | nd |
| nCohort 2 | nd | nd | nd | 16 | nd | 15 | nd | nd | nd |
| Cutoff 1 | nd | nd | nd | 0.109 | nd | 0.109 | nd | nd | nd |
| Sens 1 | nd | nd | nd | 75% | nd | 73% | nd | nd | nd |
| Spec 1 | nd | nd | nd | 35% | nd | 36% | nd | nd | nd |
| Cutoff 2 | nd | nd | nd | 0.101 | nd | 0.101 | nd | nd | nd |
| Sens 2 | nd | nd | nd | 81% | nd | 80% | nd | nd | nd |
| Spec 2 | nd | nd | nd | 31% | nd | 32% | nd | nd | nd |
| Cutoff 3 | nd | nd | nd | 0.0198 | nd | 0.0182 | nd | nd | nd |
| Sens 3 | nd | nd | nd | 94% | nd | 93% | nd | nd | nd |
| Spec 3 | nd | nd | nd | 11% | nd | 11% | nd | nd | nd |
| Cutoff 4 | nd | nd | nd | 0.855 | nd | 0.819 | nd | nd | nd |
| Sens 4 | nd | nd | nd | 38% | nd | 33% | nd | nd | nd |
| Spec 4 | nd | nd | nd | 71% | nd | 70% | nd | nd | nd |
| Cutoff 5 | nd | nd | nd | 1.56 | nd | 0.979 | nd | nd | nd |
| Sens 5 | nd | nd | nd | 31% | nd | 33% | nd | nd | nd |
| Spec 5 | nd | nd | nd | 80% | nd | 80% | nd | nd | nd |
| Cutoff 6 | nd | nd | nd | 3.11 | nd | 2.25 | nd | nd | nd |
| Sens 6 | nd | nd | nd | 19% | nd | 27% | nd | nd | nd |
| Spec 6 | nd | nd | nd | 91% | nd | 90% | nd | nd | nd |
| OR Quart 2 | nd | nd | nd | 4.3 | nd | 3.7 | nd | nd | nd |
| p Value | nd | nd | nd | 0.086 | nd | 0.14 | nd | nd | nd |
| 95% CI of | nd | nd | nd | 0.81 | nd | 0.66 | nd | nd | nd |
| OR Quart 2 | nd | nd | nd | 23 | nd | 20 | nd | nd | nd |
| OR Quart 3 | nd | nd | nd | 0.48 | nd | 1.0 | nd | nd | nd |
| p Value | nd | nd | nd | 0.56 | nd | 1.0 | nd | nd | nd |
| 95% CI of | nd | nd | nd | 0.041 | nd | 0.13 | nd | nd | nd |
| OR Quart 3 | nd | nd | nd | 5.6 | nd | 7.7 | nd | nd | nd |
| OR Quart 4 | nd | nd | nd | 3.5 | nd | 2.9 | nd | nd | nd |
| p Value | nd | nd | nd | 0.14 | nd | 0.23 | nd | nd | nd |
| 95% CI of | nd | nd | nd | 0.65 | nd | 0.50 | nd | nd | nd |
| OR Quart 4 | nd | nd | nd | 19 | nd | 17 | nd | nd | nd |

Cellular tumor antigen p53

| | | |
|---|---|---|
| 0 hr prior | 24 hr prior | 48 hr prior |
| to AKI stage | to AKI stage | to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | nd | nd | 1.00E−9 | 0.000165 | nd | nd |
| Average | nd | nd | 0.00163 | 0.00180 | nd | nd |
| Stdev | nd | nd | 0.00319 | 0.00245 | nd | nd |
| p (t-test) | nd | nd | | 0.84 | nd | nd |
| Min | nd | nd | 1.00E−9 | 1.00E−9 | nd | nd |
| Max | nd | nd | 0.0202 | 0.00839 | nd | nd |
| n (Samp) | nd | nd | 96 | 16 | nd | nd |
| n (Patient) | nd | nd | 73 | 16 | nd | nd |
| UO only | | | | | | |
| Median | nd | nd | 1.00E−9 | 3.53E−5 | nd | nd |
| Average | nd | nd | 0.00186 | 0.00145 | nd | nd |
| Stdev | nd | nd | 0.00355 | 0.00190 | nd | nd |
| p (t-test) | nd | nd | | 0.66 | nd | nd |
| Min | nd | nd | 1.00E−9 | 1.00E−9 | nd | nd |
| Max | nd | nd | 0.0202 | 0.00470 | nd | nd |
| n (Samp) | nd | nd | 81 | 15 | nd | nd |
| n (Patient) | nd | nd | 61 | 15 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | nd | nd | nd | 0.58 | nd | 0.55 | nd | nd | nd |
| SE | nd | nd | nd | 0.080 | nd | 0.083 | nd | nd | nd |
| p | nd | nd | nd | 0.30 | nd | 0.56 | nd | nd | nd |
| nCohort 1 | nd | nd | nd | 96 | nd | 81 | nd | nd | nd |
| nCohort 2 | nd | nd | nd | 16 | nd | 15 | nd | nd | nd |
| Cutoff 1 | nd | nd | nd | 0 | nd | 0 | nd | nd | nd |
| Sens 1 | nd | nd | nd | 100% | nd | 100% | nd | nd | nd |
| Spec 1 | nd | nd | nd | 0% | nd | 0% | nd | nd | nd |
| Cutoff 2 | nd | nd | nd | 0 | nd | 0 | nd | nd | nd |
| Sens 2 | nd | nd | nd | 100% | nd | 100% | nd | nd | nd |
| Spec 2 | nd | nd | nd | 0% | nd | 0% | nd | nd | nd |
| Cutoff 3 | nd | nd | nd | 0 | nd | 0 | nd | nd | nd |
| Sens 3 | nd | nd | nd | 100% | nd | 100% | nd | nd | nd |
| Spec 3 | nd | nd | nd | 0% | nd | 0% | nd | nd | nd |
| Cutoff 4 | nd | nd | nd | 0.00160 | nd | 0.00160 | nd | nd | nd |
| Sens 4 | nd | nd | nd | 38% | nd | 33% | nd | nd | nd |
| Spec 4 | nd | nd | nd | 73% | nd | 72% | nd | nd | nd |
| Cutoff 5 | nd | nd | nd | 0.00313 | nd | 0.00343 | nd | nd | nd |
| Sens 5 | nd | nd | nd | 38% | nd | 20% | nd | nd | nd |
| Spec 5 | nd | nd | nd | 81% | nd | 80% | nd | nd | nd |
| Cutoff 6 | nd | nd | nd | 0.00470 | nd | 0.00599 | nd | nd | nd |
| Sens 6 | nd | nd | nd | 6% | nd | 0% | nd | nd | nd |
| Spec 6 | nd | nd | nd | 92% | nd | 91% | nd | nd | nd |
| OR Quart 2 | nd | nd | nd | 0.46 | nd | 0.17 | nd | nd | nd |
| p Value | nd | nd | nd | 0.40 | nd | 0.11 | nd | nd | nd |
| 95% CI of | nd | nd | nd | 0.077 | nd | 0.018 | nd | nd | nd |
| OR Quart 2 | nd | nd | nd | 2.8 | nd | 1.5 | nd | nd | nd |
| OR Quart 3 | nd | nd | nd | 1.0 | nd | 0.76 | nd | nd | nd |
| p Value | nd | nd | nd | 1.0 | nd | 0.71 | nd | nd | nd |
| 95% CI of | nd | nd | nd | 0.22 | nd | 0.18 | nd | nd | nd |
| OR Quart 3 | nd | nd | nd | 4.5 | nd | 3.3 | nd | nd | nd |
| OR Quart 4 | nd | nd | nd | 1.6 | nd | 1.0 | nd | nd | nd |
| p Value | nd | nd | nd | 0.49 | nd | 1.0 | nd | nd | nd |
| 95% CI of | nd | nd | nd | 0.41 | nd | 0.25 | nd | nd | nd |
| OR Quart 4 | nd | nd | nd | 6.6 | nd | 4.0 | nd | nd | nd |

TABLE 3
Comparison of marker levels in urine samples collected within 12 hours of
reaching stage R from Cohort 1 (patients that reached, but did not progress beyond,
RIFLE stage R) and from Cohort 2 (patients that reached RIFLE stage I or F).
Apolipoprotein A-II

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 78.2 | 125 | 77.5 | 247 | 85.8 | 106 |
| Average | 156 | 187 | 167 | 307 | 149 | 132 |
| Stdev | 303 | 193 | 368 | 295 | 238 | 77.5 |
| p (t-test) | | 0.58 | | 0.30 | | 0.74 |
| Min | 4.67 | 18.9 | 4.67 | 23.0 | 21.4 | 18.9 |
| Max | 2000 | 845 | 2000 | 845 | 1790 | 323 |
| n (Samp) | 77 | 33 | 29 | 9 | 61 | 24 |
| n (Patient) | 77 | 33 | 29 | 9 | 61 | 24 |

| | At Enrollment | | |
|---|---|---|---|
| | sCr or UO | sCr only | UO only |
| AUC | 0.64 | 0.76 | 0.60 |
| SE | 0.059 | 0.10 | 0.070 |
| p | 0.016 | 0.011 | 0.16 |
| nCohort 1 | 77 | 29 | 61 |
| nCohort 2 | 33 | 9 | 24 |
| Cutoff 1 | 82.3 | 125 | 85.8 |
| Sens 1 | 73% | 78% | 71% |
| Spec 1 | 53% | 72% | 51% |
| Cutoff 2 | 67.3 | 78.5 | 65.3 |
| Sens 2 | 82% | 89% | 83% |
| Spec 2 | 47% | 55% | 43% |
| Cutoff 3 | 58.1 | 21.6 | 62.3 |
| Sens 3 | 91% | 100% | 92% |
| Spec 3 | 42% | 10% | 41% |
| Cutoff 4 | 126 | 125 | 148 |
| Sens 4 | 48% | 78% | 33% |
| Spec 4 | 70% | 72% | 70% |
| Cutoff 5 | 166 | 166 | 180 |
| Sens 5 | 33% | 56% | 21% |
| Spec 5 | 81% | 83% | 80% |
| Cutoff 6 | 314 | 195 | 314 |
| Sens 6 | 12% | 56% | 4% |
| Spec 6 | 91% | 93% | 90% |
| OR Quart 2 | 3.2 | 0.89 | 12 |
| p Value | 0.12 | 0.94 | 0.025 |
| 95% CI of | 0.75 | 0.047 | 1.4 |
| OR Quart 2 | 14 | 17 | 110 |
| OR Quart 3 | 5.5 | 2.3 | 15 |
| p Value | 0.019 | 0.53 | 0.015 |
| 95% CI of | 1.3 | 0.17 | 1.7 |
| OR Quart 3 | 23 | 31 | 130 |
| OR Quart 4 | 5.2 | 8.0 | 7.5 |
| p Value | 0.023 | 0.092 | 0.075 |
| 95% CI of | 1.3 | 0.71 | 0.82 |
| OR Quart 4 | 21 | 90 | 69 |

Myoglobin

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 0.515 | 0.0847 | nd | nd | 0.758 | 0.0561 |
| Average | 42.9 | 26.9 | nd | nd | 49.3 | 3.71 |
| Stdev | 122 | 94.2 | nd | nd | 131 | 12.0 |
| p (t-test) | | 0.57 | nd | nd | | 0.16 |
| Min | 0.000105 | 0.000105 | nd | nd | 0.0176 | 0.0145 |
| Max | 618 | 460 | nd | nd | 618 | 49.1 |
| n (Samp) | 52 | 24 | nd | nd | 44 | 17 |
| n (Patient) | 52 | 24 | nd | nd | 44 | 17 |

| | At Enrollment | | |
|---|---|---|---|
| | sCr or UO | sCr only | UO only |
| AUC | 0.40 | nd | 0.30 |
| SE | 0.072 | nd | 0.079 |
| p | 0.17 | nd | 0.011 |
| nCohort 1 | 52 | nd | 44 |
| nCohort 2 | 24 | nd | 17 |
| Cutoff 1 | 0.0365 | nd | 0.0347 |
| Sens 1 | 71% | nd | 71% |
| Spec 1 | 15% | nd | 14% |
| Cutoff 2 | 0.0324 | nd | 0.0324 |
| Sens 2 | 83% | nd | 82% |
| Spec 2 | 12% | nd | 11% |
| Cutoff 3 | 0.0176 | nd | 0.0176 |
| Sens 3 | 92% | nd | 94% |
| Spec 3 | 4% | nd | 2% |
| Cutoff 4 | 13.5 | nd | 13.9 |
| Sens 4 | 21% | nd | 6% |
| Spec 4 | 71% | nd | 70% |
| Cutoff 5 | 33.1 | nd | 34.7 |
| Sens 5 | 12% | nd | 6% |
| Spec 5 | 81% | nd | 82% |
| Cutoff 6 | 63.7 | nd | 85.7 |
| Sens 6 | 8% | nd | 0% |
| Spec 6 | 90% | nd | 91% |
| OR Quart 2 | 0.75 | nd | 1.8 |
| p Value | 0.70 | nd | 0.57 |
| 95% CI of | 0.17 | nd | 0.25 |
| OR Quart 2 | 3.4 | nd | 12 |
| OR Quart 3 | 1.6 | nd | 3.5 |
| p Value | 0.49 | nd | 0.18 |
| 95% CI of | 0.41 | nd | 0.56 |
| OR Quart 3 | 6.5 | nd | 22 |
| OR Quart 4 | 2.0 | nd | 6.1 |
| p Value | 0.31 | nd | 0.048 |
| 95% CI of | 0.52 | nd | 1.0 |
| OR Quart 4 | 8.0 | nd | 37 |

KSP-Cadherin

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 1.48 | 0.809 | nd | nd | 1.46 | 0.996 |
| Average | 1.74 | 1.25 | nd | nd | 1.73 | 1.18 |
| Stdev | 1.18 | 1.36 | nd | nd | 1.10 | 1.26 |
| p (t-test) | | 0.18 | nd | nd | | 0.21 |
| Min | 0.131 | 0.00263 | nd | nd | 0.196 | 0.00263 |
| Max | 4.63 | 4.23 | nd | nd | 4.63 | 3.88 |
| n (Samp) | 40 | 16 | nd | nd | 31 | 9 |
| n (Patient) | 40 | 16 | nd | nd | 31 | 9 |

| | At Enrollment | | |
|---|---|---|---|
| | sCr or UO | sCr only | UO only |
| AUC | 0.33 | nd | 0.31 |
| SE | 0.084 | nd | 0.11 |
| p | 0.037 | nd | 0.081 |
| nCohort 1 | 40 | nd | 31 |
| nCohort 2 | 16 | nd | 9 |
| Cutoff 1 | 0.258 | nd | 0.196 |
| Sens 1 | 75% | nd | 78% |
| Spec 1 | 5% | nd | 3% |
| Cutoff 2 | 0.196 | nd | 0.00263 |
| Sens 2 | 81% | nd | 89% |
| Spec 2 | 5% | nd | 0% |
| Cutoff 3 | 0.00263 | nd | 0 |
| Sens 3 | 94% | nd | 100% |
| Spec 3 | 0% | nd | 0% |
| Cutoff 4 | 1.84 | nd | 1.84 |
| Sens 4 | 25% | nd | 22% |
| Spec 4 | 70% | nd | 71% |
| Cutoff 5 | 2.57 | nd | 2.54 |
| Sens 5 | 19% | nd | 11% |
| Spec 5 | 80% | nd | 81% |
| Cutoff 6 | 3.39 | nd | 3.11 |
| Sens 6 | 12% | nd | 11% |
| Spec 6 | 90% | nd | 90% |
| OR Quart 2 | 0 | nd | 0 |
| p Value | na | nd | na |
| 95% CI of | na | nd | na |
| OR Quart 2 | na | nd | na |
| OR Quart 3 | 1.0 | nd | 1.7 |
| p Value | 1.0 | nd | 0.61 |
| 95% CI of | 0.19 | nd | 0.22 |
| OR Quart 3 | 5.2 | nd | 13 |
| OR Quart 4 | 3.3 | nd | 2.7 |
| p Value | 0.13 | nd | 0.34 |
| 95% CI of | 0.69 | nd | 0.36 |
| OR Quart 4 | 16 | nd | 20 |

TABLE 4
Comparison of the maximum marker levels in urine samples collected from
Cohort 1 (patients that did not progress beyond RIFLE stage 0) and the maximum values in urine
samples collected from subjects between enrollment and 0, 24 hours, and 48 hours prior to
reaching stage F in Cohort 2.
Apolipoprotein A-II

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 76.0 | 265 | 76.0 | 217 | 76.0 | 157 |
| Average | 241 | 1760 | 241 | 1460 | 241 | 225 |
| Stdev | 841 | 5360 | 841 | 5260 | 841 | 193 |
| p (t-test) | | 3.5E−4 | | 0.0035 | | 0.95 |
| Min | 5.33 | 27.6 | 5.33 | 9.51 | 5.33 | 55.6 |
| Max | 6400 | 24300 | 6400 | 24300 | 6400 | 764 |
| n (Samp) | 196 | 21 | 196 | 21 | 196 | 11 |
| n (Patient) | 196 | 21 | 196 | 21 | 196 | 11 |
| sCr only | | | | | | |
| Median | 97.8 | 268 | 97.8 | 265 | 97.8 | 216 |
| Average | 342 | 954 | 342 | 393 | 342 | 213 |
| Stdev | 1610 | 1880 | 1610 | 510 | 1610 | 68.6 |
| p (t-test) | | 0.22 | | 0.92 | | 0.84 |
| Min | 5.33 | 27.6 | 5.33 | 9.51 | 5.33 | 131 |
| Max | 24300 | 6400 | 24300 | 1860 | 24300 | 288 |
| n (Samp) | 294 | 11 | 294 | 11 | 294 | 6 |
| n (Patient) | 294 | 11 | 294 | 11 | 294 | 6 |
| UO only | | | | | | |
| Median | 87.2 | 174 | 87.2 | 174 | 87.2 | 149 |
| Average | 294 | 2240 | 294 | 1850 | 294 | 227 |
| Stdev | 972 | 6320 | 972 | 6230 | 972 | 214 |
| p (t-test) | | 0.0012 | | 0.0086 | | 0.84 |
| Min | 5.33 | 55.6 | 5.33 | 45.8 | 5.33 | 55.6 |
| Max | 6400 | 24300 | 6400 | 24300 | 6400 | 764 |
| n (Samp) | 132 | 15 | 132 | 15 | 132 | 9 |
| n (Patient) | 132 | 15 | 132 | 15 | 132 | 9 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.80 | 0.78 | 0.79 | 0.77 | 0.76 | 0.76 | 0.78 | 0.78 | 0.73 |
| SE | 0.059 | 0.084 | 0.072 | 0.062 | 0.086 | 0.075 | 0.084 | 0.11 | 0.098 |
| p | 2.8E−7 | 9.4E−4 | 4.7E−5 | 8.7E−6 | 0.0029 | 5.7E−4 | 0.0010 | 0.014 | 0.019 |
| nCohort 1 | 196 | 294 | 132 | 196 | 294 | 132 | 196 | 294 | 132 |
| nCohort 2 | 21 | 11 | 15 | 21 | 11 | 15 | 11 | 6 | 9 |
| Cutoff 1 | 148 | 165 | 146 | 148 | 165 | 146 | 146 | 157 | 111 |
| Sens 1 | 71% | 73% | 73% | 71% | 73% | 73% | 73% | 83% | 78% |
| Spec 1 | 80% | 74% | 77% | 80% | 74% | 77% | 79% | 71% | 63% |
| Cutoff 2 | 129 | 157 | 129 | 129 | 157 | 129 | 111 | 157 | 100 |
| Sens 2 | 81% | 82% | 80% | 81% | 82% | 80% | 82% | 83% | 89% |
| Spec 2 | 74% | 71% | 73% | 74% | 71% | 73% | 67% | 71% | 58% |
| Cutoff 3 | 100 | 135 | 100 | 54.2 | 129 | 54.3 | 100 | 129 | 54.3 |
| Sens 3 | 90% | 91% | 93% | 90% | 91% | 93% | 91% | 100% | 100% |
| Spec 3 | 62% | 65% | 58% | 37% | 64% | 32% | 62% | 64% | 32% |
| Cutoff 4 | 124 | 155 | 127 | 124 | 155 | 127 | 124 | 155 | 127 |
| Sens 4 | 81% | 82% | 80% | 81% | 82% | 80% | 73% | 83% | 67% |
| Spec 4 | 70% | 70% | 70% | 70% | 70% | 70% | 70% | 70% | 70% |
| Cutoff 5 | 153 | 200 | 164 | 153 | 200 | 164 | 153 | 200 | 164 |
| Sens 5 | 67% | 64% | 53% | 67% | 64% | 53% | 55% | 50% | 33% |
| Spec 5 | 80% | 80% | 80% | 80% | 80% | 80% | 80% | 80% | 80% |
| Cutoff 6 | 296 | 335 | 312 | 296 | 335 | 312 | 296 | 335 | 312 |
| Sens 6 | 29% | 27% | 20% | 24% | 18% | 20% | 9% | 0% | 11% |
| Spec 6 | 90% | 90% | 90% | 90% | 90% | 90% | 90% | 90% | 90% |
| OR Quart 2 | 1.0 | 0 | >1.0 | 2.0 | 0 | 0.97 | >1.0 | >0 | >1.0 |
| p Value | 1.0 | na | <1.0 | 0.57 | na | 0.98 | <1.0 | <na | <0.98 |
| 95% CI of | 0.061 | na | >0.060 | 0.18 | na | 0.058 | >0.061 | >na | >0.062 |
| OR Quart 2 | 16 | na | na | 23 | na | 16 | na | na | na |
| OR Quart 3 | 4.2 | 3.1 | >5.6 | 3.1 | 3.1 | 4.2 | >2.0 | >3.1 | >2.1 |
| p Value | 0.20 | 0.33 | <0.12 | 0.33 | 0.33 | 0.21 | <0.57 | <0.33 | <0.55 |
| 95% CI of | 0.46 | 0.31 | >0.62 | 0.31 | 0.31 | 0.45 | >0.18 | >0.32 | >0.18 |
| OR Quart 3 | 39 | 30 | na | 31 | 30 | 40 | na | na | na |
| OR Quart 4 | 20 | 7.5 | >12 | 20 | 7.5 | 11 | >9.3 | >3.1 | >7.0 |
| p Value | 0.0046 | 0.063 | <0.024 | 0.0046 | 0.063 | 0.026 | <0.039 | <0.33 | <0.079 |
| 95% CI of | 2.5 | 0.90 | >1.4 | 2.5 | 0.90 | 1.3 | >1.1 | >0.32 | >0.80 |
| OR Quart 4 | 160 | 63 | na | 160 | 63 | 94 | na | na | na |

Caspase-1

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 1.28 | 4.81 | 1.28 | 2.33 | 1.28 | 0.988 |
| Average | 1.56 | 10.5 | 1.56 | 4.46 | 1.56 | 1.77 |
| Stdev | 1.38 | 15.7 | 1.38 | 6.02 | 1.38 | 2.36 |
| p (t-test) | | 0.0057 | | 0.027 | | 0.78 |
| Min | 0.0223 | 0.0223 | 0.0223 | 0.0223 | 0.0223 | 0.0223 |
| Max | 4.68 | 47.1 | 4.68 | 17.3 | 4.68 | 6.25 |
| n (Samp) | 26 | 8 | 26 | 7 | 26 | 6 |
| n (Patient) | 26 | 8 | 26 | 7 | 26 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.75 | nd | nd | 0.64 | nd | nd | 0.45 | nd | nd |
| SE | 0.11 | nd | nd | 0.12 | nd | nd | 0.13 | nd | nd |
| p | 0.021 | nd | nd | 0.25 | nd | nd | 0.72 | nd | nd |
| nCohort 1 | 26 | nd | nd | 26 | nd | nd | 26 | nd | nd |
| nCohort 2 | 8 | nd | nd | 7 | nd | nd | 6 | nd | nd |
| Cutoff 1 | 2.08 | nd | nd | 1.02 | nd | nd | 0 | nd | nd |
| Sens 1 | 75% | nd | nd | 71% | nd | nd | 100% | nd | nd |
| Spec 1 | 69% | nd | nd | 46% | nd | nd | 0% | nd | nd |
| Cutoff 2 | 0.496 | nd | nd | 0.496 | nd | nd | 0 | nd | nd |
| Sens 2 | 88% | nd | nd | 86% | nd | nd | 100% | nd | nd |
| Spec 2 | 31% | nd | nd | 31% | nd | nd | 0% | nd | nd |
| Cutoff 3 | 0 | nd | nd | 0 | nd | nd | 0 | nd | nd |
| Sens 3 | 100% | nd | nd | 100% | nd | nd | 100% | nd | nd |
| Spec 3 | 0% | nd | nd | 0% | nd | nd | 0% | nd | nd |
| Cutoff 4 | 2.33 | nd | nd | 2.33 | nd | nd | 2.33 | nd | nd |
| Sens 4 | 62% | nd | nd | 43% | nd | nd | 17% | nd | nd |
| Spec 4 | 73% | nd | nd | 73% | nd | nd | 73% | nd | nd |
| Cutoff 5 | 2.59 | nd | nd | 2.59 | nd | nd | 2.59 | nd | nd |
| Sens 5 | 62% | nd | nd | 43% | nd | nd | 17% | nd | nd |
| Spec 5 | 85% | nd | nd | 85% | nd | nd | 85% | nd | nd |
| Cutoff 6 | 3.90 | nd | nd | 3.90 | nd | nd | 3.90 | nd | nd |
| Sens 6 | 50% | nd | nd | 29% | nd | nd | 17% | nd | nd |
| Spec 6 | 92% | nd | nd | 92% | nd | nd | 92% | nd | nd |
| OR Quart 2 | 0.88 | nd | nd | 1.0 | nd | nd | 1.0 | nd | nd |
| p Value | 0.93 | nd | nd | 1.0 | nd | nd | 1.0 | nd | nd |
| 95% CI of | 0.046 | nd | nd | 0.052 | nd | nd | 0.052 | nd | nd |
| OR Quart 2 | 17 | nd | nd | 19 | nd | nd | 19 | nd | nd |
| OR Quart 3 | 1.0 | nd | nd | 2.3 | nd | nd | 2.3 | nd | nd |
| p Value | 1.0 | nd | nd | 0.53 | nd | nd | 0.53 | nd | nd |
| 95% CI of | 0.052 | nd | nd | 0.17 | nd | nd | 0.17 | nd | nd |
| OR Quart 3 | 19 | nd | nd | 33 | nd | nd | 33 | nd | nd |
| OR Quart 4 | 8.8 | nd | nd | 3.5 | nd | nd | 2.3 | nd | nd |
| p Value | 0.086 | nd | nd | 0.33 | nd | nd | 0.53 | nd | nd |
| 95% CI of | 0.74 | nd | nd | 0.28 | nd | nd | 0.17 | nd | nd |
| OR Quart 4 | 100 | nd | nd | 43 | nd | nd | 33 | nd | nd |

Caspase-9

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 0.549 | 3.00 | 0.549 | 2.16 | 0.549 | 1.09 |
| Average | 1.25 | 2.65 | 1.25 | 1.79 | 1.25 | 1.35 |
| Stdev | 3.21 | 1.71 | 3.21 | 1.60 | 3.21 | 1.25 |
| p (t-test) | | 0.24 | | 0.67 | | 0.94 |
| Min | 0.0360 | 0.146 | 0.0360 | 0.146 | 0.0360 | 0.146 |
| Max | 19.5 | 4.64 | 19.5 | 4.64 | 19.5 | 3.33 |
| n (Samp) | 36 | 8 | 36 | 7 | 36 | 6 |
| n (Patient) | 36 | 8 | 36 | 7 | 36 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.79 | nd | nd | 0.69 | nd | nd | 0.64 | nd | nd |
| SE | 0.10 | nd | nd | 0.12 | nd | nd | 0.13 | nd | nd |
| p | 0.0034 | nd | nd | 0.11 | nd | nd | 0.27 | nd | nd |
| nCohort 1 | 36 | nd | nd | 36 | nd | nd | 36 | nd | nd |
| nCohort 2 | 8 | nd | nd | 7 | nd | nd | 6 | nd | nd |
| Cutoff 1 | 1.76 | nd | nd | 0.473 | nd | nd | 0.208 | nd | nd |
| Sens 1 | 75% | nd | nd | 71% | nd | nd | 83% | nd | nd |
| Spec 1 | 89% | nd | nd | 50% | nd | nd | 33% | nd | nd |
| Cutoff 2 | 0.208 | nd | nd | 0.208 | nd | nd | 0.208 | nd | nd |
| Sens 2 | 88% | nd | nd | 86% | nd | nd | 83% | nd | nd |
| Spec 2 | 33% | nd | nd | 33% | nd | nd | 33% | nd | nd |
| Cutoff 3 | 0.135 | nd | nd | 0.135 | nd | nd | 0.135 | nd | nd |
| Sens 3 | 100% | nd | nd | 100% | nd | nd | 100% | nd | nd |
| Spec 3 | 28% | nd | nd | 28% | nd | nd | 28% | nd | nd |
| Cutoff 4 | 0.868 | nd | nd | 0.868 | nd | nd | 0.868 | nd | nd |
| Sens 4 | 75% | nd | nd | 57% | nd | nd | 50% | nd | nd |
| Spec 4 | 72% | nd | nd | 72% | nd | nd | 72% | nd | nd |
| Cutoff 5 | 1.22 | nd | nd | 1.22 | nd | nd | 1.22 | nd | nd |
| Sens 5 | 75% | nd | nd | 57% | nd | nd | 50% | nd | nd |
| Spec 5 | 81% | nd | nd | 81% | nd | nd | 81% | nd | nd |
| Cutoff 6 | 2.17 | nd | nd | 2.17 | nd | nd | 2.17 | nd | nd |
| Sens 6 | 62% | nd | nd | 29% | nd | nd | 17% | nd | nd |
| Spec 6 | 92% | nd | nd | 92% | nd | nd | 92% | nd | nd |
| OR Quart 2 | >2.4 | nd | nd | >2.2 | nd | nd | >2.2 | nd | nd |
| p Value | <0.49 | nd | nd | <0.54 | nd | nd | <0.54 | nd | nd |
| 95% CI of | >0.19 | nd | nd | >0.17 | nd | nd | >0.17 | nd | nd |
| OR Quart 2 | na | nd | nd | na | nd | nd | na | nd | nd |
| OR Quart 3 | >0 | nd | nd | >1.0 | nd | nd | >1.1 | nd | nd |
| p Value | <na | nd | nd | <1.0 | nd | nd | <0.94 | nd | nd |
| 95% CI of | >na | nd | nd | >0.055 | nd | nd | >0.060 | nd | nd |
| OR Quart 3 | na | nd | nd | na | nd | nd | na | nd | nd |
| OR Quart 4 | >13 | nd | nd | >5.7 | nd | nd | >3.8 | nd | nd |
| p Value | <0.033 | nd | nd | <0.15 | nd | nd | <0.29 | nd | nd |
| 95% CI of | >1.2 | nd | nd | >0.52 | nd | nd | >0.32 | nd | nd |
| OR Quart 4 | na | nd | nd | na | nd | nd | na | nd | nd |

Cadherin-1

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 66800 | 66800 | 66800 | 66800 | nd | nd |
| Average | 99700 | 95000 | 99700 | 95000 | nd | nd |
| Stdev | 131000 | 95400 | 131000 | 95400 | nd | nd |
| p (t-test) | | 0.92 | | 0.92 | nd | nd |
| Min | 3290 | 2220 | 3290 | 2220 | nd | nd |
| Max | 744000 | 260000 | 744000 | 260000 | nd | nd |
| n (Samp) | 41 | 8 | 41 | 8 | nd | nd |
| n (Patient) | 41 | 8 | 41 | 8 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.49 | nd | nd | 0.49 | nd | nd | nd | nd | nd |
| SE | 0.11 | nd | nd | 0.11 | nd | nd | nd | nd | nd |
| p | 0.91 | nd | nd | 0.91 | nd | nd | nd | nd | nd |
| nCohort 1 | 41 | nd | nd | 41 | nd | nd | nd | nd | nd |
| nCohort 2 | 8 | nd | nd | 8 | nd | nd | nd | nd | nd |
| Cutoff 1 | 16300 | nd | nd | 16300 | nd | nd | nd | nd | nd |
| Sens 1 | 75% | nd | nd | 75% | nd | nd | nd | nd | nd |
| Spec 1 | 17% | nd | nd | 17% | nd | nd | nd | nd | nd |
| Cutoff 2 | 8380 | nd | nd | 8380 | nd | nd | nd | nd | nd |
| Sens 2 | 88% | nd | nd | 88% | nd | nd | nd | nd | nd |
| Spec 2 | 10% | nd | nd | 10% | nd | nd | nd | nd | nd |
| Cutoff 3 | 0 | nd | nd | 0 | nd | nd | nd | nd | nd |
| Sens 3 | 100% | nd | nd | 100% | nd | nd | nd | nd | nd |
| Spec 3 | 0% | nd | nd | 0% | nd | nd | nd | nd | nd |
| Cutoff 4 | 123000 | nd | nd | 123000 | nd | nd | nd | nd | nd |
| Sens 4 | 38% | nd | nd | 38% | nd | nd | nd | nd | nd |
| Spec 4 | 71% | nd | nd | 71% | nd | nd | nd | nd | nd |
| Cutoff 5 | 138000 | nd | nd | 138000 | nd | nd | nd | nd | nd |
| Sens 5 | 25% | nd | nd | 25% | nd | nd | nd | nd | nd |
| Spec 5 | 80% | nd | nd | 80% | nd | nd | nd | nd | nd |
| Cutoff 6 | 155000 | nd | nd | 155000 | nd | nd | nd | nd | nd |
| Sens 6 | 25% | nd | nd | 25% | nd | nd | nd | nd | nd |
| Spec 6 | 90% | nd | nd | 90% | nd | nd | nd | nd | nd |
| OR Quart 2 | 0.30 | nd | nd | 0.30 | nd | nd | nd | nd | nd |
| p Value | 0.33 | nd | nd | 0.33 | nd | nd | nd | nd | nd |
| 95% CI of | 0.027 | nd | nd | 0.027 | nd | nd | nd | nd | nd |
| OR Quart 2 | 3.4 | nd | nd | 3.4 | nd | nd | nd | nd | nd |
| OR Quart 3 | 0.30 | nd | nd | 0.30 | nd | nd | nd | nd | nd |
| p Value | 0.33 | nd | nd | 0.33 | nd | nd | nd | nd | nd |
| 95% CI of | 0.027 | nd | nd | 0.027 | nd | nd | nd | nd | nd |
| OR Quart 3 | 3.4 | nd | nd | 3.4 | nd | nd | nd | nd | nd |
| OR Quart 4 | 1.1 | nd | nd | 1.1 | nd | nd | nd | nd | nd |
| p Value | 0.91 | nd | nd | 0.91 | nd | nd | nd | nd | nd |
| 95% CI of | 0.18 | nd | nd | 0.18 | nd | nd | nd | nd | nd |
| OR Quart 4 | 7.0 | nd | nd | 7.0 | nd | nd | nd | nd | nd |

Cyclin-dependent kinase inhibitor 1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 3.09 | 14.9 | 3.09 | 9.35 | 3.09 | 4.76 |
| Average | 88.7 | 37.1 | 88.7 | 36.6 | 88.7 | 15.2 |
| Stdev | 688 | 81.6 | 688 | 81.8 | 688 | 16.5 |
| p (t-test) | | 0.77 | | 0.77 | | 0.75 |
| Min | 0.116 | 0.116 | 0.116 | 0.116 | 0.116 | 1.64 |
| Max | 6950 | 327 | 6950 | 327 | 6950 | 44.1 |
| n (Samp) | 102 | 15 | 102 | 15 | 102 | 9 |
| n (Patient) | 102 | 15 | 102 | 15 | 102 | 9 |
| sCr only | | | | | | |
| Median | 4.90 | 18.8 | 4.90 | 15.7 | nd | nd |
| Average | 69.2 | 21.5 | 69.2 | 20.7 | nd | nd |
| Stdev | 541 | 18.2 | 541 | 18.6 | nd | nd |
| p (t-test) | | 0.80 | | 0.80 | nd | nd |
| Min | 1.14E−14 | 0.116 | 1.14E−14 | 0.116 | nd | nd |
| Max | 6950 | 44.1 | 6950 | 44.1 | nd | nd |
| n (Samp) | 168 | 8 | 168 | 8 | nd | nd |
| n (Patient) | 168 | 8 | 168 | 8 | nd | nd |
| UO only | | | | | | |
| Median | 3.29 | 14.9 | 3.29 | 14.9 | 3.29 | 4.76 |
| Average | 101 | 50.0 | 101 | 50.0 | 101 | 14.4 |
| Stdev | 753 | 105 | 753 | 105 | 753 | 16.4 |
| p (t-test) | | 0.84 | | 0.84 | | 0.76 |
| Min | 0.116 | 2.45 | 0.116 | 2.45 | 0.116 | 2.45 |
| Max | 6950 | 327 | 6950 | 327 | 6950 | 44.1 |
| n (Samp) | 85 | 9 | 85 | 9 | 85 | 7 |
| n (Patient) | 85 | 9 | 85 | 9 | 85 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | 0.66 | 0.69 | 0.65 | 0.65 | 0.68 | 0.63 | nd | 0.63 |
| SE | 0.081 | 0.11 | 0.10 | 0.081 | 0.11 | 0.10 | 0.10 | nd | 0.12 |
| p | 0.050 | 0.15 | 0.067 | 0.062 | 0.18 | 0.078 | 0.22 | nd | 0.28 |
| nCohort 1 | 102 | 168 | 85 | 102 | 168 | 85 | 102 | nd | 85 |
| nCohort 2 | 15 | 8 | 9 | 15 | 8 | 9 | 9 | nd | 7 |
| Cutoff 1 | 2.45 | 7.35 | 2.45 | 2.45 | 7.35 | 2.45 | 2.02 | nd | 2.45 |
| Sens 1 | 73% | 75% | 78% | 73% | 75% | 78% | 89% | nd | 71% |
| Spec 1 | 46% | 59% | 47% | 46% | 59% | 47% | 46% | nd | 47% |
| Cutoff 2 | 2.02 | 1.36 | 2.02 | 2.02 | 1.36 | 2.02 | 2.02 | nd | 2.02 |
| Sens 2 | 87% | 88% | 100% | 87% | 88% | 100% | 89% | nd | 100% |
| Spec 2 | 46% | 40% | 47% | 46% | 40% | 47% | 46% | nd | 47% |
| Cutoff 3 | 1.09 | 1.14E−14 | 2.02 | 1.09 | 1.14E−14 | 2.02 | 1.09 | nd | 2.02 |
| Sens 3 | 93% | 100% | 100% | 93% | 100% | 100% | 100% | nd | 100% |
| Spec 3 | 44% | 1% | 47% | 44% | 1% | 47% | 44% | nd | 47% |
| Cutoff 4 | 12.3 | 13.6 | 12.3 | 12.3 | 13.6 | 12.3 | 12.3 | nd | 12.3 |
| Sens 4 | 53% | 62% | 56% | 47% | 50% | 56% | 44% | nd | 43% |
| Spec 4 | 71% | 70% | 71% | 71% | 70% | 71% | 71% | nd | 71% |
| Cutoff 5 | 25.7 | 26.7 | 25.7 | 25.7 | 26.7 | 25.7 | 25.7 | nd | 25.7 |
| Sens 5 | 33% | 38% | 33% | 33% | 38% | 33% | 33% | nd | 29% |
| Spec 5 | 80% | 80% | 80% | 80% | 80% | 80% | 80% | nd | 80% |
| Cutoff 6 | 55.7 | 56.1 | 44.8 | 55.7 | 56.1 | 44.8 | 55.7 | nd | 44.8 |
| Sens 6 | 7% | 0% | 11% | 7% | 0% | 11% | 0% | nd | 0% |
| Spec 6 | 90% | 90% | 91% | 90% | 90% | 91% | 90% | nd | 91% |
| OR Quart 2 | 4.5 | >2.1 | >3.3 | 4.5 | >2.1 | >3.3 | >4.5 | nd | >3.4 |
| p Value | 0.19 | <0.55 | <0.32 | 0.19 | <0.55 | <0.32 | <0.19 | nd | <0.30 |
| 95% CI of | 0.47 | >0.18 | >0.32 | 0.47 | >0.18 | >0.32 | >0.47 | nd | >0.33 |
| OR Quart 2 | 43 | na | na | 43 | na | na | na | nd | na |
| OR Quart 3 | 4.5 | >3.2 | >3.4 | 4.5 | >3.2 | >3.4 | >2.1 | nd | >2.2 |
| p Value | 0.19 | <0.32 | <0.30 | 0.19 | <0.32 | <0.30 | <0.56 | nd | <0.53 |
| 95% CI of | 0.47 | >0.32 | >0.33 | 0.47 | >0.32 | >0.33 | >0.18 | nd | >0.18 |
| OR Quart 3 | 43 | na | na | 43 | na | na | na | nd | na |
| OR Quart 4 | 7.0 | >3.2 | >3.3 | 7.0 | >3.2 | >3.3 | >3.2 | nd | >2.2 |
| p Value | 0.081 | <0.32 | <0.32 | 0.081 | <0.32 | <0.32 | <0.32 | nd | <0.53 |
| 95% CI of | 0.79 | >0.32 | >0.32 | 0.79 | >0.32 | >0.32 | >0.32 | nd | >0.18 |
| OR Quart 4 | 62 | na | na | 62 | na | na | na | nd | na |

Carcinoembryonic antigen-related cell adhesion molecule 5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.14 | 5.45 | 1.14 | 2.86 | 1.14 | 2.98 |
| Average | 3.19 | 7.89 | 3.19 | 7.48 | 3.19 | 5.77 |
| Stdev | 5.64 | 11.7 | 5.64 | 12.1 | 5.64 | 4.90 |
| p (t-test) | | 0.0094 | | 0.021 | | 0.19 |
| Min | 0.00336 | 0.282 | 0.00336 | 0.219 | 0.00336 | 0.852 |
| Max | 43.4 | 50.1 | 43.4 | 50.1 | 43.4 | 14.6 |
| n (Samp) | 102 | 17 | 102 | 16 | 102 | 9 |
| n (Patient) | 102 | 17 | 102 | 16 | 102 | 9 |
| sCr only | | | | | | |
| Median | 1.57 | 4.46 | 1.57 | 3.11 | nd | nd |
| Average | 28.3 | 4.33 | 28.3 | 3.91 | nd | nd |
| Stdev | 312 | 3.44 | 312 | 3.51 | nd | nd |
| p (t-test) | | 0.83 | | 0.83 | nd | nd |
| Min | 0.00336 | 0.282 | 0.00336 | 0.219 | nd | nd |
| Max | 4070 | 10.9 | 4070 | 10.9 | nd | nd |
| n (Samp) | 170 | 8 | 170 | 8 | nd | nd |
| n (Patient) | 170 | 8 | 170 | 8 | nd | nd |
| UO only | | | | | | |
| Median | 1.19 | 6.79 | 1.19 | 4.88 | 1.19 | 2.98 |
| Average | 3.63 | 10.4 | 3.63 | 10.3 | 3.63 | 6.19 |
| Stdev | 6.41 | 14.1 | 6.41 | 14.7 | 6.41 | 5.50 |
| p (t-test) | | 0.0070 | | 0.011 | | 0.31 |
| Min | 0.00336 | 0.356 | 0.00336 | 0.852 | 0.00336 | 0.852 |
| Max | 43.4 | 50.1 | 43.4 | 50.1 | 43.4 | 14.6 |
| n (Samp) | 85 | 11 | 85 | 10 | 85 | 7 |
| n (Patient) | 85 | 11 | 85 | 10 | 85 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.70 | 0.62 | 0.75 | 0.69 | 0.57 | 0.77 | 0.76 | nd | 0.74 |
| SE | 0.075 | 0.11 | 0.089 | 0.078 | 0.11 | 0.091 | 0.096 | nd | 0.11 |
| p | 0.0072 | 0.28 | 0.0055 | 0.015 | 0.50 | 0.0030 | 0.0080 | nd | 0.034 |
| nCohort 1 | 102 | 170 | 85 | 102 | 170 | 85 | 102 | nd | 85 |
| nCohort 2 | 17 | 8 | 11 | 16 | 8 | 10 | 9 | nd | 7 |
| Cutoff 1 | 2.56 | 2.59 | 2.88 | 2.26 | 2.26 | 2.55 | 2.53 | nd | 2.53 |
| Sens 1 | 71% | 75% | 73% | 75% | 75% | 70% | 78% | nd | 71% |
| Spec 1 | 71% | 65% | 71% | 67% | 61% | 68% | 70% | nd | 68% |
| Cutoff 2 | 0.816 | 0.450 | 1.40 | 1.44 | 0.238 | 2.53 | 1.44 | nd | 1.40 |
| Sens 2 | 82% | 88% | 82% | 81% | 88% | 80% | 89% | nd | 86% |
| Spec 2 | 42% | 20% | 56% | 58% | 8% | 68% | 58% | nd | 56% |
| Cutoff 3 | 0.339 | 0.281 | 0.816 | 0.238 | 0.214 | 1.40 | 0.816 | nd | 0.816 |
| Sens 3 | 94% | 100% | 91% | 94% | 100% | 90% | 100% | nd | 100% |
| Spec 3 | 19% | 10% | 39% | 9% | 5% | 56% | 42% | nd | 39% |
| Cutoff 4 | 2.56 | 3.33 | 2.88 | 2.56 | 3.33 | 2.88 | 2.56 | nd | 2.88 |
| Sens 4 | 71% | 62% | 73% | 56% | 50% | 60% | 67% | nd | 57% |
| Spec 4 | 71% | 70% | 71% | 71% | 70% | 71% | 71% | nd | 71% |
| Cutoff 5 | 4.03 | 5.77 | 5.67 | 4.03 | 5.77 | 5.67 | 4.03 | nd | 5.67 |
| Sens 5 | 53% | 25% | 55% | 44% | 25% | 50% | 44% | nd | 43% |
| Spec 5 | 80% | 80% | 80% | 80% | 80% | 80% | 80% | nd | 80% |
| Cutoff 6 | 8.86 | 11.0 | 9.55 | 8.86 | 11.0 | 9.55 | 8.86 | nd | 9.55 |
| Sens 6 | 29% | 0% | 45% | 25% | 0% | 40% | 33% | nd | 43% |
| Spec 6 | 90% | 90% | 91% | 90% | 90% | 91% | 90% | nd | 91% |
| OR Quart 2 | 0.30 | 0 | 1.0 | 0.47 | 0 | >1.0 | >1.0 | nd | >1.0 |
| p Value | 0.31 | na | 1.0 | 0.54 | na | <1.0 | <1.0 | nd | <0.98 |
| 95% CI of | 0.029 | na | 0.059 | 0.040 | na | >0.059 | >0.059 | nd | >0.062 |
| OR Quart 2 | 3.1 | na | 17 | 5.4 | na | na | na | nd | na |
| OR Quart 3 | 1.3 | 1.0 | 3.3 | 3.5 | 1.5 | >4.6 | >4.5 | nd | >3.4 |
| p Value | 0.72 | 1.0 | 0.32 | 0.15 | 0.65 | <0.19 | <0.19 | nd | <0.30 |
| 95% CI of | 0.27 | 0.13 | 0.32 | 0.65 | 0.24 | >0.47 | >0.47 | nd | >0.33 |
| OR Quart 3 | 6.6 | 7.4 | 34 | 19 | 9.7 | na | na | nd | na |
| OR Quart 4 | 3.7 | 2.0 | 7.7 | 4.1 | 1.5 | >6.1 | >4.5 | nd | >3.4 |
| p Value | 0.072 | 0.42 | 0.070 | 0.097 | 0.67 | <0.11 | <0.19 | nd | <0.30 |
| 95% CI of | 0.89 | 0.36 | 0.85 | 0.78 | 0.24 | >0.65 | >0.47 | nd | >0.33 |
| OR Quart 4 | 15 | 12 | 70 | 22 | 9.4 | na | na | nd | na |

Myoglobin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 5.42 | 18.9 | 5.42 | 11.9 | 5.42 | 44.9 |
| Average | 51.4 | 149 | 51.4 | 155 | 51.4 | 174 |
| Stdev | 121 | 203 | 121 | 208 | 121 | 213 |
| p (t-test) | | 0.0066 | | 0.0052 | | 0.0076 |
| Min | 0.00616 | 0.0439 | 0.00616 | 0.0439 | 0.00616 | 0.0772 |
| Max | 618 | 469 | 618 | 469 | 618 | 469 |
| n (Samp) | 102 | 17 | 102 | 16 | 102 | 9 |
| n (Patient) | 102 | 17 | 102 | 16 | 102 | 9 |
| sCr only | | | | | | |
| Median | 3.65 | 31.9 | 3.65 | 29.4 | nd | nd |
| Average | 63.1 | 142 | 63.1 | 141 | nd | nd |
| Stdev | 142 | 195 | 142 | 196 | nd | nd |
| p (t-test) | | 0.13 | | 0.14 | nd | nd |
| Min | 0.00616 | 0.0439 | 0.00616 | 0.0439 | nd | nd |
| Max | 618 | 469 | 618 | 469 | nd | nd |
| n (Samp) | 170 | 8 | 170 | 8 | nd | nd |
| n (Patient) | 170 | 8 | 170 | 8 | nd | nd |
| UO only | | | | | | |
| Median | 3.83 | 0.377 | 3.83 | 2.87 | 3.83 | 5.36 |
| Average | 56.6 | 143 | 56.6 | 152 | 56.6 | 150 |
| Stdev | 128 | 203 | 128 | 212 | 128 | 207 |
| p (t-test) | | 0.055 | | 0.042 | | 0.081 |
| Min | 0.00616 | 0.0696 | 0.00616 | 0.0772 | 0.00616 | 0.0772 |
| Max | 618 | 469 | 618 | 469 | 618 | 469 |
| n (Samp) | 85 | 11 | 85 | 10 | 85 | 7 |
| n (Patient) | 85 | 11 | 85 | 10 | 85 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.57 | 0.64 | 0.49 | 0.57 | 0.63 | 0.51 | 0.62 | nd | 0.54 |
| SE | 0.078 | 0.11 | 0.093 | 0.080 | 0.11 | 0.097 | 0.10 | nd | 0.12 |
| p | 0.36 | 0.21 | 0.95 | 0.36 | 0.24 | 0.93 | 0.25 | nd | 0.73 |
| nCohort 1 | 102 | 170 | 85 | 102 | 170 | 85 | 102 | nd | 85 |
| nCohort 2 | 17 | 8 | 11 | 16 | 8 | 10 | 9 | nd | 7 |
| Cutoff 1 | 0.288 | 10.2 | 0.117 | 0.288 | 9.39 | 0.288 | 0.288 | nd | 0.288 |
| Sens 1 | 71% | 75% | 73% | 75% | 75% | 70% | 78% | nd | 71% |
| Spec 1 | 21% | 61% | 14% | 21% | 61% | 20% | 21% | nd | 20% |
| Cutoff 2 | 0.0772 | 0.0780 | 0.0772 | 0.117 | 0.0780 | 0.117 | 0.117 | nd | 0.117 |
| Sens 2 | 82% | 88% | 82% | 81% | 88% | 80% | 89% | nd | 86% |
| Spec 2 | 13% | 14% | 11% | 16% | 14% | 14% | 16% | nd | 14% |
| Cutoff 3 | 0.0606 | 0.0411 | 0.0696 | 0.0606 | 0.0411 | 0.0772 | 0.0606 | nd | 0.0661 |
| Sens 3 | 94% | 100% | 91% | 94% | 100% | 90% | 100% | nd | 100% |
| Spec 3 | 13% | 9% | 11% | 13% | 9% | 11% | 13% | nd | 11% |
| Cutoff 4 | 17.2 | 18.6 | 17.9 | 17.2 | 18.6 | 17.9 | 17.2 | nd | 17.9 |
| Sens 4 | 53% | 62% | 45% | 44% | 50% | 40% | 56% | nd | 43% |
| Spec 4 | 71% | 70% | 71% | 71% | 70% | 71% | 71% | nd | 71% |
| Cutoff 5 | 38.9 | 45.3 | 55.8 | 38.9 | 45.3 | 55.8 | 38.9 | nd | 55.8 |
| Sens 5 | 47% | 38% | 36% | 44% | 38% | 40% | 56% | nd | 43% |
| Spec 5 | 80% | 80% | 80% | 80% | 80% | 80% | 80% | nd | 80% |
| Cutoff 6 | 222 | 234 | 232 | 222 | 234 | 232 | 222 | nd | 232 |
| Sens 6 | 29% | 25% | 27% | 31% | 25% | 30% | 33% | nd | 29% |
| Spec 6 | 90% | 90% | 91% | 90% | 90% | 91% | 90% | nd | 91% |
| OR Quart 2 | 0.13 | 0 | 0 | 0.13 | 0 | 0.21 | 0.30 | nd | 0 |
| p Value | 0.070 | na | na | 0.070 | na | 0.17 | 0.31 | nd | na |
| 95% CI of | 0.015 | na | na | 0.015 | na | 0.021 | 0.029 | nd | na |
| OR Quart 2 | 1.2 | na | na | 1.2 | na | 2.0 | 3.0 | nd | na |
| OR Quart 3 | 0.27 | 1.0 | 0.17 | 0.28 | 1.0 | 0.21 | 0 | nd | 0.30 |
| p Value | 0.13 | 1.0 | 0.11 | 0.15 | 1.0 | 0.17 | na | nd | 0.32 |
| 95% CI of | 0.050 | 0.13 | 0.018 | 0.052 | 0.13 | 0.021 | na | nd | 0.029 |
| OR Quart 3 | 1.5 | 7.4 | 1.5 | 1.5 | 7.4 | 2.0 | na | nd | 3.2 |
| OR Quart 4 | 1.4 | 2.0 | 1.0 | 1.2 | 2.0 | 0.95 | 1.7 | nd | 1.0 |
| p Value | 0.59 | 0.42 | 1.0 | 0.81 | 0.42 | 0.95 | 0.48 | nd | 1.0 |
| 95% CI of | 0.42 | 0.36 | 0.25 | 0.34 | 0.36 | 0.21 | 0.37 | nd | 0.18 |
| OR Quart 4 | 4.7 | 12 | 4.0 | 4.0 | 12 | 4.4 | 8.1 | nd | 5.6 |

Mucin-16

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 0.684 | 1.03 | 0.684 | 1.03 | nd | nd |
| Average | 1.18 | 54.9 | 1.18 | 54.9 | nd | nd |
| Stdev | 1.41 | 153 | 1.41 | 153 | nd | nd |
| p (t-test) | | 0.023 | | 0.023 | nd | nd |
| Min | 0.141 | 0.304 | 0.141 | 0.304 | nd | nd |
| Max | 6.37 | 433 | 6.37 | 433 | nd | nd |
| n (Samp) | 41 | 8 | 41 | 8 | nd | nd |
| n (Patient) | 41 | 8 | 41 | 8 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.65 | nd | nd | 0.65 | nd | nd | nd | nd | nd |
| SE | 0.11 | nd | nd | 0.11 | nd | nd | nd | nd | nd |
| p | 0.20 | nd | nd | 0.20 | nd | nd | nd | nd | nd |
| nCohort 1 | 41 | nd | nd | 41 | nd | nd | nd | nd | nd |
| nCohort 2 | 8 | nd | nd | 8 | nd | nd | nd | nd | nd |
| Cutoff 1 | 0.781 | nd | nd | 0.781 | nd | nd | nd | nd | nd |
| Sens 1 | 75% | nd | nd | 75% | nd | nd | nd | nd | nd |
| Spec 1 | 61% | nd | nd | 61% | nd | nd | nd | nd | nd |
| Cutoff 2 | 0.464 | nd | nd | 0.464 | nd | nd | nd | nd | nd |
| Sens 2 | 88% | nd | nd | 88% | nd | nd | nd | nd | nd |
| Spec 2 | 32% | nd | nd | 32% | nd | nd | nd | nd | nd |
| Cutoff 3 | 0.278 | nd | nd | 0.278 | nd | nd | nd | nd | nd |
| Sens 3 | 100% | nd | nd | 100% | nd | nd | nd | nd | nd |
| Spec 3 | 10% | nd | nd | 10% | nd | nd | nd | nd | nd |
| Cutoff 4 | 0.961 | nd | nd | 0.961 | nd | nd | nd | nd | nd |
| Sens 4 | 50% | nd | nd | 50% | nd | nd | nd | nd | nd |
| Spec 4 | 71% | nd | nd | 71% | nd | nd | nd | nd | nd |
| Cutoff 5 | 1.31 | nd | nd | 1.31 | nd | nd | nd | nd | nd |
| Sens 5 | 25% | nd | nd | 25% | nd | nd | nd | nd | nd |
| Spec 5 | 80% | nd | nd | 80% | nd | nd | nd | nd | nd |
| Cutoff 6 | 3.42 | nd | nd | 3.42 | nd | nd | nd | nd | nd |
| Sens 6 | 12% | nd | nd | 12% | nd | nd | nd | nd | nd |
| Spec 6 | 90% | nd | nd | 90% | nd | nd | nd | nd | nd |
| OR Quart 2 | 1.0 | nd | nd | 1.0 | nd | nd | nd | nd | nd |
| p Value | 1.0 | nd | nd | 1.0 | nd | nd | nd | nd | nd |
| 95% CI of | 0.055 | nd | nd | 0.055 | nd | nd | nd | nd | nd |
| OR Quart 2 | 18 | nd | nd | 18 | nd | nd | nd | nd | nd |
| OR Quart 3 | 3.7 | nd | nd | 3.7 | nd | nd | nd | nd | nd |
| p Value | 0.29 | nd | nd | 0.29 | nd | nd | nd | nd | nd |
| 95% CI of | 0.32 | nd | nd | 0.32 | nd | nd | nd | nd | nd |
| OR Quart 3 | 42 | nd | nd | 42 | nd | nd | nd | nd | nd |
| OR Quart 4 | 3.3 | nd | nd | 3.3 | nd | nd | nd | nd | nd |
| p Value | 0.33 | nd | nd | 0.33 | nd | nd | nd | nd | nd |
| 95% CI of | 0.29 | nd | nd | 0.29 | nd | nd | nd | nd | nd |
| OR Quart 4 | 37 | nd | nd | 37 | nd | nd | nd | nd | nd |

Poly [ADP-ribose] polymerase 1 (cleaved)

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.00E−9 | 0.0140 | 1.00E−9 | 0.0140 | 1.00E−9 | 1.00E−9 |
| Average | 0.00479 | 0.0101 | 0.00479 | 0.00956 | 0.00479 | 0.00605 |
| Stdev | 0.00701 | 0.00857 | 0.00701 | 0.00900 | 0.00701 | 0.00754 |
| p (t-test) | | 0.018 | | 0.034 | | 0.65 |
| Min | 1.00E−9 | 1.00E−9 | 1.00E−9 | 1.00E−9 | 1.00E−9 | 1.00E−9 |
| Max | 0.0357 | 0.0253 | 0.0357 | 0.0253 | 0.0357 | 0.0144 |
| n (Samp) | 97 | 12 | 97 | 12 | 97 | 7 |
| n (Patient) | 97 | 12 | 97 | 12 | 97 | 7 |
| sCr only | | | | | | |
| Median | 1.00E−9 | 0.00990 | 1.00E−9 | 0.00691 | nd | nd |
| Average | 0.00475 | 0.00992 | 0.00475 | 0.00892 | nd | nd |
| Stdev | 0.00651 | 0.00984 | 0.00651 | 0.0106 | nd | nd |
| p (t-test) | | 0.063 | | 0.13 | nd | nd |
| Min | 1.00E−9 | 1.00E−9 | 1.00E−9 | 1.00E−9 | nd | nd |
| Max | 0.0357 | 0.0253 | 0.0357 | 0.0253 | nd | nd |
| n (Samp) | 159 | 6 | 159 | 6 | nd | nd |
| n (Patient) | 159 | 6 | 159 | 6 | nd | nd |
| UO only | | | | | | |
| Median | 1.00E−9 | 0.0140 | 1.00E−9 | 0.0140 | 1.00E−9 | 0.00691 |
| Average | 0.00380 | 0.00938 | 0.00380 | 0.00938 | 0.00380 | 0.00706 |
| Stdev | 0.00658 | 0.00789 | 0.00658 | 0.00789 | 0.00658 | 0.00773 |
| p (t-test) | | 0.027 | | 0.027 | | 0.25 |
| Min | 1.00E−9 | 1.00E−9 | 1.00E−9 | 1.00E−9 | 1.00E−9 | 1.00E−9 |
| Max | 0.0357 | 0.0183 | 0.0357 | 0.0183 | 0.0357 | 0.0144 |
| n (Samp) | 84 | 8 | 84 | 8 | 84 | 6 |
| n (Patient) | 84 | 8 | 84 | 8 | 84 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.67 | 0.65 | 0.69 | 0.64 | 0.58 | 0.69 | 0.52 | nd | 0.60 |
| SE | 0.090 | 0.12 | 0.11 | 0.091 | 0.12 | 0.11 | 0.11 | nd | 0.13 |
| p | 0.059 | 0.23 | 0.081 | 0.13 | 0.51 | 0.081 | 0.87 | nd | 0.43 |
| nCohort 1 | 97 | 159 | 84 | 97 | 159 | 84 | 97 | nd | 84 |
| nCohort 2 | 12 | 6 | 8 | 12 | 6 | 8 | 7 | nd | 6 |
| Cutoff 1 | 0 | 0 | 0 | 0 | 0 | 0 | 0 | nd | 0 |
| Sens 1 | 100% | 100% | 100% | 100% | 100% | 100% | 100% | nd | 100% |
| Spec 1 | 0% | 0% | 0% | 0% | 0% | 0% | 0% | nd | 0% |
| Cutoff 2 | 0 | 0 | 0 | 0 | 0 | 0 | 0 | nd | 0 |
| Sens 2 | 100% | 100% | 100% | 100% | 100% | 100% | 100% | nd | 100% |
| Spec 2 | 0% | 0% | 0% | 0% | 0% | 0% | 0% | nd | 0% |
| Cutoff 3 | 0 | 0 | 0 | 0 | 0 | 0 | 0 | nd | 0 |
| Sens 3 | 100% | 100% | 100% | 100% | 100% | 100% | 100% | nd | 100% |
| Spec 3 | 0% | 0% | 0% | 0% | 0% | 0% | 0% | nd | 0% |
| Cutoff 4 | 0.00598 | 0.00598 | 0.00471 | 0.00598 | 0.00598 | 0.00471 | 0.00598 | nd | 0.00471 |
| Sens 4 | 58% | 50% | 62% | 58% | 50% | 62% | 43% | nd | 50% |
| Spec 4 | 70% | 71% | 74% | 70% | 71% | 74% | 70% | nd | 74% |
| Cutoff 5 | 0.0144 | 0.0144 | 0.0120 | 0.0144 | 0.0144 | 0.0120 | 0.0144 | nd | 0.0120 |
| Sens 5 | 17% | 17% | 62% | 17% | 17% | 62% | 0% | nd | 50% |
| Spec 5 | 94% | 96% | 81% | 94% | 96% | 81% | 94% | nd | 81% |
| Cutoff 6 | 0.0144 | 0.0144 | 0.0144 | 0.0144 | 0.0144 | 0.0144 | 0.0144 | nd | 0.0144 |
| Sens 6 | 17% | 17% | 12% | 17% | 17% | 12% | 0% | nd | 0% |
| Spec 6 | 94% | 96% | 99% | 94% | 96% | 99% | 94% | nd | 99% |
| OR Quart 2 | 1.0 | 1.0 | 2.1 | 1.6 | 2.1 | 2.1 | 3.3 | nd | 2.0 |
| p Value | 1.0 | 1.0 | 0.56 | 0.64 | 0.56 | 0.56 | 0.32 | nd | 0.58 |
| 95% CI of | 0.13 | 0.060 | 0.18 | 0.24 | 0.18 | 0.18 | 0.32 | nd | 0.17 |
| OR Quart 2 | 7.7 | 17 | 25 | 10 | 24 | 25 | 34 | nd | 24 |
| OR Quart 3 | 1.0 | 1.0 | 0 | 0 | 0 | 0 | 1.0 | nd | 0 |
| p Value | 1.0 | 1.0 | na | na | na | na | 1.0 | nd | na |
| 95% CI of | 0.13 | 0.060 | na | na | na | na | 0.059 | nd | na |
| OR Quart 3 | 7.7 | 17 | na | na | na | na | 17 | nd | na |
| OR Quart 4 | 3.4 | 3.1 | 6.1 | 4.2 | 3.1 | 6.1 | 2.1 | nd | 3.1 |
| p Value | 0.16 | 0.34 | 0.11 | 0.095 | 0.34 | 0.11 | 0.56 | nd | 0.34 |
| 95% CI of | 0.62 | 0.31 | 0.65 | 0.78 | 0.31 | 0.65 | 0.18 | nd | 0.30 |
| OR Quart 4 | 19 | 31 | 57 | 22 | 31 | 57 | 25 | nd | 33 |

KSP-Cadherin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.23 | 2.26 | 1.23 | 2.26 | nd | nd |
| Average | 1.66 | 2.48 | 1.66 | 2.48 | nd | nd |
| Stdev | 1.71 | 1.19 | 1.71 | 1.19 | nd | nd |
| p (t-test) | | 0.17 | | 0.17 | nd | nd |
| Min | 0.00263 | 0.562 | 0.00263 | 0.562 | nd | nd |
| Max | 11.9 | 4.23 | 11.9 | 4.23 | nd | nd |
| n (Samp) | 68 | 9 | 68 | 9 | nd | nd |
| n (Patient) | 68 | 9 | 68 | 9 | nd | nd |
| UO only | | | | | | |
| Median | 1.17 | 1.87 | 1.17 | 1.87 | nd | nd |
| Average | 1.76 | 2.07 | 1.76 | 2.07 | nd | nd |
| Stdev | 1.84 | 1.22 | 1.84 | 1.22 | nd | nd |
| p (t-test) | | 0.69 | | 0.69 | nd | nd |
| Min | 0.00263 | 0.562 | 0.00263 | 0.562 | nd | nd |
| Max | 11.9 | 4.23 | 11.9 | 4.23 | nd | nd |
| n (Samp) | 59 | 6 | 59 | 6 | nd | nd |
| n (Patient) | 59 | 6 | 59 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.74 | nd | 0.64 | 0.74 | nd | 0.64 | nd | nd | nd |
| SE | 0.099 | nd | 0.13 | 0.099 | nd | 0.13 | nd | nd | nd |
| p | 0.017 | nd | 0.29 | 0.017 | nd | 0.29 | nd | nd | nd |
| nCohort 1 | 68 | nd | 59 | 68 | nd | 59 | nd | nd | nd |
| nCohort 2 | 9 | nd | 6 | 9 | nd | 6 | nd | nd | nd |
| Cutoff 1 | 1.61 | nd | 1.51 | 1.61 | nd | 1.51 | nd | nd | nd |
| Sens 1 | 78% | nd | 83% | 78% | nd | 83% | nd | nd | nd |
| Spec 1 | 60% | nd | 59% | 60% | nd | 59% | nd | nd | nd |
| Cutoff 2 | 1.56 | nd | 1.51 | 1.56 | nd | 1.51 | nd | nd | nd |
| Sens 2 | 89% | nd | 83% | 89% | nd | 83% | nd | nd | nd |
| Spec 2 | 60% | nd | 59% | 60% | nd | 59% | nd | nd | nd |
| Cutoff 3 | 0.466 | nd | 0.494 | 0.466 | nd | 0.494 | nd | nd | nd |
| Sens 3 | 100% | nd | 100% | 100% | nd | 100% | nd | nd | nd |
| Spec 3 | 19% | nd | 19% | 19% | nd | 19% | nd | nd | nd |
| Cutoff 4 | 1.99 | nd | 2.08 | 1.99 | nd | 2.08 | nd | nd | nd |
| Sens 4 | 67% | nd | 50% | 67% | nd | 50% | nd | nd | nd |
| Spec 4 | 71% | nd | 71% | 71% | nd | 71% | nd | nd | nd |
| Cutoff 5 | 2.24 | nd | 2.54 | 2.24 | nd | 2.54 | nd | nd | nd |
| Sens 5 | 56% | nd | 17% | 56% | nd | 17% | nd | nd | nd |
| Spec 5 | 81% | nd | 81% | 81% | nd | 81% | nd | nd | nd |
| Cutoff 6 | 3.17 | nd | 3.77 | 3.17 | nd | 3.77 | nd | nd | nd |
| Sens 6 | 22% | nd | 17% | 22% | nd | 17% | nd | nd | nd |
| Spec 6 | 91% | nd | 92% | 91% | nd | 92% | nd | nd | nd |
| OR Quart 2 | 0 | nd | 0 | 0 | nd | 0 | nd | nd | nd |
| p Value | na | nd | na | na | nd | na | nd | nd | nd |
| 95% CI of | na | nd | na | na | nd | na | nd | nd | nd |
| OR Quart 2 | na | nd | na | na | nd | na | nd | nd | nd |
| OR Quart 3 | 3.4 | nd | 3.5 | 3.4 | nd | 3.5 | nd | nd | nd |
| p Value | 0.31 | nd | 0.31 | 0.31 | nd | 0.31 | nd | nd | nd |
| 95% CI of | 0.32 | nd | 0.32 | 0.32 | nd | 0.32 | nd | nd | nd |
| OR Quart 3 | 36 | nd | 37 | 36 | nd | 37 | nd | nd | nd |
| OR Quart 4 | 6.0 | nd | 2.0 | 6.0 | nd | 2.0 | nd | nd | nd |
| p Value | 0.12 | nd | 0.59 | 0.12 | nd | 0.59 | nd | nd | nd |
| 95% CI of | 0.63 | nd | 0.16 | 0.63 | nd | 0.16 | nd | nd | nd |
| OR Quart 4 | 57 | nd | 24 | 57 | nd | 24 | nd | nd | nd |

Tumor necrosis factor receptor superfamily member 10B

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 0.141 | 0.904 | 0.141 | 0.904 | nd | nd |
| Average | 0.957 | 1.95 | 0.957 | 1.95 | nd | nd |
| Stdev | 1.61 | 2.64 | 1.61 | 2.64 | nd | nd |
| p (t-test) | | 0.16 | | 0.16 | nd | nd |
| Min | 0.00360 | 0.104 | 0.00360 | 0.104 | nd | nd |
| Max | 6.71 | 7.54 | 6.71 | 7.54 | nd | nd |
| n (Samp) | 41 | 8 | 41 | 8 | nd | nd |
| n (Patient) | 41 | 8 | 41 | 8 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.69 | nd | nd | 0.69 | nd | nd | nd | nd | nd |
| SE | 0.11 | nd | nd | 0.11 | nd | nd | nd | nd | nd |
| p | 0.092 | nd | nd | 0.092 | nd | nd | nd | nd | nd |
| nCohort 1 | 41 | nd | nd | 41 | nd | nd | nd | nd | nd |
| nCohort 2 | 8 | nd | nd | 8 | nd | nd | nd | nd | nd |
| Cutoff 1 | 0.141 | nd | nd | 0.141 | nd | nd | nd | nd | nd |
| Sens 1 | 75% | nd | nd | 75% | nd | nd | nd | nd | nd |
| Spec 1 | 51% | nd | nd | 51% | nd | nd | nd | nd | nd |
| Cutoff 2 | 0.107 | nd | nd | 0.107 | nd | nd | nd | nd | nd |
| Sens 2 | 88% | nd | nd | 88% | nd | nd | nd | nd | nd |
| Spec 2 | 49% | nd | nd | 49% | nd | nd | nd | nd | nd |
| Cutoff 3 | 0.0796 | nd | nd | 0.0796 | nd | nd | nd | nd | nd |
| Sens 3 | 100% | nd | nd | 100% | nd | nd | nd | nd | nd |
| Spec 3 | 41% | nd | nd | 41% | nd | nd | nd | nd | nd |
| Cutoff 4 | 0.693 | nd | nd | 0.693 | nd | nd | nd | nd | nd |
| Sens 4 | 50% | nd | nd | 50% | nd | nd | nd | nd | nd |
| Spec 4 | 71% | nd | nd | 71% | nd | nd | nd | nd | nd |
| Cutoff 5 | 1.56 | nd | nd | 1.56 | nd | nd | nd | nd | nd |
| Sens 5 | 38% | nd | nd | 38% | nd | nd | nd | nd | nd |
| Spec 5 | 80% | nd | nd | 80% | nd | nd | nd | nd | nd |
| Cutoff 6 | 3.31 | nd | nd | 3.31 | nd | nd | nd | nd | nd |
| Sens 6 | 25% | nd | nd | 25% | nd | nd | nd | nd | nd |
| Spec 6 | 90% | nd | nd | 90% | nd | nd | nd | nd | nd |
| OR Quart 2 | >4.0 | nd | nd | >4.0 | nd | nd | nd | nd | nd |
| p Value | <0.26 | nd | nd | <0.26 | nd | nd | nd | nd | nd |
| 95% CI of | >0.35 | nd | nd | >0.35 | nd | nd | nd | nd | nd |
| OR Quart 2 | na | nd | nd | na | nd | nd | nd | nd | nd |
| OR Quart 3 | >1.1 | nd | nd | >1.1 | nd | nd | nd | nd | nd |
| p Value | <0.95 | nd | nd | <0.95 | nd | nd | nd | nd | nd |
| 95% CI of | >0.061 | nd | nd | >0.061 | nd | nd | nd | nd | nd |
| OR Quart 3 | na | nd | nd | na | nd | nd | nd | nd | nd |
| OR Quart 4 | >5.3 | nd | nd | >5.3 | nd | nd | nd | nd | nd |
| p Value | <0.16 | nd | nd | <0.16 | nd | nd | nd | nd | nd |
| 95% CI of | >0.51 | nd | nd | >0.51 | nd | nd | nd | nd | nd |
| OR Quart 4 | na | nd | nd | na | nd | nd | nd | nd | nd |

TABLE 5
Comparison of marker levels in EDTA samples collected from Cohort 1
(patients that did not progress beyond RIFLE stage 0) and in EDTA samples collected from
subjects at 0, 24 hours, and 48 hours prior to reaching stage R, I or F in Cohort 2.
Apolipoprotein A-II

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 50600 | 49600 | 50600 | 44300 | 50600 | 54300 |
| Average | 51700 | 52000 | 51700 | 50700 | 51700 | 57500 |
| Stdev | 22200 | 21100 | 22200 | 36000 | 22200 | 32700 |
| p (t-test) | | 0.94 | | 0.83 | | 0.29 |
| Min | 5450 | 7970 | 5450 | 7680 | 5450 | 8560 |
| Max | 105000 | 97000 | 105000 | 253000 | 105000 | 152000 |
| n (Samp) | 105 | 45 | 105 | 50 | 105 | 24 |
| n (Patient) | 97 | 45 | 97 | 50 | 97 | 24 |
| sCr only | | | | | | |
| Median | 49500 | 59900 | 49500 | 50300 | 49500 | 57100 |
| Average | 51700 | 60100 | 51700 | 64100 | 51700 | 67500 |
| Stdev | 26400 | 18100 | 26400 | 55400 | 26400 | 44100 |
| p (t-test) | | 0.26 | | 0.097 | | 0.062 |
| Min | 1810 | 34500 | 1810 | 12100 | 1810 | 10900 |
| Max | 253000 | 95300 | 253000 | 251000 | 253000 | 176000 |
| n (Samp) | 246 | 13 | 246 | 16 | 246 | 11 |
| n (Patient) | 160 | 13 | 160 | 16 | 160 | 11 |
| UO only | | | | | | |
| Median | 50200 | 49600 | 50200 | 43800 | 50200 | 51400 |
| Average | 52500 | 51300 | 52500 | 51600 | 52500 | 58800 |
| Stdev | 20200 | 21600 | 20200 | 38000 | 20200 | 32900 |
| p (t-test) | | 0.76 | | 0.86 | | 0.25 |
| Min | 8680 | 7970 | 8680 | 7680 | 8680 | 8560 |
| Max | 123000 | 97000 | 123000 | 253000 | 123000 | 152000 |
| n (Samp) | 96 | 40 | 96 | 44 | 96 | 21 |
| n (Patient) | 84 | 40 | 84 | 44 | 84 | 21 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.51 | 0.63 | 0.48 | 0.45 | 0.55 | 0.44 | 0.55 | 0.60 | 0.55 |
| SE | 0.052 | 0.085 | 0.055 | 0.050 | 0.076 | 0.053 | 0.066 | 0.092 | 0.071 |
| p | 0.89 | 0.12 | 0.75 | 0.35 | 0.55 | 0.24 | 0.50 | 0.27 | 0.49 |
| nCohort 1 | 105 | 246 | 96 | 105 | 246 | 96 | 105 | 246 | 96 |
| nCohort 2 | 45 | 13 | 40 | 50 | 16 | 44 | 24 | 11 | 21 |
| Cutoff 1 | 39500 | 43000 | 39500 | 35500 | 37700 | 35000 | 40700 | 46100 | 40900 |
| Sens 1 | 71% | 77% | 70% | 70% | 75% | 70% | 71% | 73% | 71% |
| Spec 1 | 33% | 40% | 28% | 24% | 31% | 19% | 35% | 46% | 31% |
| Cutoff 2 | 33000 | 42600 | 32500 | 29600 | 35500 | 25400 | 29600 | 37000 | 35000 |
| Sens 2 | 80% | 85% | 80% | 80% | 81% | 82% | 83% | 82% | 81% |
| Spec 2 | 22% | 39% | 16% | 19% | 24% | 6% | 19% | 29% | 19% |
| Cutoff 3 | 23400 | 37000 | 23400 | 20800 | 14000 | 20800 | 14000 | 36200 | 21600 |
| Sens 3 | 91% | 92% | 90% | 90% | 94% | 91% | 92% | 91% | 90% |
| Spec 3 | 10% | 29% | 5% | 6% | 5% | 3% | 4% | 27% | 4% |
| Cutoff 4 | 61300 | 61500 | 61500 | 61300 | 61500 | 61500 | 61300 | 61500 | 61500 |
| Sens 4 | 33% | 46% | 30% | 32% | 44% | 34% | 46% | 36% | 48% |
| Spec 4 | 70% | 70% | 71% | 70% | 70% | 71% | 70% | 70% | 71% |
| Cutoff 5 | 69000 | 69400 | 67400 | 69000 | 69400 | 67400 | 69000 | 69400 | 67400 |
| Sens 5 | 24% | 38% | 22% | 18% | 38% | 16% | 33% | 36% | 43% |
| Spec 5 | 80% | 80% | 80% | 80% | 80% | 80% | 80% | 80% | 80% |
| Cutoff 6 | 82000 | 82800 | 77900 | 82000 | 82800 | 77900 | 82000 | 82800 | 77900 |
| Sens 6 | 9% | 8% | 12% | 8% | 12% | 11% | 21% | 36% | 19% |
| Spec 6 | 90% | 90% | 91% | 90% | 90% | 91% | 90% | 90% | 91% |
| OR Quart 2 | 1.6 | 4.1 | 0.74 | 0.60 | 0.98 | 0.76 | 0.80 | 4.2 | 0.61 |
| p Value | 0.36 | 0.21 | 0.58 | 0.32 | 0.98 | 0.60 | 0.74 | 0.21 | 0.49 |
| 95% CI of | 0.59 | 0.45 | 0.25 | 0.22 | 0.24 | 0.27 | 0.22 | 0.46 | 0.15 |
| OR Quart 2 | 4.2 | 38 | 2.2 | 1.6 | 4.1 | 2.1 | 3.0 | 39 | 2.5 |
| OR Quart 3 | 0.87 | 3.0 | 1.1 | 1.1 | 0.48 | 0.65 | 0.62 | 2.0 | 0.28 |
| p Value | 0.79 | 0.34 | 0.79 | 0.81 | 0.41 | 0.42 | 0.49 | 0.57 | 0.15 |
| 95% CI of | 0.31 | 0.31 | 0.41 | 0.44 | 0.086 | 0.22 | 0.16 | 0.18 | 0.052 |
| OR Quart 3 | 2.5 | 30 | 3.2 | 2.8 | 2.7 | 1.9 | 2.4 | 23 | 1.5 |
| OR Quart 4 | 1.2 | 5.2 | 1.1 | 1.2 | 1.5 | 1.8 | 1.6 | 4.1 | 1.6 |
| p Value | 0.67 | 0.14 | 0.79 | 0.75 | 0.53 | 0.22 | 0.42 | 0.21 | 0.41 |
| 95% CI of | 0.46 | 0.60 | 0.41 | 0.46 | 0.41 | 0.69 | 0.50 | 0.45 | 0.50 |
| OR Quart 4 | 3.4 | 46 | 3.2 | 3.0 | 5.7 | 4.9 | 5.2 | 38 | 5.4 |

Bcl2 antagonist of cell death

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 0.432 | 0.221 | nd | nd | nd | nd |
| Average | 0.590 | 0.255 | nd | nd | nd | nd |
| Stdev | 0.478 | 0.204 | nd | nd | nd | nd |
| p (t-test) | | 0.10 | nd | nd | nd | nd |
| Min | 0.0873 | 0.0786 | nd | nd | nd | nd |
| Max | 2.49 | 0.651 | nd | nd | nd | nd |
| n (Samp) | 32 | 6 | nd | nd | nd | nd |
| n (Patient) | 20 | 6 | nd | nd | nd | nd |
| UO only | | | | | | |
| Median | 0.596 | 0.213 | nd | nd | nd | nd |
| Average | 0.643 | 0.243 | nd | nd | nd | nd |
| Stdev | 0.503 | 0.174 | nd | nd | nd | nd |
| p (t-test) | | 0.035 | nd | nd | nd | nd |
| Min | 0.0873 | 0.0786 | nd | nd | nd | nd |
| Max | 2.49 | 0.651 | nd | nd | nd | nd |
| n (Samp) | 27 | 8 | nd | nd | nd | nd |
| n (Patient) | 17 | 8 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.21 | nd | 0.19 | nd | nd | nd | nd | nd | nd |
| SE | 0.12 | nd | 0.098 | nd | nd | nd | nd | nd | nd |
| p | 0.012 | nd | 0.0013 | nd | nd | nd | nd | nd | nd |
| nCohort 1 | 32 | nd | 27 | nd | nd | nd | nd | nd | nd |
| nCohort 2 | 6 | nd | 8 | nd | nd | nd | nd | nd | nd |
| Cutoff 1 | 0.114 | nd | 0.181 | nd | nd | nd | nd | nd | nd |
| Sens 1 | 83% | nd | 75% | nd | nd | nd | nd | nd | nd |
| Spec 1 | 6% | nd | 15% | nd | nd | nd | nd | nd | nd |
| Cutoff 2 | 0.114 | nd | 0.114 | nd | nd | nd | nd | nd | nd |
| Sens 2 | 83% | nd | 88% | nd | nd | nd | nd | nd | nd |
| Spec 2 | 6% | nd | 7% | nd | nd | nd | nd | nd | nd |
| Cutoff 3 | 0 | nd | 0 | nd | nd | nd | nd | nd | nd |
| Sens 3 | 100% | nd | 100% | nd | nd | nd | nd | nd | nd |
| Spec 3 | 0% | nd | 0% | nd | nd | nd | nd | nd | nd |
| Cutoff 4 | 0.703 | nd | 0.771 | nd | nd | nd | nd | nd | nd |
| Sens 4 | 0% | nd | 0% | nd | nd | nd | nd | nd | nd |
| Spec 4 | 72% | nd | 70% | nd | nd | nd | nd | nd | nd |
| Cutoff 5 | 0.991 | nd | 1.01 | nd | nd | nd | nd | nd | nd |
| Sens 5 | 0% | nd | 0% | nd | nd | nd | nd | nd | nd |
| Spec 5 | 81% | nd | 81% | nd | nd | nd | nd | nd | nd |
| Cutoff 6 | 1.05 | nd | 1.13 | nd | nd | nd | nd | nd | nd |
| Sens 6 | 0% | nd | 0% | nd | nd | nd | nd | nd | nd |
| Spec 6 | 91% | nd | 93% | nd | nd | nd | nd | nd | nd |
| OR Quart 2 | >1.2 | nd | >1.1 | nd | nd | nd | nd | nd | nd |
| p Value | <0.88 | nd | <0.94 | nd | nd | nd | nd | nd | nd |
| 95% CI of | >0.067 | nd | >0.060 | nd | nd | nd | nd | nd | nd |
| OR Quart 2 | na | nd | na | nd | nd | nd | nd | nd | nd |
| OR Quart 3 | >2.5 | nd | >4.5 | nd | nd | nd | nd | nd | nd |
| p Value | <0.49 | nd | <0.24 | nd | nd | nd | nd | nd | nd |
| 95% CI of | >0.19 | nd | >0.37 | nd | nd | nd | nd | nd | nd |
| OR Quart 3 | na | nd | na | nd | nd | nd | nd | nd | nd |
| OR Quart 4 | >5.0 | nd | >9.0 | nd | nd | nd | nd | nd | nd |
| p Value | <0.20 | nd | <0.083 | nd | nd | nd | nd | nd | nd |
| 95% CI of | >0.42 | nd | >0.75 | nd | nd | nd | nd | nd | nd |
| OR Quart 4 | na | nd | na | nd | nd | nd | nd | nd | nd |

Caspase-9

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 25.5 | 14.8 | nd | nd | nd | nd |
| Average | 44.5 | 24.6 | nd | nd | nd | nd |
| Stdev | 60.6 | 24.9 | nd | nd | nd | nd |
| p (t-test) | | 0.27 | nd | nd | nd | nd |
| Min | 0.400 | 4.59 | nd | nd | nd | nd |
| Max | 366 | 78.6 | nd | nd | nd | nd |
| n (Samp) | 57 | 12 | nd | nd | nd | nd |
| n (Patient) | 37 | 12 | nd | nd | nd | nd |
| sCr only | | | | | | |
| Median | 22.8 | 13.2 | nd | nd | nd | nd |
| Average | 42.5 | 12.5 | nd | nd | nd | nd |
| Stdev | 58.2 | 7.24 | nd | nd | nd | nd |
| p (t-test) | | 0.21 | nd | nd | nd | nd |
| Min | 0.400 | 4.32 | nd | nd | nd | nd |
| Max | 366 | 21.7 | nd | nd | nd | nd |
| n (Samp) | 89 | 6 | nd | nd | nd | nd |
| n (Patient) | 61 | 6 | nd | nd | nd | nd |
| UO only | | | | | | |
| Median | 30.1 | 9.06 | nd | nd | nd | nd |
| Average | 50.3 | 20.6 | nd | nd | nd | nd |
| Stdev | 67.6 | 24.4 | nd | nd | nd | nd |
| p (t-test) | | 0.12 | nd | nd | nd | nd |
| Min | 6.05 | 3.79 | nd | nd | nd | nd |
| Max | 366 | 78.6 | nd | nd | nd | nd |
| n (Samp) | 42 | 14 | nd | nd | nd | nd |
| n (Patient) | 27 | 14 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.39 | 0.28 | 0.26 | nd | nd | nd | nd | nd | nd |
| SE | 0.094 | 0.12 | 0.083 | nd | nd | nd | nd | nd | nd |
| p | 0.25 | 0.077 | 0.0038 | nd | nd | nd | nd | nd | nd |
| nCohort 1 | 57 | 89 | 42 | nd | nd | nd | nd | nd | nd |
| nCohort 2 | 12 | 6 | 14 | nd | nd | nd | nd | nd | nd |
| Cutoff 1 | 8.91 | 3.65 | 6.05 | nd | nd | nd | nd | nd | nd |
| Sens 1 | 75% | 100% | 71% | nd | nd | nd | nd | nd | nd |
| Spec 1 | 21% | 7% | 2% | nd | nd | nd | nd | nd | nd |
| Cutoff 2 | 8.22 | 3.65 | 4.59 | nd | nd | nd | nd | nd | nd |
| Sens 2 | 83% | 100% | 86% | nd | nd | nd | nd | nd | nd |
| Spec 2 | 19% | 7% | 0% | nd | nd | nd | nd | nd | nd |
| Cutoff 3 | 4.59 | 3.65 | 3.79 | nd | nd | nd | nd | nd | nd |
| Sens 3 | 92% | 100% | 93% | nd | nd | nd | nd | nd | nd |
| Spec 3 | 9% | 7% | 0% | nd | nd | nd | nd | nd | nd |
| Cutoff 4 | 46.1 | 45.3 | 49.3 | nd | nd | nd | nd | nd | nd |
| Sens 4 | 17% | 0% | 14% | nd | nd | nd | nd | nd | nd |
| Spec 4 | 70% | 71% | 71% | nd | nd | nd | nd | nd | nd |
| Cutoff 5 | 54.5 | 57.5 | 54.5 | nd | nd | nd | nd | nd | nd |
| Sens 5 | 17% | 0% | 14% | nd | nd | nd | nd | nd | nd |
| Spec 5 | 81% | 81% | 81% | nd | nd | nd | nd | nd | nd |
| Cutoff 6 | 90.6 | 94.1 | 106 | nd | nd | nd | nd | nd | nd |
| Sens 6 | 0% | 0% | 0% | nd | nd | nd | nd | nd | nd |
| Spec 6 | 91% | 91% | 90% | nd | nd | nd | nd | nd | nd |
| OR Quart 2 | 1.1 | >1.0 | 1.0 | nd | nd | nd | nd | nd | nd |
| p Value | 0.95 | <0.98 | 1.0 | nd | nd | nd | nd | nd | nd |
| 95% CI of | 0.13 | >0.062 | 0.12 | nd | nd | nd | nd | nd | nd |
| OR Quart 2 | 8.6 | na | 8.3 | nd | nd | nd | nd | nd | nd |
| OR Quart 3 | 1.7 | >3.4 | 1.0 | nd | nd | nd | nd | nd | nd |
| p Value | 0.58 | <0.30 | 1.0 | nd | nd | nd | nd | nd | nd |
| 95% CI of | 0.25 | >0.33 | 0.12 | nd | nd | nd | nd | nd | nd |
| OR Quart 3 | 12 | na | 8.3 | nd | nd | nd | nd | nd | nd |
| OR Quart 4 | 3.3 | >2.3 | 8.0 | nd | nd | nd | nd | nd | nd |
| p Value | 0.19 | <0.51 | 0.026 | nd | nd | nd | nd | nd | nd |
| 95% CI of | 0.55 | >0.19 | 1.3 | nd | nd | nd | nd | nd | nd |
| OR Quart 4 | 20 | na | 50 | nd | nd | nd | nd | nd | nd |

Cadherin-1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 72700 | 88200 | 72700 | 92800 | 72700 | 98500 |
| Average | 102000 | 129000 | 102000 | 127000 | 102000 | 134000 |
| Stdev | 106000 | 88400 | 106000 | 90200 | 106000 | 91700 |
| p (t-test) | | 0.26 | | 0.28 | | 0.28 |
| Min | 14500 | 34900 | 14500 | 3320 | 14500 | 25800 |
| Max | 621000 | 334000 | 621000 | 340000 | 621000 | 283000 |
| n (Samp) | 52 | 28 | 52 | 31 | 52 | 16 |
| n (Patient) | 50 | 28 | 50 | 31 | 50 | 16 |
| sCr only | | | | | | |
| Median | 86800 | 130000 | 86800 | 73000 | 86800 | 109000 |
| Average | 116000 | 145000 | 116000 | 120000 | 116000 | 116000 |
| Stdev | 94600 | 90700 | 94600 | 94900 | 94600 | 73700 |
| p (t-test) | | 0.35 | | 0.87 | | 0.99 |
| Min | 3320 | 34900 | 3320 | 30700 | 3320 | 56100 |
| Max | 621000 | 334000 | 621000 | 340000 | 621000 | 283000 |
| n (Samp) | 123 | 10 | 123 | 16 | 123 | 8 |
| n (Patient) | 96 | 10 | 96 | 16 | 96 | 8 |
| UO only | | | | | | |
| Median | 81200 | 88200 | 81200 | 103000 | 81200 | 89400 |
| Average | 119000 | 122000 | 119000 | 122000 | 119000 | 139000 |
| Stdev | 114000 | 85000 | 114000 | 78600 | 114000 | 94300 |
| p (t-test) | | 0.89 | | 0.89 | | 0.59 |
| Min | 39700 | 38100 | 39700 | 3320 | 39700 | 25800 |
| Max | 621000 | 314000 | 621000 | 300000 | 621000 | 277000 |
| n (Samp) | 51 | 22 | 51 | 27 | 51 | 11 |
| n (Patient) | 44 | 22 | 44 | 27 | 44 | 11 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.63 | 0.54 | 0.59 | 0.47 | 0.57 | 0.62 | 0.56 | 0.59 |
| SE | 0.067 | 0.098 | 0.075 | 0.065 | 0.078 | 0.069 | 0.083 | 0.11 | 0.098 |
| p | 0.066 | 0.18 | 0.57 | 0.15 | 0.72 | 0.32 | 0.13 | 0.57 | 0.38 |
| nCohort 1 | 52 | 123 | 51 | 52 | 123 | 51 | 52 | 123 | 51 |
| nCohort 2 | 28 | 10 | 22 | 31 | 16 | 27 | 16 | 8 | 11 |
| Cutoff 1 | 78500 | 79100 | 75400 | 62800 | 40700 | 67700 | 61700 | 61700 | 79100 |
| Sens 1 | 71% | 70% | 73% | 71% | 75% | 70% | 75% | 75% | 73% |
| Spec 1 | 56% | 46% | 41% | 42% | 9% | 37% | 40% | 31% | 49% |
| Cutoff 2 | 52700 | 78500 | 52700 | 47600 | 39700 | 54300 | 57000 | 57000 | 70100 |
| Sens 2 | 82% | 80% | 82% | 81% | 81% | 81% | 81% | 88% | 82% |
| Spec 2 | 21% | 46% | 12% | 17% | 8% | 14% | 29% | 26% | 39% |
| Cutoff 3 | 40700 | 72100 | 47600 | 39700 | 31800 | 39700 | 48100 | 54700 | 47600 |
| Sens 3 | 93% | 90% | 91% | 90% | 94% | 93% | 94% | 100% | 91% |
| Spec 3 | 10% | 41% | 10% | 8% | 6% | 2% | 19% | 23% | 10% |
| Cutoff 4 | 97000 | 131000 | 100000 | 97000 | 131000 | 100000 | 97000 | 131000 | 100000 |
| Sens 4 | 46% | 50% | 32% | 48% | 38% | 52% | 50% | 25% | 45% |
| Spec 4 | 71% | 71% | 71% | 71% | 71% | 71% | 71% | 71% | 71% |
| Cutoff 5 | 114000 | 165000 | 138000 | 114000 | 165000 | 138000 | 114000 | 165000 | 138000 |
| Sens 5 | 36% | 40% | 27% | 42% | 38% | 33% | 38% | 12% | 36% |
| Spec 5 | 81% | 80% | 80% | 81% | 80% | 80% | 81% | 80% | 80% |
| Cutoff 6 | 153000 | 243000 | 242000 | 153000 | 243000 | 242000 | 153000 | 243000 | 242000 |
| Sens 6 | 32% | 20% | 18% | 39% | 6% | 11% | 31% | 12% | 27% |
| Spec 6 | 90% | 90% | 90% | 90% | 90% | 90% | 90% | 90% | 90% |
| OR Quart 2 | 0.78 | 3.2 | 0.74 | 0.47 | 0.14 | 0.54 | 1.0 | 2.0 | 0.93 |
| p Value | 0.72 | 0.33 | 0.70 | 0.27 | 0.079 | 0.41 | 1.0 | 0.58 | 0.94 |
| 95% CI of | 0.19 | 0.32 | 0.16 | 0.12 | 0.016 | 0.13 | 0.17 | 0.17 | 0.11 |
| OR Quart 2 | 3.1 | 32 | 3.4 | 1.8 | 1.3 | 2.3 | 5.8 | 23 | 7.6 |
| OR Quart 3 | 1.3 | 2.1 | 1.7 | 0.60 | 0.62 | 1.6 | 1.4 | 4.3 | 1.6 |
| p Value | 0.74 | 0.56 | 0.48 | 0.44 | 0.50 | 0.50 | 0.67 | 0.21 | 0.63 |
| 95% CI of | 0.33 | 0.18 | 0.41 | 0.16 | 0.16 | 0.42 | 0.27 | 0.45 | 0.23 |
| OR Quart 3 | 4.7 | 24 | 6.7 | 2.2 | 2.4 | 5.9 | 7.7 | 41 | 11 |
| OR Quart 4 | 2.3 | 4.3 | 1.2 | 2.0 | 0.83 | 1.8 | 2.5 | 0.97 | 2.2 |
| p Value | 0.20 | 0.21 | 0.80 | 0.27 | 0.78 | 0.39 | 0.25 | 0.98 | 0.42 |
| 95% CI of | 0.64 | 0.45 | 0.29 | 0.58 | 0.23 | 0.48 | 0.52 | 0.058 | 0.33 |
| OR Quart 4 | 8.5 | 40 | 4.9 | 6.9 | 3.0 | 6.6 | 13 | 16 | 14 |

Cadherin-5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 20.4 | 21.2 | nd | nd | nd | nd |
| Average | 20.5 | 23.9 | nd | nd | nd | nd |
| Stdev | 5.01 | 5.05 | nd | nd | nd | nd |
| p (t-test) | | 0.065 | nd | nd | nd | nd |
| Min | 12.4 | 18.0 | nd | nd | nd | nd |
| Max | 35.9 | 32.1 | nd | nd | nd | nd |
| n (Samp) | 48 | 9 | nd | nd | nd | nd |
| n (Patient) | 30 | 9 | nd | nd | nd | nd |
| UO only | | | | | | |
| Median | 20.5 | 21.2 | nd | nd | nd | nd |
| Average | 20.7 | 23.3 | nd | nd | nd | nd |
| Stdev | 4.09 | 5.02 | nd | nd | nd | nd |
| p (t-test) | | 0.080 | nd | nd | nd | nd |
| Min | 12.4 | 17.0 | nd | nd | nd | nd |
| Max | 29.3 | 32.1 | nd | nd | nd | nd |
| n (Samp) | 34 | 11 | nd | nd | nd | nd |
| n (Patient) | 20 | 11 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.69 | nd | 0.64 | nd | nd | nd | nd | nd | nd |
| SE | 0.10 | nd | 0.10 | nd | nd | nd | nd | nd | nd |
| p | 0.077 | nd | 0.16 | nd | nd | nd | nd | nd | nd |
| nCohort 1 | 48 | nd | 34 | nd | nd | nd | nd | nd | nd |
| nCohort 2 | 9 | nd | 11 | nd | nd | nd | nd | nd | nd |
| Cutoff 1 | 20.6 | nd | 19.5 | nd | nd | nd | nd | nd | nd |
| Sens 1 | 78% | nd | 73% | nd | nd | nd | nd | nd | nd |
| Spec 1 | 54% | nd | 44% | nd | nd | nd | nd | nd | nd |
| Cutoff 2 | 18.9 | nd | 18.9 | nd | nd | nd | nd | nd | nd |
| Sens 2 | 89% | nd | 82% | nd | nd | nd | nd | nd | nd |
| Spec 2 | 42% | nd | 38% | nd | nd | nd | nd | nd | nd |
| Cutoff 3 | 17.1 | nd | 17.1 | nd | nd | nd | nd | nd | nd |
| Sens 3 | 100% | nd | 91% | nd | nd | nd | nd | nd | nd |
| Spec 3 | 33% | nd | 26% | nd | nd | nd | nd | nd | nd |
| Cutoff 4 | 22.6 | nd | 22.7 | nd | nd | nd | nd | nd | nd |
| Sens 4 | 44% | nd | 45% | nd | nd | nd | nd | nd | nd |
| Spec 4 | 71% | nd | 71% | nd | nd | nd | nd | nd | nd |
| Cutoff 5 | 24.0 | nd | 24.0 | nd | nd | nd | nd | nd | nd |
| Sens 5 | 44% | nd | 45% | nd | nd | nd | nd | nd | nd |
| Spec 5 | 81% | nd | 82% | nd | nd | nd | nd | nd | nd |
| Cutoff 6 | 26.5 | nd | 26.2 | nd | nd | nd | nd | nd | nd |
| Sens 6 | 33% | nd | 27% | nd | nd | nd | nd | nd | nd |
| Spec 6 | 92% | nd | 91% | nd | nd | nd | nd | nd | nd |
| OR Quart 2 | >2.3 | nd | 3.8 | nd | nd | nd | nd | nd | nd |
| p Value | <0.51 | nd | 0.29 | nd | nd | nd | nd | nd | nd |
| 95% CI of | >0.19 | nd | 0.32 | nd | nd | nd | nd | nd | nd |
| OR Quart 2 | na | nd | 43 | nd | nd | nd | nd | nd | nd |
| OR Quart 3 | >3.8 | nd | 2.2 | nd | nd | nd | nd | nd | nd |
| p Value | <0.27 | nd | 0.54 | nd | nd | nd | nd | nd | nd |
| 95% CI of | >0.35 | nd | 0.17 | nd | nd | nd | nd | nd | nd |
| OR Quart 3 | na | nd | 29 | nd | nd | nd | nd | nd | nd |
| OR Quart 4 | >5.1 | nd | 7.1 | nd | nd | nd | nd | nd | nd |
| p Value | <0.17 | nd | 0.10 | nd | nd | nd | nd | nd | nd |
| 95% CI of | >0.50 | nd | 0.68 | nd | nd | nd | nd | nd | nd |
| OR Quart 4 | na | nd | 75 | nd | nd | nd | nd | nd | nd |

Cyclin-dependent kinase inhibitor 1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 208 | 138 | nd | nd | nd | nd |
| Average | 1230 | 1120 | nd | nd | nd | nd |
| Stdev | 2090 | 1790 | nd | nd | nd | nd |
| p (t-test) | | 0.87 | nd | nd | nd | nd |
| Min | 0.116 | 28.5 | nd | nd | nd | nd |
| Max | 6840 | 5430 | nd | nd | nd | nd |
| n (Samp) | 56 | 12 | nd | nd | nd | nd |
| n (Patient) | 37 | 12 | nd | nd | nd | nd |
| sCr only | | | | | | |
| Median | 197 | 171 | nd | nd | nd | nd |
| Average | 1140 | 463 | nd | nd | nd | nd |
| Stdev | 1940 | 716 | nd | nd | nd | nd |
| p (t-test) | | 0.40 | nd | nd | nd | nd |
| Min | 0.116 | 73.9 | nd | nd | nd | nd |
| Max | 6840 | 1910 | nd | nd | nd | nd |
| n (Samp) | 88 | 6 | nd | nd | nd | nd |
| n (Patient) | 61 | 6 | nd | nd | nd | nd |
| UO only | | | | | | |
| Median | 381 | 151 | nd | nd | nd | nd |
| Average | 1460 | 1310 | nd | nd | nd | nd |
| Stdev | 2310 | 2210 | nd | nd | nd | nd |
| p (t-test) | | 0.84 | nd | nd | nd | nd |
| Min | 0.116 | 28.5 | nd | nd | nd | nd |
| Max | 6840 | 6400 | nd | nd | nd | nd |
| n (Samp) | 41 | 14 | nd | nd | nd | nd |
| n (Patient) | 27 | 14 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.49 | 0.46 | 0.44 | nd | nd | nd | nd | nd | nd |
| SE | 0.093 | 0.12 | 0.091 | nd | nd | nd | nd | nd | nd |
| p | 0.91 | 0.78 | 0.52 | nd | nd | nd | nd | nd | nd |
| nCohort 1 | 56 | 88 | 41 | nd | nd | nd | nd | nd | nd |
| nCohort 2 | 12 | 6 | 14 | nd | nd | nd | nd | nd | nd |
| Cutoff 1 | 116 | 123 | 108 | nd | nd | nd | nd | nd | nd |
| Sens 1 | 75% | 83% | 71% | nd | nd | nd | nd | nd | nd |
| Spec 1 | 32% | 33% | 27% | nd | nd | nd | nd | nd | nd |
| Cutoff 2 | 96.8 | 123 | 73.1 | nd | nd | nd | nd | nd | nd |
| Sens 2 | 83% | 83% | 86% | nd | nd | nd | nd | nd | nd |
| Spec 2 | 30% | 33% | 17% | nd | nd | nd | nd | nd | nd |
| Cutoff 3 | 47.1 | 73.1 | 42.3 | nd | nd | nd | nd | nd | nd |
| Sens 3 | 92% | 100% | 93% | nd | nd | nd | nd | nd | nd |
| Spec 3 | 12% | 17% | 12% | nd | nd | nd | nd | nd | nd |
| Cutoff 4 | 715 | 715 | 902 | nd | nd | nd | nd | nd | nd |
| Sens 4 | 33% | 17% | 21% | nd | nd | nd | nd | nd | nd |
| Spec 4 | 71% | 70% | 71% | nd | nd | nd | nd | nd | nd |
| Cutoff 5 | 1430 | 1430 | 1760 | nd | nd | nd | nd | nd | nd |
| Sens 5 | 25% | 17% | 21% | nd | nd | nd | nd | nd | nd |
| Spec 5 | 80% | 82% | 80% | nd | nd | nd | nd | nd | nd |
| Cutoff 6 | 5560 | 5160 | 6560 | nd | nd | nd | nd | nd | nd |
| Sens 6 | 0% | 0% | 0% | nd | nd | nd | nd | nd | nd |
| Spec 6 | 91% | 91% | 90% | nd | nd | nd | nd | nd | nd |
| OR Quart 2 | 0.62 | 2.2 | 1.0 | nd | nd | nd | nd | nd | nd |
| p Value | 0.63 | 0.53 | 1.0 | nd | nd | nd | nd | nd | nd |
| 95% CI of | 0.090 | 0.18 | 0.16 | nd | nd | nd | nd | nd | nd |
| OR Quart 2 | 4.3 | 26 | 6.1 | nd | nd | nd | nd | nd | nd |
| OR Quart 3 | 1.9 | 2.1 | 2.0 | nd | nd | nd | nd | nd | nd |
| p Value | 0.42 | 0.56 | 0.41 | nd | nd | nd | nd | nd | nd |
| 95% CI of | 0.38 | 0.18 | 0.38 | nd | nd | nd | nd | nd | nd |
| OR Quart 3 | 9.9 | 25 | 11 | nd | nd | nd | nd | nd | nd |
| OR Quart 4 | 0.62 | 1.0 | 1.1 | nd | nd | nd | nd | nd | nd |
| p Value | 0.63 | 0.98 | 0.92 | nd | nd | nd | nd | nd | nd |
| 95% CI of | 0.090 | 0.062 | 0.18 | nd | nd | nd | nd | nd | nd |
| OR Quart 4 | 4.3 | 18 | 6.8 | nd | nd | nd | nd | nd | nd |

Carcinoembryonic antigen-related cell adhesion molecule 5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.66 | 2.13 | 1.66 | 2.36 | 1.66 | 2.26 |
| Average | 2.32 | 3.66 | 2.32 | 3.88 | 2.32 | 3.00 |
| Stdev | 2.32 | 4.90 | 2.32 | 5.35 | 2.32 | 2.72 |
| p (t-test) | | 0.0028 | | 6.3E−4 | | 0.17 |
| Min | 0.183 | 0.453 | 0.183 | 0.562 | 0.183 | 0.363 |
| Max | 20.8 | 30.1 | 20.8 | 33.6 | 20.8 | 12.5 |
| n (Samp) | 260 | 51 | 260 | 56 | 260 | 25 |
| n (Patient) | 110 | 51 | 110 | 56 | 110 | 25 |
| sCr only | | | | | | |
| Median | 1.77 | 1.68 | 1.77 | 2.00 | 1.77 | 1.91 |
| Average | 2.76 | 3.12 | 2.76 | 3.22 | 2.76 | 1.68 |
| Stdev | 3.35 | 4.09 | 3.35 | 3.30 | 3.35 | 0.833 |
| p (t-test) | | 0.66 | | 0.54 | | 0.25 |
| Min | 0.183 | 0.245 | 0.183 | 0.355 | 0.183 | 0.363 |
| Max | 33.6 | 17.1 | 33.6 | 14.5 | 33.6 | 2.69 |
| n (Samp) | 466 | 18 | 466 | 21 | 466 | 13 |
| n (Patient) | 180 | 18 | 180 | 21 | 180 | 13 |
| UO only | | | | | | |
| Median | 1.62 | 2.22 | 1.62 | 2.36 | 1.62 | 2.26 |
| Average | 2.47 | 3.81 | 2.47 | 3.82 | 2.47 | 3.39 |
| Stdev | 2.58 | 4.85 | 2.58 | 5.27 | 2.58 | 2.98 |
| p (t-test) | | 0.0067 | | 0.0083 | | 0.11 |
| Min | 0.183 | 0.622 | 0.183 | 0.562 | 0.183 | 0.591 |
| Max | 20.8 | 30.1 | 20.8 | 33.6 | 20.8 | 12.5 |
| n (Samp) | 213 | 51 | 213 | 53 | 213 | 23 |
| n (Patient) | 89 | 51 | 89 | 53 | 89 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | 0.48 | 0.63 | 0.63 | 0.54 | 0.62 | 0.60 | 0.43 | 0.62 |
| SE | 0.045 | 0.070 | 0.046 | 0.043 | 0.066 | 0.045 | 0.062 | 0.084 | 0.065 |
| p | 0.026 | 0.73 | 0.0052 | 0.0029 | 0.51 | 0.0056 | 0.12 | 0.44 | 0.073 |
| nCohort 1 | 260 | 466 | 213 | 260 | 466 | 213 | 260 | 466 | 213 |
| nCohort 2 | 51 | 18 | 51 | 56 | 21 | 53 | 25 | 13 | 23 |
| Cutoff 1 | 1.35 | 1.17 | 1.52 | 1.70 | 1.22 | 1.77 | 1.43 | 1.05 | 1.57 |
| Sens 1 | 71% | 72% | 71% | 71% | 71% | 72% | 72% | 77% | 74% |
| Spec 1 | 40% | 31% | 47% | 53% | 32% | 58% | 43% | 26% | 49% |
| Cutoff 2 | 1.10 | 0.591 | 1.11 | 1.02 | 1.01 | 1.02 | 0.855 | 0.971 | 0.855 |
| Sens 2 | 80% | 83% | 80% | 80% | 81% | 81% | 80% | 85% | 83% |
| Spec 2 | 32% | 8% | 32% | 30% | 25% | 30% | 23% | 23% | 22% |
| Cutoff 3 | 0.893 | 0.448 | 0.919 | 0.787 | 0.731 | 0.787 | 0.679 | 0.417 | 0.699 |
| Sens 3 | 90% | 94% | 90% | 91% | 90% | 91% | 92% | 92% | 91% |
| Spec 3 | 25% | 4% | 23% | 20% | 13% | 18% | 16% | 3% | 15% |
| Cutoff 4 | 2.42 | 2.71 | 2.52 | 2.42 | 2.71 | 2.52 | 2.42 | 2.71 | 2.52 |
| Sens 4 | 39% | 28% | 45% | 45% | 43% | 42% | 48% | 0% | 48% |
| Spec 4 | 70% | 70% | 71% | 70% | 70% | 71% | 70% | 70% | 71% |
| Cutoff 5 | 3.34 | 3.88 | 3.74 | 3.34 | 3.88 | 3.74 | 3.34 | 3.88 | 3.74 |
| Sens 5 | 31% | 22% | 31% | 32% | 29% | 28% | 32% | 0% | 30% |
| Spec 5 | 80% | 80% | 80% | 80% | 80% | 80% | 80% | 80% | 80% |
| Cutoff 6 | 4.93 | 5.28 | 5.88 | 4.93 | 5.28 | 5.88 | 4.93 | 5.28 | 5.88 |
| Sens 6 | 20% | 17% | 12% | 18% | 14% | 11% | 12% | 0% | 17% |
| Spec 6 | 90% | 90% | 90% | 90% | 90% | 90% | 90% | 90% | 90% |
| OR Quart 2 | 2.0 | 0.59 | 1.1 | 0.67 | 0.99 | 0.45 | 0.48 | >7.4 | 0.79 |
| p Value | 0.17 | 0.48 | 0.80 | 0.44 | 0.99 | 0.17 | 0.31 | <0.063 | 0.73 |
| 95% CI of | 0.75 | 0.14 | 0.41 | 0.24 | 0.28 | 0.15 | 0.11 | >0.90 | 0.20 |
| OR Quart 2 | 5.3 | 2.5 | 3.2 | 1.9 | 3.5 | 1.4 | 2.0 | na | 3.1 |
| OR Quart 3 | 2.2 | 1.0 | 2.5 | 2.3 | 0.58 | 2.4 | 1.2 | >3.1 | 1.0 |
| p Value | 0.11 | 1.0 | 0.050 | 0.046 | 0.47 | 0.041 | 0.77 | <0.33 | 1.0 |
| 95% CI of | 0.83 | 0.28 | 1.0 | 1.0 | 0.14 | 1.0 | 0.38 | >0.32 | 0.27 |
| OR Quart 3 | 5.8 | 3.5 | 6.3 | 5.4 | 2.5 | 5.7 | 3.7 | na | 3.7 |
| OR Quart 4 | 2.8 | 1.0 | 2.5 | 2.2 | 1.6 | 2.1 | 1.5 | >3.1 | 1.9 |
| p Value | 0.033 | 1.0 | 0.050 | 0.068 | 0.40 | 0.10 | 0.43 | <0.33 | 0.26 |
| 95% CI of | 1.1 | 0.28 | 1.0 | 0.94 | 0.52 | 0.87 | 0.52 | >0.32 | 0.61 |
| OR Quart 4 | 7.2 | 3.5 | 6.3 | 5.1 | 5.1 | 4.9 | 4.6 | na | 6.2 |

Myoglobin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 26.8 | 36.2 | 26.8 | 41.7 | 26.8 | 54.3 |
| Average | 66.0 | 106 | 66.0 | 168 | 66.0 | 113 |
| Stdev | 145 | 166 | 145 | 301 | 145 | 194 |
| p (t-test) | | 0.078 | | 1.8E−4 | | 0.13 |
| Min | 4.60 | 6.86 | 4.60 | 4.40 | 4.60 | 7.57 |
| Max | 1720 | 737 | 1720 | 1470 | 1720 | 988 |
| n (Samp) | 260 | 51 | 260 | 56 | 260 | 25 |
| n (Patient) | 110 | 51 | 110 | 56 | 110 | 25 |
| sCr only | | | | | | |
| Median | 33.0 | 62.6 | 33.0 | 64.7 | 33.0 | 69.7 |
| Average | 83.7 | 266 | 83.7 | 272 | 83.7 | 94.9 |
| Stdev | 191 | 465 | 191 | 433 | 191 | 93.0 |
| p (t-test) | | 2.8E−4 | | 5.2E−5 | | 0.83 |
| Min | 3.68 | 8.01 | 3.68 | 4.40 | 3.68 | 7.57 |
| Max | 2130 | 1880 | 2130 | 1470 | 2130 | 308 |
| n (Samp) | 466 | 18 | 466 | 21 | 466 | 13 |
| n (Patient) | 180 | 18 | 180 | 21 | 180 | 13 |
| UO only | | | | | | |
| Median | 27.4 | 37.3 | 27.4 | 41.0 | 27.4 | 62.1 |
| Average | 72.4 | 102 | 72.4 | 147 | 72.4 | 165 |
| Stdev | 153 | 158 | 153 | 260 | 153 | 320 |
| p (t-test) | | 0.22 | | 0.0071 | | 0.017 |
| Min | 4.60 | 6.86 | 4.60 | 8.81 | 4.60 | 8.40 |
| Max | 1720 | 737 | 1720 | 1410 | 1720 | 1310 |
| n (Samp) | 213 | 51 | 213 | 53 | 213 | 23 |
| n (Patient) | 89 | 51 | 89 | 53 | 89 | 23 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.60 | 0.65 | 0.59 | 0.65 | 0.61 | 0.64 | 0.63 | 0.62 | 0.63 |
| SE | 0.045 | 0.072 | 0.046 | 0.043 | 0.067 | 0.045 | 0.062 | 0.084 | 0.065 |
| p | 0.034 | 0.041 | 0.039 | 7.0E−4 | 0.086 | 0.0019 | 0.036 | 0.15 | 0.051 |
| nCohort 1 | 260 | 466 | 213 | 260 | 466 | 213 | 260 | 466 | 213 |
| nCohort 2 | 51 | 18 | 51 | 56 | 21 | 53 | 25 | 13 | 23 |
| Cutoff 1 | 21.3 | 26.6 | 20.9 | 27.5 | 24.5 | 27.5 | 20.6 | 25.6 | 20.4 |
| Sens 1 | 71% | 72% | 71% | 71% | 71% | 72% | 72% | 77% | 74% |
| Spec 1 | 40% | 43% | 40% | 52% | 38% | 50% | 39% | 41% | 40% |
| Cutoff 2 | 18.6 | 25.4 | 18.6 | 18.6 | 19.2 | 18.9 | 17.4 | 17.4 | 16.3 |
| Sens 2 | 80% | 83% | 80% | 80% | 81% | 81% | 80% | 85% | 83% |
| Spec 2 | 33% | 41% | 34% | 33% | 31% | 35% | 30% | 25% | 29% |
| Cutoff 3 | 12.5 | 8.56 | 14.8 | 12.3 | 12.9 | 13.2 | 9.64 | 8.39 | 10.6 |
| Sens 3 | 90% | 94% | 90% | 91% | 90% | 91% | 92% | 92% | 91% |
| Spec 3 | 18% | 8% | 26% | 18% | 17% | 21% | 13% | 8% | 15% |
| Cutoff 4 | 50.2 | 60.4 | 56.0 | 50.2 | 60.4 | 56.0 | 50.2 | 60.4 | 56.0 |
| Sens 4 | 43% | 50% | 41% | 46% | 52% | 45% | 52% | 54% | 52% |
| Spec 4 | 70% | 70% | 70% | 70% | 70% | 70% | 70% | 70% | 70% |
| Cutoff 5 | 74.3 | 93.4 | 84.8 | 74.3 | 93.4 | 84.8 | 74.3 | 93.4 | 84.8 |
| Sens 5 | 35% | 39% | 35% | 43% | 38% | 42% | 44% | 31% | 43% |
| Spec 5 | 80% | 80% | 80% | 80% | 80% | 80% | 80% | 80% | 80% |
| Cutoff 6 | 138 | 171 | 174 | 138 | 171 | 174 | 138 | 171 | 174 |
| Sens 6 | 22% | 33% | 16% | 29% | 29% | 26% | 20% | 15% | 13% |
| Spec 6 | 90% | 90% | 90% | 90% | 90% | 90% | 90% | 90% | 90% |
| OR Quart 2 | 1.6 | 2.6 | 2.9 | 0.88 | 0.99 | 2.0 | 1.0 | 0.99 | 0.74 |
| p Value | 0.36 | 0.27 | 0.038 | 0.80 | 0.99 | 0.16 | 1.0 | 0.99 | 0.70 |
| 95% CI of | 0.60 | 0.49 | 1.1 | 0.32 | 0.24 | 0.75 | 0.24 | 0.14 | 0.16 |
| OR Quart 2 | 4.1 | 13 | 8.1 | 2.4 | 4.1 | 5.5 | 4.2 | 7.2 | 3.4 |
| OR Quart 3 | 1.7 | 1.5 | 2.0 | 1.8 | 0.99 | 1.7 | 1.5 | 1.5 | 1.3 |
| p Value | 0.26 | 0.65 | 0.20 | 0.19 | 0.99 | 0.31 | 0.51 | 0.66 | 0.73 |
| 95% CI of | 0.67 | 0.25 | 0.69 | 0.75 | 0.24 | 0.61 | 0.42 | 0.25 | 0.32 |
| OR Quart 3 | 4.4 | 9.2 | 5.8 | 4.5 | 4.1 | 4.7 | 5.7 | 9.1 | 5.0 |
| OR Quart 4 | 2.6 | 4.2 | 4.0 | 3.4 | 2.3 | 4.1 | 3.0 | 3.1 | 3.2 |
| p Value | 0.039 | 0.073 | 0.0059 | 0.0045 | 0.17 | 0.0030 | 0.070 | 0.17 | 0.063 |
| 95% CI of | 1.1 | 0.88 | 1.5 | 1.5 | 0.70 | 1.6 | 0.91 | 0.61 | 0.94 |
| OR Quart 4 | 6.4 | 20 | 11 | 7.9 | 7.8 | 10 | 10.0 | 16 | 11 |

Mucin-16

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.11 | 1.51 | 1.11 | 2.02 | 1.11 | 1.59 |
| Average | 3.53 | 10.4 | 3.53 | 13.9 | 3.53 | 3.72 |
| Stdev | 14.4 | 41.9 | 14.4 | 46.3 | 14.4 | 6.27 |
| p (t-test) | | 0.19 | | 0.063 | | 0.95 |
| Min | 0.117 | 0.204 | 0.117 | 0.122 | 0.117 | 0.122 |
| Max | 131 | 253 | 131 | 248 | 131 | 31.2 |
| n (Samp) | 82 | 36 | 82 | 45 | 82 | 26 |
| n (Patient) | 75 | 36 | 75 | 45 | 75 | 26 |
| sCr only | | | | | | |
| Median | 1.41 | 1.27 | 1.41 | 1.83 | 1.41 | 1.24 |
| Average | 8.87 | 1.81 | 8.87 | 5.69 | 8.87 | 1.52 |
| Stdev | 35.3 | 1.57 | 35.3 | 9.55 | 35.3 | 1.06 |
| p (t-test) | | 0.49 | | 0.74 | | 0.53 |
| Min | 0.117 | 0.481 | 0.117 | 0.631 | 0.117 | 0.631 |
| Max | 263 | 5.56 | 263 | 37.0 | 263 | 4.16 |
| n (Samp) | 197 | 12 | 197 | 14 | 197 | 9 |
| n (Patient) | 131 | 12 | 131 | 14 | 131 | 9 |
| UO only | | | | | | |
| Median | 1.24 | 1.60 | 1.24 | 2.52 | 1.24 | 2.44 |
| Average | 3.82 | 12.7 | 3.82 | 14.8 | 3.82 | 4.48 |
| Stdev | 15.1 | 46.6 | 15.1 | 47.3 | 15.1 | 6.81 |
| p (t-test) | | 0.14 | | 0.067 | | 0.84 |
| Min | 0.122 | 0.204 | 0.122 | 0.122 | 0.122 | 0.122 |
| Max | 131 | 253 | 131 | 248 | 131 | 31.2 |
| n (Samp) | 75 | 29 | 75 | 43 | 75 | 21 |
| n (Patient) | 62 | 29 | 62 | 43 | 62 | 21 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.62 | 0.47 | 0.61 | 0.65 | 0.61 | 0.64 | 0.62 | 0.45 | 0.66 |
| SE | 0.058 | 0.087 | 0.064 | 0.052 | 0.082 | 0.054 | 0.066 | 0.10 | 0.071 |
| p | 0.036 | 0.72 | 0.084 | 0.0055 | 0.18 | 0.0073 | 0.068 | 0.64 | 0.024 |
| nCohort 1 | 82 | 197 | 75 | 82 | 197 | 75 | 82 | 197 | 75 |
| nCohort 2 | 36 | 12 | 29 | 45 | 14 | 43 | 26 | 9 | 21 |
| Cutoff 1 | 1.06 | 1.09 | 0.999 | 1.06 | 1.33 | 1.12 | 1.06 | 1.01 | 1.25 |
| Sens 1 | 72% | 75% | 72% | 71% | 71% | 72% | 73% | 78% | 71% |
| Spec 1 | 50% | 41% | 44% | 50% | 47% | 47% | 50% | 38% | 52% |
| Cutoff 2 | 0.803 | 0.821 | 0.761 | 0.821 | 1.02 | 0.803 | 0.999 | 0.754 | 1.06 |
| Sens 2 | 81% | 83% | 83% | 80% | 86% | 81% | 85% | 89% | 81% |
| Spec 2 | 46% | 36% | 33% | 46% | 38% | 43% | 48% | 29% | 47% |
| Cutoff 3 | 0.464 | 0.481 | 0.379 | 0.334 | 1.01 | 0.334 | 0.630 | 0.630 | 0.999 |
| Sens 3 | 92% | 92% | 93% | 91% | 93% | 91% | 92% | 100% | 90% |
| Spec 3 | 16% | 16% | 11% | 10% | 38% | 8% | 24% | 22% | 44% |
| Cutoff 4 | 2.09 | 3.28 | 1.96 | 2.09 | 3.28 | 1.96 | 2.09 | 3.28 | 1.96 |
| Sens 4 | 39% | 17% | 45% | 49% | 43% | 56% | 38% | 11% | 52% |
| Spec 4 | 72% | 70% | 71% | 72% | 70% | 71% | 72% | 70% | 71% |
| Cutoff 5 | 2.78 | 5.21 | 2.98 | 2.78 | 5.21 | 2.98 | 2.78 | 5.21 | 2.98 |
| Sens 5 | 36% | 8% | 41% | 44% | 29% | 47% | 27% | 0% | 38% |
| Spec 5 | 80% | 80% | 80% | 80% | 80% | 80% | 80% | 80% | 80% |
| Cutoff 6 | 4.88 | 8.80 | 5.21 | 4.88 | 8.80 | 5.21 | 4.88 | 8.80 | 5.21 |
| Sens 6 | 28% | 0% | 31% | 29% | 14% | 28% | 12% | 0% | 14% |
| Spec 6 | 90% | 90% | 91% | 90% | 90% | 91% | 90% | 90% | 91% |
| OR Quart 2 | 2.4 | 1.0 | 1.5 | 2.9 | 5.3 | 2.2 | 3.4 | 1.0 | 2.9 |
| p Value | 0.16 | 0.98 | 0.51 | 0.071 | 0.13 | 0.18 | 0.10 | 0.99 | 0.23 |
| 95% CI of | 0.70 | 0.14 | 0.42 | 0.91 | 0.60 | 0.69 | 0.78 | 0.062 | 0.50 |
| OR Quart 2 | 8.2 | 7.5 | 5.7 | 8.9 | 47 | 7.1 | 14 | 17 | 17 |
| OR Quart 3 | 1.8 | 3.3 | 1.0 | 1.2 | 3.1 | 1.7 | 2.3 | 6.7 | 3.7 |
| p Value | 0.35 | 0.15 | 1.0 | 0.80 | 0.34 | 0.37 | 0.28 | 0.085 | 0.14 |
| 95% CI of | 0.52 | 0.64 | 0.25 | 0.34 | 0.31 | 0.52 | 0.51 | 0.77 | 0.66 |
| OR Quart 3 | 6.5 | 17 | 4.0 | 4.0 | 30 | 5.7 | 10 | 57 | 20 |
| OR Quart 4 | 3.7 | 1.0 | 3.6 | 6.1 | 5.3 | 5.0 | 4.0 | 1.0 | 5.5 |
| p Value | 0.034 | 0.98 | 0.043 | 0.0018 | 0.13 | 0.0061 | 0.060 | 0.99 | 0.046 |
| 95% CI of | 1.1 | 0.14 | 1.0 | 2.0 | 0.60 | 1.6 | 0.95 | 0.062 | 1.0 |
| OR Quart 4 | 12 | 7.5 | 12 | 19 | 47 | 16 | 17 | 17 | 29 |

Tumor necrosis factor receptor superfamily member 10B

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.00E−9 | 1.00E−9 | 1.00E−9 | 0.00240 | 1.00E−9 | 1.00E−9 |
| Average | 0.00715 | 0.0102 | 0.00715 | 0.0133 | 0.00715 | 0.0150 |
| Stdev | 0.0203 | 0.0434 | 0.0203 | 0.0446 | 0.0203 | 0.0622 |
| p (t-test) | | 0.60 | | 0.30 | | 0.34 |
| Min | 1.00E−9 | 1.00E−9 | 1.00E−9 | 1.00E−9 | 1.00E−9 | 1.00E−9 |
| Max | 0.114 | 0.262 | 0.114 | 0.286 | 0.114 | 0.300 |
| n (Samp) | 79 | 36 | 79 | 41 | 79 | 23 |
| n (Patient) | 72 | 36 | 72 | 41 | 72 | 23 |
| sCr only | | | | | | |
| Median | 1.00E−9 | 1.00E−9 | 1.00E−9 | 0.00507 | 1.00E−9 | 1.00E−9 |
| Average | 0.00582 | 0.0263 | 0.00582 | 0.0316 | 0.00582 | 0.0334 |
| Stdev | 0.0146 | 0.0783 | 0.0146 | 0.0849 | 0.0146 | 0.0938 |
| p (t-test) | | 0.0040 | | 6.0E−4 | | 7.1E−4 |
| Min | 1.00E−9 | 1.00E−9 | 1.00E−9 | 1.00E−9 | 1.00E−9 | 1.00E−9 |
| Max | 0.114 | 0.262 | 0.114 | 0.286 | 0.114 | 0.300 |
| n (Samp) | 187 | 11 | 187 | 11 | 187 | 10 |
| n (Patient) | 127 | 11 | 127 | 11 | 127 | 10 |
| UO only | | | | | | |
| Median | 0.00131 | 1.00E−9 | 0.00131 | 0.00202 | 0.00131 | 1.00E−9 |
| Average | 0.00817 | 0.00387 | 0.00817 | 0.0104 | 0.00817 | 0.00218 |
| Stdev | 0.0213 | 0.00693 | 0.0213 | 0.0213 | 0.0213 | 0.00328 |
| p (t-test) | | 0.28 | | 0.60 | | 0.25 |
| Min | 1.00E−9 | 1.00E−9 | 1.00E−9 | 1.00E−9 | 1.00E−9 | 1.00E−9 |
| Max | 0.114 | 0.0300 | 0.114 | 0.108 | 0.114 | 0.00903 |
| n (Samp) | 70 | 30 | 70 | 40 | 70 | 17 |
| n (Patient) | 56 | 30 | 56 | 40 | 56 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.47 | 0.44 | 0.45 | 0.61 | 0.62 | 0.57 | 0.47 | 0.51 | 0.43 |
| SE | 0.059 | 0.092 | 0.064 | 0.055 | 0.093 | 0.058 | 0.069 | 0.094 | 0.080 |
| p | 0.65 | 0.52 | 0.43 | 0.038 | 0.21 | 0.22 | 0.72 | 0.91 | 0.35 |
| nCohort 1 | 79 | 187 | 70 | 79 | 187 | 70 | 79 | 187 | 70 |
| nCohort 2 | 36 | 11 | 30 | 41 | 11 | 40 | 23 | 10 | 17 |
| Cutoff 1 | 0 | 0 | 0 | 0 | 0 | 0 | 0 | 0 | 0 |
| Sens 1 | 100% | 100% | 100% | 100% | 100% | 100% | 100% | 100% | 100% |
| Spec 1 | 0% | 0% | 0% | 0% | 0% | 0% | 0% | 0% | 0% |
| Cutoff 2 | 0 | 0 | 0 | 0 | 0 | 0 | 0 | 0 | 0 |
| Sens 2 | 100% | 100% | 100% | 100% | 100% | 100% | 100% | 100% | 100% |
| Spec 2 | 0% | 0% | 0% | 0% | 0% | 0% | 0% | 0% | 0% |
| Cutoff 3 | 0 | 0 | 0 | 0 | 0 | 0 | 0 | 0 | 0 |
| Sens 3 | 100% | 100% | 100% | 100% | 100% | 100% | 100% | 100% | 100% |
| Spec 3 | 0% | 0% | 0% | 0% | 0% | 0% | 0% | 0% | 0% |
| Cutoff 4 | 0.00274 | 0.00377 | 0.00377 | 0.00274 | 0.00377 | 0.00377 | 0.00274 | 0.00377 | 0.00377 |
| Sens 4 | 33% | 27% | 33% | 49% | 64% | 42% | 30% | 40% | 24% |
| Spec 4 | 71% | 71% | 71% | 71% | 71% | 71% | 71% | 71% | 71% |
| Cutoff 5 | 0.00507 | 0.00819 | 0.00813 | 0.00507 | 0.00819 | 0.00813 | 0.00507 | 0.00819 | 0.00813 |
| Sens 5 | 28% | 27% | 17% | 37% | 27% | 35% | 26% | 30% | 12% |
| Spec 5 | 81% | 82% | 80% | 81% | 82% | 80% | 81% | 82% | 80% |
| Cutoff 6 | 0.0166 | 0.0136 | 0.0165 | 0.0166 | 0.0136 | 0.0165 | 0.0166 | 0.0136 | 0.0165 |
| Sens 6 | 6% | 18% | 7% | 17% | 18% | 18% | 4% | 20% | 0% |
| Spec 6 | 91% | 90% | 90% | 91% | 90% | 90% | 91% | 90% | 90% |
| OR Quart 2 | 0.12 | 0 | 0.24 | 6.0 | 0 | 1.1 | 1.1 | 0.48 | 0.71 |
| p Value | 0.011 | na | 0.057 | 0.012 | na | 0.84 | 0.94 | 0.41 | 0.68 |
| 95% CI of | 0.024 | na | 0.056 | 1.5 | na | 0.36 | 0.29 | 0.084 | 0.14 |
| OR Quart 2 | 0.61 | na | 1.0 | 24 | na | 3.5 | 3.8 | 2.7 | 3.6 |
| OR Quart 3 | 2.0 | 1.4 | 1.2 | 5.2 | 1.0 | 1.2 | 2.4 | 0 | 1.7 |
| p Value | 0.19 | 0.70 | 0.77 | 0.021 | 1.0 | 0.77 | 0.14 | na | 0.47 |
| 95% CI of | 0.71 | 0.29 | 0.38 | 1.3 | 0.24 | 0.38 | 0.74 | na | 0.40 |
| OR Quart 3 | 5.7 | 6.4 | 3.7 | 21 | 4.2 | 3.8 | 8.1 | na | 7.1 |
| OR Quart 4 | 0.55 | 1.4 | 0.84 | 9.0 | 0.72 | 2.4 | 0 | 0.98 | 1.1 |
| p Value | 0.30 | 0.68 | 0.77 | 0.0020 | 0.68 | 0.13 | na | 0.98 | 0.94 |
| 95% CI of | 0.17 | 0.30 | 0.26 | 2.2 | 0.15 | 0.78 | na | 0.23 | 0.23 |
| OR Quart 4 | 1.7 | 6.6 | 2.7 | 36 | 3.4 | 7.2 | na | 4.2 | 4.9 |

TABLE 6
Comparison of marker levels in EDTA samples collected from Cohort 1
(patients that did not progress beyond RIFLE stage 0 or R) and in EDTA samples collected from
subjects at 0, 24 hours, and 48 hours prior to reaching stage I or F in Cohort 2.
Apolipoprotein A-II

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 54100 | 49700 | 54100 | 45500 | 54100 | 43300 |
| Average | 55400 | 59300 | 55400 | 51900 | 55400 | 46600 |
| Stdev | 29200 | 19900 | 29200 | 32000 | 29200 | 33800 |
| p(t-test) | | 0.57 | | 0.57 | | 0.26 |
| Min | 5450 | 32800 | 5450 | 10700 | 5450 | 1810 |
| Max | 253000 | 97000 | 253000 | 176000 | 253000 | 152000 |
| n (Samp) | 230 | 19 | 230 | 26 | 230 | 15 |
| n (Patient) | 158 | 19 | 158 | 26 | 158 | 15 |
| UO only | | | | | | |
| Median | 52600 | 51800 | 52600 | 45500 | 52600 | 43200 |
| Average | 54900 | 58300 | 54900 | 51000 | 54900 | 47000 |
| Stdev | 29800 | 21200 | 29800 | 31800 | 29800 | 35000 |
| p (t-test) | | 0.62 | | 0.54 | | 0.35 |
| Min | 7680 | 23300 | 7680 | 10700 | 7680 | 1810 |
| Max | 253000 | 97000 | 253000 | 176000 | 253000 | 152000 |
| n (Samp) | 201 | 19 | 201 | 26 | 201 | 14 |
| n (Patient) | 133 | 19 | 133 | 26 | 133 | 14 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.56 | nd | 0.56 | 0.44 | nd | 0.43 | 0.36 | nd | 0.37 |
| SE | 0.071 | nd | 0.071 | 0.061 | nd | 0.062 | 0.079 | nd | 0.082 |
| p | 0.38 | nd | 0.40 | 0.30 | nd | 0.24 | 0.069 | nd | 0.11 |
| nCohort 1 | 230 | nd | 201 | 230 | nd | 201 | 230 | nd | 201 |
| nCohort 2 | 19 | nd | 19 | 26 | nd | 26 | 15 | nd | 14 |
| Cutoff 1 | 43600 | nd | 43600 | 35400 | nd | 35000 | 36000 | nd | 36000 |
| Sens 1 | 74% | nd | 74% | 73% | nd | 73% | 73% | nd | 71% |
| Spec 1 | 35% | nd | 36% | 21% | nd | 21% | 21% | nd | 21% |
| Cutoff 2 | 43000 | nd | 40500 | 30600 | nd | 30600 | 35900 | nd | 31700 |
| Sens 2 | 84% | nd | 84% | 81% | nd | 81% | 80% | nd | 86% |
| Spec 2 | 34% | nd | 30% | 17% | nd | 18% | 21% | nd | 19% |
| Cutoff 3 | 40200 | nd | 32500 | 28300 | nd | 26600 | 7970 | nd | 7970 |
| Sens 3 | 95% | nd | 95% | 92% | nd | 92% | 93% | nd | 93% |
| Spec 3 | 30% | nd | 20% | 16% | nd | 14% | 1% | nd | 1% |
| Cutoff 4 | 64400 | nd | 64800 | 64400 | nd | 64800 | 64400 | nd | 64800 |
| Sens 4 | 42% | nd | 42% | 19% | nd | 15% | 13% | nd | 14% |
| Spec 4 | 70% | nd | 70% | 70% | nd | 70% | 70% | nd | 70% |
| Cutoff 5 | 72600 | nd | 72300 | 72600 | nd | 72300 | 72600 | nd | 72300 |
| Sens 5 | 26% | nd | 26% | 12% | nd | 12% | 13% | nd | 14% |
| Spec 5 | 80% | nd | 80% | 80% | nd | 80% | 80% | nd | 80% |
| Cutoff 6 | 87100 | nd | 83600 | 87100 | nd | 83600 | 87100 | nd | 83600 |
| Sens 6 | 16% | nd | 16% | 8% | nd | 8% | 7% | nd | 7% |
| Spec 6 | 90% | nd | 90% | 90% | nd | 90% | 90% | nd | 90% |
| OR Quart 2 | 12 | nd | 5.2 | 1.2 | nd | 1.9 | 0 | nd | 0 |
| p Value | 0.021 | nd | 0.041 | 0.75 | nd | 0.35 | na | nd | na |
| 95% CI of | 1.5 | nd | 1.1 | 0.35 | nd | 0.51 | na | nd | na |
| OR Quart 2 | 95 | nd | 25 | 4.2 | nd | 6.7 | na | nd | na |
| OR Quart 3 | 1.0 | nd | 0.49 | 1.2 | nd | 1.6 | 3.9 | nd | 3.9 |
| p Value | 1.0 | nd | 0.57 | 0.75 | nd | 0.51 | 0.099 | nd | 0.10 |
| 95% CI of | 0.061 | nd | 0.043 | 0.35 | nd | 0.42 | 0.77 | nd | 0.77 |
| OR Quart 3 | 16 | nd | 5.6 | 4.2 | nd | 5.8 | 20 | nd | 20 |
| OR Quart 4 | 7.6 | nd | 3.9 | 1.9 | nd | 2.5 | 3.3 | nd | 2.7 |
| p Value | 0.061 | nd | 0.10 | 0.26 | nd | 0.14 | 0.16 | nd | 0.25 |
| 95% CI of | 0.91 | nd | 0.77 | 0.61 | nd | 0.73 | 0.63 | nd | 0.50 |
| OR Quart 4 | 64 | nd | 20 | 6.1 | nd | 8.8 | 17 | nd | 15 |

Caspase-1

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 70.5 | 58.2 | nd | nd | nd | nd |
| Average | 90.5 | 69.4 | nd | nd | nd | nd |
| Stdev | 59.1 | 45.7 | nd | nd | nd | nd |
| p (t-test) | | 0.31 | nd | nd | nd | nd |
| Min | 20.6 | 36.3 | nd | nd | nd | nd |
| Max | 326 | 188 | nd | nd | nd | nd |
| n (Samp) | 60 | 9 | nd | nd | nd | nd |
| n (Patient) | 37 | 9 | nd | nd | nd | nd |
| UO only | | | | | | |
| Median | 72.3 | 58.2 | nd | nd | nd | nd |
| Average | 91.4 | 69.4 | nd | nd | nd | nd |
| Stdev | 54.9 | 45.7 | nd | nd | nd | nd |
| p (t-test) | | 0.27 | nd | nd | nd | nd |
| Min | 33.8 | 36.3 | nd | nd | nd | nd |
| Max | 271 | 188 | nd | nd | nd | nd |
| n (Samp) | 45 | 9 | nd | nd | nd | nd |
| n (Patient) | 26 | 9 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.32 | nd | 0.31 | nd | nd | nd | nd | nd | nd |
| SE | 0.10 | nd | 0.10 | nd | nd | nd | nd | nd | nd |
| p | 0.089 | nd | 0.066 | nd | nd | nd | nd | nd | nd |
| nCohort 1 | 60 | nd | 45 | nd | nd | nd | nd | nd | nd |
| nCohort 2 | 9 | nd | 9 | nd | nd | nd | nd | nd | nd |
| Cutoff 1 | 45.8 | nd | 45.8 | nd | nd | nd | nd | nd | nd |
| Sens 1 | 78% | nd | 78% | nd | nd | nd | nd | nd | nd |
| Spec 1 | 15% | nd | 11% | nd | nd | nd | nd | nd | nd |
| Cutoff 2 | 42.1 | nd | 42.0 | nd | nd | nd | nd | nd | nd |
| Sens 2 | 89% | nd | 89% | nd | nd | nd | nd | nd | nd |
| Spec 2 | 13% | nd | 9% | nd | nd | nd | nd | nd | nd |
| Cutoff 3 | 35.1 | nd | 33.8 | nd | nd | nd | nd | nd | nd |
| Sens 3 | 100% | nd | 100% | nd | nd | nd | nd | nd | nd |
| Spec 3 | 5% | nd | 2% | nd | nd | nd | nd | nd | nd |
| Cutoff 4 | 95.6 | nd | 95.9 | nd | nd | nd | nd | nd | nd |
| Sens 4 | 11% | nd | 11% | nd | nd | nd | nd | nd | nd |
| Spec 4 | 70% | nd | 73% | nd | nd | nd | nd | nd | nd |
| Cutoff 5 | 110 | nd | 103 | nd | nd | nd | nd | nd | nd |
| Sens 5 | 11% | nd | 11% | nd | nd | nd | nd | nd | nd |
| Spec 5 | 80% | nd | 80% | nd | nd | nd | nd | nd | nd |
| Cutoff 6 | 147 | nd | 186 | nd | nd | nd | nd | nd | nd |
| Sens 6 | 11% | nd | 11% | nd | nd | nd | nd | nd | nd |
| Spec 6 | 90% | nd | 91% | nd | nd | nd | nd | nd | nd |
| OR Quart 2 | 1.1 | nd | 1.1 | nd | nd | nd | nd | nd | nd |
| p Value | 0.97 | nd | 0.96 | nd | nd | nd | nd | nd | nd |
| 95% CI of | 0.061 | nd | 0.061 | nd | nd | nd | nd | nd | nd |
| OR Quart 2 | 18 | nd | 19 | nd | nd | nd | nd | nd | nd |
| OR Quart 3 | 5.2 | nd | 5.2 | nd | nd | nd | nd | nd | nd |
| p Value | 0.16 | nd | 0.17 | nd | nd | nd | nd | nd | nd |
| 95% CI of | 0.52 | nd | 0.50 | nd | nd | nd | nd | nd | nd |
| OR Quart 3 | 53 | nd | 54 | nd | nd | nd | nd | nd | nd |
| OR Quart 4 | 3.6 | nd | 3.9 | nd | nd | nd | nd | nd | nd |
| p Value | 0.29 | nd | 0.27 | nd | nd | nd | nd | nd | nd |
| 95% CI of | 0.34 | nd | 0.35 | nd | nd | nd | nd | nd | nd |
| OR Quart 4 | 39 | nd | 43 | nd | nd | nd | nd | nd | nd |

Caspase-9

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 21.3 | 7.75 | nd | nd | nd | nd |
| Average | 42.0 | 23.4 | nd | nd | nd | nd |
| Stdev | 59.4 | 35.2 | nd | nd | nd | nd |
| p (t-test) | | 0.36 | nd | nd | nd | nd |
| Min | 0.400 | 3.79 | nd | nd | nd | nd |
| Max | 366 | 114 | nd | nd | nd | nd |
| n (Samp) | 82 | 9 | nd | nd | nd | nd |
| n (Patient) | 59 | 9 | nd | nd | nd | nd |
| UO only | | | | | | |
| Median | 23.9 | 7.75 | nd | nd | nd | nd |
| Average | 46.6 | 23.4 | nd | nd | nd | nd |
| Stdev | 65.1 | 35.2 | nd | nd | nd | nd |
| p (t-test) | | 0.30 | nd | nd | nd | nd |
| Min | 0.400 | 3.79 | nd | nd | nd | nd |
| Max | 366 | 114 | nd | nd | nd | nd |
| n (Samp) | 64 | 9 | nd | nd | nd | nd |
| n (Patient) | 46 | 9 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.32 | nd | 0.28 | nd | nd | nd | nd | nd | nd |
| SE | 0.10 | nd | 0.10 | nd | nd | nd | nd | nd | nd |
| p | 0.085 | nd | 0.032 | nd | nd | nd | nd | nd | nd |
| nCohort 1 | 82 | nd | 64 | nd | nd | nd | nd | nd | nd |
| nCohort 2 | 9 | nd | 9 | nd | nd | nd | nd | nd | nd |
| Cutoff 1 | 4.69 | nd | 4.69 | nd | nd | nd | nd | nd | nd |
| Sens 1 | 78% | nd | 78% | nd | nd | nd | nd | nd | nd |
| Spec 1 | 9% | nd | 3% | nd | nd | nd | nd | nd | nd |
| Cutoff 2 | 4.59 | nd | 4.59 | nd | nd | nd | nd | nd | nd |
| Sens 2 | 89% | nd | 89% | nd | nd | nd | nd | nd | nd |
| Spec 2 | 9% | nd | 3% | nd | nd | nd | nd | nd | nd |
| Cutoff 3 | 3.65 | nd | 0.400 | nd | nd | nd | nd | nd | nd |
| Sens 3 | 100% | nd | 100% | nd | nd | nd | nd | nd | nd |
| Spec 3 | 6% | nd | 2% | nd | nd | nd | nd | nd | nd |
| Cutoff 4 | 43.6 | nd | 45.3 | nd | nd | nd | nd | nd | nd |
| Sens 4 | 11% | nd | 11% | nd | nd | nd | nd | nd | nd |
| Spec 4 | 71% | nd | 70% | nd | nd | nd | nd | nd | nd |
| Cutoff 5 | 52.2 | nd | 54.5 | nd | nd | nd | nd | nd | nd |
| Sens 5 | 11% | nd | 11% | nd | nd | nd | nd | nd | nd |
| Spec 5 | 80% | nd | 81% | nd | nd | nd | nd | nd | nd |
| Cutoff 6 | 87.8 | nd | 106 | nd | nd | nd | nd | nd | nd |
| Sens 6 | 11% | nd | 11% | nd | nd | nd | nd | nd | nd |
| Spec 6 | 90% | nd | 91% | nd | nd | nd | nd | nd | nd |
| OR Quart 2 | 2.1 | nd | 2.2 | nd | nd | nd | nd | nd | nd |
| p Value | 0.56 | nd | 0.52 | nd | nd | nd | nd | nd | nd |
| 95% CI of | 0.18 | nd | 0.19 | nd | nd | nd | nd | nd | nd |
| OR Quart 2 | 25 | nd | 27 | nd | nd | nd | nd | nd | nd |
| OR Quart 3 | 1.0 | nd | 1.1 | nd | nd | nd | nd | nd | nd |
| p Value | 1.0 | nd | 0.97 | nd | nd | nd | nd | nd | nd |
| 95% CI of | 0.059 | nd | 0.061 | nd | nd | nd | nd | nd | nd |
| OR Quart 3 | 17 | nd | 18 | nd | nd | nd | nd | nd | nd |
| OR Quart 4 | 6.5 | nd | 6.9 | nd | nd | nd | nd | nd | nd |
| p Value | 0.10 | nd | 0.094 | nd | nd | nd | nd | nd | nd |
| 95% CI of | 0.69 | nd | 0.72 | nd | nd | nd | nd | nd | nd |
| OR Quart 4 | 61 | nd | 67 | nd | nd | nd | nd | nd | nd |

Cadherin-1

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 77700 | 123000 | 77700 | 103000 | 77700 | 117000 |
| Average | 113000 | 130000 | 113000 | 122000 | 113000 | 146000 |
| Stdev | 97300 | 57100 | 97300 | 77800 | 97300 | 81700 |
| p (t-test) | | 0.64 | | 0.70 | | 0.26 |
| Min | 14500 | 51600 | 14500 | 3320 | 14500 | 50500 |
| Max | 621000 | 231000 | 621000 | 340000 | 621000 | 285000 |
| n (Samp) | 123 | 7 | 123 | 20 | 123 | 12 |
| n (Patient) | 97 | 7 | 97 | 20 | 97 | 12 |
| UO only | | | | | | |
| Median | nd | nd | 86600 | 95800 | 86600 | 105000 |
| Average | nd | nd | 123000 | 105000 | 123000 | 128000 |
| Stdev | nd | nd | 101000 | 52100 | 101000 | 80100 |
| p (t-test) | nd | nd | | 0.44 | | 0.88 |
| Min | nd | nd | 25800 | 3320 | 25800 | 50500 |
| Max | nd | nd | 621000 | 209000 | 621000 | 277000 |
| n (Samp) | nd | nd | 104 | 20 | 104 | 10 |
| n (Patient) | nd | nd | 81 | 20 | 81 | 10 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.67 | nd | nd | 0.60 | nd | 0.54 | 0.69 | nd | 0.57 |
| SE | 0.12 | nd | nd | 0.071 | nd | 0.072 | 0.088 | nd | 0.098 |
| p | 0.14 | nd | nd | 0.16 | nd | 0.54 | 0.031 | nd | 0.47 |
| nCohort 1 | 123 | nd | nd | 123 | nd | 104 | 123 | nd | 104 |
| nCohort 2 | 7 | nd | nd | 20 | nd | 20 | 12 | nd | 10 |
| Cutoff 1 | 108000 | nd | nd | 81200 | nd | 85700 | 100000 | nd | 79100 |
| Sens 1 | 71% | nd | nd | 70% | nd | 70% | 75% | nd | 70% |
| Spec 1 | 70% | nd | nd | 53% | nd | 50% | 67% | nd | 45% |
| Cutoff 2 | 91400 | nd | nd | 69300 | nd | 70100 | 79100 | nd | 75400 |
| Sens 2 | 86% | nd | nd | 80% | nd | 80% | 83% | nd | 80% |
| Spec 2 | 63% | nd | nd | 45% | nd | 38% | 52% | nd | 40% |
| Cutoff 3 | 51000 | nd | nd | 54100 | nd | 63600 | 75400 | nd | 54100 |
| Sens 3 | 100% | nd | nd | 90% | nd | 90% | 92% | nd | 90% |
| Spec 3 | 20% | nd | nd | 24% | nd | 33% | 48% | nd | 18% |
| Cutoff 4 | 110000 | nd | nd | 110000 | nd | 114000 | 110000 | nd | 114000 |
| Sens 4 | 57% | nd | nd | 40% | nd | 30% | 50% | nd | 40% |
| Spec 4 | 71% | nd | nd | 71% | nd | 70% | 71% | nd | 70% |
| Cutoff 5 | 165000 | nd | nd | 165000 | nd | 184000 | 165000 | nd | 184000 |
| Sens 5 | 29% | nd | nd | 25% | nd | 10% | 25% | nd | 20% |
| Spec 5 | 80% | nd | nd | 80% | nd | 81% | 80% | nd | 81% |
| Cutoff 6 | 248000 | nd | nd | 248000 | nd | 257000 | 248000 | nd | 257000 |
| Sens 6 | 0% | nd | nd | 5% | nd | 0% | 25% | nd | 20% |
| Spec 6 | 90% | nd | nd | 90% | nd | 90% | 90% | nd | 90% |
| OR Quart 2 | 0 | nd | nd | 0.97 | nd | 3.5 | 2.0 | nd | 0.96 |
| p Value | na | nd | nd | 0.97 | nd | 0.15 | 0.58 | nd | 0.97 |
| 95% CI of | na | nd | nd | 0.18 | nd | 0.64 | 0.17 | nd | 0.13 |
| OR Quart 2 | na | nd | nd | 5.2 | nd | 19 | 23 | nd | 7.4 |
| OR Quart 3 | 4.4 | nd | nd | 3.0 | nd | 5.0 | 6.9 | nd | 2.2 |
| p Value | 0.19 | nd | nd | 0.12 | nd | 0.054 | 0.083 | nd | 0.40 |
| 95% CI of | 0.47 | nd | nd | 0.74 | nd | 0.98 | 0.78 | nd | 0.36 |
| OR Quart 3 | 42 | nd | nd | 13 | nd | 26 | 60 | nd | 13 |
| OR Quart 4 | 2.0 | nd | nd | 2.1 | nd | 2.1 | 3.1 | nd | 0.96 |
| p Value | 0.58 | nd | nd | 0.31 | nd | 0.40 | 0.34 | nd | 0.97 |
| 95% CI of | 0.17 | nd | nd | 0.49 | nd | 0.36 | 0.31 | nd | 0.13 |
| OR Quart 4 | 23 | nd | nd | 9.3 | nd | 13 | 31 | nd | 7.4 |

Cyclin-dependent kinase inhibitor 1

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 178 | 415 | nd | nd | nd | nd |
| Average | 989 | 2040 | nd | nd | nd | nd |
| Stdev | 1850 | 2760 | nd | nd | nd | nd |
| p (t-test) | | 0.13 | nd | nd | nd | nd |
| Min | 0.116 | 55.2 | nd | nd | nd | nd |
| Max | 6840 | 6400 | nd | nd | nd | nd |
| n (Samp) | 80 | 9 | nd | nd | nd | nd |
| n (Patient) | 58 | 9 | nd | nd | nd | nd |
| UO only | | | | | | |
| Median | 182 | 415 | nd | nd | nd | nd |
| Average | 1090 | 2040 | nd | nd | nd | nd |
| Stdev | 2000 | 2760 | nd | nd | nd | nd |
| p (t-test) | | 0.21 | nd | nd | nd | nd |
| Min | 0.116 | 55.2 | nd | nd | nd | nd |
| Max | 6840 | 6400 | nd | nd | nd | nd |
| n (Samp) | 62 | 9 | nd | nd | nd | nd |
| n (Patient) | 45 | 9 | nd | nd | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.58 | nd | 0.56 | nd | nd | nd | nd | nd | nd |
| SE | 0.10 | nd | 0.11 | nd | nd | nd | nd | nd | nd |
| p | 0.44 | nd | 0.55 | nd | nd | nd | nd | nd | nd |
| nCohort 1 | 80 | nd | 62 | nd | nd | nd | nd | nd | nd |
| nCohort 2 | 9 | nd | 9 | nd | nd | nd | nd | nd | nd |
| Cutoff 1 | 82.7 | nd | 82.7 | nd | nd | nd | nd | nd | nd |
| Sens 1 | 78% | nd | 78% | nd | nd | nd | nd | nd | nd |
| Spec 1 | 20% | nd | 19% | nd | nd | nd | nd | nd | nd |
| Cutoff 2 | 61.7 | nd | 55.2 | nd | nd | nd | nd | nd | nd |
| Sens 2 | 89% | nd | 89% | nd | nd | nd | nd | nd | nd |
| Spec 2 | 18% | nd | 16% | nd | nd | nd | nd | nd | nd |
| Cutoff 3 | 53.6 | nd | 52.0 | nd | nd | nd | nd | nd | nd |
| Sens 3 | 100% | nd | 100% | nd | nd | nd | nd | nd | nd |
| Spec 3 | 15% | nd | 15% | nd | nd | nd | nd | nd | nd |
| Cutoff 4 | 429 | nd | 470 | nd | nd | nd | nd | nd | nd |
| Sens 4 | 44% | nd | 44% | nd | nd | nd | nd | nd | nd |
| Spec 4 | 70% | nd | 71% | nd | nd | nd | nd | nd | nd |
| Cutoff 5 | 1300 | nd | 1410 | nd | nd | nd | nd | nd | nd |
| Sens 5 | 33% | nd | 33% | nd | nd | nd | nd | nd | nd |
| Spec 5 | 80% | nd | 81% | nd | nd | nd | nd | nd | nd |
| Cutoff 6 | 3790 | nd | 5160 | nd | nd | nd | nd | nd | nd |
| Sens 6 | 33% | nd | 22% | nd | nd | nd | nd | nd | nd |
| Spec 6 | 90% | nd | 90% | nd | nd | nd | nd | nd | nd |
| OR Quart 2 | 0.30 | nd | 0.27 | nd | nd | nd | nd | nd | nd |
| p Value | 0.32 | nd | 0.29 | nd | nd | nd | nd | nd | nd |
| 95% CI of | 0.029 | nd | 0.026 | nd | nd | nd | nd | nd | nd |
| OR Quart 2 | 3.2 | nd | 2.9 | nd | nd | nd | nd | nd | nd |
| OR Quart 3 | 0.30 | nd | 0.58 | nd | nd | nd | nd | nd | nd |
| p Value | 0.32 | nd | 0.58 | nd | nd | nd | nd | nd | nd |
| 95% CI of | 0.029 | nd | 0.085 | nd | nd | nd | nd | nd | nd |
| OR Quart 3 | 3.2 | nd | 4.0 | nd | nd | nd | nd | nd | nd |
| OR Quart 4 | 1.3 | nd | 0.93 | nd | nd | nd | nd | nd | nd |
| p Value | 0.73 | nd | 0.94 | nd | nd | nd | nd | nd | nd |
| 95% CI of | 0.26 | nd | 0.16 | nd | nd | nd | nd | nd | nd |
| OR Quart 4 | 6.8 | nd | 5.4 | nd | nd | nd | nd | nd | nd |

Carcinoembryonic antigen-related cell adhesion molecule 5

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.71 | 2.13 | 1.71 | 2.32 | 1.71 | 2.36 |
| Average | 2.73 | 2.72 | 2.73 | 2.84 | 2.73 | 2.41 |
| Stdev | 3.35 | 2.76 | 3.35 | 2.87 | 3.35 | 1.73 |
| p (t-test) | | 0.99 | | 0.86 | | 0.69 |
| Min | 0.183 | 0.324 | 0.183 | 0.245 | 0.183 | 0.355 |
| Max | 33.6 | 14.1 | 33.6 | 16.6 | 33.6 | 7.86 |
| n (Samp) | 434 | 27 | 434 | 34 | 434 | 17 |
| n (Patient) | 173 | 27 | 173 | 34 | 173 | 17 |
| sCr only | | | | | | |
| Median | 1.74 | 2.90 | 1.74 | 3.41 | 1.74 | 2.55 |
| Average | 2.76 | 4.92 | 2.76 | 2.99 | 2.76 | 3.08 |
| Stdev | 3.28 | 6.27 | 3.28 | 1.97 | 3.28 | 1.69 |
| p (t-test) | | 0.11 | | 0.84 | | 0.80 |
| Min | 0.183 | 0.324 | 0.183 | 0.245 | 0.183 | 0.355 |
| Max | 33.6 | 17.1 | 33.6 | 5.37 | 33.6 | 5.44 |
| n (Samp) | 535 | 6 | 535 | 9 | 535 | 7 |
| n (Patient) | 207 | 6 | 207 | 9 | 207 | 7 |
| UO only | | | | | | |
| Median | 1.77 | 2.36 | 1.77 | 2.32 | 1.77 | 1.93 |
| Average | 2.89 | 2.85 | 2.89 | 2.92 | 2.89 | 2.26 |
| Stdev | 3.55 | 2.71 | 3.55 | 2.91 | 3.55 | 1.79 |
| p (t-test) | | 0.95 | | 0.97 | | 0.47 |
| Min | 0.183 | 0.491 | 0.183 | 0.710 | 0.183 | 0.324 |
| Max | 33.6 | 14.1 | 33.6 | 16.6 | 33.6 | 7.86 |
| n (Samp) | 359 | 27 | 359 | 32 | 359 | 17 |
| n (Patient) | 139 | 27 | 139 | 32 | 139 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.53 | 0.57 | 0.55 | 0.55 | 0.58 | 0.55 | 0.55 | 0.65 | 0.48 |
| SE | 0.058 | 0.12 | 0.059 | 0.053 | 0.10 | 0.054 | 0.073 | 0.11 | 0.072 |
| p | 0.56 | 0.57 | 0.43 | 0.35 | 0.42 | 0.37 | 0.54 | 0.20 | 0.81 |
| nCohort 1 | 434 | 535 | 359 | 434 | 535 | 359 | 434 | 535 | 359 |
| nCohort 2 | 27 | 6 | 27 | 34 | 9 | 32 | 17 | 7 | 17 |
| Cutoff 1 | 1.26 | 0.825 | 1.49 | 1.23 | 1.22 | 1.27 | 1.68 | 2.44 | 1.62 |
| Sens 1 | 70% | 83% | 70% | 71% | 78% | 72% | 71% | 71% | 71% |
| Spec 1 | 35% | 16% | 41% | 34% | 32% | 34% | 48% | 66% | 45% |
| Cutoff 2 | 1.04 | 0.825 | 1.09 | 0.971 | 0.562 | 1.06 | 1.04 | 2.28 | 0.679 |
| Sens 2 | 81% | 83% | 81% | 82% | 89% | 81% | 82% | 86% | 82% |
| Spec 2 | 28% | 16% | 28% | 25% | 7% | 27% | 28% | 63% | 10% |
| Cutoff 3 | 0.768 | 0.314 | 0.883 | 0.795 | 0.241 | 0.872 | 0.611 | 0.314 | 0.562 |
| Sens 3 | 93% | 100% | 93% | 91% | 100% | 91% | 94% | 100% | 94% |
| Spec 3 | 15% | 2% | 18% | 17% | 1% | 18% | 10% | 2% | 6% |
| Cutoff 4 | 2.71 | 2.71 | 3.17 | 2.71 | 2.71 | 3.17 | 2.71 | 2.71 | 3.17 |
| Sens 4 | 26% | 50% | 22% | 38% | 56% | 34% | 29% | 43% | 12% |
| Spec 4 | 70% | 70% | 70% | 70% | 70% | 70% | 70% | 70% | 70% |
| Cutoff 5 | 3.99 | 3.88 | 4.22 | 3.99 | 3.88 | 4.22 | 3.99 | 3.88 | 4.22 |
| Sens 5 | 11% | 33% | 11% | 18% | 33% | 16% | 12% | 29% | 12% |
| Spec 5 | 80% | 80% | 80% | 80% | 80% | 80% | 80% | 80% | 80% |
| Cutoff 6 | 5.44 | 5.46 | 5.97 | 5.44 | 5.46 | 5.97 | 5.44 | 5.46 | 5.97 |
| Sens 6 | 7% | 17% | 7% | 12% | 0% | 3% | 6% | 0% | 6% |
| Spec 6 | 90% | 90% | 90% | 90% | 90% | 90% | 90% | 90% | 90% |
| OR Quart 2 | 1.2 | 0 | 1.5 | 0.85 | 0.50 | 0.99 | 0.99 | 0 | 4.3 |
| p Value | 0.76 | na | 0.53 | 0.78 | 0.57 | 0.99 | 0.99 | na | 0.071 |
| 95% CI of | 0.36 | na | 0.41 | 0.28 | 0.044 | 0.31 | 0.20 | na | 0.88 |
| OR Quart 2 | 4.1 | na | 5.6 | 2.6 | 5.5 | 3.2 | 5.0 | na | 21 |
| OR Quart 3 | 2.1 | 0.50 | 3.0 | 1.8 | 0.50 | 2.3 | 3.1 | 3.0 | 1.5 |
| p Value | 0.19 | 0.57 | 0.070 | 0.24 | 0.57 | 0.10 | 0.092 | 0.34 | 0.65 |
| 95% CI of | 0.69 | 0.044 | 0.91 | 0.68 | 0.044 | 0.84 | 0.83 | 0.31 | 0.25 |
| OR Quart 3 | 6.3 | 5.5 | 9.7 | 4.7 | 5.5 | 6.4 | 12 | 30 | 9.3 |
| OR Quart 4 | 1.2 | 1.5 | 1.5 | 1.3 | 2.6 | 1.2 | 0.65 | 3.0 | 2.0 |
| p Value | 0.77 | 0.66 | 0.53 | 0.61 | 0.27 | 0.79 | 0.65 | 0.34 | 0.42 |
| 95% CI of | 0.36 | 0.25 | 0.41 | 0.47 | 0.49 | 0.38 | 0.11 | 0.31 | 0.37 |
| OR Quart 4 | 4.0 | 9.1 | 5.6 | 3.6 | 13 | 3.6 | 4.0 | 29 | 11 |

Myoglobin

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 28.5 | 65.8 | 28.5 | 80.9 | 28.5 | 82.0 |
| Average | 82.5 | 171 | 82.5 | 252 | 82.5 | 221 |
| Stdev | 182 | 372 | 182 | 471 | 182 | 396 |
| p (t-test) | | 0.024 | | 1.2E−5 | | 0.0040 |
| Min | 3.55 | 4.22 | 3.55 | 3.96 | 3.55 | 5.12 |
| Max | 2130 | 1880 | 2130 | 1880 | 2130 | 1310 |
| n (Samp) | 434 | 27 | 434 | 34 | 434 | 17 |
| n (Patient) | 173 | 27 | 173 | 34 | 173 | 17 |
| sCr only | | | | | | |
| Median | 32.7 | 142 | 32.7 | 125 | 32.7 | 151 |
| Average | 87.4 | 640 | 87.4 | 653 | 87.4 | 396 |
| Stdev | 195 | 849 | 195 | 793 | 195 | 429 |
| p (t-test) | | 3.2E−10 | | 3.3E−14 | | 5.1E−5 |
| Min | 3.55 | 35.2 | 3.55 | 13.0 | 3.55 | 17.5 |
| Max | 2130 | 1880 | 2130 | 1880 | 2130 | 1180 |
| n (Samp) | 535 | 6 | 535 | 9 | 535 | 7 |
| n (Patient) | 207 | 6 | 207 | 9 | 207 | 7 |
| UO only | | | | | | |
| Median | 30.3 | 56.9 | 30.3 | 86.9 | 30.3 | 79.1 |
| Average | 83.3 | 103 | 83.3 | 171 | 83.3 | 261 |
| Stdev | 185 | 149 | 185 | 306 | 185 | 518 |
| p (t-test) | | 0.59 | | 0.016 | | 7.1E−4 |
| Min | 4.60 | 4.22 | 4.60 | 3.96 | 4.60 | 5.12 |
| Max | 2130 | 646 | 2130 | 1410 | 2130 | 1880 |
| n (Samp) | 359 | 27 | 359 | 32 | 359 | 17 |
| n (Patient) | 139 | 27 | 139 | 32 | 139 | 17 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | 0.83 | 0.64 | 0.66 | 0.70 | 0.67 | 0.64 | 0.81 | 0.65 |
| SE | 0.059 | 0.10 | 0.059 | 0.053 | 0.098 | 0.054 | 0.074 | 0.099 | 0.074 |
| p | 0.0056 | 0.0015 | 0.015 | 0.0030 | 0.041 | 0.0022 | 0.050 | 0.0018 | 0.040 |
| nCohort 1 | 434 | 535 | 359 | 434 | 535 | 359 | 434 | 535 | 359 |
| nCohort 2 | 27 | 6 | 27 | 34 | 9 | 32 | 17 | 7 | 17 |
| Cutoff 1 | 42.7 | 66.9 | 42.7 | 34.8 | 31.4 | 38.6 | 34.8 | 101 | 34.8 |
| Sens 1 | 70% | 83% | 70% | 71% | 78% | 72% | 71% | 71% | 71% |
| Spec 1 | 64% | 73% | 63% | 57% | 50% | 59% | 57% | 82% | 55% |
| Cutoff 2 | 22.9 | 66.9 | 22.9 | 16.2 | 19.2 | 18.9 | 17.5 | 87.6 | 18.7 |
| Sens 2 | 81% | 83% | 81% | 82% | 89% | 81% | 82% | 86% | 82% |
| Spec 2 | 40% | 73% | 39% | 25% | 30% | 31% | 27% | 78% | 30% |
| Cutoff 3 | 18.3 | 34.8 | 18.3 | 12.9 | 12.9 | 12.9 | 11.1 | 17.4 | 11.1 |
| Sens 3 | 93% | 100% | 93% | 91% | 100% | 91% | 94% | 100% | 94% |
| Spec 3 | 30% | 53% | 30% | 18% | 17% | 18% | 13% | 25% | 12% |
| Cutoff 4 | 56.0 | 60.1 | 57.8 | 56.0 | 60.1 | 57.8 | 56.0 | 60.1 | 57.8 |
| Sens 4 | 52% | 83% | 48% | 62% | 56% | 69% | 65% | 86% | 65% |
| Spec 4 | 70% | 70% | 70% | 70% | 70% | 70% | 70% | 70% | 70% |
| Cutoff 5 | 93.4 | 95.1 | 95.1 | 93.4 | 95.1 | 95.1 | 93.4 | 95.1 | 95.1 |
| Sens 5 | 33% | 67% | 22% | 44% | 56% | 47% | 35% | 71% | 35% |
| Spec 5 | 80% | 80% | 80% | 80% | 80% | 80% | 80% | 80% | 80% |
| Cutoff 6 | 182 | 180 | 188 | 182 | 180 | 188 | 182 | 180 | 188 |
| Sens 6 | 15% | 33% | 7% | 24% | 44% | 19% | 18% | 43% | 18% |
| Spec 6 | 90% | 90% | 90% | 90% | 90% | 90% | 90% | 90% | 90% |
| OR Quart 2 | 2.6 | >0 | 2.6 | 0.27 | 2.0 | 0.32 | 1.5 | 0 | 1.0 |
| p Value | 0.27 | <na | 0.27 | 0.11 | 0.57 | 0.16 | 0.66 | na | 1.0 |
| 95% CI of | 0.49 | >na | 0.48 | 0.056 | 0.18 | 0.062 | 0.25 | na | 0.14 |
| OR Quart 2 | 14 | na | 13 | 1.3 | 22 | 1.6 | 9.2 | na | 7.3 |
| OR Quart 3 | 4.8 | >2.0 | 4.9 | 1.0 | 1.0 | 1.2 | 0.99 | 0 | 2.0 |
| p Value | 0.048 | <0.57 | 0.047 | 1.0 | 1.0 | 0.79 | 0.99 | na | 0.42 |
| 95% CI of | 1.0 | >0.18 | 1.0 | 0.34 | 0.062 | 0.38 | 0.14 | na | 0.37 |
| OR Quart 3 | 23 | na | 23 | 2.9 | 16 | 3.6 | 7.2 | na | 11 |
| OR Quart 4 | 5.9 | >4.1 | 6.0 | 2.9 | 5.2 | 3.2 | 5.3 | 6.2 | 4.9 |
| p Value | 0.023 | <0.21 | 0.022 | 0.024 | 0.14 | 0.020 | 0.033 | 0.094 | 0.047 |
| 95% CI of | 1.3 | >0.45 | 1.3 | 1.1 | 0.59 | 1.2 | 1.1 | 0.73 | 1.0 |
| OR Quart 4 | 27 | na | 28 | 7.1 | 45 | 8.5 | 25 | 52 | 23 |

Mucin-16

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.34 | 1.72 | 1.34 | 1.78 | 1.34 | 2.02 |
| Average | 4.05 | 40.6 | 4.05 | 23.6 | 4.05 | 19.4 |
| Stdev | 10.9 | 98.2 | 10.9 | 66.0 | 10.9 | 61.0 |
| p (t-test) | | 4.2E−6 | | 2.0E−4 | | 0.0027 |
| Min | 0.117 | 1.12 | 0.117 | 0.191 | 0.117 | 0.549 |
| Max | 131 | 263 | 131 | 253 | 131 | 248 |
| n (Samp) | 196 | 7 | 196 | 22 | 196 | 16 |
| n (Patient) | 130 | 7 | 130 | 22 | 130 | 16 |
| UO only | | | | | | |
| Median | nd | nd | 1.46 | 2.02 | 1.46 | 3.28 |
| Average | nd | nd | 4.51 | 27.2 | 4.51 | 23.4 |
| Stdev | nd | nd | 11.7 | 70.6 | 11.7 | 67.5 |
| p (t-test) | nd | nd | | 1.8E−4 | | 0.0018 |
| Min | nd | nd | 0.122 | 0.191 | 0.122 | 0.549 |
| Max | nd | nd | 131 | 253 | 131 | 248 |
| n (Samp) | nd | nd | 170 | 19 | 170 | 13 |
| n (Patient) | nd | nd | 108 | 19 | 108 | 13 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | nd | nd | 0.60 | nd | 0.59 | 0.64 | nd | 0.66 |
| SE | 0.11 | nd | nd | 0.067 | nd | 0.072 | 0.077 | nd | 0.085 |
| p | 0.16 | nd | nd | 0.12 | nd | 0.23 | 0.078 | nd | 0.059 |
| nCohort 1 | 196 | nd | nd | 196 | nd | 170 | 196 | nd | 170 |
| nCohort 2 | 7 | nd | nd | 22 | nd | 19 | 16 | nd | 13 |
| Cutoff 1 | 1.33 | nd | nd | 1.09 | nd | 1.12 | 1.23 | nd | 1.28 |
| Sens 1 | 71% | nd | nd | 73% | nd | 74% | 75% | nd | 77% |
| Spec 1 | 50% | nd | nd | 41% | nd | 39% | 46% | nd | 44% |
| Cutoff 2 | 1.23 | nd | nd | 1.01 | nd | 0.633 | 1.01 | nd | 1.01 |
| Sens 2 | 86% | nd | nd | 82% | nd | 84% | 81% | nd | 85% |
| Spec 2 | 46% | nd | nd | 38% | nd | 22% | 38% | nd | 35% |
| Cutoff 3 | 1.09 | nd | nd | 0.464 | nd | 0.424 | 0.761 | nd | 0.821 |
| Sens 3 | 100% | nd | nd | 91% | nd | 95% | 94% | nd | 92% |
| Spec 3 | 41% | nd | nd | 14% | nd | 12% | 29% | nd | 32% |
| Cutoff 4 | 2.77 | nd | nd | 2.77 | nd | 3.55 | 2.77 | nd | 3.55 |
| Sens 4 | 43% | nd | nd | 41% | nd | 37% | 44% | nd | 46% |
| Spec 4 | 70% | nd | nd | 70% | nd | 70% | 70% | nd | 70% |
| Cutoff 5 | 4.41 | nd | nd | 4.41 | nd | 4.88 | 4.41 | nd | 4.88 |
| Sens 5 | 29% | nd | nd | 32% | nd | 32% | 38% | nd | 38% |
| Spec 5 | 80% | nd | nd | 80% | nd | 80% | 80% | nd | 80% |
| Cutoff 6 | 6.95 | nd | nd | 6.95 | nd | 7.65 | 6.95 | nd | 7.65 |
| Sens 6 | 29% | nd | nd | 27% | nd | 32% | 31% | nd | 38% |
| Spec 6 | 90% | nd | nd | 90% | nd | 90% | 90% | nd | 90% |
| OR Quart 2 | >2.0 | nd | nd | 0.98 | nd | 1.0 | 6.6 | nd | 3.1 |
| p Value | <0.57 | nd | nd | 0.98 | nd | 1.0 | 0.085 | nd | 0.34 |
| 95% CI of | >0.18 | nd | nd | 0.23 | nd | 0.23 | 0.77 | nd | 0.31 |
| OR Quart 2 | na | nd | nd | 4.1 | nd | 4.3 | 57 | nd | 31 |
| OR Quart 3 | >3.1 | nd | nd | 1.6 | nd | 1.0 | 3.1 | nd | 3.1 |
| p Value | <0.33 | nd | nd | 0.51 | nd | 1.0 | 0.33 | nd | 0.34 |
| 95% CI of | >0.31 | nd | nd | 0.42 | nd | 0.23 | 0.31 | nd | 0.31 |
| OR Quart 3 | na | nd | nd | 5.9 | nd | 4.3 | 31 | nd | 31 |
| OR Quart 4 | >2.0 | nd | nd | 2.1 | nd | 1.8 | 6.6 | nd | 6.6 |
| p Value | <0.57 | nd | nd | 0.24 | nd | 0.36 | 0.085 | nd | 0.087 |
| 95% CI of | >0.18 | nd | nd | 0.60 | nd | 0.50 | 0.77 | nd | 0.76 |
| OR Quart 4 | na | nd | nd | 7.5 | nd | 6.7 | 57 | nd | 57 |

Tumor necrosis factor receptor superfamily member 10B

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.00E−9 | 0.00692 | 1.00E−9 | 0.00181 | 1.00E−9 | 0.00572 |
| Average | 0.00543 | 0.0417 | 0.00543 | 0.0242 | 0.00543 | 0.0256 |
| Stdev | 0.0150 | 0.0972 | 0.0150 | 0.0668 | 0.0150 | 0.0736 |
| p (t-test) | | 4.5E−5 | | 0.0018 | | 0.0020 |
| Min | 1.00E−9 | 1.00E−9 | 1.00E−9 | 1.00E−9 | 1.00E−9 | 1.00E−9 |
| Max | 0.114 | 0.262 | 0.114 | 0.286 | 0.114 | 0.300 |
| n (Samp) | 189 | 7 | 189 | 18 | 189 | 16 |
| n (Patient) | 125 | 7 | 125 | 18 | 125 | 16 |
| UO only | | | | | | |
| Median | nd | nd | 0.000420 | 0.00142 | 0.000420 | 0.00220 |
| Average | nd | nd | 0.00637 | 0.0151 | 0.00637 | 0.00604 |
| Stdev | nd | nd | 0.0161 | 0.0277 | 0.0161 | 0.00762 |
| p (t-test) | nd | nd | | 0.053 | | 0.94 |
| Min | nd | nd | 1.00E−9 | 1.00E−9 | 1.00E−9 | 1.00E−9 |
| Max | nd | nd | 0.114 | 0.108 | 0.114 | 0.0222 |
| n (Samp) | nd | nd | 162 | 17 | 162 | 13 |
| n (Patient) | nd | nd | 101 | 17 | 101 | 13 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.63 | nd | nd | 0.61 | nd | 0.59 | 0.65 | nd | 0.57 |
| SE | 0.12 | nd | nd | 0.073 | nd | 0.076 | 0.077 | nd | 0.086 |
| p | 0.25 | nd | nd | 0.12 | nd | 0.25 | 0.060 | nd | 0.38 |
| nCohort 1 | 189 | nd | nd | 189 | nd | 162 | 189 | nd | 162 |
| nCohort 2 | 7 | nd | nd | 18 | nd | 17 | 16 | nd | 13 |
| Cutoff 1 | 0 | nd | nd | 0 | nd | 0 | 0 | nd | 0 |
| Sens 1 | 100% | nd | nd | 100% | nd | 100% | 100% | nd | 100% |
| Spec 1 | 0% | nd | nd | 0% | nd | 0% | 0% | nd | 0% |
| Cutoff 2 | 0 | nd | nd | 0 | nd | 0 | 0 | nd | 0 |
| Sens 2 | 100% | nd | nd | 100% | nd | 100% | 100% | nd | 100% |
| Spec 2 | 0% | nd | nd | 0% | nd | 0% | 0% | nd | 0% |
| Cutoff 3 | 0 | nd | nd | 0 | nd | 0 | 0 | nd | 0 |
| Sens 3 | 100% | nd | nd | 100% | nd | 100% | 100% | nd | 100% |
| Spec 3 | 0% | nd | nd | 0% | nd | 0% | 0% | nd | 0% |
| Cutoff 4 | 0.00367 | nd | nd | 0.00367 | nd | 0.00444 | 0.00367 | nd | 0.00444 |
| Sens 4 | 57% | nd | nd | 44% | nd | 47% | 50% | nd | 38% |
| Spec 4 | 70% | nd | nd | 70% | nd | 70% | 70% | nd | 70% |
| Cutoff 5 | 0.00692 | nd | nd | 0.00692 | nd | 0.00819 | 0.00692 | nd | 0.00819 |
| Sens 5 | 43% | nd | nd | 39% | nd | 35% | 50% | nd | 31% |
| Spec 5 | 81% | nd | nd | 81% | nd | 82% | 81% | nd | 82% |
| Cutoff 6 | 0.0124 | nd | nd | 0.0124 | nd | 0.0136 | 0.0124 | nd | 0.0136 |
| Sens 6 | 29% | nd | nd | 28% | nd | 29% | 31% | nd | 23% |
| Spec 6 | 91% | nd | nd | 91% | nd | 90% | 91% | nd | 90% |
| OR Quart 2 | >3.2 | nd | nd | >7.9 | nd | 1.3 | >5.5 | nd | 4.2 |
| p Value | <0.32 | nd | nd | <0.057 | nd | 0.72 | <0.12 | nd | 0.21 |
| 95% CI of | >0.32 | nd | nd | >0.94 | nd | 0.28 | >0.62 | nd | 0.45 |
| OR Quart 2 | na | nd | nd | na | nd | 6.3 | na | nd | 39 |
| OR Quart 3 | >0 | nd | nd | >4.2 | nd | 0.98 | >3.2 | nd | 3.1 |
| p Value | <na | nd | nd | <0.20 | nd | 0.98 | <0.32 | nd | 0.34 |
| 95% CI of | >na | nd | nd | >0.46 | nd | 0.19 | >0.32 | nd | 0.31 |
| OR Quart 3 | na | nd | nd | na | nd | 5.1 | na | nd | 31 |
| OR Quart 4 | >4.4 | nd | nd | >7.9 | nd | 2.5 | >9.3 | nd | 5.4 |
| p Value | <0.20 | nd | nd | <0.057 | nd | 0.20 | <0.039 | nd | 0.13 |
| 95% CI of | >0.47 | nd | nd | >0.94 | nd | 0.61 | >1.1 | nd | 0.60 |
| OR Quart 4 | na | nd | nd | na | nd | 10 | na | nd | 48 |

Cellular tumor antigen p53

| | | |
|---|---|---|
| 0 hr prior to | 24 hr prior to | 48 hr prior to |
| AKI stage | AKI stage | AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.00E−9 | 1.00E−9 | 1.00E−9 | 1.00E−9 | 1.00E−9 | 1.00E−9 |
| Average | 0.0690 | 0.289 | 0.0690 | 0.143 | 0.0690 | 0.172 |
| Stdev | 0.419 | 0.698 | 0.419 | 0.551 | 0.419 | 0.595 |
| p (t-test) | | 0.19 | | 0.49 | | 0.36 |
| Min | 1.00E−9 | 1.00E−9 | 1.00E−9 | 1.00E−9 | 1.00E−9 | 1.00E−9 |
| Max | 3.70 | 1.87 | 3.70 | 2.35 | 3.70 | 2.39 |
| n (Samp) | 189 | 7 | 189 | 18 | 189 | 16 |
| n (Patient) | 125 | 7 | 125 | 18 | 125 | 16 |
| UO only | | | | | | |
| Median | nd | nd | 1.00E−9 | 1.00E−9 | 1.00E−9 | 1.00E−9 |
| Average | nd | nd | 0.0804 | 0.0131 | 0.0804 | 0.0283 |
| Stdev | nd | nd | 0.452 | 0.0316 | 0.452 | 0.0790 |
| p (t-test) | nd | nd | | 0.54 | | 0.68 |
| Min | nd | nd | 1.00E−9 | 1.00E−9 | 1.00E−9 | 1.00E−9 |
| Max | nd | nd | 3.70 | 0.117 | 3.70 | 0.285 |
| n (Samp) | nd | nd | 162 | 17 | 162 | 13 |
| n (Patient) | nd | nd | 101 | 17 | 101 | 13 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.64 | nd | nd | 0.62 | nd | 0.59 | 0.54 | nd | 0.52 |
| SE | 0.11 | nd | nd | 0.073 | nd | 0.076 | 0.077 | nd | 0.084 |
| p | 0.21 | nd | nd | 0.091 | nd | 0.23 | 0.57 | nd | 0.80 |
| nCohort 1 | 189 | nd | nd | 189 | nd | 162 | 189 | nd | 162 |
| nCohort 2 | 7 | nd | nd | 18 | nd | 17 | 16 | nd | 13 |
| Cutoff 1 | 0 | nd | nd | 0 | nd | 0 | 0 | nd | 0 |
| Sens 1 | 100% | nd | nd | 100% | nd | 100% | 100% | nd | 100% |
| Spec 1 | 0% | nd | nd | 0% | nd | 0% | 0% | nd | 0% |
| Cutoff 2 | 0 | nd | nd | 0 | nd | 0 | 0 | nd | 0 |
| Sens 2 | 100% | nd | nd | 100% | nd | 100% | 100% | nd | 100% |
| Spec 2 | 0% | nd | nd | 0% | nd | 0% | 0% | nd | 0% |
| Cutoff 3 | 0 | nd | nd | 0 | nd | 0 | 0 | nd | 0 |
| Sens 3 | 100% | nd | nd | 100% | nd | 100% | 100% | nd | 100% |
| Spec 3 | 0% | nd | nd | 0% | nd | 0% | 0% | nd | 0% |
| Cutoff 4 | 1.00E−9 | nd | nd | 1.00E−9 | nd | 1.00E−9 | 1.00E−9 | nd | 1.00E−9 |
| Sens 4 | 43% | nd | nd | 44% | nd | 41% | 25% | nd | 23% |
| Spec 4 | 81% | nd | nd | 81% | nd | 80% | 81% | nd | 80% |
| Cutoff 5 | 1.00E−9 | nd | nd | 1.00E−9 | nd | 0.000790 | 1.00E−9 | nd | 0.000790 |
| Sens 5 | 43% | nd | nd | 44% | nd | 35% | 25% | nd | 23% |
| Spec 5 | 81% | nd | nd | 81% | nd | 81% | 81% | nd | 81% |
| Cutoff 6 | 0.0225 | nd | nd | 0.0225 | nd | 0.0267 | 0.0225 | nd | 0.0267 |
| Sens 6 | 43% | nd | nd | 17% | nd | 12% | 25% | nd | 15% |
| Spec 6 | 90% | nd | nd | 90% | nd | 90% | 90% | nd | 90% |
| OR Quart 2 | 3.1 | nd | nd | 10 | nd | >13 | >16 | nd | >13 |
| p Value | 0.33 | nd | nd | 0.029 | nd | <0.018 | <0.0096 | nd | <0.018 |
| 95% CI of | 0.31 | nd | nd | 1.3 | nd | >1.5 | >2.0 | nd | >1.5 |
| OR Quart 2 | 31 | nd | nd | 86 | nd | na | na | nd | na |
| OR Quart 3 | 0 | nd | nd | 0 | nd | >0 | >0 | nd | >0 |
| p Value | na | nd | nd | na | nd | <na | <na | nd | <na |
| 95% CI of | na | nd | nd | na | nd | >na | >na | nd | >na |
| OR Quart 3 | na | nd | nd | na | nd | na | na | nd | na |
| OR Quart 4 | 3.1 | nd | nd | 9.1 | nd | >8.1 | >4.2 | nd | >3.1 |
| p Value | 0.33 | nd | nd | 0.041 | nd | <0.055 | <0.20 | nd | <0.33 |
| 95% CI of | 0.31 | nd | nd | 1.1 | nd | >0.95 | >0.46 | nd | >0.31 |
| OR Quart 4 | 31 | nd | nd | 76 | nd | na | na | nd | na |

TABLE 7
Comparison of marker levels in EDTA samples collected within 12 hours of
reaching stage R from Cohort 1 (patients that reached, but did not progress
beyond, RIFLE stage R) and from Cohort 2 (patients that reached RIFLE stage I or F).
Apolipoprotein A-II

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 57100 | 48800 | nd | nd | 57100 | 45400 |
| Average | 57200 | 51600 | nd | nd | 57000 | 46300 |
| Stdev | 36400 | 24300 | nd | nd | 39000 | 21400 |
| p (t-test) | | 0.53 | nd | nd | | 0.34 |
| Min | 7970 | 1810 | nd | nd | 7970 | 1810 |
| Max | 251000 | 100000 | nd | nd | 251000 | 75500 |
| n (Samp) | 50 | 19 | nd | nd | 41 | 14 |
| n (Patient) | 50 | 19 | nd | nd | 41 | 14 |

| | At Enrollment | | |
|---|---|---|---|
| | sCr or UO | sCr only | UO only |
| AUC | 0.47 | nd | 0.44 |
| SE | 0.079 | nd | 0.091 |
| p | 0.69 | nd | 0.49 |
| nCohort 1 | 50 | nd | 41 |
| nCohort 2 | 19 | nd | 14 |
| Cutoff 1 | 37400 | nd | 32500 |
| Sens 1 | 74% | nd | 71% |
| Spec 1 | 26% | nd | 24% |
| Cutoff 2 | 28800 | nd | 27600 |
| Sens 2 | 84% | nd | 86% |
| Spec 2 | 18% | nd | 22% |
| Cutoff 3 | 23900 | nd | 23900 |
| Sens 3 | 95% | nd | 93% |
| Spec 3 | 16% | nd | 20% |
| Cutoff 4 | 64400 | nd | 66800 |
| Sens 4 | 26% | nd | 21% |
| Spec 4 | 70% | nd | 71% |
| Cutoff 5 | 72300 | nd | 72300 |
| Sens 5 | 21% | nd | 14% |
| Spec 5 | 80% | nd | 80% |
| Cutoff 6 | 83600 | nd | 85700 |
| Sens 6 | 11% | nd | 0% |
| Spec 6 | 90% | nd | 90% |
| OR Quart 2 | 0.80 | nd | 1.0 |
| p Value | 0.77 | nd | 1.0 |
| 95% CI of | 0.17 | nd | 0.16 |
| OR Quart 2 | 3.7 | nd | 6.1 |
| OR Quart 3 | 1.1 | nd | 1.5 |
| p Value | 0.91 | nd | 0.66 |
| 95% CI of | 0.25 | nd | 0.26 |
| OR Quart 3 | 4.7 | nd | 8.2 |
| OR Quart 4 | 1.1 | nd | 1.6 |
| p Value | 0.91 | nd | 0.58 |
| 95% CI of | 0.25 | nd | 0.29 |
| OR Quart 4 | 4.7 | nd | 9.3 |

Myoglobin

| | sCr or UO | | sCr only | | UO only | |
|---|---|---|---|---|---|---|
| | Cohort 1 | Cohort 2 | Cohort 1 | Cohort 2 | Cohort 1 | Cohort 2 |
| Median | 40.1 | 64.7 | nd | nd | 35.8 | 79.1 |
| Average | 139 | 182 | nd | nd | 132 | 88.9 |
| Stdev | 314 | 344 | nd | nd | 329 | 98.2 |
| p (t-test) | | 0.60 | nd | nd | | 0.60 |
| Min | 7.19 | 4.16 | nd | nd | 7.19 | 4.16 |
| Max | 2130 | 1310 | nd | nd | 2130 | 397 |
| n (Samp) | 54 | 23 | nd | nd | 46 | 17 |
| n (Patient) | 54 | 23 | nd | nd | 46 | 17 |

| | At Enrollment | | |
|---|---|---|---|
| | sCr or UO | sCr only | UO only |
| AUC | 0.55 | nd | 0.54 |
| SE | 0.073 | nd | 0.083 |
| p | 0.48 | nd | 0.66 |
| nCohort 1 | 54 | nd | 46 |
| nCohort 2 | 23 | nd | 17 |
| Cutoff 1 | 30.3 | nd | 34.2 |
| Sens 1 | 74% | nd | 71% |
| Spec 1 | 43% | nd | 50% |
| Cutoff 2 | 17.8 | nd | 15.3 |
| Sens 2 | 83% | nd | 82% |
| Spec 2 | 19% | nd | 13% |
| Cutoff 3 | 12.9 | nd | 4.16 |
| Sens 3 | 91% | nd | 94% |
| Spec 3 | 13% | nd | 0% |
| Cutoff 4 | 91.7 | nd | 90.0 |
| Sens 4 | 35% | nd | 41% |
| Spec 4 | 70% | nd | 72% |
| Cutoff 5 | 189 | nd | 141 |
| Sens 5 | 17% | nd | 18% |
| Spec 5 | 81% | nd | 80% |
| Cutoff 6 | 328 | nd | 315 |
| Sens 6 | 13% | nd | 6% |
| Spec 6 | 91% | nd | 91% |
| OR Quart 2 | 0.41 | nd | 0.29 |
| p Value | 0.26 | nd | 0.18 |
| 95% CI of | 0.085 | nd | 0.046 |
| OR Quart 2 | 1.9 | nd | 1.8 |
| OR Quart 3 | 1.6 | nd | 0.91 |
| p Value | 0.50 | nd | 0.90 |
| 95% CI of | 0.42 | nd | 0.20 |
| OR Quart 3 | 5.9 | nd | 4.1 |
| OR Quart 4 | 0.93 | nd | 0.91 |
| p Value | 0.91 | nd | 0.90 |
| 95% CI of | 0.24 | nd | 0.20 |
| OR Quart 4 | 3.6 | nd | 4.1 |

TABLE 8
Comparison of the maximum marker levels in EDTA samples collected from
Cohort 1 (patients that did not progress beyond RIFLE stage 0) and the maximum values in
EDTA samples collected from subjects between enrollment and 0, 24 hours, and 48 hours prior
to reaching stage F in Cohort 2.
Apolipoprotein A-II

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 52600 | 43600 | 52600 | 41100 | 52600 | 57700 |
| Average | 52700 | 57800 | 52700 | 57100 | 52700 | 72600 |
| Stdev | 22500 | 48900 | 22500 | 51100 | 22500 | 58900 |
| p (t-test) | | 0.55 | | 0.62 | | 0.068 |
| Min | 5450 | 10700 | 5450 | 10700 | 5450 | 9430 |
| Max | 105000 | 176000 | 105000 | 176000 | 105000 | 176000 |
| n (Samp) | 97 | 11 | 97 | 10 | 97 | 6 |
| n (Patient) | 97 | 11 | 97 | 10 | 97 | 6 |
| UO only | | | | | | |
| Median | 53400 | 57800 | 53400 | 52600 | 53400 | 57700 |
| Average | 53900 | 66500 | 53900 | 64700 | 53900 | 72600 |
| Stdev | 20600 | 54800 | 20600 | 54700 | 20600 | 58900 |
| p (t-test) | | 0.18 | | 0.25 | | 0.073 |
| Min | 8680 | 10700 | 8680 | 10700 | 8680 | 9430 |
| Max | 123000 | 176000 | 123000 | 176000 | 123000 | 176000 |
| n (Samp) | 84 | 8 | 84 | 8 | 84 | 6 |
| n (Patient) | 84 | 8 | 84 | 8 | 84 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.46 | nd | 0.53 | 0.45 | nd | 0.51 | 0.57 | nd | 0.56 |
| SE | 0.094 | nd | 0.11 | 0.098 | nd | 0.11 | 0.13 | nd | 0.13 |
| p | 0.69 | nd | 0.78 | 0.61 | nd | 0.94 | 0.59 | nd | 0.66 |
| nCohort 1 | 97 | nd | 84 | 97 | nd | 84 | 97 | nd | 84 |
| nCohort 2 | 11 | nd | 8 | 10 | nd | 8 | 6 | nd | 6 |
| Cutoff 1 | 37600 | nd | 37600 | 37600 | nd | 37600 | 37600 | nd | 37600 |
| Sens 1 | 73% | nd | 75% | 70% | nd | 75% | 83% | nd | 83% |
| Spec 1 | 27% | nd | 19% | 27% | nd | 19% | 27% | nd | 19% |
| Cutoff 2 | 14000 | nd | 10700 | 14000 | nd | 10700 | 37600 | nd | 37600 |
| Sens 2 | 82% | nd | 88% | 80% | nd | 88% | 83% | nd | 83% |
| Spec 2 | 4% | nd | 1% | 4% | nd | 1% | 27% | nd | 19% |
| Cutoff 3 | 10700 | nd | 8680 | 10700 | nd | 8680 | 8680 | nd | 8680 |
| Sens 3 | 91% | nd | 100% | 90% | nd | 100% | 100% | nd | 100% |
| Spec 3 | 2% | nd | 1% | 2% | nd | 1% | 2% | nd | 1% |
| Cutoff 4 | 61600 | nd | 61700 | 61600 | nd | 61700 | 61600 | nd | 61700 |
| Sens 4 | 36% | nd | 50% | 40% | nd | 50% | 50% | nd | 50% |
| Spec 4 | 70% | nd | 70% | 70% | nd | 70% | 70% | nd | 70% |
| Cutoff 5 | 70000 | nd | 69100 | 70000 | nd | 69100 | 70000 | nd | 69100 |
| Sens 5 | 36% | nd | 50% | 30% | nd | 38% | 50% | nd | 50% |
| Spec 5 | 80% | nd | 81% | 80% | nd | 81% | 80% | nd | 81% |
| Cutoff 6 | 82900 | nd | 77900 | 82900 | nd | 77900 | 82900 | nd | 77900 |
| Sens 6 | 18% | nd | 25% | 20% | nd | 25% | 33% | nd | 33% |
| Spec 6 | 91% | nd | 90% | 91% | nd | 90% | 91% | nd | 90% |
| OR Quart 2 | 0 | nd | 0.30 | 0.31 | nd | 0.30 | 2.0 | nd | 0.45 |
| p Value | na | nd | 0.32 | 0.32 | nd | 0.32 | 0.58 | nd | 0.53 |
| 95% CI of | na | nd | 0.029 | 0.030 | nd | 0.029 | 0.17 | nd | 0.038 |
| OR Quart 2 | na | nd | 3.2 | 3.2 | nd | 3.2 | 24 | nd | 5.4 |
| OR Quart 3 | 1.0 | nd | 0 | 1.0 | nd | 0.30 | 0 | nd | 0 |
| p Value | 1.0 | nd | na | 1.0 | nd | 0.32 | na | nd | na |
| 95% CI of | 0.22 | nd | na | 0.18 | nd | 0.029 | na | nd | na |
| OR Quart 3 | 4.5 | nd | na | 5.5 | nd | 3.2 | na | nd | na |
| OR Quart 4 | 0.72 | nd | 1.4 | 1.0 | nd | 1.0 | 3.1 | nd | 1.5 |
| p Value | 0.69 | nd | 0.68 | 0.96 | nd | 1.0 | 0.34 | nd | 0.67 |
| 95% CI of | 0.14 | nd | 0.28 | 0.19 | nd | 0.18 | 0.30 | nd | 0.23 |
| OR Quart 4 | 3.6 | nd | 7.1 | 5.7 | nd | 5.6 | 32 | nd | 10.0 |

Caspase-1

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 91.6 | 138 | 91.6 | 90.7 | 91.6 | 83.0 |
| Average | 108 | 154 | 108 | 121 | 108 | 101 |
| Stdev | 74.7 | 96.4 | 74.7 | 71.9 | 74.7 | 53.3 |
| p (t-test) | | 0.17 | | 0.69 | | 0.83 |
| Min | 33.8 | 44.7 | 33.8 | 44.7 | 33.8 | 44.7 |
| Max | 326 | 328 | 326 | 241 | 326 | 192 |
| n (Samp) | 26 | 8 | 26 | 7 | 26 | 6 |
| n (Patient) | 26 | 8 | 26 | 7 | 26 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.67 | nd | nd | 0.56 | nd | nd | 0.51 | nd | nd |
| SE | 0.12 | nd | nd | 0.13 | nd | nd | 0.13 | nd | nd |
| p | 0.15 | nd | nd | 0.62 | nd | nd | 0.94 | nd | nd |
| nCohort 1 | 26 | nd | nd | 26 | nd | nd | 26 | nd | nd |
| nCohort 2 | 8 | nd | nd | 7 | nd | nd | 6 | nd | nd |
| Cutoff 1 | 71.0 | nd | nd | 70.7 | nd | nd | 66.1 | nd | nd |
| Sens 1 | 75% | nd | nd | 71% | nd | nd | 83% | nd | nd |
| Spec 1 | 38% | nd | nd | 38% | nd | nd | 31% | nd | nd |
| Cutoff 2 | 70.7 | nd | nd | 66.1 | nd | nd | 66.1 | nd | nd |
| Sens 2 | 88% | nd | nd | 86% | nd | nd | 83% | nd | nd |
| Spec 2 | 38% | nd | nd | 31% | nd | nd | 31% | nd | nd |
| Cutoff 3 | 42.1 | nd | nd | 42.1 | nd | nd | 42.1 | nd | nd |
| Sens 3 | 100% | nd | nd | 100% | nd | nd | 100% | nd | nd |
| Spec 3 | 15% | nd | nd | 15% | nd | nd | 15% | nd | nd |
| Cutoff 4 | 103 | nd | nd | 103 | nd | nd | 103 | nd | nd |
| Sens 4 | 62% | nd | nd | 43% | nd | nd | 33% | nd | nd |
| Spec 4 | 73% | nd | nd | 73% | nd | nd | 73% | nd | nd |
| Cutoff 5 | 121 | nd | nd | 121 | nd | nd | 121 | nd | nd |
| Sens 5 | 62% | nd | nd | 43% | nd | nd | 33% | nd | nd |
| Spec 5 | 81% | nd | nd | 81% | nd | nd | 81% | nd | nd |
| Cutoff 6 | 254 | nd | nd | 254 | nd | nd | 254 | nd | nd |
| Sens 6 | 12% | nd | nd | 0% | nd | nd | 0% | nd | nd |
| Spec 6 | 92% | nd | nd | 92% | nd | nd | 92% | nd | nd |
| OR Quart 2 | 2.0 | nd | nd | 2.3 | nd | nd | 2.3 | nd | nd |
| p Value | 0.60 | nd | nd | 0.53 | nd | nd | 0.53 | nd | nd |
| 95% CI of | 0.15 | nd | nd | 0.17 | nd | nd | 0.17 | nd | nd |
| OR Quart 2 | 27 | nd | nd | 33 | nd | nd | 33 | nd | nd |
| OR Quart 3 | 0 | nd | nd | 1.0 | nd | nd | 1.0 | nd | nd |
| p Value | na | nd | nd | 1.0 | nd | nd | 1.0 | nd | nd |
| 95% CI of | na | nd | nd | 0.052 | nd | nd | 0.052 | nd | nd |
| OR Quart 3 | na | nd | nd | 19 | nd | nd | 19 | nd | nd |
| OR Quart 4 | 8.8 | nd | nd | 3.5 | nd | nd | 2.3 | nd | nd |
| p Value | 0.086 | nd | nd | 0.33 | nd | nd | 0.53 | nd | nd |
| 95% CI of | 0.74 | nd | nd | 0.28 | nd | nd | 0.17 | nd | nd |
| OR Quart 4 | 100 | nd | nd | 43 | nd | nd | 33 | nd | nd |

Caspase-9

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 28.7 | 12.2 | 28.7 | 6.83 | 28.7 | 7.20 |
| Average | 50.0 | 16.5 | 50.0 | 13.7 | 50.0 | 14.2 |
| Stdev | 69.0 | 14.3 | 69.0 | 15.0 | 69.0 | 16.2 |
| p (t-test) | | 0.16 | | 0.18 | | 0.22 |
| Min | 2.58 | 4.32 | 2.58 | 3.79 | 2.58 | 3.79 |
| Max | 366 | 46.4 | 366 | 46.4 | 366 | 46.4 |
| n (Samp) | 37 | 9 | 37 | 7 | 37 | 6 |
| n (Patient) | 37 | 9 | 37 | 7 | 37 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.29 | nd | nd | 0.22 | nd | nd | 0.23 | nd | nd |
| SE | 0.10 | nd | nd | 0.11 | nd | nd | 0.12 | nd | nd |
| p | 0.040 | nd | nd | 0.012 | nd | nd | 0.024 | nd | nd |
| nCohort 1 | 37 | nd | nd | 37 | nd | nd | 37 | nd | nd |
| nCohort 2 | 9 | nd | nd | 7 | nd | nd | 6 | nd | nd |
| Cutoff 1 | 6.05 | nd | nd | 6.05 | nd | nd | 4.34 | nd | nd |
| Sens 1 | 78% | nd | nd | 71% | nd | nd | 83% | nd | nd |
| Spec 1 | 11% | nd | nd | 11% | nd | nd | 8% | nd | nd |
| Cutoff 2 | 4.34 | nd | nd | 4.34 | nd | nd | 4.34 | nd | nd |
| Sens 2 | 89% | nd | nd | 86% | nd | nd | 83% | nd | nd |
| Spec 2 | 8% | nd | nd | 8% | nd | nd | 8% | nd | nd |
| Cutoff 3 | 3.30 | nd | nd | 3.30 | nd | nd | 3.30 | nd | nd |
| Sens 3 | 100% | nd | nd | 100% | nd | nd | 100% | nd | nd |
| Spec 3 | 5% | nd | nd | 5% | nd | nd | 5% | nd | nd |
| Cutoff 4 | 50.7 | nd | nd | 50.7 | nd | nd | 50.7 | nd | nd |
| Sens 4 | 0% | nd | nd | 0% | nd | nd | 0% | nd | nd |
| Spec 4 | 70% | nd | nd | 70% | nd | nd | 70% | nd | nd |
| Cutoff 5 | 71.4 | nd | nd | 71.4 | nd | nd | 71.4 | nd | nd |
| Sens 5 | 0% | nd | nd | 0% | nd | nd | 0% | nd | nd |
| Spec 5 | 81% | nd | nd | 81% | nd | nd | 81% | nd | nd |
| Cutoff 6 | 106 | nd | nd | 106 | nd | nd | 106 | nd | nd |
| Sens 6 | 0% | nd | nd | 0% | nd | nd | 0% | nd | nd |
| Spec 6 | 92% | nd | nd | 92% | nd | nd | 92% | nd | nd |
| OR Quart 2 | >4.5 | nd | nd | >1.1 | nd | nd | >1.1 | nd | nd |
| p Value | <0.23 | nd | nd | <0.95 | nd | nd | <0.95 | nd | nd |
| 95% CI of | >0.39 | nd | nd | >0.060 | nd | nd | >0.060 | nd | nd |
| OR Quart 2 | na | nd | nd | na | nd | nd | na | nd | nd |
| OR Quart 3 | >2.4 | nd | nd | >2.4 | nd | nd | >1.1 | nd | nd |
| p Value | <0.50 | nd | nd | <0.49 | nd | nd | <0.95 | nd | nd |
| 95% CI of | >0.19 | nd | nd | >0.19 | nd | nd | >0.060 | nd | nd |
| OR Quart 3 | na | nd | nd | na | nd | nd | na | nd | nd |
| OR Quart 4 | >6.9 | nd | nd | >6.3 | nd | nd | >7.3 | nd | nd |
| p Value | <0.11 | nd | nd | <0.13 | nd | nd | <0.10 | nd | nd |
| 95% CI of | >0.63 | nd | nd | >0.58 | nd | nd | >0.66 | nd | nd |
| OR Quart 4 | na | nd | nd | na | nd | nd | na | nd | nd |

Cadherin-1

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 72700 | 112000 | 72700 | 112000 | 72700 | 162000 |
| Average | 103000 | 157000 | 103000 | 154000 | 103000 | 174000 |
| Stdev | 108000 | 103000 | 108000 | 105000 | 108000 | 96900 |
| p (t-test) | | 0.13 | | 0.15 | | 0.13 |
| Min | 14500 | 38300 | 14500 | 38300 | 14500 | 50500 |
| Max | 621000 | 340000 | 621000 | 340000 | 621000 | 285000 |
| n (Samp) | 50 | 12 | 50 | 12 | 50 | 6 |
| n (Patient) | 50 | 12 | 50 | 12 | 50 | 6 |
| sCr only | | | | | | |
| Median | 77600 | 151000 | 77600 | 132000 | nd | nd |
| Average | 110000 | 170000 | 110000 | 163000 | nd | nd |
| Stdev | 96000 | 127000 | 96000 | 132000 | nd | nd |
| p (t-test) | | 0.15 | | 0.20 | nd | nd |
| Min | 14500 | 38300 | 14500 | 38300 | nd | nd |
| Max | 621000 | 340000 | 621000 | 340000 | nd | nd |
| n (Samp) | 96 | 6 | 96 | 6 | nd | nd |
| n (Patient) | 96 | 6 | 96 | 6 | nd | nd |
| UO only | | | | | | |
| Median | 80200 | 162000 | 80200 | 162000 | 80200 | 162000 |
| Average | 114000 | 170000 | 114000 | 170000 | 114000 | 174000 |
| Stdev | 117000 | 86300 | 117000 | 86300 | 117000 | 96900 |
| p (t-test) | | 0.21 | | 0.21 | | 0.24 |
| Min | 39700 | 64400 | 39700 | 64400 | 39700 | 50500 |
| Max | 621000 | 285000 | 621000 | 285000 | 621000 | 285000 |
| n (Samp) | 44 | 8 | 44 | 8 | 44 | 6 |
| n (Patient) | 44 | 8 | 44 | 8 | 44 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.69 | 0.62 | 0.77 | 0.66 | 0.56 | 0.77 | 0.77 | nd | 0.74 |
| SE | 0.092 | 0.13 | 0.10 | 0.093 | 0.13 | 0.10 | 0.12 | nd | 0.12 |
| p | 0.038 | 0.35 | 0.0087 | 0.094 | 0.65 | 0.0087 | 0.021 | nd | 0.050 |
| nCohort 1 | 50 | 96 | 44 | 50 | 96 | 44 | 50 | nd | 44 |
| nCohort 2 | 12 | 6 | 8 | 12 | 6 | 8 | 6 | nd | 6 |
| Cutoff 1 | 88900 | 53300 | 105000 | 62800 | 53300 | 105000 | 105000 | nd | 105000 |
| Sens 1 | 75% | 83% | 75% | 75% | 83% | 75% | 83% | nd | 83% |
| Spec 1 | 62% | 22% | 75% | 42% | 22% | 75% | 76% | nd | 75% |
| Cutoff 2 | 62800 | 53300 | 88900 | 54100 | 53300 | 88900 | 105000 | nd | 105000 |
| Sens 2 | 83% | 83% | 88% | 83% | 83% | 88% | 83% | nd | 83% |
| Spec 2 | 42% | 22% | 59% | 24% | 22% | 59% | 76% | nd | 75% |
| Cutoff 3 | 52600 | 38100 | 62800 | 52600 | 38100 | 62800 | 48100 | nd | 47600 |
| Sens 3 | 92% | 100% | 100% | 92% | 100% | 100% | 100% | nd | 100% |
| Spec 3 | 22% | 6% | 34% | 22% | 6% | 34% | 20% | nd | 11% |
| Cutoff 4 | 97000 | 108000 | 97500 | 97000 | 108000 | 97500 | 97000 | nd | 97500 |
| Sens 4 | 58% | 50% | 75% | 58% | 50% | 75% | 83% | nd | 83% |
| Spec 4 | 70% | 71% | 70% | 70% | 71% | 70% | 70% | nd | 70% |
| Cutoff 5 | 114000 | 151000 | 131000 | 114000 | 151000 | 131000 | 114000 | nd | 131000 |
| Sens 5 | 50% | 50% | 50% | 50% | 50% | 50% | 67% | nd | 50% |
| Spec 5 | 80% | 80% | 82% | 80% | 80% | 82% | 80% | nd | 82% |
| Cutoff 6 | 153000 | 242000 | 153000 | 153000 | 242000 | 153000 | 153000 | nd | 153000 |
| Sens 6 | 42% | 33% | 50% | 42% | 33% | 50% | 50% | nd | 50% |
| Spec 6 | 90% | 91% | 91% | 90% | 91% | 91% | 90% | nd | 91% |
| OR Quart 2 | 0.43 | 0 | >1.1 | 0.27 | 0 | >1.1 | 0 | nd | 0 |
| p Value | 0.51 | na | <0.96 | 0.28 | na | <0.96 | na | nd | na |
| 95% CI of | 0.035 | na | >0.061 | 0.025 | na | >0.061 | na | nd | na |
| OR Quart 2 | 5.3 | na | na | 2.9 | na | na | na | nd | na |
| OR Quart 3 | 1.6 | 0.48 | >3.9 | 0.62 | 0 | >3.9 | 1.0 | nd | 1.0 |
| p Value | 0.63 | 0.56 | <0.27 | 0.63 | na | <0.27 | 1.0 | nd | 1.0 |
| 95% CI of | 0.23 | 0.041 | >0.35 | 0.087 | na | >0.35 | 0.056 | nd | 0.055 |
| OR Quart 3 | 11 | 5.7 | na | 4.3 | na | na | 18 | nd | 18 |
| OR Quart 4 | 3.9 | 1.5 | >5.8 | 2.4 | 0.96 | >5.8 | 5.2 | nd | 4.9 |
| p Value | 0.14 | 0.67 | <0.14 | 0.29 | 0.96 | <0.14 | 0.17 | nd | 0.19 |
| 95% CI of | 0.64 | 0.23 | >0.55 | 0.47 | 0.17 | >0.55 | 0.50 | nd | 0.46 |
| OR Quart 4 | 24 | 9.8 | na | 12 | 5.3 | na | 54 | nd | 52 |

Cyclin-dependent kinase inhibitor 1

| | | |
|---|---|---|
| 0 hr prior to AKI stage | 24 hr prior to AKI stage | 48 hr prior to A |

| sCr or UO | Cohort 1 | Cohort 2 | Cohort 1 | Cohort 2 | Cohort 1 | Cohort 2 |
|---|---|---|---|---|---|---|
| Median | 212 | 243 | 212 | 255 | 212 | 299 |
| Average | 1210 | 333 | 1210 | 363 | 1210 | 402 |
| Stdev | 1930 | 337 | 1930 | 383 | 1930 | 405 |
| p (t-test) | | 0.19 | | 0.26 | | 0.32 |
| Min | 42.3 | 73.9 | 42.3 | 68.7 | 42.3 | 68.7 |
| Max | 6840 | 1190 | 6840 | 1190 | 6840 | 1190 |
| n (Samp) | 37 | 9 | 37 | 7 | 37 | 6 |
| n (Patient) | 37 | 9 | 37 | 7 | 37 | 6 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.46 | nd | nd | 0.45 | nd | nd | 0.47 | nd | nd |
| SE | 0.11 | nd | nd | 0.12 | nd | nd | 0.13 | nd | nd |
| p | 0.71 | nd | nd | 0.68 | nd | nd | 0.81 | nd | nd |
| nCohort 1 | 37 | nd | nd | 37 | nd | nd | 37 | nd | nd |
| nCohort 2 | 9 | nd | nd | 7 | nd | nd | 6 | nd | nd |
| Cutoff 1 | 138 | nd | nd | 138 | nd | nd | 138 | nd | nd |
| Sens 1 | 78% | nd | nd | 71% | nd | nd | 83% | nd | nd |
| Spec 1 | 38% | nd | nd | 38% | nd | nd | 38% | nd | nd |
| Cutoff 2 | 125 | nd | nd | 125 | nd | nd | 138 | nd | nd |
| Sens 2 | 89% | nd | nd | 86% | nd | nd | 83% | nd | nd |
| Spec 2 | 32% | nd | nd | 32% | nd | nd | 38% | nd | nd |
| Cutoff 3 | 73.1 | nd | nd | 61.7 | nd | nd | 61.7 | nd | nd |
| Sens 3 | 100% | nd | nd | 100% | nd | nd | 100% | nd | nd |
| Spec 3 | 14% | nd | nd | 11% | nd | nd | 11% | nd | nd |
| Cutoff 4 | 866 | nd | nd | 866 | nd | nd | 866 | nd | nd |
| Sens 4 | 11% | nd | nd | 14% | nd | nd | 17% | nd | nd |
| Spec 4 | 70% | nd | nd | 70% | nd | nd | 70% | nd | nd |
| Cutoff 5 | 1760 | nd | nd | 1760 | nd | nd | 1760 | nd | nd |
| Sens 5 | 0% | nd | nd | 0% | nd | nd | 0% | nd | nd |
| Spec 5 | 81% | nd | nd | 81% | nd | nd | 81% | nd | nd |
| Cutoff 6 | 5160 | nd | nd | 5160 | nd | nd | 5160 | nd | nd |
| Sens 6 | 0% | nd | nd | 0% | nd | nd | 0% | nd | nd |
| Spec 6 | 92% | nd | nd | 92% | nd | nd | 92% | nd | nd |
| OR Quart 2 | 6.3 | nd | nd | 3.8 | nd | nd | 3.8 | nd | nd |
| p Value | 0.13 | nd | nd | 0.29 | nd | nd | 0.29 | nd | nd |
| 95% CI of | 0.58 | nd | nd | 0.32 | nd | nd | 0.32 | nd | nd |
| OR Quart 2 | 68 | nd | nd | 43 | nd | nd | 43 | nd | nd |
| OR Quart 3 | 3.7 | nd | nd | 2.2 | nd | nd | 1.0 | nd | nd |
| p Value | 0.29 | nd | nd | 0.54 | nd | nd | 1.0 | nd | nd |
| 95% CI of | 0.32 | nd | nd | 0.17 | nd | nd | 0.055 | nd | nd |
| OR Quart 3 | 42 | nd | nd | 29 | nd | nd | 18 | nd | nd |
| OR Quart 4 | 1.1 | nd | nd | 1.0 | nd | nd | 1.1 | nd | nd |
| p Value | 0.95 | nd | nd | 1.0 | nd | nd | 0.94 | nd | nd |
| 95% CI of | 0.060 | nd | nd | 0.055 | nd | nd | 0.060 | nd | nd |
| OR Quart 4 | 20 | nd | nd | 18 | nd | nd | 20 | nd | nd |

Carcinoembryonic antigen-related cell adhesion molecule 5

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.72 | 3.47 | 1.72 | 3.47 | 1.72 | 2.26 |
| Average | 2.52 | 4.00 | 2.52 | 3.11 | 2.52 | 2.81 |
| Stdev | 2.90 | 3.87 | 2.90 | 1.92 | 2.90 | 1.94 |
| p (t-test) | | 0.065 | | 0.42 | | 0.77 |
| Min | 0.183 | 0.726 | 0.183 | 0.563 | 0.183 | 0.726 |
| Max | 20.8 | 17.1 | 20.8 | 5.94 | 20.8 | 5.94 |
| n (Samp) | 110 | 17 | 110 | 17 | 110 | 9 |
| n (Patient) | 110 | 17 | 110 | 17 | 110 | 9 |
| sCr only | | | | | | |
| Median | 1.88 | 2.35 | 1.88 | 2.35 | nd | nd |
| Average | 3.12 | 2.81 | 3.12 | 2.75 | nd | nd |
| Stdev | 4.09 | 2.17 | 4.09 | 2.23 | nd | nd |
| p (t-test) | | 0.83 | | 0.80 | nd | nd |
| Min | 0.183 | 0.726 | 0.183 | 0.563 | nd | nd |
| Max | 33.6 | 5.44 | 33.6 | 5.44 | nd | nd |
| n (Samp) | 180 | 8 | 180 | 8 | nd | nd |
| n (Patient) | 180 | 8 | 180 | 8 | nd | nd |
| UO only | | | | | | |
| Median | 1.71 | 5.01 | 1.71 | 3.72 | 1.71 | 3.47 |
| Average | 2.73 | 5.08 | 2.73 | 3.75 | 2.73 | 3.38 |
| Stdev | 3.23 | 4.31 | 3.23 | 1.66 | 3.23 | 1.82 |
| p (t-test) | | 0.030 | | 0.30 | | 0.60 |
| Min | 0.183 | 1.30 | 0.183 | 1.30 | 0.183 | 1.30 |
| Max | 20.8 | 17.1 | 20.8 | 5.94 | 20.8 | 5.94 |
| n (Samp) | 89 | 11 | 89 | 11 | 89 | 7 |
| n (Patient) | 89 | 11 | 89 | 11 | 89 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.66 | 0.50 | 0.78 | 0.63 | 0.48 | 0.75 | 0.60 | nd | 0.70 |
| SE | 0.076 | 0.10 | 0.086 | 0.077 | 0.11 | 0.088 | 0.10 | nd | 0.11 |
| p | 0.033 | 1.00 | 0.0012 | 0.086 | 0.83 | 0.0047 | 0.34 | nd | 0.085 |
| nCohort 1 | 110 | 180 | 89 | 110 | 180 | 89 | 110 | nd | 89 |
| nCohort 2 | 17 | 8 | 11 | 17 | 8 | 11 | 9 | nd | 7 |
| Cutoff 1 | 1.44 | 0.826 | 2.71 | 1.44 | 0.710 | 2.52 | 1.28 | nd | 2.23 |
| Sens 1 | 71% | 75% | 73% | 71% | 75% | 73% | 78% | nd | 71% |
| Spec 1 | 45% | 16% | 73% | 45% | 12% | 72% | 40% | nd | 64% |
| Cutoff 2 | 0.886 | 0.825 | 2.52 | 0.886 | 0.649 | 2.23 | 0.886 | nd | 1.49 |
| Sens 2 | 82% | 88% | 82% | 82% | 88% | 82% | 89% | nd | 86% |
| Spec 2 | 25% | 16% | 72% | 25% | 8% | 64% | 25% | nd | 46% |
| Cutoff 3 | 0.825 | 0.710 | 1.49 | 0.649 | 0.504 | 1.49 | 0.710 | nd | 1.28 |
| Sens 3 | 94% | 100% | 91% | 94% | 100% | 91% | 100% | nd | 100% |
| Spec 3 | 23% | 12% | 46% | 14% | 6% | 46% | 17% | nd | 40% |
| Cutoff 4 | 2.42 | 2.71 | 2.52 | 2.42 | 2.71 | 2.52 | 2.42 | nd | 2.52 |
| Sens 4 | 65% | 50% | 82% | 59% | 50% | 73% | 44% | nd | 57% |
| Spec 4 | 70% | 70% | 72% | 70% | 70% | 72% | 70% | nd | 72% |
| Cutoff 5 | 3.25 | 3.88 | 3.74 | 3.25 | 3.88 | 3.74 | 3.25 | nd | 3.74 |
| Sens 5 | 53% | 38% | 55% | 53% | 38% | 45% | 44% | nd | 29% |
| Spec 5 | 80% | 80% | 81% | 80% | 80% | 81% | 80% | nd | 81% |
| Cutoff 6 | 5.56 | 6.28 | 6.77 | 5.56 | 6.28 | 6.77 | 5.56 | nd | 6.77 |
| Sens 6 | 12% | 0% | 9% | 6% | 0% | 0% | 11% | nd | 0% |
| Spec 6 | 90% | 90% | 91% | 90% | 90% | 91% | 90% | nd | 91% |
| OR Quart 2 | 0.97 | 0 | >2.2 | 0.97 | 0 | >2.2 | 3.1 | nd | >2.2 |
| p Value | 0.97 | na | <0.54 | 0.97 | na | <0.54 | 0.34 | nd | <0.54 |
| 95% CI of | 0.18 | na | >0.18 | 0.18 | na | >0.18 | 0.30 | nd | >0.18 |
| OR Quart 2 | 5.2 | na | na | 5.2 | na | na | 32 | nd | na |
| OR Quart 3 | 0.62 | 0 | >2.2 | 0.62 | 0 | >2.2 | 0.97 | nd | >1.0 |
| p Value | 0.62 | na | <0.54 | 0.62 | na | <0.54 | 0.98 | nd | <0.98 |
| 95% CI of | 0.097 | na | >0.18 | 0.097 | na | >0.18 | 0.058 | nd | >0.062 |
| OR Quart 3 | 4.0 | na | na | 4.0 | na | na | 16 | nd | na |
| OR Quart 4 | 3.7 | 1.0 | >9.7 | 3.7 | 1.0 | >9.7 | 4.3 | nd | >4.8 |
| p Value | 0.073 | 1.0 | <0.041 | 0.073 | 1.0 | <0.041 | 0.20 | nd | <0.18 |
| 95% CI of | 0.88 | 0.23 | >1.1 | 0.88 | 0.23 | >1.1 | 0.45 | nd | >0.50 |
| OR Quart 4 | 15 | 4.3 | na | 15 | 4.3 | na | 41 | nd | na |

Myoglobin

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 48.8 | 222 | 48.8 | 145 | 48.8 | 122 |
| Average | 109 | 574 | 109 | 498 | 109 | 407 |
| Stdev | 211 | 653 | 211 | 658 | 211 | 480 |
| p (t-test) | | 4.0E−8 | | 3.3E−6 | | 4.7E−4 |
| Min | 5.55 | 16.2 | 5.55 | 16.2 | 5.55 | 16.2 |
| Max | 1720 | 1880 | 1720 | 1880 | 1720 | 1180 |
| n (Samp) | 110 | 17 | 110 | 17 | 110 | 9 |
| n (Patient) | 110 | 17 | 110 | 17 | 110 | 9 |
| sCr only | | | | | | |
| Median | 56.7 | 475 | 56.7 | 475 | nd | nd |
| Average | 143 | 694 | 143 | 683 | nd | nd |
| Stdev | 283 | 718 | 283 | 729 | nd | nd |
| p (t-test) | | 2.1E−6 | | 3.5E−6 | nd | nd |
| Min | 5.55 | 16.2 | 5.55 | 16.2 | nd | nd |
| Max | 2130 | 1880 | 2130 | 1880 | nd | nd |
| n (Samp) | 180 | 8 | 180 | 8 | nd | nd |
| n (Patient) | 180 | 8 | 180 | 8 | nd | nd |
| UO only | | | | | | |
| Median | 46.2 | 222 | 46.2 | 145 | 46.2 | 122 |
| Average | 120 | 547 | 120 | 438 | 120 | 352 |
| Stdev | 223 | 581 | 223 | 574 | 223 | 423 |
| p (t-test) | | 6.9E−6 | | 5.8E−4 | | 0.016 |
| Min | 5.55 | 86.7 | 5.55 | 69.7 | 5.55 | 69.7 |
| Max | 1720 | 1660 | 1720 | 1660 | 1720 | 1160 |
| n (Samp) | 89 | 11 | 89 | 11 | 89 | 7 |
| n (Patient) | 89 | 11 | 89 | 11 | 89 | 7 |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.82 | 0.75 | 0.86 | 0.76 | 0.68 | 0.82 | 0.76 | nd | 0.79 |
| SE | 0.064 | 0.10 | 0.072 | 0.071 | 0.11 | 0.080 | 0.095 | nd | 0.10 |
| p | 5.2E−7 | 0.016 | 3.9E−7 | 3.1E−4 | 0.096 | 8.3E−5 | 0.0061 | nd | 0.0048 |
| nCohort 1 | 110 | 180 | 89 | 110 | 180 | 89 | 110 | nd | 89 |
| nCohort 2 | 17 | 8 | 11 | 17 | 8 | 11 | 9 | nd | 7 |
| Cutoff 1 | 124 | 101 | 138 | 101 | 30.6 | 105 | 79.5 | nd | 87.6 |
| Sens 1 | 71% | 75% | 73% | 71% | 75% | 73% | 78% | nd | 71% |
| Spec 1 | 82% | 69% | 80% | 77% | 32% | 75% | 71% | nd | 69% |
| Cutoff 2 | 101 | 34.3 | 124 | 66.9 | 19.2 | 101 | 66.9 | nd | 79.5 |
| Sens 2 | 82% | 88% | 82% | 82% | 88% | 82% | 89% | nd | 86% |
| Spec 2 | 77% | 35% | 78% | 62% | 19% | 74% | 62% | nd | 67% |
| Cutoff 3 | 33.6 | 15.7 | 108 | 19.2 | 15.7 | 87.6 | 15.0 | nd | 66.9 |
| Sens 3 | 94% | 100% | 91% | 94% | 100% | 91% | 100% | nd | 100% |
| Spec 3 | 38% | 12% | 76% | 21% | 12% | 69% | 14% | nd | 61% |
| Cutoff 4 | 78.9 | 105 | 97.8 | 78.9 | 105 | 97.8 | 78.9 | nd | 97.8 |
| Sens 4 | 88% | 62% | 91% | 76% | 62% | 82% | 78% | nd | 57% |
| Spec 4 | 70% | 70% | 71% | 70% | 70% | 71% | 70% | nd | 71% |
| Cutoff 5 | 112 | 150 | 146 | 112 | 150 | 146 | 112 | nd | 146 |
| Sens 5 | 71% | 62% | 64% | 59% | 62% | 45% | 56% | nd | 43% |
| Spec 5 | 80% | 80% | 81% | 80% | 80% | 81% | 80% | nd | 81% |
| Cutoff 6 | 212 | 315 | 323 | 212 | 315 | 323 | 212 | nd | 323 |
| Sens 6 | 53% | 50% | 36% | 41% | 50% | 27% | 44% | nd | 29% |
| Spec 6 | 90% | 90% | 91% | 90% | 90% | 91% | 90% | nd | 91% |
| OR Quart 2 | 0.97 | 1.0 | >0 | 0.47 | 0.49 | >0 | 0 | nd | >0 |
| p Value | 0.98 | 1.0 | <na | 0.54 | 0.56 | <na | na | nd | <na |
| 95% CI of | 0.058 | 0.061 | >na | 0.040 | 0.043 | >na | na | nd | >na |
| OR Quart 2 | 16 | 16 | na | 5.4 | 5.6 | na | na | nd | na |
| OR Quart 3 | 3.1 | 1.0 | >4.8 | 2.1 | 0 | >6.2 | 3.1 | nd | >4.8 |
| p Value | 0.34 | 1.0 | <0.18 | 0.42 | na | <0.11 | 0.34 | nd | <0.18 |
| 95% CI of | 0.30 | 0.061 | >0.49 | 0.35 | na | >0.67 | 0.30 | nd | >0.50 |
| OR Quart 3 | 32 | 16 | na | 12 | na | na | 32 | nd | na |
| OR Quart 4 | 18 | 5.5 | >9.7 | 6.6 | 2.7 | >7.9 | 5.6 | nd | >3.4 |
| p Value | 0.0075 | 0.13 | <0.041 | 0.022 | 0.25 | <0.066 | 0.13 | nd | <0.30 |
| 95% CI of | 2.2 | 0.61 | >1.1 | 1.3 | 0.49 | >0.88 | 0.61 | nd | >0.33 |
| OR Quart 4 | 150 | 49 | na | 33 | 15 | na | 51 | nd | na |

Mucin-16

| | | |
|---|---|---|
| 0 hr prior to AKI stage | 24 hr prior to AKI stage | 48 hr prior to AKI stage |

| | Cohort 1 | Cohort 2 | Cohort 1 | Cohort 2 | Cohort 1 | Cohort 2 |
|---|---|---|---|---|---|---|
| sCr or UO | | | | | | |
| Median | 1.17 | 8.40 | 1.17 | 7.52 | nd | nd |
| Average | 3.77 | 31.2 | 3.77 | 30.6 | nd | nd |
| Stdev | 15.1 | 67.4 | 15.1 | 67.6 | nd | nd |
| p (t-test) | | 0.0035 | | 0.0043 | nd | nd |
| Min | 0.117 | 2.02 | 0.117 | 2.02 | nd | nd |
| Max | 131 | 198 | 131 | 198 | nd | nd |
| n (Samp) | 75 | 8 | 75 | 8 | nd | nd |
| n (Patient) | 75 | 8 | 75 | 8 | nd | nd |
| UO only | | | | | | |
| Median | 1.36 | 7.47 | 1.36 | 7.47 | nd | nd |
| Average | 4.38 | 38.3 | 4.38 | 38.3 | nd | nd |
| Stdev | 16.6 | 78.2 | 16.6 | 78.2 | nd | nd |
| p (t-test) | | 0.0042 | | 0.0042 | nd | nd |
| Min | 0.122 | 2.02 | 0.122 | 2.02 | nd | nd |
| Max | 131 | 198 | 131 | 198 | nd | nd |
| n (Samp) | 62 | 6 | 62 | 6 | nd | nd |
| n (Patient) | 62 | 6 | 62 | 6 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.89 | nd | 0.85 | 0.88 | nd | 0.85 | nd | nd | nd |
| SE | 0.079 | nd | 0.100 | 0.080 | nd | 0.100 | nd | nd | nd |
| p | 9.8E−7 | nd | 3.7E−4 | 1.6E−6 | nd | 3.7E−4 | nd | nd | nd |
| nCohort 1 | 75 | nd | 62 | 75 | nd | 62 | nd | nd | nd |
| nCohort 2 | 8 | nd | 6 | 8 | nd | 6 | nd | nd | nd |
| Cutoff 1 | 4.24 | nd | 2.09 | 4.24 | nd | 2.09 | nd | nd | nd |
| Sens 1 | 75% | nd | 83% | 75% | nd | 83% | nd | nd | nd |
| Spec 1 | 88% | nd | 68% | 88% | nd | 68% | nd | nd | nd |
| Cutoff 2 | 2.09 | nd | 2.09 | 2.09 | nd | 2.09 | nd | nd | nd |
| Sens 2 | 88% | nd | 83% | 88% | nd | 83% | nd | nd | nd |
| Spec 2 | 69% | nd | 68% | 69% | nd | 68% | nd | nd | nd |
| Cutoff 3 | 1.96 | nd | 1.96 | 1.96 | nd | 1.96 | nd | nd | nd |
| Sens 3 | 100% | nd | 100% | 100% | nd | 100% | nd | nd | nd |
| Spec 3 | 67% | nd | 66% | 67% | nd | 66% | nd | nd | nd |
| Cutoff 4 | 2.20 | nd | 2.56 | 2.20 | nd | 2.56 | nd | nd | nd |
| Sens 4 | 75% | nd | 67% | 75% | nd | 67% | nd | nd | nd |
| Spec 4 | 72% | nd | 71% | 72% | nd | 71% | nd | nd | nd |
| Cutoff 5 | 2.98 | nd | 3.41 | 2.98 | nd | 3.41 | nd | nd | nd |
| Sens 5 | 75% | nd | 67% | 75% | nd | 67% | nd | nd | nd |
| Spec 5 | 80% | nd | 81% | 80% | nd | 81% | nd | nd | nd |
| Cutoff 6 | 5.21 | nd | 6.93 | 5.21 | nd | 6.93 | nd | nd | nd |
| Sens 6 | 62% | nd | 50% | 62% | nd | 50% | nd | nd | nd |
| Spec 6 | 91% | nd | 90% | 91% | nd | 90% | nd | nd | nd |
| OR Quart 2 | >0 | nd | >0 | >0 | nd | >0 | nd | nd | nd |
| p Value | <na | nd | <na | <na | nd | <na | nd | nd | nd |
| 95% CI of | >na | nd | >na | >na | nd | >na | nd | nd | nd |
| OR Quart 2 | na | nd | na | na | nd | na | nd | nd | nd |
| OR Quart 3 | >2.1 | nd | >2.3 | >2.1 | nd | >2.3 | nd | nd | nd |
| p Value | <0.56 | nd | <0.52 | <0.56 | nd | <0.52 | nd | nd | nd |
| 95% CI of | >0.18 | nd | >0.19 | >0.18 | nd | >0.19 | nd | nd | nd |
| OR Quart 3 | na | nd | na | na | nd | na | nd | nd | nd |
| OR Quart 4 | >8.0 | nd | >5.2 | >8.0 | nd | >5.2 | nd | nd | nd |
| p Value | <0.066 | nd | <0.16 | <0.066 | nd | <0.16 | nd | nd | nd |
| 95% CI of | >0.87 | nd | >0.52 | >0.87 | nd | >0.52 | nd | nd | nd |
| OR Quart 4 | na | nd | na | na | nd | na | nd | nd | nd |

Tumor necrosis factor receptor superfamily member 10B

| | | |
|---|---|---|
| 0 hr prior to AKI stage | 24 hr prior to AKI stage | 48 hr prior to AKI stage |

| sCr or UO | Cohort 1 | Cohort 2 | Cohort 1 | Cohort 2 | Cohort 1 | Cohort 2 |
|---|---|---|---|---|---|---|
| Median | 1.00E−9 | 0.0138 | 1.00E−9 | 0.0129 | nd | nd |
| Average | 0.00697 | 0.0265 | 0.00697 | 0.0259 | nd | nd |
| Stdev | 0.0202 | 0.0355 | 0.0202 | 0.0359 | nd | nd |
| p (t-test) | | 0.020 | | 0.024 | nd | nd |
| Min | 1.00E−9 | 1.00E−9 | 1.00E−9 | 1.00E−9 | nd | nd |
| Max | 0.114 | 0.108 | 0.114 | 0.108 | nd | nd |
| n (Samp) | 72 | 8 | 72 | 8 | nd | nd |
| n (Patient) | 72 | 8 | 72 | 8 | nd | nd |

| | 0 hr prior to AKI stage | | | 24 hr prior to AKI stage | | | 48 hr prior to AKI stage | | |
|---|---|---|---|---|---|---|---|---|---|
| | sCr or UO | sCr only | UO only | sCr or UO | sCr only | UO only | sCr or UO | sCr only | UO only |
| AUC | 0.81 | nd | nd | 0.79 | nd | nd | nd | nd | nd |
| SE | 0.095 | nd | nd | 0.098 | nd | nd | nd | nd | nd |
| p | 0.0012 | nd | nd | 0.0031 | nd | nd | nd | nd | nd |
| nCohort 1 | 72 | nd | nd | 72 | nd | nd | nd | nd | nd |
| nCohort 2 | 8 | nd | nd | 8 | nd | nd | nd | nd | nd |
| Cutoff 1 | 0.00462 | nd | nd | 0.00438 | nd | nd | nd | nd | nd |
| Sens 1 | 75% | nd | nd | 75% | nd | nd | nd | nd | nd |
| Spec 1 | 79% | nd | nd | 79% | nd | nd | nd | nd | nd |
| Cutoff 2 | 0.00438 | nd | nd | 0.00207 | nd | nd | nd | nd | nd |
| Sens 2 | 88% | nd | nd | 88% | nd | nd | nd | nd | nd |
| Spec 2 | 79% | nd | nd | 65% | nd | nd | nd | nd | nd |
| Cutoff 3 | 0 | nd | nd | 0 | nd | nd | nd | nd | nd |
| Sens 3 | 100% | nd | nd | 100% | nd | nd | nd | nd | nd |
| Spec 3 | 0% | nd | nd | 0% | nd | nd | nd | nd | nd |
| Cutoff 4 | 0.00274 | nd | nd | 0.00274 | nd | nd | nd | nd | nd |
| Sens 4 | 88% | nd | nd | 75% | nd | nd | nd | nd | nd |
| Spec 4 | 71% | nd | nd | 71% | nd | nd | nd | nd | nd |
| Cutoff 5 | 0.00507 | nd | nd | 0.00507 | nd | nd | nd | nd | nd |
| Sens 5 | 62% | nd | nd | 50% | nd | nd | nd | nd | nd |
| Spec 5 | 81% | nd | nd | 81% | nd | nd | nd | nd | nd |
| Cutoff 6 | 0.0124 | nd | nd | 0.0124 | nd | nd | nd | nd | nd |
| Sens 6 | 50% | nd | nd | 50% | nd | nd | nd | nd | nd |
| Spec 6 | 90% | nd | nd | 90% | nd | nd | nd | nd | nd |
| OR Quart 2 | >1.1 | nd | nd | >1.1 | nd | nd | nd | nd | nd |
| p Value | <0.97 | nd | nd | <0.97 | nd | nd | nd | nd | nd |
| 95% CI of | >0.061 | nd | nd | >0.061 | nd | nd | nd | nd | nd |
| OR Quart 2 | na | nd | nd | na | nd | nd | nd | nd | nd |
| OR Quart 3 | >2.2 | nd | nd | >2.2 | nd | nd | nd | nd | nd |
| p Value | <0.53 | nd | nd | <0.53 | nd | nd | nd | nd | nd |
| 95% CI of | >0.19 | nd | nd | >0.19 | nd | nd | nd | nd | nd |
| OR Quart 3 | na | nd | nd | na | nd | nd | nd | nd | nd |
| OR Quart 4 | >6.7 | nd | nd | >6.7 | nd | nd | nd | nd | nd |
| p Value | <0.098 | nd | nd | <0.098 | nd | nd | nd | nd | nd |
| 95% CI of | >0.70 | nd | nd | >0.70 | nd | nd | nd | nd | nd |
| OR Quart 4 | na | nd | nd | na | nd | nd | nd | nd | nd |

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for evaluating renal status in a subject, comprising:
obtaining a urine sample from a subject selected for evaluation based on a determination that the subject is at risk of a future or current acute renal injury;
performing an assay configured to detect Apolipoprotein A-II by introducing the urine sample obtained from the subject into an assay instrument which (i) contacts all or a portion of the urine sample with a binding reagent which specifically binds for detection Apolipoprotein A-II, and (ii) generates an assay result indicative of binding of Apolipoprotein A-II to the binding reagent to provide an assay result;
correlating the assay result to the renal status of the subject by using the assay result to assign the patient to a predetermined subpopulation of individuals having a known predisposition of a future acute renal injury occurring within 72 hours of the time at which the body fluid sample is obtained; and
treating the patient based on the predetermined subpopulation of individuals to which the patient is assigned, wherein the treatment comprises one or more of initiating renal replacement therapy, withdrawing delivery of compounds that are known to be damaging to the kidney, delaying or avoiding procedures that are known to be damaging to the kidney, and modifying diuretic administration.

2. A method according to claim 1, wherein said risk of a future acute renal injury is a risk of future acute renal failure (ARF).

3. A method according to claim 1, wherein the subject is not in acute renal failure.

4. A method according to claim 1, wherein the subject has not experienced a 1.5-fold or greater increase in serum creatinine over a baseline value determined prior to the time at which the body fluid sample is obtained.

5. A method according to claim 1, wherein the subject has a urine output of at least 0.5 ml/kg/hr over the 12 hours preceding the time at which the body fluid sample is obtained.

6. A method according to claim 1, wherein the subject has not experienced an increase of 0.3 mg/dL or greater in serum creatinine over a baseline value determined prior to the time at which the body fluid sample is obtained.

7. A method according to claim 1, wherein the subject (i) has not experienced a 1.5-fold or greater increase in serum creatinine over a baseline value determined prior to the time at which the body fluid sample is obtained, (ii) has a urine output of at least 0.5 ml/kg/hr over the 12 hours preceding the time at which the body fluid sample is obtained, and (iii) has not experienced an increase of 0.3 mg/dL or greater in serum creatinine over a baseline value determined prior to the time at which the body fluid sample is obtained.

8. A method according to claim 1, wherein the subject is in RIFLE stage 0 or R.

9. A method according to claim 8, wherein the subject is in RIFLE stage 0, and said correlating step comprises assigning a likelihood that the subject will reach RIFLE stage F within 72 hours.

10. A method according to claim 8, wherein the subject is in RIFLE stage R, and said correlating step comprises assigning a likelihood that the subject will reach RIFLE stage I or F within 72 hours.

11. A method according to claim 8, wherein said correlating step comprises assigning a likelihood that the subject will reach RIFLE stage I or F within 48 hours.

12. A method according to claim 8, wherein said correlating step comprises assigning a likelihood that the subject will reach RIFLE stage I or F within 24 hours.

13. A method according to claim 1, wherein said assay result comprises a measured urine or plasma concentration of Apolipoprotein A-II.

14. A method according to claim 1, wherein the subject is selected for evaluation of renal status based on the pre-existence in the subject of one or more known risk factors for prerenal, intrinsic renal, or postrenal ARF.

15. A method according to claim 1, wherein the subject is selected for evaluation of renal status based on an existing diagnosis of one or more of congestive heart failure, preeclampsia, eclampsia, diabetes mellitus, hypertension, coronary artery disease, proteinuria, renal insufficiency, glomerular filtration below the normal range, cirrhosis, serum creatinine above the normal range, sepsis, injury to renal function, reduced renal function, or ARF, or based on undergoing or having undergone major vascular surgery, coronary artery bypass, or other cardiac surgery, or based on exposure to NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin.
